SEQUENCE LISTING (231 sequences)

SEQ ID NO: 1 - protein, 12 aa, synthetic construct
RASQSVRSSYLA

SEQ ID NO: 2 - protein, 7 aa, synthetic construct
GASSRAT

SEQ ID NO: 3 - protein, 9 aa, synthetic construct
QQYVSYPRT

SEQ ID NO: 4 - protein, 23 aa, synthetic construct
EIVLTQSPGTLSLSPGERATLSC

SEQ ID NO: 5 - protein, 15 aa, synthetic construct
WYQQKPGQAPRLLIY

SEQ ID NO: 6 - protein, 32 aa, synthetic construct
GIPDRFSGSGSGTDFTLTISRLEPEDFAVYYC

SEQ ID NO: 7 - protein, 10 aa, synthetic construct
FGGGTKVEIK

SEQ ID NO: 8 - protein, 108 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYVSYPRTFGGGTKVEIK

SEQ ID NO: 9 - protein, 215 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYVSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttaggagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtacgtcagttaccctaggacttttggcggagggaccaaggttgagatcaaa

SEQ ID NO: 11 - protein, 12 aa, synthetic construct
RASQSVSSSYLA

SEQ ID NO: 12 - protein, 9 aa, synthetic construct
QQYLSYPRT

SEQ ID NO: 13 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYLSYPRTFGGGTKVEIK

SEQ ID NO: 14 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYLSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtacctgagttaccctaggacttttggcggagggaccaaggttgagatcaaa

SEQ ID NO: 16 - protein, 7 aa, synthetic construct
GASSRAG

SEQ ID NO: 17 - protein, 9 aa, synthetic construct
QQYVAYPRT

SEQ ID NO: 18 - protein, 108 aa, synthetic construct
EIVLTQSPGTLSLSPGERATLSCRASQSVSSSYLAWYQQKPGQAPRLLIYGASSRAGGIPDRFSGSGSGTDFTLTISRLEPEDFAVYYCQQYVAYPRTFGGGTKVEIK

SEQ ID NO: 19 - protein, 215 aa, synthetic construct
EIVLTQSPGTLSLSPGERATLSCRASQSVSSSYLAWYQQKPGQAPRLLIYGASSRAGGIPDRFSGSGSGTDFTLTISRLEPEDFAVYYCQQYVAYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccggaggcatcccagacaggttcagtggcagtgggtctgggacagacttcactctcaccatcagcagactggagcctgaagattttgcagtgtattactgtcagcagtacgtcgcgtaccctaggacttttggcggagggaccaaggttgagatcaaa

SEQ ID NO: 21 - protein, 9 aa, synthetic construct
LQYGSYPRT

SEQ ID NO: 22 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCLQYGSYPRTFGGGTKVEIK

SEQ ID NO: 23 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCLQYGSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtttgcagtacgggagttaccctaggacttttggcggagggaccaaggttgagatcaaa

SEQ ID NO: 25-27 - skipped (3 entries)

SEQ ID NO: 28 - protein, 11 aa, synthetic construct
RASQSVSSNLA

SEQ ID NO: 29 - protein, 7 aa, synthetic construct
GASDRAT

SEQ ID NO: 30 - protein, 9 aa, synthetic construct
IQYSNYPRT

SEQ ID NO: 31 - protein, 23 aa, synthetic construct
EIVLTQSPATLSVSPGERATLSC

SEQ ID NO: 32 - protein, 32 aa, synthetic construct
GIPARFSGSGSGTEFTLTISSLQSEDFAVYYC

SEQ ID NO: 33 - protein, 107 aa, synthetic construct
EIVLTQSPATLSVSPGERATLSCRASQSVSSNLAWYQQKPGQAPRLLIYGASDRATGIPARFSGSGSGTEFTLTISSLQSEDFAVYYCIQYSNYPRTFGGGTKVEIK

SEQ ID NO: 34 - protein, 214 aa, synthetic construct
EIVLTQSPATLSVSPGERATLSCRASQSVSSNLAWYQQKPGQAPRLLIYGASDRATGIPARFSGSGSGTEFTLTISSLQSEDFAVYYCIQYSNYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 321 bp, synthetic construct
  source 1..321: mol_type = other DNA
gaaatagtgttgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccgacagggccactggtatcccagccaggttcagtggcagtgggtctgggacagagttcactctcaccatcagcagcctgcagtctgaagattttgcagtttattactgtattcagtactccaactaccctaggacttttggcggagggaccaaggttgagatcaaa

SEQ ID NO: 36 - protein, 9 aa, synthetic construct
QQYSNYPRT

SEQ ID NO: 37 - protein, 108 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYSNYPRTFGGGTKVEIK

SEQ ID NO: 38 - protein, 215 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYSNYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttaggagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtactccaactaccctaggacttttggcggagggaccaaggttgagatcaaa

SEQ ID NO: 40-42 - skipped (3 entries)

SEQ ID NO: 43 - protein, 7 aa, synthetic construct
GASYRAT

SEQ ID NO: 44 - protein, 9 aa, synthetic construct
LQSGVFPYT

SEQ ID NO: 45 - protein, 108 aa, synthetic construct
EIVLTQSPGTLSLSPGERATLSCRASQSVSSSYLAWYQQKPGQAPRLLIYGASYRATGIPDRFSGSGSGTDFTLTISRLEPEDFAVYYCLQSGVFPYTFGGGTKVEIK

SEQ ID NO: 46 - protein, 215 aa, synthetic construct
EIVLTQSPGTLSLSPGERATLSCRASQSVSSSYLAWYQQKPGQAPRLLIYGASYRATGIPDRFSGSGSGTDFTLTISRLEPEDFAVYYCLQSGVFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatcctacagggccactggcatcccagacaggttcagtggcagtgggtctgggacagacttcactctcaccatcagcagactggagcctgaagattttgcagtgtattactgtttgcagtccggagtcttcccttacacttttggcggagggaccaaggttgagatcaaa

SEQ ID NO: 48 - protein, 7 aa, synthetic construct
GAFSRAT

SEQ ID NO: 49 - protein, 9 aa, synthetic construct
QQSGVFPYT

SEQ ID NO: 50 - protein, 108 aa, synthetic construct
EIVLTQSPGTLSLSPGERATLSCRASQSVSSSYLAWYQQKPGQAPRLLIYGAFSRATGIPDRFSGSGSGTDFTLTISRLEPEDFAVYYCQQSGVFPYTFGGGTKVEIK

SEQ ID NO: 51 - protein, 215 aa, synthetic construct
EIVLTQSPGTLSLSPGERATLSCRASQSVSSSYLAWYQQKPGQAPRLLIYGAFSRATGIPDRFSGSGSGTDFTLTISRLEPEDFAVYYCQQSGVFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtccggagtcttcccttacacttttggcggagggaccaaggttgagatcaaa

SEQ ID NO: 53 - protein, 11 aa, synthetic construct
RASQSVSSYLA

SEQ ID NO: 54 - protein, 7 aa, synthetic construct
DASNRAT

SEQ ID NO: 55 - protein, 10 aa, synthetic construct
QQDSIFPPYT

SEQ ID NO: 56 - protein, 23 aa, synthetic construct
EIVLTQSPATLSLSPGERATLSC

SEQ ID NO: 57 - protein, 32 aa, synthetic construct
GIPARFSGSGSGTDFTLTISSLEPEDFAVYYC

SEQ ID NO: 58 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQDSIFPPYTFGGGTKVEIK

SEQ ID NO: 59 - protein, 215 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQDSIFPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 324 bp, synthetic construct
  source 1..324: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaggacagtatcttcccgccttacacttttggcggagggaccaaggttgagatcaaa

SEQ ID NO: 61 - protein, 7 aa, synthetic construct
GASTRAT

SEQ ID NO: 62 - protein, 9 aa, synthetic construct
QQSDDWPWT

SEQ ID NO: 63 - protein, 23 aa, synthetic construct
EIVLTQSPATLSVSPGERATLSC

SEQ ID NO: 64 - skipped

SEQ ID NO: 65 - protein, 107 aa, synthetic construct
EIVLTQSPATLSVSPGERATLSCRASQSVSSNLAWYQQKPGQAPRLLIYGASTRATGIPARFSGSGSGTEFTLTISSLQSEDFAVYYCQQSDDWPWTFGGGTKVEIK

SEQ ID NO: 66 - protein, 214 aa, synthetic construct
EIVLTQSPATLSVSPGERATLSCRASQSVSSNLAWYQQKPGQAPRLLIYGASTRATGIPARFSGSGSGTEFTLTISSLQSEDFAVYYCQQSDDWPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 321 bp, synthetic construct
  source 1..321: mol_type = other DNA
gaaatagtgttgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtattccagccaggttcagtggcagtgggtctgggacagagttcactctcaccatcagcagcctgcagtctgaagattttgcagtttattactgtcagcagtccgacgactggccttggacttttggcggagggaccaaggttgagatcaaa

SEQ ID NO: 68 - protein, 11 aa, synthetic construct
QASQDISNYLN

SEQ ID NO: 69 - protein, 7 aa, synthetic construct
DASNLET

SEQ ID NO: 70 - protein, 9 aa, synthetic construct
QQSDHLPFT

SEQ ID NO: 71 - protein, 23 aa, synthetic construct
DIQMTQSPSSLSASVGDRVTITC

SEQ ID NO: 72 - protein, 15 aa, synthetic construct
WYQQKPGKAPKLLIY

SEQ ID NO: 73 - protein, 32 aa, synthetic construct
GVPSRFSGSGSGTDFTFTISSLQPEDIATYYC

SEQ ID NO: 74 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQSDHLPFTFGGGTKVEIK

SEQ ID NO: 75 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQSDHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcagcagtccgaccacctccctttcacttttggcggagggaccaaggttgagatcaaa

SEQ ID NO: 77-79 - skipped (3 entries)

SEQ ID NO: 80 - protein, 9 aa, synthetic construct
QQYNNWPIT

SEQ ID NO: 81 - protein, 23 aa, synthetic construct
EIVMTQSPATLSVSPGERATLSC

SEQ ID NO: 82 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPITFGGGTKVEIK

SEQ ID NO: 83 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctatcacctttggcggagggaccaaggttgagatcaaa

SEQ ID NO: 85 - protein, 11 aa, synthetic construct
RASQGIDSWLA

SEQ ID NO: 86 - protein, 7 aa, synthetic construct
AASSLQS

SEQ ID NO: 87 - protein, 9 aa, synthetic construct
QQVHSFPIT

SEQ ID NO: 88 - protein, 23 aa, synthetic construct
DIQMTQSPSSVSASVGDRVTITC

SEQ ID NO: 89 - protein, 32 aa, synthetic construct
GVPSRFSGSGSGTDFTLTISSLQPEDFATYYC

SEQ ID NO: 90 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQVHSFPITFGGGTKVEIK

SEQ ID NO: 91 - protein, 214 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQVHSFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattgacagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaggtacacagtttccctatcacttttggcggagggaccaaggttgagatcaaa

SEQ ID NO: 93 - protein, 16 aa, synthetic construct
RSSQSLLHSNGYNYLD

SEQ ID NO: 94 - protein, 7 aa, synthetic construct
LGSNRAS

SEQ ID NO: 95 - protein, 9 aa, synthetic construct
MQALQTPIT

SEQ ID NO: 96 - protein, 23 aa, synthetic construct
DIVMTQSPLSLPVTPGEPASISC

SEQ ID NO: 97 - protein, 15 aa, synthetic construct
WYLQKPGQSPQLLIY

SEQ ID NO: 98 - protein, 32 aa, synthetic construct
GVPDRFSGSGSGTDFTLKISRVEAEDVGVYYC

SEQ ID NO: 99 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGGGTKVEIK

SEQ ID NO: 100 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctatcacctttggcggagggaccaaggttgagatcaaa

SEQ ID NO: 102 - protein, 107 aa, synthetic construct [sequence omitted]

SEQ ID NO: 103 - protein, 9 aa, synthetic construct
YTFTGYYMH

SEQ ID NO: 104 - protein, 17 aa, synthetic construct
SINPSSGGTNYAQKFQG

SEQ ID NO: 105 - protein, 16 aa, synthetic construct
ARARDIAAAGLGAFDY

SEQ ID NO: 106 - protein, 26 aa, synthetic construct
QVQLVQSGAEVKKPGASVKVSCKASG

SEQ ID NO: 107 - protein, 14 aa, synthetic construct
WVRQAPGQGLEWMG

SEQ ID NO: 108 - protein, 30 aa, synthetic construct
RVTMTTDTSTSTAYMELRSLRSDDTAVYYC

SEQ ID NO: 109 - protein, 11 aa, synthetic construct
WGQGTLVTVSS

SEQ ID NO: 110 - protein, 123 aa, synthetic construct
QVQLVQSGAEVKKPGASVKVSCKASGYTFTGYYMHWVRQAPGQGLEWMGSINPSSGGTNYAQKFQGRVTMTTDTSTSTAYMELRSLRSDDTAVYYCARARDIAAAGLGAFDYWGQGTLVTVSS

SEQ ID NO: 111 - protein, 453 aa, synthetic construct
QVQLVQSGAEVKKPGASVKVSCKASGYTFTGYYMHWVRQAPGQGLEWMGSINPSSGGTNYAQKFQGRVTMTTDTSTSTAYMELRSLRSDDTAVYYCARARDIAAAGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggaagcatcaaccctagcagtggtggcacaaactatgcacagaagtttcagggcagggtcaccatgaccacagacacatccacgagcacagcctacatggagctgaggagcctgagatctgacgacacggcggtgtactactgcgcaagggctagagacatagcagcagcaggcttgggagcttttgactactggggacagggtacattggtcaccgtctcctca

SEQ ID NO: 113 - protein, 9 aa, synthetic construct
YTFIGYDMH

SEQ ID NO: 114 - protein, 17 aa, synthetic construct
VINPSSGGTNYAQKFQG

SEQ ID NO: 115 - protein, 16 aa, synthetic construct
ARARDIAALGLGAFDY

SEQ ID NO: 116 - protein, 30 aa, synthetic construct
RVTMTRDTSISTAYMELSRLRSDDTAVYYC

SEQ ID NO: 117 - protein, 123 aa, synthetic construct
QVQLVQSGAEVKKPGASVKVSCKASGYTFIGYDMHWVRQAPGQGLEWMGVINPSSGGTNYAQKFQGRVTMTRDTSISTAYMELSRLRSDDTAVYYCARARDIAALGLGAFDYWGQGTLVTVSS

SEQ ID NO: 118 - protein, 453 aa, synthetic construct
QVQLVQSGAEVKKPGASVKVSCKASGYTFIGYDMHWVRQAPGQGLEWMGVINPSSGGTNYAQKFQGRVTMTRDTSISTAYMELSRLRSDDTAVYYCARARDIAALGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttcataggctacgacatgcactgggtgcgacaggcccctggacaagggcttgagtggatgggagtcatcaaccctagcagtggtggcacaaactatgcacagaagtttcagggcagggtcaccatgaccagggacacgtccatcagcacagcctacatggagctgagcaggctgagatctgacgacacggcggtgtactactgcgcaagggctagagacatagcagcattgggcttgggagcttttgactactggggacagggtacattggtcaccgtctcctca

SEQ ID NO: 120 - protein, 9 aa, synthetic construct
YTFPGYHMH

SEQ ID NO: 121 - protein, 17 aa, synthetic construct
VINPSSGDTNYAQKFQG

SEQ ID NO: 122 - protein, 123 aa, synthetic construct
QVQLVQSGAEVKKPGASVKVSCKASGYTFPGYHMHWVRQAPGQGLEWMGVINPSSGDTNYAQKFQGRVTMTRDTSISTAYMELSRLRSDDTAVYYCARARDIAALGLGAFDYWGQGTLVTVSS

SEQ ID NO: 123 - protein, 453 aa, synthetic construct
QVQLVQSGAEVKKPGASVKVSCKASGYTFPGYHMHWVRQAPGQGLEWMGVINPSSGDTNYAQKFQGRVTMTRDTSISTAYMELSRLRSDDTAVYYCARARDIAALGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttccccggctaccacatgcactgggtgcgacaggcccctggacaagggcttgagtggatgggagtcatcaaccctagcagtggtgacacaaactatgcacagaagtttcagggcagggtcaccatgaccagggacacgtccatcagcacagcctacatggagctgagcaggctgagatctgacgacacggcggtgtactactgcgcaagggctagagacatagcagcattgggcttgggagcttttgactactggggacagggtacattggtcaccgtctcctca

SEQ ID NO: 125 - protein, 9 aa, synthetic construct
YTFFGYSMH

SEQ ID NO: 126 - protein, 123 aa, synthetic construct
QVQLVQSGAEVKKPGASVKVSCKASGYTFFGYSMHWVRQAPGQGLEWMGVINPSSGDTNYAQKFQGRVTMTRDTSISTAYMELSRLRSDDTAVYYCARARDIAALGLGAFDYWGQGTLVTVSS

SEQ ID NO: 127 - protein, 453 aa, synthetic construct
QVQLVQSGAEVKKPGASVKVSCKASGYTFFGYSMHWVRQAPGQGLEWMGVINPSSGDTNYAQKFQGRVTMTRDTSISTAYMELSRLRSDDTAVYYCARARDIAALGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttcttcggctactccatgcactgggtgcgacaggcccctggacaagggcttgagtggatgggagtcatcaaccctagcagtggtgacacaaactatgcacagaagtttcagggcagggtcaccatgaccagggacacgtccatcagcacagcctacatggagctgagcaggctgagatctgacgacacggcggtgtactactgcgcaagggctagagacatagcagcattgggcttgggagcttttgactactggggacagggtacattggtcaccgtctcctca

SEQ ID NO: 129 - protein, 9 aa, synthetic construct
YTFHGYNMH

SEQ ID NO: 130 - protein, 17 aa, synthetic construct
SINPYSGYTIYAQKFQG

SEQ ID NO: 131 - protein, 123 aa, synthetic construct
QVQLVQSGAEVKKPGASVKVSCKASGYTFHGYNMHWVRQAPGQGLEWMGSINPYSGYTIYAQKFQGRVTMTRDTSISTAYMELSRLRSDDTAVYYCARARDIAAAGLGAFDYWGQGTLVTVSS

SEQ ID NO: 132 - protein, 453 aa, synthetic construct
QVQLVQSGAEVKKPGASVKVSCKASGYTFHGYNMHWVRQAPGQGLEWMGSINPYSGYTIYAQKFQGRVTMTRDTSISTAYMELSRLRSDDTAVYYCARARDIAAAGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttccacggctacaacatgcactgggtgcgacaggcccctggacaagggcttgagtggatgggatcgatcaacccttacagtggttacacaatctatgcacagaagtttcagggcagggtcaccatgaccagggacacgtccatcagcacagcctacatggagctgagcaggctgagatctgacgacacggcggtgtactactgcgcaagggctagagacatagcagcagcaggcttgggagcttttgactactggggacagggtacattggtcaccgtctcctca

SEQ ID NO: 134 - protein, 9 aa, synthetic construct
YTFTGYHIH

SEQ ID NO: 135 - protein, 17 aa, synthetic construct
VISAYSGDTNYAQKLQG

SEQ ID NO: 136 - protein, 14 aa, synthetic construct
WVRQVPGQGLEWMG

SEQ ID NO: 137 - protein, 123 aa, synthetic construct
QVQLVQSGAEVKKPGASVKVSCKASGYTFTGYHIHWVRQVPGQGLEWMGVISAYSGDTNYAQKLQGRVTMTTDTSTSTAYMELRSLRSDDTAVYYCARARDIAALGLGAFDYWGQGTLVTVSS

SEQ ID NO: 138 - protein, 453 aa, synthetic construct
QVQLVQSGAEVKKPGASVKVSCKASGYTFTGYHIHWVRQVPGQGLEWMGVISAYSGDTNYAQKLQGRVTMTTDTSTSTAYMELRSLRSDDTAVYYCARARDIAALGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - DNA, 369 bp, synthetic construct
  source 1..369: mol_type = other DNA
caggttcagctggtgcagtctggagctgaggtgaagaagcctggggcctcagtgaaggtctcctgcaaggcttctggttacacctttaccggatatcacatccactgggtgcgacaggtccctggacaagggcttgagtggatgggagtcatcagcgcttacagtggtgacacaaactatgcacagaagctccagggcagagtcaccatgaccacagacacatccacgagcacagcctacatggagctgaggagcctgagatctgacgacacggcggtgtactactgcgcaagggctagagacatagcagcattgggcttgggagcttttgactactggggacagggtacattggtcaccgtctcctca

SEQ ID NO: 140 - protein, 9 aa, synthetic construct
YTFTSYYIH

SEQ ID NO: 141 - protein, 17 aa, synthetic construct
AIYAYSGDTNYAQKLQG

SEQ ID NO: 142 - protein, 123 aa, synthetic construct
QVQLVQSGAEVKKPGASVKVSCKASGYTFTSYYIHWVRQAPGQGLEWMGAIYAYSGDTNYAQKLQGRVTMTTDTSTSTAYMELRSLRSDDTAVYYCARARDIAAAGLGAFDYWGQGTLVTVSS

SEQ ID NO: 143 - protein, 453 aa, synthetic construct
QVQLVQSGAEVKKPGASVKVSCKASGYTFTSYYIHWVRQAPGQGLEWMGAIYAYSGDTNYAQKLQGRVTMTTDTSTSTAYMELRSLRSDDTAVYYCARARDIAAAGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - DNA, 369 bp, synthetic construct
  source 1..369: mol_type = other DNA
caggttcagctggtgcagtctggagctgaggtgaagaagcctggggcctcagtgaaggtctcctgcaaggcttctggttacacctttaccagctattacatccactgggtgcgacaggcccctggacaagggcttgagtggatgggagccatctacgcttacagtggtgacacaaactatgcacagaagctccagggcagagtcaccatgaccacagacacatccacgagcacagcctacatggagctgaggagcctgagatctgacgacacggcggtgtactactgcgcaagggctagagacatagcagcagcaggcttgggagcttttgactactggggacagggtacattggtcaccgtctcctca

SEQ ID NO: 145 - protein, 17 aa, synthetic construct
HISAYSGTTNYAQKLQG

SEQ ID NO: 146 - protein, 123 aa, synthetic construct
QVQLVQSGAEVKKPGASVKVSCKASGYTFTGYHIHWVRQAPGQGLEWMGHISAYSGTTNYAQKLQGRVTMTTDTSTSTAYMELRSLRSDDTAVYYCARARDIAAAGLGAFDYWGQGTLVTVSS

SEQ ID NO: 147 - protein, 453 aa, synthetic construct
QVQLVQSGAEVKKPGASVKVSCKASGYTFTGYHIHWVRQAPGQGLEWMGHISAYSGTTNYAQKLQGRVTMTTDTSTSTAYMELRSLRSDDTAVYYCARARDIAAAGLGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - DNA, 369 bp, synthetic construct
  source 1..369: mol_type = other DNA
caggttcagctggtgcagtctggagctgaggtgaagaagcctggggcctcagtgaaggtctcctgcaaggcttctggttacacctttaccggatatcacatccactgggtgcgacaggcccctggacaagggcttgagtggatgggacacatcagcgcttacagtggtaccacaaactatgcacagaagctccagggcagagtcaccatgaccacagacacatccacgagcacagcctacatggagctgaggagcctgagatctgacgacacggcggtgtactactgcgcaagggctagagacatagcagcagcaggcttgggagcttttgactactggggacagggtacattggtcaccgtctcctca

SEQ ID NO: 149 - protein, 9 aa, synthetic construct
GTFSNYAIS

SEQ ID NO: 150 - protein, 17 aa, synthetic construct
YIIPFFGTANYAQKFQG

SEQ ID NO: 151 - protein, 16 aa, synthetic construct
ARGGSGYSTSWGSSLY

SEQ ID NO: 152 - protein, 26 aa, synthetic construct
QVQLVQSGAEVKKPGSSVKVSCKASG

SEQ ID NO: 153 - protein, 30 aa, synthetic construct
RVTITADESTSTAYMELSSLRSEDTAVYYC

SEQ ID NO: 154 - protein, 123 aa, synthetic construct
QVQLVQSGAEVKKPGSSVKVSCKASGGTFSNYAISWVRQAPGQGLEWMGYIIPFFGTANYAQKFQGRVTITADESTSTAYMELSSLRSEDTAVYYCARGGSGYSTSWGSSLYWGQGTLVTVSS

SEQ ID NO: 155 - protein, 453 aa, synthetic construct
QVQLVQSGAEVKKPGSSVKVSCKASGGTFSNYAISWVRQAPGQGLEWMGYIIPFFGTANYAQKFQGRVTITADESTSTAYMELSSLRSEDTAVYYCARGGSGYSTSWGSSLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - DNA, 369 bp, synthetic construct
  source 1..369: mol_type = other DNA
caggtgcagctggtgcagtctggggctgaggtgaagaagcctgggtcctcggtgaaggtctcctgcaaggcttctggaggcaccttcagcaactatgctatctcctgggtgcgacaggcccctggacaagggcttgagtggatgggatacatcatccctttcttcggtacagcaaactacgcacagaagttccagggcagagtcacgattaccgcggacgaatccacgagcacagcctacatggagctgagcagcctgagatctgaggacacggcggtgtactactgcgccagaggcggttctggatacagcacgagctggggttcaagcctatactggggacagggtacattggtcaccgtctcctca

SEQ ID NO: 157 - protein, 9 aa, synthetic construct
GTFSLYAIS

SEQ ID NO: 158 - protein, 17 aa, synthetic construct
GIIPYFGTANYAQKFQG

SEQ ID NO: 159 - protein, 26 aa, synthetic construct
EVQLVQSGAEVKKPGSSVKVSCKASG

SEQ ID NO: 160 - protein, 123 aa, synthetic construct
EVQLVQSGAEVKKPGSSVKVSCKASGGTFSLYAISWVRQAPGQGLEWMGGIIPYFGTANYAQKFQGRVTITADESTSTAYMELSSLRSEDTAVYYCARGGSGYSTSWGSSLYWGQGTLVTVSS

SEQ ID NO: 161 - protein, 453 aa, synthetic construct
EVQLVQSGAEVKKPGSSVKVSCKASGGTFSLYAISWVRQAPGQGLEWMGGIIPYFGTANYAQKFQGRVTITADESTSTAYMELSSLRSEDTAVYYCARGGSGYSTSWGSSLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - DNA, 369 bp, synthetic construct
  source 1..369: mol_type = other DNA
gaggtgcagctggtgcagtctggggctgaggtgaagaagcctgggtcctcggtgaaggtctcctgcaaggcttctggaggcaccttcagcctctatgcaatcagctgggtgcgacaggcccctggacaagggcttgagtggatgggagggatcatcccttacttcggtacagcaaactacgcacagaagttccagggcagagtcacgattaccgcggacgaatccacgagcacagcctacatggagctgagcagcctgagatctgaggacacggcggtgtactactgcgccagaggcggttctggatacagcactagctggggttcaagcctatactggggacagggtacattggtcaccgtctcctca

SEQ ID NO: 163 - protein, 9 aa, synthetic construct
GTSSSYAIS

SEQ ID NO: 164 - protein, 17 aa, synthetic construct
YIIPAFGTANYAQKFQG

SEQ ID NO: 165 - protein, 15 aa, synthetic construct
ARPVRTYPLGKYFYH

SEQ ID NO: 166 - protein, 122 aa, synthetic construct
QVQLVQSGAEVKKPGSSVKVSCKASGGTSSSYAISWVRQAPGQGLEWMGYIIPAFGTANYAQKFQGRVTITADESTSTAYMELSSLRSEDTAVYYCARPVRTYPLGKYFYHWGQGTLVTVSS

SEQ ID NO: 167 - protein, 452 aa, synthetic construct
QVQLVQSGAEVKKPGSSVKVSCKASGGTSSSYAISWVRQAPGQGLEWMGYIIPAFGTANYAQKFQGRVTITADESTSTAYMELSSLRSEDTAVYYCARPVRTYPLGKYFYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - DNA, 366 bp, synthetic construct
  source 1..366: mol_type = other DNA
caggtgcagctggtgcagtctggggctgaggtgaagaagcctgggtcctcggtgaaggtctcctgcaaggcttctggaggcacctccagcagctatgctatcagctgggtgcgacaggcccctggacaagggcttgagtggatgggatacatcatccctgccttcggtacagcaaactacgcacagaagttccagggcagagtcacgattaccgcggacgaatccacgagcacagcctacatggagctgagcagcctgagatctgaggacacggcggtgtactactgcgccagaccggtgagaacctaccctctaggcaagtacttctatcactggggacagggtacattggtcaccgtctcctca

SEQ ID NO: 169 - protein, 10 aa, synthetic construct
YSISSGYYWG

SEQ ID NO: 170 - protein, 16 aa, synthetic construct
SIYHSGSTYYNPSLKS

SEQ ID NO: 171 - protein, 18 aa, synthetic construct
AREAGSGYWYLSVYGMDV

SEQ ID NO: 172 - protein, 26 aa, synthetic construct
QVQLQESGPGLVKPSETLSLTCAVSG

SEQ ID NO: 173 - protein, 14 aa, synthetic construct
WIRQPPGKGLEWIG

SEQ ID NO: 174 - protein, 30 aa, synthetic construct
RVTISVDTSKNQFSLKLSSVTAADTAVYYC

SEQ ID NO: 175 - protein, 11 aa, synthetic construct
WGQGTTVTVSS

SEQ ID NO: 176 - protein, 125 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREAGSGYWYLSVYGMDVWGQGTTVTVSS

SEQ ID NO: 177 - protein, 455 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREAGSGYWYLSVYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - DNA, 375 bp, synthetic construct
  source 1..375: mol_type = other DNA
caggtgcagctgcaggagtcgggcccaggactggtgaagccttcggagaccctgtccctcacctgcgctgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagttctgtgaccgccgcagacacggcggtgtactactgcgccagagaagccggaagcggatactggtacttgagtgtatacggaatggacgtatggggccagggaacaactgtcaccgtctcctca

SEQ ID NO: 179 - protein, 11 aa, synthetic construct
GSISSSDYYWG

SEQ ID NO: 180 - protein, 16 aa, synthetic construct
SIYYPANTYYNPSLKS

SEQ ID NO: 181 - protein, 11 aa, synthetic construct
ARGYWNYGMDV

SEQ ID NO: 182 - protein, 26 aa, synthetic construct
QLQLQESGPGLVKPSETLSLTCTVSG

SEQ ID NO: 183 - protein, 119 aa, synthetic construct
QLQLQESGPGLVKPSETLSLTCTVSGGSISSSDYYWGWIRQPPGKGLEWIGSIYYPANTYYNPSLKSRVTISVDTSKNQFSLKLSSVTAADTAVYYCARGYWNYGMDVWGQGTTVTVSS

SEQ ID NO: 184 - protein, 449 aa, synthetic construct
QLQLQESGPGLVKPSETLSLTCTVSGGSISSSDYYWGWIRQPPGKGLEWIGSIYYPANTYYNPSLKSRVTISVDTSKNQFSLKLSSVTAADTAVYYCARGYWNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - DNA, 357 bp, synthetic construct
  source 1..357: mol_type = other DNA
cagctgcagctgcaggagtcgggcccaggactggtgaagccttcggagaccctgtccctcacctgcactgtctctggtggctccatcagcagtagtgactactactggggctggatccgccagcccccagggaaggggctggagtggattgggagtatctactatcccgcaaacacctactacaacccgtccctcaagagtcgagtcaccatatccgtagacacgtccaagaaccagttctccctgaagctgagttctgtgaccgccgcagacacggcggtgtactactgcgccagaggatattggaactatggcatggatgtgtggggccagggaacaactgtcaccgtctcctca

SEQ ID NO: 186 - protein, 16 aa, synthetic construct
SIYYPASTYYNPSLKS

SEQ ID NO: 187 - protein, 119 aa, synthetic construct
QLQLQESGPGLVKPSETLSLTCTVSGGSISSSDYYWGWIRQPPGKGLEWIGSIYYPASTYYNPSLKSRVTISVDTSKNQFSLKLSSVTAADTAVYYCARGYWNYGMDVWGQGTTVTVSS

SEQ ID NO: 188 - protein, 449 aa, synthetic construct
QLQLQESGPGLVKPSETLSLTCTVSGGSISSSDYYWGWIRQPPGKGLEWIGSIYYPASTYYNPSLKSRVTISVDTSKNQFSLKLSSVTAADTAVYYCARGYWNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - DNA, 357 bp, synthetic construct
  source 1..357: mol_type = other DNA
cagctgcagctgcaggagtcgggcccaggactggtgaagccttcggagaccctgtccctcacctgcactgtctctggtggctccatcagcagtagtgactactactggggctggatccgccagcccccagggaaggggctggagtggattgggagtatctactatcccgcaagcacctactacaacccgtccctcaagagtcgagtcaccatatccgtagacacgtccaagaaccagttctccctgaagctgagttctgtgaccgccgcagacacggcggtgtactactgcgccagaggatattggaactatggcatggatgtgtggggccagggaacaactgtcaccgtctcctca

SEQ ID NO: 190 - protein, 9 aa, synthetic construct
GTFSNYDIN

SEQ ID NO: 191 - protein, 17 aa, synthetic construct
GIIPVFGTAAYAQKFQG

SEQ ID NO: 192 - protein, 14 aa, synthetic construct
AREARDGYRYAFQH

SEQ ID NO: 193 - protein, 121 aa, synthetic construct
QVQLVQSGAEVKKPGSSVKVSCKASGGTFSNYDINWVRQAPGQGLEWMGGIIPVFGTAAYAQKFQGRVTITADESTSTAYMELSSLRSEDTAVYYCAREARDGYRYAFQHWGQGTLVTVSS

SEQ ID NO: 194 - protein, 451 aa, synthetic construct
QVQLVQSGAEVKKPGSSVKVSCKASGGTFSNYDINWVRQAPGQGLEWMGGIIPVFGTAAYAQKFQGRVTITADESTSTAYMELSSLRSEDTAVYYCAREARDGYRYAFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 363 bp, synthetic construct
  source 1..363: mol_type = other DNA
caggtgcagctggtgcagtctggggctgaggtgaagaagcctgggtcctcggtgaaggtctcctgcaaggcttctggaggcaccttcagcaactatgacatcaactgggtgcgacaggcccctggacaagggcttgagtggatgggagggatcatccctgtcttcggtacagcagcctacgcacagaagttccagggcagagtcacgattaccgcggacgaatccacgagcacagcctacatggagctgagcagcctgagatctgaggacacggcggtgtactactgcgccagagaagctagagacggatacagatacgccttccaacactggggacagggtacattggtcaccgtctcctca

SEQ ID NO: 196 - protein, 9 aa, synthetic construct
GTFDDYAIS

SEQ ID NO: 197 - protein, 17 aa, synthetic construct
GIIPRFGIANYAQKFQG

SEQ ID NO: 198 - protein, 9 aa, synthetic construct
ARPPSSGSD

SEQ ID NO: 199 - protein, 116 aa, synthetic construct
QVQLVQSGAEVKKPGSSVKVSCKASGGTFDDYAISWVRQAPGQGLEWMGGIIPRFGIANYAQKFQGRVTITADESTSTAYMELSSLRSEDTAVYYCARPPSSGSDWGQGTLVTVSS

SEQ ID NO: 200 - protein, 446 aa, synthetic construct
QVQLVQSGAEVKKPGSSVKVSCKASGGTFDDYAISWVRQAPGQGLEWMGGIIPRFGIANYAQKFQGRVTITADESTSTAYMELSSLRSEDTAVYYCARPPSSGSDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - DNA, 348 bp, synthetic construct
  source 1..348: mol_type = other DNA
caggtgcagctggtgcagtctggggctgaggtgaagaagcctgggtcctcggtgaaggtctcctgcaaggcttctggaggcaccttcgacgactatgctatcagctgggtgcgacaggcccctggacaagggcttgagtggatgggagggatcatccctagattcggtatcgcaaactacgcacagaagttccagggcagagtcacgattaccgcggacgaatccacgagcacagcctacatggagctgagcagcctgagatctgaggacacggcggtgtactactgcgccagaccacctagttctggttctgattggggacagggtacattggtcaccgtctcctca

SEQ ID NO: 202 - protein, 9 aa, synthetic construct
GSFSPYTWS

SEQ ID NO: 203 - protein, 17 aa, synthetic construct
GIIPISGTANTAQKFQG

SEQ ID NO: 204 - protein, 15 aa, synthetic construct
ARGGPLSGWRASYYY

SEQ ID NO: 205 - skipped

SEQ ID NO: 206 - protein, 122 aa, synthetic construct
QVQLVQSGAEVKKPGSSVKVSCKASGGSFSPYTWSWVRQAPGQGLEWMGGIIPISGTANTAQKFQGRVTITADESTSTAYMELSSLRSEDTAVYYCARGGPLSGWRASYYYWGQGTLVTVSS

SEQ ID NO: 207 - protein, 452 aa, synthetic construct
QVQLVQSGAEVKKPGSSVKVSCKASGGSFSPYTWSWVRQAPGQGLEWMGGIIPISGTANTAQKFQGRVTITADESTSTAYMELSSLRSEDTAVYYCARGGPLSGWRASYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - DNA, 366 bp, synthetic construct
  source 1..366: mol_type = other DNA
caggtgcagctggtgcagtctggggctgaggtgaagaagcctgggtcctcggtgaaggtctcctgcaaggcttctggaggctcgttcagcccctatacatggagctgggtgcgacaggcccctggacaagggcttgagtggatgggagggatcatccctatctctggtacagcaaacacggcacagaagttccagggcagagtcacgattaccgcggacgaatccacgagcacagcctacatggagctgagcagcctgagatctgaggacacggcggtgtactactgcgccagaggtgggcctttgagcggttggagagccagctactactattggggacagggtacattggtcaccgtctcctca

SEQ ID NO: 209 - protein, 9 aa, synthetic construct
GSFSPYTIS

SEQ ID NO: 210 - protein, 17 aa, synthetic construct
GIILTSGTANYAQKFQG

SEQ ID NO: 211 - protein, 122 aa, synthetic construct
QVQLVQSGAEVKKPGSSVKVSCKASGGSFSPYTISWVRQAPGQGLEWMGGIILTSGTANYAQKFQGRVTITADESTSTAYMELSSLRSEDTAVYYCARGGPLSGWRASYYYWGQGTLVTVSS

SEQ ID NO: 212 - protein, 452 aa, synthetic construct
QVQLVQSGAEVKKPGSSVKVSCKASGGSFSPYTISWVRQAPGQGLEWMGGIILTSGTANYAQKFQGRVTITADESTSTAYMELSSLRSEDTAVYYCARGGPLSGWRASYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - DNA, 366 bp, synthetic construct
  source 1..366: mol_type = other DNA
caggtgcagctggtgcagtctggggctgaggtgaagaagcctgggtcctcggtgaaggtctcctgcaaggcttctggaggcagtttcagcccctatacaatcagctgggtgcgacaggcccctggacaagggcttgagtggatgggagggatcatcctgacttctggtacagcaaactacgcacagaagttccagggcagagtcacgattaccgcggacgaatccacgagcacagcctacatggagctgagcagcctgagatctgaggacacggcggtgtactactgcgccagaggtgggcctttgagcggttggagagccagctactactattggggacagggtacattggtcaccgtctcctca

SEQ ID NO: 214 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 216 - DNA, 6105 bp, Homo sapiens
  source 1..6105: mol_type = genomic DNA
agtacagtataaaacttcacagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaaaacaaacaacattaaaaaattgagaacaagtatggaaagaatgaaagatcaaggaaaaaagaataccaagttacatttcgttaaggtgcttatgatcttagaactatgcaacctaataggtttgaaactgtttacctgagagagaacaaaaagagagacttttttgtattggaagtaatctgattaatttttattttcttcaaggagagatacttgaaaggaatatgtttgtccatctgttggatccaaacatttctatattttgtaaatgttgttgttgttttttttttaatcgtttactatttgcactacaatggtgtttgacctgtctaatccttatttaacaagtattttctttggttgggggtgggggtggggtttaagagctgcacttaatgtgagctataaaagaactgctacagcacacaaaatagctatttttattattataattataattattattattattttgtaccttaaaaaatagacacatacaccaaagacatttgtgtgagcctttaaacagtctgtctgtggttggtatcattcaccatcaatgagtcaggggttgggattcaaggttgagtagtgtggattgtgttcaggcttaaaagacctgagaagtttggtttttgactccttttacatccatgaaacaggacatttcatactggatgtacagtagttgtacactgttggatatcaagttcaatcaaattcatggaactacatgcttgtatgtgtatatatacattgcttgtgcatatgcatatctgtatgtatatatacatgtattgtaccatgtccatacacattttaagcacttcaggctgtcattttttaatgttcttaaagcaatgaatgtttgtgtgcaaaacacagtatttttaagaaggataggctatagtttttgcttttactctgaactaggtgggcgcatttcaaaaattcggatgggaaaaagcctggaaattccagtgaatattcagcaaggccctctttcattgtacagggatcaaatttcctcctcttttttgtgccccctcccacttctacaagttatcccctgtggggaaaacaggatgataatcaaaactctgggctgatgtttttccaacttagtgtctattggaatcaatcttaaatcagaagctttttcagaaaaataatatttaggccagaattagagttgagtgtattttttaaaaatgattaaggcttggttgtgagaaatattacctgtaccagctgggaaaaataatgtcatcactaactaaaagataattaatttgagagaaagtgttaagagagggagagtaaggaagagaacagttaagaggaggcagaggtgagggcagtagtaaaaatctctaaaattttaatttacagccaaaattcttcatgtgtaaatttgtattgattcagatgcagaaatgaaaaaaaaacacctttgttttataaatatcaaagtacatgcttaaagccaagtttttatctagtttattctagtacttagcttgcctggaatagctaataaattattcatgtatgtgcttttgaaaatccagagccctatttttacacacttgtgtgaagttggcaaacattttgaaaaatggaaaaaagtttctaataattgggaacaattacattaattaatattttgtaaaatattgaagcttttagccctatgtcaatttgtagattaaaataaattaattataggaaaggaagataacagtgagaaaccaaacattacaaaaggtggtttagctctccttgaaaaatatactaagttggtatactataacacttggctatatgtaggcaatgtcactactgggcaaatacacttactgtgttctagaggcagccctttcttatgcagaaaatacaatacgcactgcatgagaagcttgagagtggattctaatccaggtctgtcgaccttggatatcatgcatgtgggaaggtgggtgtggtgagaaaagttttaaggcaagagtagatggccatgttcaactttacaaaatttcttggaaaactggcagtattttgaactgcatcttctttggtaccggaacctgcagaaacagtgtgagaaattaagtcctggttcactgcgcagtagcaaagatggtcaaggccatggaaaaagcagaaatttaccaagaaagctgatacccatgtatagttcccactcatctcaaatacatctgctatctttttaagctaagtcctagacatatcggggataacatgggggttgattagtgaccacagttatcagaagcagagaaatgtaattccatattttatttgaaacttattccatattttaattggatattgagtgattgggttatcaaacacccacaaactttaattttgttaaatttatatggctttgaaatagaagtataagttgctaccattttttgataacattgaaagatagtattttaccatctttaatcatcttggaaaatacaagtcctgtgaacaaccactctttcacctagcagcatgaggccaaaagtaaaggctttaaattataacatatgggattcttagtagtatgtttttttcttgaaactcagtggctctatctaaccttactatctcctcactctttctctaagactaaactctaggctcttaaaaatctgcccacaccaatcttagaagctctgaaaagaatttgtctttaaatatcttttaatagtaacatgtattttatggaccaaattgacattttcgactattttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaaggtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgtaaaaaaaaaaaaaaaaaaaaaaaaaaaaaaaaaaaaaaaaaa

SEQ ID NO: 217 - protein, 20 aa, Homo sapiens
MPLLWLRGFLLASCWIIVRS

SEQ ID NO: 218 - protein, 116 aa, Homo sapiens [sequence omitted]

SEQ ID NO: 219 - protein, 426 aa, Macaca mulatta
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTTRKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGDGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 220 - protein, 424 aa, Canis lupus
MPLLWLRGFLVASCWIIVRSSPTPGPEGPGAAPACPACALTALPRDAPNSQPEMVEAVKKHILNMLHLKKRPEVTQPVPKAALLNAIRKLHVGKVGENGFVEIEDDIGRRAEMNELMEQTSEIITFAESGTARKTLHFEISKEGSDLSVVERAEVWLFLKVPKANRTRTKVTIRLLQKHPQGSLDAGEEAEDMGFPEERNEVLISEKVVDARKSTWHIFPVSSSIQRLLDQGRSSLDVRIACEQCHETGASLVLLGKKKKKEEEGEGKKKDGGDAGAGGDEDKEQSHRPFLMLQARQSEDHPHRRRRRGLECDGKVNICCKKQFFVSFKDIGWNDWIIAPSGYHANYCEGGCPSHIAGTSGSSLSFHSTVINHYRLRGHSPFTNLKSCCVPTKLRPMSMLYYDDGQNIIKKDIQNMIVEECGCS

SEQ ID NO: 221 - protein, 424 aa, Felis catus
MPLLWLRGFLLASCWIIVRSSPTPGSEGPGAAPDCPSCALATLPKDVPNSQPEMVEAVKKHILNMLHLKKRPEVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIQLLQKQPQGGVDAGEEAEEMGLMEERNEVLISEKVVDARKSTWHIFPVSSSIQRLLDQGKSSLDVRIACEQCHETGASLVLLGKKKKKEEEGEGKKKDGGDGGAGADEDKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 222 - protein, 21 aa, synthetic construct
CPPCPAPELLGGPSVFLFPPK

SEQ ID NO: 223 - protein, 21 aa, synthetic construct
CPPCPAPELAGAPSVFLFPPK

SEQ ID NO: 224 - protein, 21 aa, synthetic construct
CPPCPAPEFEGGPSVFLFPPK

SEQ ID NO: 225 - protein, 21 aa, synthetic construct
CPPCPAPEAAGGPSVFLFPPK

SEQ ID NO: 226 - protein, 21 aa, synthetic construct
CPPCPAPEAAGAPSVFLFPPK

SEQ ID NO: 227 - protein, 106 aa, synthetic construct [sequence omitted]

SEQ ID NO: 228 - protein, 4 aa, synthetic construct
LAGA

SEQ ID NO: 229 - protein, 4 aa, synthetic construct
FEGG

SEQ ID NO: 230 - protein, 4 aa, synthetic construct
AAGG

SEQ ID NO: 231 - protein, 4 aa, synthetic construct
AAGA

(19) United States
(12) Patent Application Publication
(10) Pub. No.: US 2026/0265358 A1
(43) Pub. Date: Sep. 10, 2026

(54) ANTI-ACTIVIN A ANTIBODIES, COMPOSITIONS AND USES THEREOF

(71) Applicants: BYOMass Inc., Sudbury, MA (US); Adimab, LLC, Lebanon, NH (US)

(72) Inventors: Vivienne Margaret Jackson, Sudbury, MA (US); Nels P. Nielson, Lebanon, NH (US)

(21) Appl. No.: 18/702,065

(22) PCT Filed: Oct. 18, 2022
(86) PCT No.: PCT/US2022/047001

§ 371 Date: Apr. 17, 2024

Related U.S. Application Data
(60) Provisional application No. 63/257,067, filed on Oct. 18, 2021.

(51) Int. Cl. C07K 16/22; A61K 39/00; A61P 21/00

(52) U.S. Cl. CPC C07K 16/22; A61P 21/00; A61K 2039/505; C07K 2317/569

(57) ABSTRACT

Provided herein are antibody agents that bind specifically to Activin A, as well as compositions comprising Activin A antibody agents, and methods of making and using the same.

### CROSS-REFERENCE TO RELATED APPLICATIONS

This application is a national stage application of international application PCT/US2022/047001 filed under 35 U.S.C. § 371, and claims priority to U.S. Provisional Patent Application 63/257,067 filed on Oct. 18, 2021, the entire contents of each of which is hereby incorporated by reference.

### SEQUENCE LISTING

The instant application contains a Sequence Listing which has been submitted electronically in XML file format and is hereby incorporated by reference in its entirety. Said XML copy, created on Apr. 15, 2026, is named 2014039-0030_SL.xml and is 229,376 bytes in size.

### BACKGROUND

Activin A is a member of the transforming growth factor beta (TGFb) superfamily. Activin A can be expressed in a variety of cells and the expression of Activin A can be upregulated in response to stimuli such as inflammation.

### SUMMARY

The present disclosure provides new, high-affinity Activin A antibody agents. Among other things, provided agents can be used, e.g., to bind to Activin A and/or to reduce an activity and/or level of free/active Activin A in a relevant system (e.g., in vitro, in a cell, in a tissue and/or in a subject).

For example, in some embodiments, the present disclosure provides novel Activin A antibody agents which have improved binding kinetics, binding affinity, pharmacokinetics and/or function. In some embodiments, an Activin A antibody agent disclosed herein binds to Activin A with high specificity. In some embodiments, an Activin A antibody agent binds to Activin A with a K_{D} of about 5 pM to about 1000 pM (e.g., about 6.17 pM to about 960 pM), e.g., as assessed in an assay that measures antibody binding affinity, e.g., a Surface Plasmon Resonance assay (Biacore). In some embodiments, a provided Activin A antibody agent may show preferential binding to Activin A relative to one or more TGFbeta family members other than Activin A. In some such embodiments, preferential binding may be assessed, for example, by simultaneously contacting an Activin A antibody agent with Activin A and one or more other TGFbeta family members. Alternatively or additionally, in some embodiments, preferential binding may be assessed relative to an appropriate reference Activin A antibody agent and, e.g., may reflect a higher level of binding to Activin A relative to the one or more other TGFbeta family member than is observed with the reference antibody.

In some embodiments, an Activin A antibody agent disclosed herein inhibits an activity of Activin A and/or reduces a level of Activin A (e.g., a level of free and/or active Activin A, e.g., in a blood, plasma, serum and/or urine sample) when administered to a cell, tissue or subject.

In some embodiments, an Activin A antibody agent can be used to prevent, reduce, treat, and/or reverse any one, all, or a combination of: cachexia, mass loss (e.g., muscle mass loss, lean mass loss, fat mass loss, and/or bone mass loss, and/or organ mass loss), weight loss (e.g., unintentional weight loss due to cachexia), senescence, liver damage (e.g., fibrosis, cirrhosis, degeneration), kidney damage (e.g., fibrosis), or cancer, cancer metastases, chemotherapy induced toxicity (e.g., nephrotoxicity), chemoresistance and/or hypertension. In some embodiments, cachexia, mass loss (e.g., muscle mass loss, lean mass loss, fat mass loss, bone mass loss, and/or organ mass loss), weight loss (e.g., unintentional weight loss due to cachexia), senescence, liver damage (e.g., fibrosis, degeneration, cirrhosis [e.g., alcoholic and/or non-alcoholic cirrhosis]), kidney damage (e.g., fibrosis), cancer, cancer metastases, chemotherapy induced toxicity (e.g., nephrotoxicity and/or hepatotoxicity), chemoresistance and/or hypertension is associated with increased Activin A.

In some embodiments, an Activin A antibody agent disclosed herein can be used to prevent and/or treat a condition or disease associated with increased Activin A, e.g., any one or all or a combination of: (i) disorders of the hypothalamic pituitary gonadal axis (e.g., with increased FSH levels); (ii) fibrodysplasia ossificans progressive (FOP); (iii) pulmonary arterial hypertension and/or hypertension; (iv) Anorexia-Cachexia Disorders associated with chronic diseases such as cancer, chronic kidney disease, chronic heart failure, COPD, elderly and muscle immobility, ICU patients (e.g., loss of body weight, loss of food intake, fat mass loss, muscle wasting, loss of functional muscle mass and strength, bone loss, anemia and/or fatigue); (v) anemia; (vi) metabolic disorders (e.g., obesity, type 2 diabetes, and/or metabolic syndrome); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis); (viii) auto-immune disorders (e.g., SLE or rheumatoid arthritis); (ix) fibrotic diseases (e.g. idiopathic pulmonary fibrosis, NASH, liver cirrhosis, chronic kidney disease, Alport Syndrome, Uterine Fibroids, Endometriosis) (x) cancer (xi) cancer-treatment related toxicities (e.g., chemotherapy-induced nephrotoxicity, and/or hepatotoxicity); (xii) aging (e.g., senescence) or (xiii) chemoresistance. Among other things, this disclosure provides compositions comprising new and improved Activin A antibody agents, as well as methods of making and using the same.

In some embodiments, provided herein is an antibody agent comprising a polypeptide that binds to Activin A comprising at least one light chain complementarity determining region (LC CDR) and/or at least one heavy chain complementary determining region (HC CDR).

In some embodiments, an antibody agent is or comprises: (i) an intact IgA, IgG, IgD, IgE or IgM antibody; (ii) an antibody fragment; (iii) a single domain antibody; (iv) a single chain Fv; or (v) a polypeptide comprising antigen binding specific fused to a Fc domain.

In some embodiments, an antibody agent binds to human Activin A with a binding affinity (K_{D}) of about 6.17×10(−12)M to about 960×10(−12)M, e.g., with a Fab format. In some embodiments, a binding affinity is determined with a binding affinity determining assay such as a surface plasmon resonance assay, an Octet assay or a comparable assay.

In some embodiments, an antibody agent comprises one, two, or three LC CDRs, e.g., as provided in Table 1 e.g., an LC CDR1, LC CDR2 and/or LC CDR3 of any one of clones A-R. In some embodiments, an antibody agent comprising a LC CDR1, LC CDR2 and/or LC CDR3 is capable of binding specifically to Activin A.

In some embodiments, an antibody agent comprises (i) an LC CDR1, LC CDR2, and LC CDR3 sequence provided in Table 1; (ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or (iii) a sequence having at least 5, 10, or 20 substitutions relative to an LC CDR1, LC CDR2, and LC CDR3 sequence provided in Table 1.

In some embodiments, an antibody agent comprises a light chain comprising: (i) an LC CDR1 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR3 provided in Table 1.

In some embodiments, an antibody agent comprises one, two or three HC CDRs, e.g., as provided in Table 2, e.g., an HC CDR1, an HC CDR2, and/or an HC CDR3 of any one of clones A-R. In some embodiments, an antibody agent comprising a HC CDR1, HC CDR2 and/or HC CDR3 is capable of binding specifically to Activin A.

In some embodiments, an antibody agent comprises (i) an HC CDR1, HC CDR2, and HC CDR3 sequence provided in Table 1; (ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 1; (iii) a sequence having at least 5, 10, or 20 substitutions relative to an HC CDR1, HC CDR2, and HC CDR3 sequence provided in Table 1.

In some embodiments, an antibody agent comprises a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR3 provided in Table 2.

In some embodiments, an antibody agent comprises one, two, or three LC CDRs (e.g., as provided in Table 1, e.g., an LC CDR1, LC CDR2 and/or LC CDR3 of any one of clones A-R) and one, two, or three HC CDRs (e.g., as provided in Table 2, e.g., an HC CDR1, an HC CDR2, and/or an HC CDR3 of any one of clones A-R). In some embodiments, an antibody agent comprising a LC CDR1, LC CDR2 and/or LC CDR3; and a HC CDR1, HC CDR2 and/or HC CDR3 is capable of binding specifically to Activin A.

In some embodiments, an antibody agent comprises one, two, or three LC CDRs provided in Table 1 or a sequence with at least 85%, 86%, 87%, 88%, 89%, 90%, 91%, 92%, 93%, 94%, 95%, 96%, 97%, 98%, or 99% thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% thereto.

In some embodiments, an antibody agent comprises: (a) a light chain comprising: (i) an LC CDR1 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR3 provided in Table 1; and (b) a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR3 provided in Table 2.

In some embodiments, an antibody agent comprising a light chain comprising a variable region (VL) comprising at least one LC CDR provided in Table 1 comprises at least one framework region (FR) provided in Table 1 or a sequence with at least 92% identity thereto. In some embodiments, an antibody agent comprising a VL comprises one, two, three or four FR regions, e.g., as provided in Table 1, or a sequence with at least 92% identity thereto.

In some embodiments, an antibody agent comprising a light chain comprising a variable region (VL) comprises: (i) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; (ii) the sequence of SEQ ID NO: 13, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 13; (iii) the sequence of SEQ ID NO: 18, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 18; (iv) the sequence of SEQ ID NO: 22, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 22; (v) the sequence of SEQ ID NO: 33, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 33; (vi) the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; (vii) the sequence of SEQ ID NO: 45, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 45; (viii) the sequence of SEQ ID NO: 50, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 50; (ix) the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58; (x) the sequence of SEQ ID NO: 65, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 65; (xi) the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; (xii) the sequence of SEQ ID NO: 82, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 82; (xiii) the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90; or; (xiv) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99.

In some embodiments, an antibody agent comprising a heavy chain comprising a variable region (VH) comprising at least one HC CDR provided in Table 2 comprises at least one framework region (FR) provided in Table 2 or a sequence with at least 92% identity thereto. In some embodiments, an antibody agent comprising a VH comprises one, two, three or four FR regions, e.g., as provided in Table 2, or a sequence with at least 92% identity thereto.

In some embodiments, an antibody agent comprising a VH comprises: (i) the sequence of SEQ ID NO: 110, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 110; (ii) the sequence of SEQ ID NO: 117, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 117; (iii) the sequence of SEQ ID NO: 122, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 122; (iv) the sequence of SEQ ID NO: 126, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 126; (v) the sequence of SEQ ID NO: 131, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 131; (vi) the sequence of SEQ ID NO: 137, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 137; (vii) the sequence of SEQ ID NO: 142, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 142; (viii) the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146; (ix) the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154; (x) the sequence of SEQ ID NO: 160, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 160; (xi) the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166; (xii) the sequence of SEQ ID NO: 176, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 176; (xiii) the sequence of SEQ ID NO: 183, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 183; (xiv) the sequence of SEQ ID NO: 187, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 187; (xv) the sequence of SEQ ID NO: 193, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 193; (xvi) the sequence of SEQ ID NO: 199 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 199; (xvii) the sequence of SEQ ID NO: 206 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206; or (xviii) the sequence of SEQ ID NO: 211 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 211.

In some embodiments, an antibody agent comprises a VL polypeptide and a VH polypeptide. In some embodiments, an antibody agent comprises: (i) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; and the sequence of SEQ ID NO: 110, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 110; (ii) the sequence of SEQ ID NO: 13, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 13; and the sequence of SEQ ID NO: 117, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 117; (iii) the sequence of SEQ ID NO: 18, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 18; and the sequence of SEQ ID NO: 122, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 122; (iv) the sequence of SEQ ID NO: 22, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 22; and the sequence of SEQ ID NO: 126, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 126; (v) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; and the sequence of SEQ ID NO: 131, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 131; (vi) the sequence of SEQ ID NO: 33, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 33; and the sequence of SEQ ID NO:137, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO:137; (vii) the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; and the sequence of SEQ ID NO: 142, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 142; (viii) the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; and the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146; (ix) the sequence of SEQ ID NO: 45, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 45; and the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154; (x) the sequence of SEQ ID NO: 50, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 50; and the sequence of SEQ ID NO: 160, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 160; (xi) the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166; (xii) the sequence of SEQ ID NO: 65, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 65; and the sequence of SEQ ID NO: 176, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 176; (xiii) the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; and the sequence of SEQ ID NO: 183, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 183; (xiv) the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; and the sequence of SEQ ID NO: 187, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 187; (xv) the sequence of SEQ ID NO: 82, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 82; and the sequence of SEQ ID NO: 193, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 193; (xvi) the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90; and the sequence of SEQ ID NO: 199, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 199; (xvii) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206; or (xviii) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 211, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 211.

In some embodiments, an antibody agent comprising a VH comprises a sequence for at least one constant region (CH). In some embodiments, at least one constant region comprises an Fc domain. In some embodiments, an Fc domain comprises a mammalian Fc domain, e.g., a mouse, a rat, a rabbit, a primate, a human, or a domestic animal Fc domain (e.g., a dog, a cat, a cow, or a horse Fc domain). In some embodiments, an Fc domain is chosen from an Fc domain of an immunoglobulin isotype. In some embodiments, an immunoglobulin isotype comprises IgA, IgG, IgM, or IgE.

In some embodiments, an antibody agent comprises (i) a light chain (LC) comprising: (a) one, two or three LC CDRs provided in Table 1 or a sequence with at least 85%, 86%, 87%, 88%, 89%, 90%, 91%, 92%, 93%, 94%, 95%, 96%, 97%, 98%, or 99% thereto; (b) at least one FR provided in Table 1 or sequence with at least 92% identity thereto; (c) a constant region (CL); and (ii) a heavy chain (HC) comprising: (a) one, two or three HC CDRs provided in Table 2 or a sequence with at least 85%, 86%, 87%, 88%, 89%, 90%, 91%, 92%, 93%, 94%, 95%, 96%, 97%, 98%, or 99% thereto; (b) at least one FR provided in Table 1 or a sequence with at least 92% identity thereto; (c) at least one constant region.

In some embodiments, an antibody agent comprises (i) the sequence of SEQ ID NO: 9, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 9; and the sequence of SEQ ID NO: 111, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 111; (ii) the sequence of SEQ ID NO: 14, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 14; and the sequence of SEQ ID NO: 118, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 118; (iii) the sequence of SEQ ID NO: 19, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 19; and the sequence of SEQ ID NO: 123, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 123; (iv) the sequence of SEQ ID NO: 23, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 23; and the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127; (v) the sequence of SEQ ID NO: 9, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 9; and the sequence of SEQ ID NO: 132, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 132; (vi) the sequence of SEQ ID NO: 34, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 34; and the sequence of SEQ ID NO: 138, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 138; (vii) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38; and the sequence of SEQ ID NO: 143, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143; (viii) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38; and the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; (ix) the sequence of SEQ ID NO: 46, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 46; and the sequence of SEQ ID NO: 155, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155; (x) the sequence of SEQ ID NO: 51, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 51; and the sequence of SEQ ID NO: 161, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 161; (xi) the sequence of SEQ ID NO: 59, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 59; and the sequence of SEQ ID NO: 167, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 167; (xii) the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66; and the sequence of SEQ ID NO: 177, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 177; (xiii) the sequence of SEQ ID NO: 75, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 75; and the sequence of SEQ ID NO: 184, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 184; (xiv) the sequence of SEQ ID NO: 75, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 75; and the sequence of SEQ ID NO: 188, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 188; (xv) the sequence of SEQ ID NO: 83, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 83; and the sequence of SEQ ID NO: 194, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 194; (xvi) the sequence of SEQ ID NO: 91, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 91; and the sequence of SEQ ID NO: 200, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 200; (xvii) the sequence of SEQ ID NO: 100, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 100; and the sequence of SEQ ID NO: 207, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 207; or (xviii) the sequence of SEQ ID NO: 100, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 100; and the sequence of SEQ ID NO: 212, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 212.

In some embodiments, an Activin A antibody agent is characterized in that when tested in an assay that evaluates Activin A activity and/or level, the antibody agent reduces Activin A activity and/or level, or prevents an increase in Activin A activity and/or level relative to a comparator.

In some embodiments, Activin A is or comprises free and/or active Activin A.

In some embodiments, an Activin A antibody agent reduces a plasma, blood, serum and/or urine level of Activin A. In some embodiments, an Activin A antibody agent reduces a level of Activin A to less than about 500 pg/mL.

In some embodiments, an Activin A antibody agent prevents an increase in Activin A levels in plasma, blood, serum and/or urine. In some embodiments, an Activin A antibody agent prevents an increase in Activin A level above about 500 pg/mL.

In some embodiments, an Activin A antibody agent reduces, e.g., inhibits, an Activin A activity. In some embodiments, an Activin A antibody agent reduces, e.g., inhibits, the activity and/or level of Activin A (e.g., plasma and/or urine Activin A, e.g., free and/or active Activin A) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, disclosed herein is a nucleic acid encoding an Activin A antibody agent. In some embodiments, a nucleic acid encoding an Activin A antibody agent is a DNA. In some embodiments, a nucleic acid encoding an Activin A antibody agent is an RNA, e.g., mRNA.

In some embodiments, also disclosed herein is a vector comprising a nucleic acid encoding an Activin A antibody.

In some embodiments, this disclosure provides a host cell comprising a vector disclosed herein.

In some embodiments, disclosed herein is a composition comprising an Activin A antibody agent. In some embodiments, a composition is or comprises a pharmaceutical composition.

In some embodiments, disclosed herein is a method comprising contacting an Activin A antibody agent (e.g., a composition comprising an Activin A antibody agent, e.g., a pharmaceutical composition), to a cell, tissue or subject. In some embodiments, a method comprises administering an Activin A antibody agent (e.g., a composition comprising an Activin A antibody agent, e.g., a pharmaceutical composition), to a cell, tissue or subject.

In some embodiments a method is a treatment method.

In some embodiments, a method is a prevention method.

In some embodiments, a subject disclosed herein has a condition or disorder associated with increased Activin A. In some embodiments, condition or disorder is chosen from: cachexia, mass loss (e.g., muscle mass loss, lean mass loss, fat mass loss, bone mass loss, and/or organ mass loss), weight loss (e.g., unintentional weight loss due to cachexia), senescence, liver damage (e.g., fibrosis, degeneration, cirrhosis [e.g., alcoholic and/or non-alcoholic cirrhosis]), kidney damage (e.g., fibrosis), cancer, cancer metastases, chemotherapy induced toxicity (e.g., nephrotoxicity and/or hepatotoxicity), chemoresistance and/or hypertension.

In some embodiments, condition or disorder is chosen from (i) disorders of the hypothalamic pituitary gonadal axis (e.g., with increased FSH levels); (ii) fibrodysplasia ossificans progressive (FOP); (iii) pulmonary arterial hypertension and/or hypertension; (iv) Anorexia-Cachexia Disorders associated with chronic diseases such as cancer, chronic kidney disease, chronic heart failure, COPD, elderly and muscle immobility, ICU patient (e.g., loss of body weight, loss of food intake, fat mass loss, muscle wasting, loss of functional muscle, loss of muscle strength, and bone loss, anemia and/or fatigue); (v) anemia; (vi) metabolic disorders (e.g., obesity, type 2 diabetes, and/or metabolic syndrome); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis); (viii) auto-immune disorders (e.g., SLE or rheumatoid arthritis); (ix) fibrotic diseases (e.g. idiopathic pulmonary fibrosis, NASH, liver cirrhosis, chronic kidney disease, Alport Syndrome, Uterine Fibroids, Endometriosis) (x) cancer (xi) cancer-treatment related toxicities (e.g., chemotherapy-induced nephrotoxicity and/or hepatotoxicity); (xii) aging (e.g., senescence) or (xiii) chemoresistance.

In some embodiments, disclosed herein is a method of treating and/or reversing mass loss in a subject, comprising administering to a subject an Activin A antibody agent (e.g., a composition comprising an Activin A antibody agent, e.g., a pharmaceutical composition). In some embodiments, mass comprises fat mass, lean mass, muscle mass, bone mass, organ mass or a combination thereof.

In some embodiments, disclosed herein is a method of treating and/or reversing weight loss in a subject, comprising administering to a subject an Activin A antibody agent (e.g., a composition comprising an Activin A antibody agent, e.g., a pharmaceutical composition). In some embodiments, weight loss is unintentional weight loss, e.g., as observed in cachexia.

In some embodiments, disclosed herein is a method of treating and/or reversing senescence in a subject, comprising administering to a subject an Activin A antibody agent (e.g., a composition comprising an Activin A antibody agent, e.g., a pharmaceutical composition).

In some embodiments, disclosed herein is a method of treating and/or preventing liver damage in a subject, comprising administering to a subject an Activin A antibody agent (e.g., a composition comprising an Activin A antibody agent, e.g., a pharmaceutical composition). In some embodiments, liver damage comprises: acute liver injury, liver necrosis, liver fibrosis, liver inflammation, liver degeneration, cirrhosis [e.g., alcoholic and/or non-alcoholic cirrhosis], alteration in liver function, elevation one or more liver enzymes, or a combination thereof.

In some embodiments, disclosed herein is a method of treating and/or preventing kidney damage in a subject, comprising administering to a subject an Activin A antibody agent (e.g., a composition comprising an Activin A antibody agent, e.g., a pharmaceutical composition). In some embodiments, kidney damage comprises acute kidney injury, kidney fibrosis, kidney inflammation, alteration in kidney function, or a combination thereof.

In some embodiments, disclosed herein is a method of treating and/or preventing cachexia in a subject, comprising administering to a subject an Activin A antibody agent (e.g., a composition comprising an Activin A antibody agent, e.g., a pharmaceutical composition). In some embodiments, cachexia is induced by a tumor. In some embodiments, cachexia comprises loss of appetite, muscle loss, fat mass loss, weight loss, or a combination thereof.

In some embodiments, disclosed herein is a method of treating and/or preventing SAR-COV-2 infection in a subject, comprising administering to a subject an Activin A antibody agent (e.g., a composition comprising an Activin A antibody agent, e.g., a pharmaceutical composition).

In some embodiments, disclosed herein is a method of reducing and/or preventing cancer growth in a subject, comprising administering to a subject an Activin A antibody agent (e.g., a composition comprising an Activin A antibody agent, e.g., a pharmaceutical composition). In some embodiments, a cancer comprises a colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer. In some embodiments, a cancer is a colorectal cancer.

In some embodiments, disclosed herein is a method of reducing and/or preventing chemoresistance in a subject, comprising administering to a subject an Activin A antibody agent (e.g., a composition comprising an Activin A antibody agent, e.g., a pharmaceutical composition). In some embodiments, chemoresistance is associated with Activin A expression. In some embodiments, chemoresistance is reduced and/or prevented in a subject having cancer who has been or is being administered a chemotherapy (e.g., as described herein). In some embodiments, a cancer comprises a colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer. In some embodiments, a cancer is a colorectal cancer.

In some embodiments, disclosed herein is a method of preventing and/or reducing migration of cells in vivo in a subject, comprising administering to a subject an Activin A antibody agent (e.g., a composition comprising an Activin A antibody agent, e.g., a pharmaceutical composition).

In some embodiments, disclosed herein is a method of reducing and/or preventing metastasis in a subject, comprising administering to a subject an Activin A antibody agent (e.g., a composition comprising an Activin A antibody agent, e.g., a pharmaceutical composition).

In some embodiments, disclosed herein is a method of reducing and/or preventing chemotherapy-induced toxicity in a subject, comprising administering to a subject an Activin A antibody agent (e.g., a composition comprising an Activin A antibody agent, e.g., a pharmaceutical composition). In some embodiments, a chemotherapy-induced toxicity is or comprises nephrotoxicity. In some embodiments, a chemotherapy is a Cisplatin based chemotherapy. In some embodiments, a chemotherapy is not a Cisplatin based chemotherapy.

In some embodiments, disclosed herein is a method of reducing and/or preventing hypertension in a subject, comprising administering to a subject an Activin A antibody agent (e.g., a composition comprising an Activin A antibody agent, e.g., a pharmaceutical composition).

In some embodiments of any of the methods disclosed herein, an Activin A antibody agent reduces, e.g., inhibits, an Activity of Activin A. In some embodiments, an Activin A antibody agent reduces, e.g., inhibits, the activity and/or level of Activin A (e.g., plasma and/or urine Activin A, e.g., free and/or active Activin A) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments of any of the methods disclosed herein, Activin A is or comprises free and/or active Activin A.

In some embodiments of any of the methods disclosed herein, an Activin A antibody agent reduces a plasma, blood, serum and/or urine level of Activin A. In some embodiments, an Activin A antibody agent reduces a level of Activin A to less than about 500 pg/mL.

In some embodiments of any of the methods disclosed herein, an Activin A antibody agent prevents an increase in Activin A levels in plasma, blood, serum and/or urine. In some embodiments, an Activin A antibody agent prevents an increase in Activin A level above about 500 pg/mL.

### BRIEF DESCRIPTION OF THE DRAWING

FIGS. 1A-1C are graphs showing binding affinity of three exemplary Activin A antibody agents (Clones B-D) to biotinylated Activin A as measured with a surface plasmon resonance assay. FIG. 1A is a graph showing data for biotinylated human Activin A Fc bound to the chip with 9 nM, 3 nM, 1 nM, 0.33 nM, or 0.11 nM Fab of Clone B in solution. FIG. 1B is a graph showing data for biotinylated human Activin A Fc bound to the chip with 9 nM, 3 nM, 1 nM, 0.33 nM, or 0.11 nM Fab of Clone C in solution. FIG. 1C is a graph showing data for biotinylated human Activin A Fc bound to the chip with 9 nM, 3 nM, 1 nM, 0.33 nM, or 0.11 nM Fab of Clone D in solution.

FIGS. 2A-2D depict inhibition of Activin A activity with two exemplary anti-Activin A antibodies. The assay used to measure Activin A activity is the Activin 2B Receptor/SMAD reporter assay as described in Example 2. FIG. 2A is a graph showing Activin A activity. FIG. 2B is a graph showing the activity of an IgG1 isotype control. FIG. 2C is a graph showing the inhibition of Activin A activity with anti-Activin antibody clone B. FIG. 2D is a graph showing the inhibition of Activin A activity with anti-Activin antibody clone C.

FIGS. 3A-3D depict no inhibition of BMP9 with activity with two exemplary anti-Activin A antibodies. The assay used to measure BMP9 activity is the SMAD reporter assay as described in Example 2. FIG. 3A is a graph showing BMP9 activity. FIG. 3B is a graph showing the activity of an IgG1 isotype control. FIG. 3C is a graph showing no inhibition of BMP9 activity with anti-Activin antibody clone B. FIG. 3D is a graph showing no inhibition of BMP9 activity with anti-Activin antibody clone C.

FIGS. 4A-4B show pharmacokinetic data with intravenous administration of two exemplary anti-Activin A antibodies in mice. FIG. 4A shows data with anti-Activin A antibody Clone B and FIG. 4B shows data with anti-Activin A antibody Clone C.

FIGS. 5A-5B show pharmacokinetic data with subcutaneous administration of two exemplary anti-Activin A antibodies in mice. FIG. 5A shows data with anti-Activin A antibody Clone B and FIG. 5B shows data with anti-Activin A antibody Clone C.

FIGS. 6A-6B show pharmacokinetic data with intravenous administration of two exemplary anti-Activin A antibodies in primates. FIG. 6A shows data with anti-Activin A antibody Clone B and FIG. 6B shows data with anti-Activin A antibody Clone C.

FIGS. 7A-7D show prevention of weight loss and stopping of weight loss with exemplary anti-Activin A antibodies in mice with Activin A-induced weight loss. For FIGS. 7A-7B, animals were grouped into 4 groups and dosed as follows: Null vector and IgG1 isotype (black circles), null vector and Clone C (purple boxes), high dose AAV-Activin A and IgG1 isotype (black triangles), and high dose AAV-Activin A and Clone C (purple triangles). FIG. 7A is a graph from the prevention study showing reduced weight loss in mice administered an exemplary anti-Activin A antibody one day before administration of high dose AAV-Activin A (compare purple downward-facing triangles with black upward-facing triangles). FIG. 7B is a graph from the therapeutic study showing stopping of weight loss in mice administered an exemplary anti-Activin A antibody after mice achieved 10% loss in weight (compare purple downward-facing triangles with black upward-facing triangles. Data analyzed by ANCOVA with body weight on Day 0 as co-variate followed by multiple t-test. *P<0.05, ***P<0.005 compared to null vector. ***P<0.005 compared to AAV-Activin and IgG1 Isotype control. For FIGS. 7C-7D mice were administered a lower dose of AAV-INHBA to increase circulating Activin A levels reported in patients (~3 ng/ml). This dose enabled mice to be studied chronically. For FIGS. 7C, mice were grouped into 4 groups and dosed as follows: Null vector and IgG1 isotype 10 mg/Kg (gray diamonds), AAV-Activin A and IgG1 isotope control 10 mg/Kg (black diamond), AAV-Activin A and clone C 1 mg/Kg (red upside down triangle) and AAV-Activin A and clone C 10 mg/Kg (purple circle). FIG. 7C is a graph from a therapeutic study showing full reversibly of weight loss in mice in a dose-dependent manner, subcutaneously administered an exemplary anti-Activin A antibody (Clone C) after mice achieved 20% loss in weight. Arrows denote dosing of Clone C. For FIG. 7D, mice were grouped into 4 groups and dosed as follows: Null vector and IgG1 isotype 10 mg/Kg (gray diamonds), AAV-Activin A and IgG1 isotope control 10 mg/Kg (black diamond), AAV-Activin A and clone C 1 mg/Kg (red diamond) and AAV-Activin A and clone C 10 mg/Kg (purple triangle). FIG. 7D is a graph from a therapeutic study showing full reversibility of weight loss in mice, in a dose-dependent manner, subcutaneously administered an exemplary anti-Activin A antibody (Clone B) after mice achieved 20% loss in weight. Arrows denote dosing of Clone B. ***P<0.005 compared to null vector and ###P<0.005 compared to AAV-Activin A and IgG1 isotype control.

FIGS. 8A-8H show prevention and reversal of organ mass loss with a single dose of an exemplary anti-Activin A antibody. FIGS. 8A-8D were administered a high dose of AAV-Activin A. FIG. 8A shows that in a prevention setting a single dose of an exemplary anti-Activin A antibody prevents heart muscle loss. FIG. 8B shows that in a therapeutic setting a single dose of an exemplary anti-Activin A antibody reverses heart muscle loss. FIG. 8C shows that in a prevention setting a single dose of an exemplary anti-Activin A antibody prevents liver mass loss after 6 days. FIG. 8D shows that in a therapeutic setting a single dose of an exemplary anti-Activin A antibody given within 2 days reverses liver mass loss. Means adjusted for differences between treatment groups in terminal body weight. Analysis by ANCOVA with terminal body weight as co-variate, followed by multiple t test. ***P<0.005 compared to null vector. ††P<0.01 †††P<0.005 compared to AAV-Activin A and IgG1 isotype control. FIGS. 8E-8H were administered a lower dose of AAV-Activin A chronically. Exemplary anti-Activin A antibodies were administered 12 days post AAV injection and dosed once weekly over 2 weeks. FIG. 8E shows reversal of heart mass loss with administration of 10 mg/kg of an exemplary anti-Activin A antibody. Similarly, FIG. 8F shows reversal of liver mass loss and FIG. 8H shows reversal of kidney mass loss with administration of 10 mg/kg of an exemplary anti-Activin A antibody. Reversal of pancreas mass loss (FIG. 8G) was observed with 1 mg/kg of an exemplary anti-Activin A antibody with a greater effect observed with 10 mg/kg of an exemplary anti-Activin A antibody. *P<0.05, ***P<0.006 compared to null vector. #P<0.05, ###P<0.005 compared to AAV-Activin A and IgG1 isotype control.

FIGS. 9A-9L depict over-expression of Activin A causes fibrosis and necrosis in mouse liver. FIG. 9A is a graph from acute over-expression of Activin A (6 days) showing inflammatory cell infiltration in control mice and Activin A overexpressing mice. FIG. 9B is a graph from acute over-expression of Activin A (6 days) showing fibrosis (collagen deposition) in control mice and Activin A overexpressing mice. FIG. 9C is a graph from acute over-expression of Activin A (6 days) showing hepatocellular necrosis in control mice and Activin A overexpressing mice. ***P<0.005 compared to null vector via exact Wilcoxon rank sum test for histology. FIGS. 9D-9L show gene expression data from chronic over-expression of Activin A. COL1A (FIG. 9D), COL3A (FIG. 9E), COL4A (FIG. 9F), smooth muscle actin (ATAC2, FIG. 9G), Fibronectin (FN1, FIG. 9H), and TGF-beta (FIG. 9I) levels were increased in the liver of mice chronically overexpressing Activin A for 39-51 days. FIGS. 9J-9L show levels of senescence markers p16 and p21 in the liver of mice chronically overexpressing Activin A for 39-51 days. The acute senescence marker p15 was unchanged. Comparisons to the null vector group are by the multiple t test at Day 39, *P<0.05, **P<0.01, ***P<0.001.

FIGS. 10A-10D show acute overexpression of high dose of AAV-Activin A (6 days) increases liver enzymes. FIG. 10A is a graph showing increased circulating ALT in control mice or mice overexpressing Activin A (AAV-Activin A). FIG. 10B is a graph showing increased circulating AST in control mice or mice overexpressing Activin A (AAV-Activin A). **P<0.01; ***P<0.005 compared to null vector via Kruskal-Wallis (non-parametric ANOVA). FIGS. 10C-D is chronic overexpression of a low dose of AAV-Activin A. FIGS. 10C-D is chronic overexpression of a low dose of AAV-Activin A. FIG. 10C is a graph showing circulating ALT and FIG. 10D is a graph showing circulating AST in control mice or mice overexpressing Activin A (AAV-Activin A). The AAV-Activin A animals were either therapeutically treated with a control antibody or were treated with an exemplary anti-Activin A antibody. Exemplary Anti-Activin A mAb fully reversed increased circulating ALT and AST induced by AAV-Activin A.***P<0.005 comparison to null vector or ###P<0.005 comparison to AAV-Activin A isotype control via ANOVA.

FIGS. 11A-11B show acute overexpression of Activin A (6 days) causes fibrosis and tubular degeneration in mouse kidneys. FIG. 11A is a graph showing collagen deposition (fibrosis) in control mice or mice overexpressing Activin A. FIG. 11B is a graph showing tubular degeneration in control mice or mice overexpressing Activin A. ***P<0.005 compared to null vector via exact Wilcoxon rank sum test for histology.

FIGS. 12A-12F show prevention of weight loss, fat mass loss and muscle mass loss in an orthotopic MC38 colorectal cancer model. Mice were either left untreated (sham), intrasplenically injected with MC38 cancer cells and administered an exemplary anti-Activin A antibody, or intrasplenically injected with MC38 cancer cells and administered a control antibody. FIG. 12A shows body weight changes, FIG. 12B shows carcass weight, FIG. 12C shows adipose tissue weight, FIG. 12D shows gastrocnemius muscle weight, FIG. 12E shows tibialis muscle weight, and FIG. 12F shows food intake. Data analysis using ANOVA*P<0.05, **P<0.01 and ***P<0.005 compared to MC38 mice with IgG1 isotype control.

FIGS. 13A-13C show primary tumor and liver metastasis in the MC38 colorectal cancer model. FIG. 13A shows tumor weight in the spleen of mice not inoculated with MC38 cells (left bar), mice inoculated with MC38 tumor cells into the spleen and treated with a control antibody (middle bar) and mice inoculated with MC38 tumor cells and treated with an exemplary anti-Activin A antibody (right bar). Exemplary anti-Activin A reduced primary tumor weight. Data analysis using ANOVA, *P<0.05 compared to MC38 mice with IgG1 isotype control. FIG. 13B and FIG. 13C each contain three image panels showing representative livers from: two sham mice (left panels), two MC38 inoculated mice treated with anti-Activin A antibody (middle panels), and two MC38 inoculated mice treated with a control antibody (right panels). Exemplary anti-Activin A antibody prevented liver metastases.

FIGS. 14A-14B show prevention of chemotherapy-induced nephrotoxicity in Wistar rats. An exemplary anti-Activin A antibody (20 mg/Kg, Day 1 & 3) or a control antibody was administered (pre-treatment) prior to cisplatin treatment (5 mg/Kg IP). FIG. 14A shows serum creatinine and FIG. 14B shows serum blood urea nitrogen (BUN) 5 days post cisplatin treatment in adult (6-7 weeks). Exemplary anti-Activin A antibody partially prevented nephrotoxicity, *P<0.05; ****P<0.0001 via one way ANOVA.

FIGS. 15A-15B show atrophy and anti-apoptosis in liver cells with chronic overexpression of Activin-A. FIG. 15A shows liver muRF1 gene expression and FIG. 15B shows BCL2 gene expression in control mice or mice overexpressing Activin A for 39-51 days. Comparisons to the null vector group are by the multiple t test at Day 39, *P<0.05 and ***P<0.001.

FIG. 16 shows chronic overexpression of Activin causes bone loss. FIG. 16 shows femur bone density images using X-ray computed tomography from individual control mice with a null vector (n=8) or mice overexpressing Activin A for 39 (n=7) or 51 (n=3) days.

FIGS. 17A-17E summarizes the chronic overexpression of Activin A causes bone loss as measured by X-ray computed tomography and Dual-Energy X-ray Absorptiometry (DEXA). Using X-ray computed tomography, FIG. 17A shows reduction in femur trabecular bone volume/total volume, FIG. 17B shows reduction of femur trabecular number, FIG. 17C shows reduction in femur trabecular thickness and FIG. 17D shows reduction in femur trabecular spacing induced by Activin A overexpression compared to mice expressing the null vector. Using DEXA, FIG. 17E shows reduction in whole body bone density in mice caused by Activin A overexpression compared to null vector control mice.

FIG. 18 shows that chronic overexpression of Activin-A caused a sustained increase in water intake in mice compared to control mice without Activin-A overexpression. Increased water intake is attributed to Activin A causing thirst, dehydration and a surrogate for hypertension.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Activin A: The term “Activin A” is used herein in reference to Activin A polypeptides as understood in the art. Activin A is a member of the TGFbeta superfamily. Activin A is also known as Activin beta-A chain or Inhibin beta-A chain. Under physiological conditions, Activin A can form a homodimer composed of two β subunits of inhibin linked by a disulfide bridge referred to as a βA/βA homodimer. Activin A protein is encoded by the INHBA gene. Amino acid sequences for full-length Activin A, and/or for nucleic acids that encode it can be found in a public database such as GenBank, UniProt and Swiss-Prot. For example, an amino acid sequence of human Activin A (SEQ ID NO: 215, for which residues 1-20 represent the signal peptide, and residues 311-426 represent a TGF beta family signature sequence, can be found as UniProt/Swiss-Prot Accession No. P08476; a nucleic acid sequence (SEQ ID NO: 216) encoding human Activin A can be found at Accession No. NM_002192.3. Those skilled in the art will appreciate that sequences presented in SEQ ID NOs: 215 and 216 are exemplary, and certain variations (including, for example, conservative substitutions in SEQ ID NO:215, codon-optimized variants of SEQ ID NO:216, etc) are understood to also be or encode human Activin A; additionally, those skilled in the art will appreciate that homologs and orthologs of human Activin A are known and/or knowable through the exercise or ordinary skill, for example, based on degree of sequence identity, presence of one or more characteristic sequence elements, and/or one or more shared activities. As will be clear from context, the term “Activin A” can be used in reference to monomeric Activin A and/or to homodimeric Activin A.

Activin A polypeptide: The phrase “Activin A polypeptide”, is used herein to refer to polypeptides that share significant sequence identity and/or at least one characteristic sequence element with an appropriate reference polypeptide such as, for example: (a) human Activin A, for example, as set forth in SEQ ID NO:215; (b) Rhesus macaque Activin A, for example as set forth in SEQ ID NO: 219 (see XP_028701686.1); (c) dog Activin A, for example as set forth in SEQ ID NO: 220 (see XP_038279632.1); and/or (d) cat Activin A for example as set forth in SEQ ID NO: 221 (see NP_001009856.1). In some embodiments, a Activin A polypeptide is or comprises a fragment of a parental Activin A polypeptide (e.g., of SEQ ID NO:215 or a homolog, ortholog, or variant [e.g., a functional variant] thereof). In some embodiments, a Activin A polypeptide shares at least one characteristic sequence element with a reference Activin A polypeptide (e.g., of SEQ ID NO:215 or a homolog, ortholog, or variant [e.g., a functional variant] thereof). Alternatively or additionally, in some embodiments, a Activin A polypeptide shares significant amino acid sequence identity with a relevant reference polypeptide (e.g., of SEQ ID NO:215 or a homolog, ortholog, or variant [e.g., a functional variant] thereof). For example, in some embodiments, a Activin A polypeptide shares at least 50% with a reference Activin A. In some embodiments, a Activin A polypeptide is characterized by an ability to activate a receptor that binds Activin A, e.g., a Type II receptor or a Type I receptor; in some such embodiments, such ability is comparable to that of an appropriate reference Activin A (e.g., of SEQ ID NO:215 or a homolog, ortholog, or variant [e.g., a functional variant] thereof). For example, in some embodiments, a Activin A polypeptide activates a Type II receptor or a Type I receptor with a binding affinity that is reasonably comparable to that of an appropriate reference Activin A (e.g., of SEQ ID NO:215 or a homolog, ortholog, or variant [e.g., a functional variant] thereof); in some embodiments, a Activin A polypeptide is characterized in that it competes with an appropriate reference Activin A (e.g., of SEQ ID NO:215 or a homolog, ortholog, or variant [e.g., a functional variant] thereof) for binding and/or activation of a Type II receptor or a Type I receptor; in some such embodiments, such competition is observed over a range of concentrations (e.g., which range may, for example, extend over 2 fold, 3 fold, 4 fold, 5 fold, 10 fold, or more). In some embodiments, a Activin A polypeptide is or comprises a polypeptide with at least 50% identity to SEQ ID NO: 215.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent that is, or is included in or otherwise delivered by, the composition. Those of ordinary skill in the art will be aware of a variety of routes that may, in appropriate circumstances, be utilized for administration to a subject, for example an animal or a human. In some embodiments, an animal is a domestic animal, such as a companion animal, e.g., a dog or a cat; in some embodiments, an animal is an animal used in agriculture (e.g., farming [e.g., a cow, a sheep or a horse]) or for recreation. For example, in some embodiments, administration may be systemic or local. Those skilled in the art will be aware of appropriate administration routes for use with particular therapies described herein, for example which include bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, an antibody agent can be formulated for oral delivery. For example, one with skill in the art will understand that an antibody agent disclosed herein can be formulated for oral delivery using technologies developed by Oramed (https://www.oramed.com/) or Premas (https://www.premasbiotech.com/).

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference”]. In some embodiments, affinity may be assessed relative to a contemporaneous reference; in some embodiments, affinity may be assessed relative to a historical reference. Typically, when affinity is assessed relative to a reference, it is assessed under comparable conditions.

Affinity matured” (or “affinity matured antibody”): as used herein, refers to an antibody with one or more alterations in one or more CDRs or FR thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., BioTechnology 10:779-783 (1992) describes affinity maturation by VH and VL domain shuffling. Random mutagenesis of CDR and/or framework residues is described by: Barbas et al. Proc. Nat. Acad. Sci. U.S.A 91:3809-3813 (1994); Schier et al., Gene 169:147-155 (1995); Yelton et al., J. Immunol. 155:1994-2004 (1995); Jackson et al., J. Immunol. 154(7): 3310-9 (1995); and Hawkins et al., J. Mol. Biol. 226:889-896 (1992).

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to a member of the animal kingdom. In some embodiments, “animal” refers to humans; unless otherwise specified, in many embodiments, a human may be of either gender and/or at any stage of development. In some embodiments, “animal” refers to non-human animals; unless otherwise specified, in many embodiments, a non-human animal may be of any gender and/or at any stage of development. In certain embodiments, a non-human animal is a mammal (e.g., a rodent, a mouse, a rat, a rabbit, a monkey, a dog, a cat, a sheep, cattle, a primate, and/or a pig). In some embodiments, an animal may be, for example, a mammals, a bird, a reptile, an amphibian, a fish, an insect, a worm, etc. In some embodiments, an animal may be a transgenic animal, genetically engineered animal, and/or a clone.

Antagonist: Those skilled in the art will appreciate that the term “antagonist”, as used herein, may be used to refer to an agent, condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain, e.g., an effector null mutation, e.g., a LALA, LAGA, FEGG, AAGG, or AAGA mutation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies, etc. In some embodiments, relevant formats may be or include: Adnectins®; Affibodies®; Affilins®; Anticalins®; Avimers®; BiTERs; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-Bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of dog, cat, mouse, rabbit, primate, or human antibodies. In some embodiments, an antibody agent may include one or more sequence elements that are human, humanized, primatized, chimeric, etc, as is known in the art. In some embodiments, an antibody agent may include one or more complementarity determining regions that are human and/or one or more constant region sequences that are characteristic of human antibodies. In many embodiments, the term “antibody agent” is used to refer to one or more of the art-known or developed constructs or formats for utilizing antibody structural and functional features in alternative presentation. For example, in some embodiments, an antibody agent utilized in accordance with the present disclosure is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide comprising an antigen binding specificity fused to an Fc; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINS®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, we observe that ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antibody polypeptide: As used herein, the term “antibody polypeptide” refers to a polypeptide(s) that includes characteristic sequence element(s) (e.g., one or more CDRs, or a set of CDRs such as each of a CDR1, 2, and 3 as found in reference antibody chain and/or one or more FR regions and/or a set of FR regions, such as, for example, a complete variable region of a heavy or light chain of a reference antibody) of an antibody; in many embodiments, an antibody polypeptide includes sufficient such sequence element(s) that it binds to an epitope (e.g., an epitope bound by a reference antibody including the characteristic sequence element). In some embodiments, an antibody polypeptide is a full-length antibody or heavy or light chain thereof. In some embodiments, an antibody polypeptide is or comprises a complete heavy and/or light chain variable region of a reference antibody; in some such embodiments, an antibody polypeptide includes characteristic antibody sequence element(s) sufficient to confer specific binding to a relevant epitope—i.e., so that the antibody polypeptide includes at least one binding site. In some embodiments, an “antibody polypeptide” may include a binding domain which is homologous or largely homologous (e.g., shows significant sequence homology and/or in some embodiments significant sequence identity) to an immunoglobulin-binding domain. In some embodiments, an antibody polypeptide shows at least 99% identity with an immunoglobulin binding domain. In some embodiments, an “antibody polypeptide” has a binding domain that shows at least 70%, 80%, 85%, 90%, or 95% identity with an immuglobulin binding domain, for example a reference immunoglobulin binding domain. In some embodiments, an “antibody polypeptide” may have an amino acid sequence identical to that of an antibody, or chain, or variable region thereof (or combination of variable region(s)) that is found in a natural source. In some embodiments, an antibody polypeptide may be prepared by, for example, isolation from a natural source or antibody library, recombinant production in or with a host system, chemical synthesis, etc., or combinations thereof. In some embodiments, an antibody polypeptide is an antibody agent as described herein.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, ovarian and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed subject matter.

CDR-grafted antibody: as used herein, refers to an antibody whose amino acid sequence comprises heavy and light chain variable region sequences from one species but in which the sequences of one or more of the CDR regions of VH and/or VL are replaced with CDR sequences of another species, such as antibodies having murine VH and VL regions in which one or more of the murine CDRs (e.g., CDR3) has been replaced with human CDR sequences. Likewise, a “CDR-grafted antibody” may also refer to antibodies having human VH and VL regions in which one or more of the human CDRs (e.g., CDR3) has been replaced with mouse CDR sequences.

Child: As used herein, the term “child” refers to a human between 1 day and 18 years of age. In some embodiments, a child may be an infant (e.g., may be less than or equal to about 12 months, 11 months, 10 months, 9 months, 8 months, 7 months, 6 months, 5 months, 4 months, 3 months, 2 months or 1 month old); in some embodiments, a child may be older than an infant. In some embodiments, a child may be a toddler (e.g., about 1 to about 3 years old); in some embodiments, a child may be younger than or older than a toddler. In some embodiments, a child may be a teen (e.g., between about 12 and about 18 years old); in some embodiments, a child may be younger than a teen (and/or older or younger than a toddler or older than an infant). Body weight can vary widely across ages and specific children, with a typical range being 4 pounds to 150 pounds.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, alpha-sheet character, coiled-coil character, random coil character, etc.), and/or by a particular functional feature (e.g., binding activity, enzymatic activity, folding activity, signaling activity, etc.).

Effector function: as used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: as used herein refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Framework” or “framework region: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

High affinity binding: The term “high affinity binding”, as used herein refers to a high degree of tightness with which a particular ligand binds to its partner. Affinities can be measured by any available method, including those known in the art. In some embodiments, binding is considered to be high affinity if the K_{d} is about 500 pM or less (e.g., below about 400 pM, about 300 pM, about 200 pM, about 100 pM, about 90 pM, about 80 pM, about 70 pM, about 60 pM, about 50 pM, about 40 pM, about 30 pM, about 20 pM, about 10 pM, about 5 pM, about 4 pM, about 3 pM, about 2 pM, etc.) in binding assays. In some embodiments, binding is considered to be high affinity if the affinity is stronger (e.g., the K_{d} is lower) for a polypeptide of interest than for a selected reference polypeptide. In some embodiments, binding is considered to be high affinity if the ratio of the K_{d} for a polypeptide of interest to the K_{d} for a selected reference polypeptide is 1:1 or less (e.g., 0.9:1, 0.8:1, 0.7:1, 0.6:1, 0.5:1. 0.4:1, 0.3:1, 0.2:1, 0.1:1, 0.05:1, 0.01:1, or less). In some embodiments, binding is considered to be high affinity if the K_{d} for a polypeptide of interest is about 100% or less (e.g., about 99%, about 98%, about 97%, about 96%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1% or less) of the K_{d} for a selected reference polypeptide.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes VH and VL region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized VL region. In some embodiments, a humanized antibody only contains a humanized VH region. In some certain embodiments, a humanized antibody contains humanized VH and VL regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

K_{D}: as used herein, refers to the dissociation constant of a binding agent (e.g., an antibody or binding component thereof) from a complex with its partner (e.g., the epitope to which the antibody or binding component thereof binds).

Low affinity binding: The term “low affinity binding”, as used herein refers to a low degree of tightness with which a particular ligand binds to its partner. As described herein, affinities can be measured by any available method, including methods known in the art. In some embodiments, binding is considered to be low affinity if the K_{d} is about 501 pM or more (e.g., above about 501 pM, 600 pM, 700 pM, 800 pM, 900 pM, 1 nM, 1.1.nM, 1.2 nM, 1.3 nM, 1.4 nM, 1.5 nM, etc.) In some embodiments, binding is considered to be low affinity if the affinity is the same or lower (e.g., the K_{d} is about the same or higher) for a polypeptide of interest than for a selected reference polypeptide. In some embodiments, binding is considered to be low affinity if the ratio of the K_{d} for a polypeptide of interest to the K_{d} for a selected reference polypeptide is 1:1 or more (e.g., 1.1:1, 1.2:1, 1.3:1, 1.4:1, 1.5:1. 1.6:1, 1.7:1, 1.8:1, 1.9:1, 2:1, 3:1, 4:1, 5:1, 10:1 or more). In some embodiments, binding is considered to be low affinity if the K_{d} for a polypeptide of interest is 100% or more (e.g., 100%, 105%, 110%, 115%, 120%, 125%, 130%, 135%, 140%, 145%, 150%, 155%, 160%, 165%, 170%, 175%, 180%, 185%, 190%, 195%, 200%, 300%, 400%, 500%, 1000%, or more) of the K_{d} for a selected reference polypeptide.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR; in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Treat: As used herein, the term “treat,” “treatment,” or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits only early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a variant also differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION

The present disclosure provides, among other things, novel Activin A antibody agents which have improved binding kinetics, binding affinity, pharmacokinetics, and/or function, e.g., compared to an appropriate reference anti-Activin A antibody. In some embodiments, an Activin A antibody agent disclosed herein binds to Activin A with high affinity and/or high specificity. For example, in some embodiments, an Activin A antibody agent binds to Activin A with a K_{D} of about 5 pM to about 1000 pM (e.g., about 6.17 pM to about 960 pM), e.g., as assessed in an assay that measures antibody binding affinity, e.g., a Surface Plasmon Resonance assay (Biacore).

In some embodiments, a provided Activin A antibody agent may show preferential binding to Activin A relative to one or more TGFbeta family members other than Activin A. In some such embodiments, preferential binding may be assessed, for example, by simultaneously contacting a Activin A antibody agent with Activin A and one or more other TGFbeta family members. Alternatively or additionally, in some embodiments, preferential binding may be assessed relative to an appropriate reference Activin A antibody agent and, e.g., may reflect a higher level of binding to Activin A relative to the one or more other TGFbeta family member than is observed with the reference antibody. In some embodiments, a Activin A antibody agent does not bind to one or more TGFbeta family members other than Activin A. In some embodiments, a Activin A antibody agent disclosed herein binds to Activin A and also binds to one or more other TGFbeta family members. For example, in some embodiments, a provided Activin A antibody agent binds to Activin A and to Activin B or GDF11, or both.

In some embodiments, an Activin A antibody agent disclosed herein inhibits an activity of Activin A and/or reduces a level of Activin A (e.g., reduces a level of Activin A in blood, plasma, serum, and/or urine) when administered to a cell, tissue or subject. In some embodiments, a Activin A antibody agent disclosed herein can be used to treat a condition or disease associated with increased Activin A. In some embodiments, a Activin A antibody agent disclosed herein can be used to treat a symptom of a condition or disease associated with increased Activin A. In some embodiments, a symptom comprises any one, all, or a combination of: weight loss, loss of appetite, fatigue, muscle mass loss, fat mass loss, lean mass loss, lean mass atrophy, functional muscle mass loss, loss of muscle strength, bone loss, anemia, or fibrosis. Also provided herein are compositions comprising Activin A antibody agents disclosed herein, as well as methods of making and using the same.

### Activin A

Activin A is a protein of approximately 25 kDa that forms a homodimer via a disulfide bridge with another Activin A protein and is a member of the transforming growth factor beta (TGFbeta) superfamily (Morianos I. et al (2019) Journal of Autoimmunity, Volume 104). Activin A protein is synthesized as a precursor polypeptide contain an amino terminal prodomain with 250-350 residues and a carboxy terminal mature domain (Bloise E. et al., (2018) Physiological Reviews, 99(1)). The precursor polypeptide is cleaved by a furin-like protease releasing the mature protein which is biologically active (see Bloise E et al (2018)).

A human Activin A polypeptide sequence is provided herein as SEQ ID NO: 215:

MPLLWLRGFLLASCWIIVRSSPTPGSEGHSAAPDCPSCALAALPKDVPNSQPE MVEAVKKHILNMLHLKKRPDVTQPVPKAALLNAIRKLHVGKVGENGYVEIEDDIGRRA EMNELMEQTSEIITFAESGTARKTLHFEISKEGSDLSVVERAEVWLFLKVPKANRTRTKV TIRLFQQQKHPQGSLDTGEEAEEVGLKGERSELLLSEKVVDARKSTWHVFPVSSSIQRLL DQGKSSLDVRIACEQCQESGASLVLLGKKKKKEEEGEGKKKGGGEGGAGADEEKEQSH RPFLMLQARQSEDHPHRRRRRGLECDGKVNICCKKQFFVSFKDIGWNDWIIAPSGYHAN YCEGECPSHIAGTSGSSLSFHSTVINHYRMRGHSPFANLKSCCVPTKLRPMSMLYYDDG QNIIKKDIQNMIVEECGCS The amino acid sequence of human Activin A (SEQ ID NO: 215) comprises a signal peptide (residues 1-20) and a mature Activin A sequence (residues 311-426).

Human Activin A signal peptide: MPLLWLRGFLLASCWIIVRS (SEQ ID NO: 217)

Mature Human Activin A sequence:

(SEQ ID NO: 218)
GLECDGKVNICCKKQFFVSFKDIGWNDWIIAPSGYHANYCEGECPSHIAG
TSGSSLSFHSTVINHYRMRGHSPFANLKSCCVPTKLRPMSMLYYDDGQNI
IKKDIQNMIVEECGCS

Human Activin A can be encoded by the following nucleic acid sequence from the INHBA gene (SEQ ID NO: 216):

| | |
|---|---|
| 1 | agtacagtat aaaacttcac agtgccaata ccatgaagag gagctcagac agctcttacc |
| 61 | acatgataca agagccggct ggtggaagag tggggaccag aaagagaatt tgctgaagag |
| 121 | gagaaggaaa aaaaaaacac caaaaaaaaa aataaaaaaa tccacacaca caaaaaaacc |
| 181 | tgcgcgtgag gggggaggaa aagcagggcc ttttaaaaag gcaatcacaa caacttttgc |
| 241 | tgccaggatg cccttgcttt ggctgagagg atttctgttg gcaagttgct ggattatagt |
| 301 | gaggagttcc cccaccccag gatccgaggg gcacagcgcg gcccccgact gtccgtcctg |
| 361 | tgcgctggcc gccctcccaa aggatgtacc caactctcag ccagagatgg tggaggccgt |
| 421 | caagaagcac attttaaaca tgctgcactt gaagaagaga cccgatgtca cccagccggt |
| 481 | acccaaggcg gcgcttctga acgcgatcag aaagcttcat gtgggcaaag tcggggagaa |
| 541 | cgggtatgtg gagatagagg atgacattgg aaggagggca gaaatgaatg aacttatgga |
| 601 | gcagacctcg gagatcatca cgtttgccga gtcaggaaca gccaggaaga cgctgcactt |
| 661 | cgagatttcc aaggaaggca gtgacctgtc agtggtggag cgtgcagaag tctggctctt |
| 721 | cctaaaagtc cccaaggcca acaggaccag gaccaaagtc accatccgcc tottccagca |
| 781 | gcagaagcac ccgcagggca gcttggacac aggggaagag gccgaggaag tgggcttaaa |
| 841 | gggggagagg agtgaactgt tgctctctga aaaagtagta gacgctcgga agagcacctg |
| 901 | gcatgtcttc cctgtctcca gcagcatcca gcggttgctg gaccagggca agagctccct |
| 961 | ggacgttcgg attgcctgtg agcagtgcca ggagagtggc gccagcttgg ttctcctggg |
| 1021 | caagaagaag aagaaagaag aggaggggga agggaaaaag aagggcggag gtgaaggtgg |
| 1081 | ggcaggagca gatgaggaaa aggagcagtc gcacagacct ttcctcatgc tgcaggcccg |
| 1141 | gcagtctgaa gaccaccctc atcgccggcg tcggcggggc ttggagtgtg atggcaaggt |
| 1201 | caacatctgc tgtaagaaac agttctttgt cagtttcaag gacatcggct ggaatgactg |
| 1261 | gatcattgct ccctctggct atcatgccaa ctactgcgag ggtgagtgcc cgagccatat |
| 1321 | agcaggcacg tccgggtcct cactgtcctt ccactcaaca gtcatcaacc actaccgcat |
| 1381 | gcggggccat agcccctttg ccaacctcaa atcgtgctgt gtgcccacca agctgagacc |
| 1441 | catgtccatg ttgtactatg atgatggtca aaacatcatc aaaaaggaca ttcagaacat |
| 1501 | gatcgtggag gagtgtgggt gctcatagag ttgcccagcc cagggggaaa gggagcaaga |
| 1561 | gttgtccaga gaagacagtg gcaaaatgaa gaaattttta aggtttctga gttaaccaga |
| 1621 | aaaatagaaa ttaaaaacaa aacaaaaaaa aaaacaaaaa aaaacaaaag taaattaaaa |
| 1681 | acaaaacctg atgaaacaga tgaaggaaga tgtggaaaaa atccttagcc agggctcaga |
| 1741 | gatgaagcag tgaaagagac aggaattggg agggaaaggg agaatggtgt accctttatt |
| 1801 | tcttctgaaa tcacactgat gacatcagtt gtttaaacgg ggtattgtcc tttcccccct |
| 1861 | tgaggttccc ttgtgagcct tgaatcaacc aatctagtct gcagtagtgt ggactagaac |
| 1921 | aacccaaata gcatctagaa agccatgagt ttgaaagggc ccatcacagg cactttccta |
| 1981 | cccaattacc caggtcataa ggtatgtctg tgtgacactt atctctgtgt atatcagcat |
| 2041 | acacacacac acacacacac acacacacac acacaggcat ttccacacat tacatatata |
| 2101 | cacatactgg taaaagaaca atcgtgtgca ggtggtcaca cttccttttt ctgtaccact |
| 2161 | tttgcaacaa aacaaaacaa acaacattaa aaaattgaga acaagtatgg aaagaatgaa |
| 2221 | agatcaagga aaaaagaata ccaagttaca tttcgttaag gtgcttatga tcttagaact |
| 2281 | atgcaaccta ataggtttga aactgtttac ctgagagaga acaaaaagag agactttttt |
| 2341 | gtattggaag taatctgatt aatttttatt ttcttcaagg agagatactt gaaaggaata |
| 2401 | tgtttgtcca tctgttggat ccaaacattt ctatattttg taaatgttgt tgttgttttt |
| 2461 | tttttaatcg tttactattt gcactacaat ggtgtttgac ctgtctaatc cttatttaac |
| 2521 | aagtattttc tttggttggg ggtgggggtg gggtttaaga gctgcactta atgtgagcta |
| 2581 | taaaagaact gctacagcac acaaaatagc tatttttatt attataatta taattattat |
| 2641 | tattattttg taccttaaaa aatagacaca tacaccaaag acatttgtgt gagcctttaa |
| 2701 | acagtctgtc tgtggttggt atcattcacc atcaatgagt caggggttgg gattcaaggt |
| 2761 | tgagtagtgt ggattgtgtt caggcttaaa agacctgaga agtttggttt ttgactcctt |
| 2821 | ttacatccat gaaacaggac atttcatact ggatgtacag tagttgtaca ctgttggata |
| 2881 | tcaagttcaa tcaaattcat ggaactacat gcttgtatgt gtatatatac attgcttgtg |
| 2941 | catatgcata tctgtatgta tatatacatg tattgtacca tgtccataca cattttaagc |
| 3001 | acttcaggct gtcatttttt aatgttotta aagcaatgaa tgtttgtgtg caaaacacag |
| 3061 | tatttttaag aaggataggc tatagttttt gcttttactc tgaactaggt gggcgcattt |
| 3121 | caaaaattcg gatgggaaaa agcctggaaa ttccagtgaa tattcagcaa ggccctcttt |
| 3181 | cattgtacag ggatcaaatt tcctcctctt ttttgtgccc cctcccactt ctacaagtta |
| 3241 | tcccctgtgg ggaaaacagg atgataatca aaactctggg ctgatgtttt tccaacttag |
| 3301 | tgtctattgg aatcaatctt aaatcagaag ctttttcaga aaaataatat ttaggccaga |
| 3361 | attagagttg agtgtatttt ttaaaaatga ttaaggcttg gttgtgagaa atattacctg |
| 3421 | taccagctgg gaaaaataat gtcatcacta actaaaagat aattaatttg agagaaagtg |
| 3481 | ttaagagagg gagagtaagg aagagaacag ttaagaggag gcagaggtga gggcagtagt |
| 3541 | aaaaatctct aaaattttaa tttacagcca aaattcttca tgtgtaaatt tgtattgatt |
| 3601 | cagatgcaga aatgaaaaaa aaacaccttt gttttataaa tatcaaagta catgottaaa |
| 3661 | gccaagtttt tatctagttt attctagtac ttagcttgcc tggaatagct aataaattat |
| 3721 | tcatgtatgt gcttttgaaa atccagagcc ctatttttac acacttgtgt gaagttggca |
| 3781 | aacattttga aaaatggaaa aaagtttcta ataattggga acaattacat taattaatat |
| 3841 | tttgtaaaat attgaagctt ttagccctat gtcaatttgt agattaaaat aaattaatta |
| 3901 | taggaaagga agataacagt gagaaaccaa acattacaaa aggtggttta gctctccttg |
| 3961 | aaaaatatac taagttggta tactataaca cttggctata tgtaggcaat gtcactactg |
| 4021 | ggcaaataca cttactgtgt tctagaggca gccctttctt atgcagaaaa tacaatacgc |
| 4081 | actgcatgag aagcttgaga gtggattcta atccaggtct gtcgaccttg gatatcatgc |
| 4141 | atgtgggaag gtgggtgtgg tgagaaaagt tttaaggcaa gagtagatgg ccatgttcaa |
| 4201 | ctttacaaaa tttcttggaa aactggcagt attttgaact gcatcttctt tggtaccgga |
| 4261 | acctgcagaa acagtgtgag aaattaagtc ctggttcact gcgcagtagc aaagatggtc |
| 4321 | aaggccatgg aaaaagcaga aatttaccaa gaaagctgat acccatgtat agttcccact |
| 4381 | catctcaaat acatctgcta tctttttaag ctaagtccta gacatatcgg ggataacatg |
| 4441 | ggggttgatt agtgaccaca gttatcagaa gcagagaaat gtaattccat attttatttg |
| 4501 | aaacttattc catattttaa ttggatattg agtgattggg ttatcaaaca cccacaaact |
| 4561 | ttaattttgt taaatttata tggctttgaa atagaagtat aagttgctac cattttttga |
| 4621 | taacattgaa agatagtatt ttaccatctt taatcatctt ggaaaataca agtcctgtga |
| 4681 | acaaccactc tttcacctag cagcatgagg ccaaaagtaa aggctttaaa ttataacata |
| 4741 | tgggattctt agtagtatgt ttttttcttg aaactcagtg gctctatcta accttactat |
| 4801 | ctcctcactc tttctctaag actaaactct aggctcttaa aaatctgccc acaccaatct |
| 4861 | tagaagctct gaaaagaatt tgtctttaaa tatcttttaa tagtaacatg tattttatgg |
| 4921 | accaaattga cattttcgac tattttttcc aaaaaagtca ggtgaatttc agcacactga |
| 4981 | gttgggaatt tcttatccca gaagaccaac caatttcata tttatttaag attgattcca |
| 5041 | tactccgttt tcaaggagaa tccctgcagt ctccttaaag gtagaacaaa tactttctat |
| 5101 | ttttttttca ccattgtggg attggacttt aagaggtgac tctaaaaaaa cagagaacaa |
| 5161 | atatgtctca gttgtattaa gcacggaccc atattatcat attcacttaa aaaaatgatt |
| 5221 | tcctgtgcac cttttggcaa cttctctttt caatgtaggg aaaaacttag tcaccctgaa |
| 5281 | aacccacaaa ataaataaaa cttgtagatg tgggcagaag gtttgggggt ggacattgta |
| 5341 | tgtgtttaaa ttaaaccctg tatcactgag aagctgttgt atgggtcaga gaaaatgaat |
| 5401 | gcttagaagc tgttcacatc ttcaagagca gaagcaaacc acatgtctca gctatattat |
| 5461 | tatttatttt ttatgcataa agtgaatcat ttcttctgta ttaatttcca aagggtttta |
| 5521 | ccctctattt aaatgctttg aaaaacagtg cattgacaat gggttgatat ttttctttaa |
| 5581 | aagaaaaata taattatgaa agccaagata atctgaagcc tgttttattt taaaactttt |
| 5641 | tatgttctgt ggttgatgtt gtttgtttgt ttgtttctat tttgttggtt ttttactttg |
| 5701 | ttttttgttt tgttttgttt tgttttgcat actacatgca gttctttaac caatgtctgt |
| 5761 | ttggctaatg taattaaagt tgttaattta tatgagtgca tttcaactat gtcaatggtt |
| 5821 | tcttaatatt tattgtgtag aagtactggt aattttttta tttacaatat gtttaaagag |
| 5881 | ataacagttt gatatgtttt catgtgttta tagcagaagt tatttatttc tatggcattc |
| 5941 | cagcggatat tttggtgttt gcgaggcatg cagtcaatat tttgtacagt tagtggacag |
| 6001 | tattcagcaa cgcctgatag cttctttggc cttatgttaa ataaaaagac ctgtttggga |
| 6061 | tgtaaaaaaa aaaaaaaaaa aaaaaaaaaa aaaaaaaaaa aaaaa |

Activin A can bind to a Type I receptor or a Type II receptor. Binding of Activin A to a Type II receptor, e.g., ActRIIA or AcRIIB, recruits and phosphorylates a Type I receptor (e.g., ActRI or ALK4). Upon activation of a Type I receptor, Smad2 and Smad 3 proteins are phosphorylated and appropriate downstream signal cascades are activated. In addition, activation of a Type I receptor can also result in activation of pathways such as the p38 MAPK pathway, ERK1/2 pathway, and JNK pathway.

Without wishing to be bound by any particular theory, in some embodiments, an Activin A antibody agent disclosed herein binds to Activin A and inhibits binding of endogenous Activin A (e.g., active Activin A) to an Activin A receptor (e.g., a Type II receptor [e.g., ACTRIIA or ACTRIIB] and/or a Type I receptor [e.g., ActRI or ALK4]) thus preventing activation of a Type II and/or Type I receptor and/or one or more downstream signaling pathways. In some embodiments, modulation of an Activin A receptor with an Activin A antibody agent disclosed herein allows for treatment of conditions, diseases or disorder associate with (e.g., mediated by) Activin A.

Further, without wishing to be bound by any particular theory, in some embodiments, an Activin A antibody agent disclosed herein binds to Activin A and inhibits binding of endogenous Activin A to a receptor, e.g., a receptor that binds to Activin A, e.g., an Activin A receptor (e.g., a Type II receptor [e.g., ACTRIIA or ACTRIIB] and/or a Type I receptor [e.g., ActRI or ALK4]). In some embodiments, an Activin A receptor is accessible to an antibody agent, e.g., as disclosed herein.

While certain anti-Activin A antibodies have been previously developed (see, e.g., WO2015017576), none of the art known antibodies have so far been demonstrated as feasible therapeutic options for preventing and treating conditions, diseases or disorders, or symptoms thereof associated with (e.g., mediated by) elevated levels of Activin A; moreover, none could teach or suggest the particular antibodies or antibody agents described herein, which show remarkable and unexpected properties, including as documented herein. In some embodiments, an elevated level of Activin A is about 500 pg/ml or more, e.g., as evaluated in a sample from a subject, e.g., a blood, plasma, serum or urine sample. In some embodiments, a provided antibody agent blocks and/or reverses weight loss even in dramatic weight loss contexts (e.g., models) including, for example, potently and/or completely blocks functional signaling via the Activin A receptor; alternatively or additionally, in some embodiments, a provided antibody agent prevents or reverses mass loss (e.g., muscle mass loss, lean mass loss, fat mass loss, and/or bone mass loss), weight loss, senescence, liver damage, kidney damage, or cancer. Thus, the present disclosure provides novel Activin A antibody agents which can be used to meet the unmet need of treating and/or preventing conditions, diseases or disorders, or symptoms thereof, that are associated with elevated Activin A.

### Activin A Antibody Agent

Disclosed herein are Activin A antibody agents which bind, e.g., specifically bind, to Activin A, e.g., with high affinity. An anti-Activin A antibody disclosed herein, may be effective in the plasma and/or multiple tissue compartments, where Activin A can act on its target cells, e.g., a cell expressing a receptor that binds to Activin A.

In some embodiments, an Activin A target cell is or comprises a cell expressing an Activin A receptor (e.g., a Type II receptor [e.g., ACTRIIA or ACTRIIB] and/or a Type I receptor [e.g., ActRI or ALK4]). In some embodiments, an Activin A antibody agent can modulate an Activin A receptor pathway to inhibit one or more activities of Activin A or to reduce a level of Activin A, e.g., reduce a level of free and/or active Activin A, e.g., in blood, plasma, serum and/or urine. In some embodiments, an Activin A antibody agent disclosed herein binds to Activin A and inhibits an activity and/or reduces a level of Activin A, e.g., reduce a level of free and/or active Activin A, e.g., in blood, plasma, serum and/or urine. In some embodiments, an Activin A antibody agent disclosed herein binds to Activin A and prevents binding of Activin A to an Activin A receptor (e.g., a Type II receptor [e.g., ACTRIIA or ACTRIIB] and/or a Type I receptor [e.g., ActRI or ALK4]). In some embodiments, binding of an Activin A antibody agent to Activin A prevents activation of an Activin A receptor (e.g., a Type II receptor [e.g., ACTRIIA or ACTRIIB] and/or a Type I receptor [e.g., ActRI or ALK4]).

In some embodiments, an Activin A target cell is or comprises a cell expressing an Activin A receptor. In some embodiments, an Activin A antibody agent disclosed herein binds to Activin A and inhibits an activity and/or reduces a level of Activin A, e.g., reduce a level of free and/or active Activin A. In some embodiments, an Activin A antibody agent disclosed herein binds to Activin A and prevents binding of Activin A to an Activin A receptor (e.g., a Type II receptor [e.g., ACTRIIA or ACTRIIB] and/or a Type I receptor [e.g., ActRI or ALK4]).

In some embodiments, an Activin A antibody agent disclosed herein binds to any or all forms of Activin A, e.g., homodimeric Activin A, monomeric Activin A, intracellular Activin A, soluble Activin A, ECM-bound Activin A, mature Activin A, pro-protein Activin A (e.g., preprocessed) and/or active Activin A.

In some embodiments, an Activin A binding agent specifically binds Activin A and reduces a level of Activin A (e.g., free and/or active Activin A). In some embodiments, a level of homodimeric Activin A is reduced. In some embodiments, a level of circulating Activin A is reduced. In some embodiments, a level of free and/or active Activin A is reduced. In some embodiments, a level of free and active Activin A is reduced.

In some embodiments, a level of Activin A (e.g., free and/or active Activin A) is reduced relative to a comparator. In some embodiments, a comparator comprises a cell, tissue or subject which has not been contacted with an Activin A antibody agent disclosed herein.

In some embodiments, a level of Activin A (e.g., free and/or active Activin A) is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or about 100%. In some embodiments, a level of Activin A (e.g., free and/or active Activin A) is reduced by about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 90% to about 100%, or about 95% to about 100%.

In some embodiments, a level of Activin A (e.g., free and/or active Activin A) is reduced by about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, or about 5% to about 10%.

In some embodiments, an Activin A activity comprises an activity of an Activin receptor. In some embodiments, an Activin A activity comprises an activity of one or more signaling pathways activated by an Activin receptor. In some embodiments, an Activin receptor mediated signaling pathway comprises one or more of: (i) a SMAD pathway, (ii) an ERK/MAPK pathway, (iii) a p38 MAPK pathway, (iv) NOX-2 dependent signaling; or (v) Snail and/or Slug mediated pathways.

Disclosed herein are Activin A antibody agents that can modulate an activity of Activin A. In some embodiments, an Activin A antibody agent specifically binds Activin A and modulates one or more, or all, or any combination of detectable Activin A activities such that the antibody agent: modulates an Activin A receptor activity, modulates a SMAD pathway, modulates an ERK/MAPK pathway, modulates a p38 MAPK pathway, modulates NOX-2 dependent signaling, modulates Snail and/or Slug mediated pathways, or a combination thereof.

One with skill in the art will appreciate that, in some embodiments, an activity of Activin A, e.g., as described herein can be assessed using one or more art recognized assays. For example, as disclosed in Example 2 herein, an Activin 2B Receptor/SMAD reporter assay can be used to evaluate Activin A activity.

Human Activin A protein sequence is highly conserved with primate, rodents, domestic mammals, birds, and other species such as panda, seal, sloth and whale. Table 15 provides a summary of the sequence homology between human Activin A and Activin A in various species. The list provided in Table 15 is an exemplary list of species having highly conserved Activin A as compared to human Activin A. Many other animals and birds, in addition to those disclosed in Table 15 have Activin A protein sequences that are highly homologous (e.g., >95%) to human Activin A. One with skill in the art will understand in reading this disclosure that known methods to determine sequence homology can be used to identify Activin A from other species which share a high sequence homology with human Activin A, e.g., as described herein.

TABLE 15
Activin A homology across various species

| | | Sequence | Homology | | |
|---|---|---|---|---|---|
| | Identifier | Length | to Human | Species | |
| | XP_016867664.1 | 116 | | Homo sapiens | Human |
| Primates | XP_519063.2 | 116 | 100% | Pan troglodytes | Chimpanzee |
| and | XP_003808743.1 | 116 | 100% | Pan paniscus | Chimpanzee, Pygmy |
| Monkeys | XP_028701686.1 | 116 | 100% | Macaca mulatta | Rhesus macaque |
| | XP_011760680.1 | 116 | 100% | Macaca nemestrina | Southern pig-tailed macaque |
| | XP_002751400.2 | 116 | 100% | Callithrix jacchus | Marmoset monkey |
| | XP_017369490.1 | 116 | 100% | Cebus imitator | Panamanian White-faced Capuchin |
| | XP_007979729.1 | 116 | 100% | Chlorocebus sabaeus | green monkey |
| Rodents | NP_058824.1 | 116 | 100% | Rattus norvegicus | Rat |
| | CAJ18587.1 | 116 | 100% | Mus musculus | Mouse |
| | XP_012970113.1 | 116 | 100% | Mesocricetus auratus | Golden hamster |
| | NP_001315998.1 | 116 | 100% | Oryctolagus cuniculus | Rabbit |
| | XP_013008060.1 | 116 | 100% | Cavia porcellus | Guinea pig |
| | XP_005397780.1 | 116 | 100% | Chinchilla lanigera | Chinchilla |
| Domestic | NP_001009856.1 | 116 | 100% | Felis catus | Cat |
| Mammals | XP_038279632.1 | 116 | 97% | Canis lupus familiaris | Dog |
| | XP_004739411.1 | 116 | 100% | Mustela putorius furo | Ferret |
| | NP_001075378.1 | 116 | 97% | Equus caballus | Horse |
| | AAA97415.1 | 116 | 100% | Bos taurus | Cow |
| | KAG5210458.1 | 116 | 100% | Ovis aries | Sheep |
| | NP_999193.1 | 116 | 100% | Sus scrofa | Pig |
| | XP_010945482.1 | 116 | 100% | Camelus bactrianus | Bactrian camel |
| | MXQ80611.1 | 116 | 100% | Bos mutus | Yak |
| Birds | XP_015136806.2 | 116 | 98% | Gallus gallus | Chicken |
| | NWZ62453.1 | 116 | 100% | Acrocephalus arundinaceus | Great Reed Warbler |
| | NXT88066.1 | 116 | 100% | Anhinga rufa | African darter (bird) |
| Other | XP_002914183.1 | 116 | 100% | Ailuropoda melanoleuca | Giant Panda |
| (from | XP_025708917.1 | 116 | 100% | Callorhinus ursinus | Northern Fur Seal |
| >100) | XP_037693065.1 | 116 | 100% | Choloepus didactylus | Two-toed Sloth |
| | MBV94806.1 | 116 | 100% | Eschrichtius robustus | California grey whale |

Based on the high homology between human Activin A and Activin A from other species as shown in Table 14, one with skill in the art would understand that an Activin A antibody agent disclosed herein would bind to one or more species provided in Table 14.

In some embodiments, an Activin A antibody agent disclosed herein binds specifically to human Activin A. In some embodiments, an Activin A antibody agent disclosed herein binds specifically to primate Activin A. In some embodiments, an Activin A antibody agent disclosed herein binds specifically to domestic mammals Activin A. In some embodiments, domestic mammals are chosen from: dog, cat, ferret, horse, cow, sheep, pig, Bactrian camel, and yak.

In some embodiments, a provided Activin A antibody agent may show preferential binding to Activin A relative to one or more TGFbeta family members other than Activin A. In some such embodiments, preferential binding may be assessed, for example, by simultaneously contacting an Activin A antibody agent with Activin A and one or more other TGFbeta family members. Alternatively or additionally, in some embodiments, preferential binding may be assessed relative to an appropriate reference Activin A antibody agent (e.g., as described in WO2015017576) and, e.g., may reflect a higher level of binding to Activin A relative to the one or more other TGFbeta family member than is observed with the reference antibody.

In some embodiments, an Activin A antibody agent disclosed herein preferentially binds to Activin A. In some embodiments, an Activin A antibody agent disclosed herein does not bind to one or more members of the TGFbeta super family other than Activin A. In some embodiments, an Activin A antibody agent disclosed herein does not bind to GDNF, GDF8, GDF10, GDF11, BMP9, BMP 10, Activin B or a combination thereof.

In some embodiments, an Activin A antibody agent disclosed herein preferentially binds to Activin A. In some embodiments, an Activin A antibody agent disclosed herein binds to one or more members of the TGFbeta super family in addition to Activin A. In some embodiments, an Activin A antibody agent disclosed herein binds to Activin A and Activin B, or GDF11, or both. In some embodiments, an Activin A antibody agent disclosed herein binds to Activin A and Activin B. In some embodiments, an Activin A antibody agent disclosed herein binds to Activin A and GDF11. In some embodiments, an Activin A antibody agent disclosed herein binds to Activin A, Activin B and GDF11.

In some embodiments, an Activin A binding agent which binds to Activin A and Activin B does not modulate an activity and/or level of Activin B, e.g., when characterized in an assay that evaluates an Activin B activity and/or level.

In some embodiments, an Activin A binding agent which binds to Activin A and GDF11 does not modulate an activity and/or level of GDF11, e.g., when characterized in an assay that evaluates a GDF11 activity and/or level.

One with skill in the art will appreciate that, in some embodiments, an activity of Activin B, or GDF11, e.g., as described herein can be assessed using one or more art recognized assays. For example, as disclosed in Example 2 herein, an assay using cells expressing an Activin 2B Receptor/SMAD reporter can be used to evaluate Activin B, or GDF11 activity. Several assays can be used to measure activation of the Activin 2B Receptor and induction of SMAD signaling following stimulation with Activin B, or GDF11, e.g., a luciferase-based reporter system.

Without wishing to be bound by theory, even though provided Activin A antibody agents can bind to Activin A and one or more members of the TGFbeta super family, a modulatory impact of provided Activin A antibody agents towards Activin A may be independent of binding to one or more TGFbeta super family members.

Those skilled in the art, reading the present disclosure will appreciate that, in some embodiments, an antibody agent provided by the present disclosure comprises: (i) an intact IgA, IgG, IgD, IgE or IgM antibody; (ii) an antibody fragment (e.g., an antibody variable region, containing both heavy and light chain sequences, e.g., a Fab); (iii) a single domain antibody (e.g., a light chain antibody or a heavy chain antibody); (iv) a single chain antibody (e.g., a single chain Fv, a camelid antibody, etc); (v) an antibody-drug conjugate; (vi) a bi- or other multispecific antibody; (vii) a polypeptide comprising antigen binding specificity fused to an Fc region; etc.

Those skilled in the art, reading the present disclosure will further appreciate that provided contributions are not limited to intact antibodies or fragments thereof (e.g., including both heavy and light chain sequences).

For example, those skilled in the art will appreciate that individual light chains and/or individual heavy chains, or variable region sequences thereof, as described herein (e.g., as exemplified herein, for example as presented in Table 1) may be useful in combination with other light chains and/or heavy chains. In some embodiments, a single light chain described herein (or variable region sequences thereof) may be utilized together with two (or more) different heavy chains (e.g., which may be or comprise heavy chains exemplified herein), or variable region sequences thereof, in a “common light chain” bispecific format. In some embodiments, exemplified light and heavy chains (e.g., variable region sequences thereof) may be “mixed and matched” with one another in antibody agents provided by the present disclosure (e.g., antibody agents that specifically bind to Activin A and/or have one or more other structural and/or functional properties as described herein.

Still further, those skilled in the art will appreciate that the present disclosure provides useful heavy and light chain antibody sequences, specifically including useful variable region sequences, including for example useful CDR and/or framework (FR) sequences.

In some embodiments, the present disclosure provides polypeptides (which may, for example, be, or be included in, an antibody agent that binds specifically to Activin A) including one or more CDR and/or FR sequences as set forth in Table 1 or 2. In some embodiments, the present disclosure provides polypeptides including two or more CDR elements from Table 1 or 2, and in particular the present disclosure provides polypeptides including three or six CDR elements from Table 1 or 2.

In some embodiments, the present disclosure provides polypeptides (which may, for example, be, or be included in, an antibody agent that binds specifically to Activin A) including one LC CDR1, one LC CDR2, and one LC CDR3 from Table 1; in some such embodiments, two or three of the CDRs are from the same LC in Table 1.

In some embodiments, the present disclosure provides polypeptides (which may, for example, be, or be included in, an antibody agent that binds specifically to Activin A) including one HC CDR1, one HC CDR2, and one HC CDR3 from Table 2; in some such embodiments, two or three of the CDRs are from the same HC in Table 2.

In some embodiments, the present disclosure provides polypeptides (which may, for example, be, or be included in, an antibody agent that binds specifically to Activin A) including one each of a LC CDR1, a LC CDR2, a LC CDR3, a HC CDR1, a HC CDR2, and a HC CDR3 from Table 1 or 2; in some such embodiments, two or more CDRs, and in some embodiments all LC CDRs, all HC CDRS, or both, are from the same antibody in Table 1 or 2.

Those skilled in the art will further appreciate that, in some embodiments, useful polypeptides as described herein that include one or more CDRs from Table 1 or 2 may include a heavy or light chain CDR set (i.e., each of a CDR1, a CDR2, and a CDR3) that includes one or two CDRs from a first antibody chain (i.e., LC or HC) in Table 1 or 2, and at least one from a second antibody chain (e.g., of the same type) in Table 1 or 2. Alternatively or additionally, in some embodiments, those skilled in the art will appreciate that useful polypeptides as described herein that include one or more CDRs from Table 1 or 2 may include a heavy or light chain CDR set (i.e., each of a CDR1, a CDR2, and a CDR3) that includes at least one CDR from a first antibody chain (i.e., LC or HC) in Table 1 or 2 and at least one other CDR that differs from its corresponding CDR in the relevant chain in Table 1 or 2. In some such embodiments, the differing CDR(s) will differ at not more than 3, not more than 2, or not more than 1 positions relative to the corresponding CDR(s); alternatively or additionally, in some such embodiments, the differing CDR(s) will differ only at terminal residue(s) relative to the corresponding CDR(s).

In some embodiments, an Activin A antibody agent disclosed herein which binds to Activin A comprises a LC CDR1, a LC CDR2 and a LC CDR3 provided in Table 1. In some embodiments, the presence of a LC CDR1, a LC CDR2 and a LC CDR3 is sufficient to confer binding and/or is otherwise useful in an antibody agent disclosed herein (i.e., that specifically binds to Activin A). In some embodiments, an antibody agent comprising a LC CDR1, a LC CDR2 and a LC CDR3 can be in any format disclosed herein. For example, in some embodiments, an antibody agent comprising a LC CDR1, a LC CDR2 and a LC CDR3 can be a single chain antibody, and is capable of binding Activin A.

In some embodiments, an Activin A antibody agent disclosed herein which binds to Activin A comprises a HC CDR1, a HC CDR2 and a HC CDR3 is sufficient to confer binding and/or is otherwise useful in an antibody agent disclosed herein to Activin A. In some embodiments, an antibody agent comprising a HC CDR1, a HC CDR2 and a HC CDR3 can be in any format disclosed herein. For example, in some embodiments, an antibody agent comprising a HC CDR1, a HC CDR2 and a HC CDR3 can be a single chain antibody, and is capable of binding Activin A.

In some embodiments, an Activin A antibody agent disclosed herein which binds to Activin A comprises a set of any three LC CDRs (e.g., LC CDR1, LC CDR2 and LC CDR3) provided in Table 1, and a set of any three HC CDRs (e.g., HC CDR1, HC CDR2 and HC CDR3) provided in Table 2. In some embodiments, the presence of a set of any three LC CDRs and a set of any three HC CDRs is sufficient to confer binding of any antibody agent disclosed herein to Activin A. In some embodiments, such an Activin A antibody agent can be a fragment (e.g., an scFv, a Fab or other fragments), or an intact antibody, or a polypeptide comprising antigen binding specificity fused to an Fc.

In some embodiments, disclosed herein is an Activin A antibody agent that competes (e.g., when tested in a standard competition assay) for binding to human Activin A with a different Activin A antibody agent, e.g., an Activin A antibody agent disclosed in WO2015017576. In some embodiments, an Activin A antibody agent disclosed herein competes for binding to human Activin A with a different Activin A antibody agent when assessed at more than one concentration (e.g., over a concentration range of at least 2-, 4-, 6-, 8-, 10-fold or more).

In some embodiments, disclosed herein is an Activin A antibody agent that does not compete (e.g., when tested in a standard competition assay) for binding to human Activin A with a different Activin A antibody agent, e.g., an Activin A antibody agent disclosed in WO2015017576

In some embodiments, disclosed herein is an Activin A antibody agent that binds to a sterically overlapping (e.g., partially or completely overlapping) epitope as an Activin A antibody agent disclosed in WO2015017576.

### Light Chain (e.g., Light Chain Variable Region) Polypeptides (LC Polypeptides)

The present disclosure provides polypeptides comprising light chain (LC) sequences (e.g., light chain variable region sequence(s)) that, for example, may be useful in antibody agents as described herein targeting Activin A; in some such embodiments, such provided polypeptides are useful and/or included in such antibody agents as described herein. In some embodiments, a LC polypeptide comprises at least one LC CDR provided in Table 1 or a sequence with at least 85% identity thereto. In some embodiments, a LC polypeptide comprises one, two or three LC CDRs (e.g., a LC CDR1, a LC CDR2 and/or a LC CDR3). In some embodiments, a LC polypeptide comprises a LC CDR1. In some embodiments, a LC polypeptide comprises a LC CDR2. In some embodiments, a LC polypeptide comprises a LC CDR3. In some embodiments, a LC polypeptide comprises a LC CDR1, a LC CDR2 and a LC CDR3.

In some embodiments, a LC polypeptide having a LC CDR1, a LC CDR2 and a LC CDR3, e.g., in an Activin A antibody agent, is capable of binding (e.g., specifically binding) to Activin A.

In some embodiments, a LC polypeptide further comprises one or more framework regions, and/or a constant region.

In some embodiments, a LC polypeptide comprises a light chain constant region and/or a heavy chain constant region. In some embodiments, a LC polypeptide comprises a light chain constant region or a portion thereof, (e.g., a lambda light chain constant region or a variant or portion thereof; or a kappa light chain constant region or a variant or a portion thereof).

In some embodiments, a light chain kappa constant region comprises the sequence of SEQ ID NO: 102, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 102.

(SEQ ID NO: 102, light chain kappa)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, a LC polypeptide comprises the sequence of SEQ ID NO: 102, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 102.

In some embodiments, a LC polypeptide disclosed herein further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, a LC polypeptide comprises: (i) an LC CDR1 sequence provided in Table 1, e.g., any one of SEQ ID NOs: 1, 11, 28, 53, 68, 85, or 93; (ii) a sequence with at least 85%, 86%, 87%, 88%, 89%, 90%, 91%, 92%, 93%, 94%, 95%, 96%, 97%, 98%, 99% identity to an LC CDR1 sequence provided in Table 1, e.g., any one of SEQ ID NOs: 1, 11, 28, 53, 68, 85, or 93; or (iii) a sequence having at least 5, 10, or 20 substitutions relative to an LC CDR1 sequence provided in Table 1, e.g., any one of SEQ ID NOs: 1, 11, 28, 53, 68, 85, or 93.

In some embodiments, a LC polypeptide comprises: (i) an LC CDR2 sequence provided in Table 1, e.g., any one of SEQ ID NOs: 2, 16, 29, 43, 48, 54, 61, 69, 86 or 94; (ii) a sequence with at least 85%, 86%, 87%, 88%, 89%, 90%, 91%, 92%, 93%, 94%, 95%, 96%, 97%, 98%, 99% identity to an LC CDR2 sequence provided in Table 1 e.g., any one of SEQ ID NOs: 2, 16, 29, 43, 48, 54, 61, 69, 86 or 94; or (iii) a sequence having at least 5, 10, or 20 substitutions relative to an LC CDR2 sequence provided in Table 1, e.g., any one of SEQ ID NOs: 2, 16, 29, 43, 48, 54, 61, 69, 86 or 94.

In some embodiments, a LC polypeptide comprises: (i) an LC CDR3 sequence provided in Table 1, e.g., any one of SEQ ID NOs: 3, 12, 17, 21, 30, 36, 44, 49, 55, 62, 70, 80, 87 or 95; (ii) a sequence with at least 85%, 86%, 87%, 88%, 89%, 90%, 91%, 92%, 93%, 94%, 95%, 96%, 97%, 98%, 99% identity to an LC CDR3 sequence provided in Table 1, e.g., any one of SEQ ID NOs: 3, 12, 17, 21, 30, 36, 44, 49, 55, 62, 70, 80, 87 or 95; or (iii) a sequence having at least 5, 10, or 20 substitutions relative to an LC CDR3 sequence provided in Table 1, e.g., any one of SEQ ID NOs: 3, 12, 17, 21, 30, 36, 44, 49, 55, 62, 70, 80, 87 or 95.

In some embodiments, an antibody agent comprising a LC CDR1, LC CDR2 and/or LC CDR3 is capable of binding specifically to Activin A.

In some embodiments, a LC polypeptide comprises (i) an LC CDR1, LC CDR2, and LC CDR3 sequence provided in Table 1; (ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or (iii) a sequence having at least 5, 10, or 20 substitutions relative to an LC CDR1, LC CDR2, and LC CDR3 sequence provided in Table 1.

In some embodiments, a LC polypeptide comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; (ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or (iii) an LC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3.

In some embodiments, a LC polypeptide comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; (ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or (iii) an LC CDR3 of SEQ ID NO: 12, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 12.

In some embodiments, a LC polypeptide comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; (ii) an LC CDR2 of SEQ ID NO: 16, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 16; and/or (iii) an LC CDR3 of SEQ ID NO: 17, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 17.

In some embodiments, a LC polypeptide comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; (ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or (iii) an LC CDR3 of SEQ ID NO: 21, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 21.

In some embodiments, a LC polypeptide comprises: (i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; (ii) an LC CDR2 of SEQ ID NO: 29 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 29; and/or (iii) an LC CDR3 of SEQ ID NO: 30, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 30.

In some embodiments, a LC polypeptide comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; (ii) an LC CDR2 of SEQ ID NO: 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or (iii) an LC CDR3 of SEQ ID NO: 36, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 36.

In some embodiments, a LC polypeptide comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; (ii) an LC CDR2 of SEQ ID NO: 43 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 43; and/or (iii) an LC CDR3 of SEQ ID NO: 44, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 44.

In some embodiments, a LC polypeptide comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; (ii) an LC CDR2 of SEQ ID NO: 48 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 48; and/or (iii) an LC CDR3 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49.

In some embodiments, a LC polypeptide comprises: (i) an LC CDR1 of SEQ ID NO: 53, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 53; (ii) an LC CDR2 of SEQ ID NO: 54 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 54; and/or (iii) an LC CDR3 of SEQ ID NO: 55, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 55.

In some embodiments, a LC polypeptide comprises: (i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; (ii) an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and/or (iii) an LC CDR3 of SEQ ID NO: 62, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 62.

In some embodiments, a LC polypeptide comprises: (i) an LC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; (ii) an LC CDR2 of SEQ ID NO: 69 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and/or (iii) an LC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70.

In some embodiments, a LC polypeptide comprises: (i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; (ii) an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and/or (iii) an LC CDR3 of SEQ ID NO: 80, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 80.

In some embodiments, a LC polypeptide comprises: (i) an LC CDR1 of SEQ ID NO: 85, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 85; (ii) an LC CDR2 of SEQ ID NO: 86 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 86; and/or (iii) an LC CDR3 of SEQ ID NO: 87, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 87.

In some embodiments, a LC polypeptide comprises: (i) an LC CDR1 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; (ii) an LC CDR2 of SEQ ID NO: 94 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and/or (iii) an LC CDR3 of SEQ ID NO: 95, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 95.

In some embodiments, a LC polypeptide further comprises one or more framework regions (FR), e.g., as described herein. In some embodiments, a LC polypeptide comprises one, two, three or four FRs, e.g., as described herein. In some embodiments, a FR comprises a LC FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a LC FR sequence as described herein, or a sequence having at least 5, 10 or 20 alterations relative to a LC FR sequence as described herein.

In some embodiments, a LC polypeptide comprises: (i) a FR sequence provided in Table 1; (ii) a sequence with at least 92%, 93%, 94%, 95%, 96%, 97%, 98%, or 99% identity to a FR sequence provided in Table 1; or (iii) a sequence having at least 5, 10, or 20 alterations (e.g., substitutions, deletions or insertions (e.g., conservative substitutions)) compared to a FR sequence provided in Table 1.

In some embodiments, a LC polypeptide comprises a LC FR1 provided in Table 1, a sequence with at least 92%, 93%, 94%, 95%, 96%, 97%, 98%, or 99% identity to a LC FR1 sequence provided in Table 1, or a sequence having at least 5, 10, or 20 alterations (e.g., substitutions, deletions or insertions (e.g., conservative substitutions)) compared to a LC FR1 sequence provided in Table 1.

In some embodiments, a LC polypeptide comprises a LC FR2 provided in Table 1, a sequence with at least 92%, 93%, 94%, 95%, 96%, 97%, 98%, or 99% identity to a LC FR2 sequence provided in Table 1, or a sequence having at least 5, 10, or 20 alterations (e.g., substitutions, deletions or insertions (e.g., conservative substitutions)) compared to a LC FR2 sequence provided in Table 1.

In some embodiments a LC polypeptide comprises a LC FR3 provided in Table 1, a sequence with at least 92%, 93%, 94%, 95%, 96%, 97%, 98%, or 99% identity to a LC FR3 sequence provided in Table 1, or a sequence having at least 5, 10, or 20 alterations (e.g., substitutions, deletions or insertions (e.g., conservative substitutions)) compared to a LC FR3 sequence provided in Table 1.

In some embodiments, a LC polypeptide comprises a LC FR4 provided in Table 1, a sequence with at least 92%, 93%, 94%, 95%, 96%, 97%, 98%, or 99% identity to a LC FR4 sequence provided in Table 1, or a sequence having at least 5, 10, or 20 alterations (e.g., substitutions, deletions or insertions (e.g., conservative substitutions)) compared to a LC FR4 sequence provided in Table 1.

In some embodiments, a LC polypeptide comprises a LC CDR1, a LC CDR2 and LC CDR3 provided in Table 1 or a sequence with at least 85% identity thereto, and a LC FR1, LC FR2, LC FR3 and a LC FR4 of a provided in Table 1 or a sequence with at least 92% identity thereto.

In some embodiments, a LC polypeptide comprises the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8.

In some embodiments, a LC polypeptide comprises the sequence of SEQ ID NO: 13, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 13.

In some embodiments, a LC polypeptide comprises the sequence of SEQ ID NO: 18, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 18.

In some embodiments, a LC polypeptide comprises the sequence of SEQ ID NO: 22, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 22.

In some embodiments, a LC polypeptide comprises the sequence of SEQ ID NO: 33, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 33.

In some embodiments, a LC polypeptide comprises the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37.

In some embodiments, a LC polypeptide comprises the sequence of SEQ ID NO: 45, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 45.

In some embodiments, a LC polypeptide comprises the sequence of SEQ ID NO: 50, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 50.

In some embodiments, a LC polypeptide comprises the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58.

In some embodiments, a LC polypeptide comprises the sequence of SEQ ID NO: 65, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 65.

In some embodiments, a LC polypeptide comprises the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74.

In some embodiments, a LC polypeptide comprises the sequence of SEQ ID NO: 82, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 82.

In some embodiments, a LC polypeptide comprises the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90.

In some embodiments, a LC polypeptide comprises the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99.

In some embodiments, a LC polypeptide comprises an LC sequence provided in Table 1, e.g., any one of SEQ ID NOs: 9, 14, 19, 23, 34, 38, 41, 46, 51, 59, 66, 75, 83, 91, 100, or a sequence with at least 85%, 86%, 87%, 88%, 89%, 90%, 91%, 92%, 93%, 94%, 95%, 96%, 97%, 98%, 99% identity thereto; or a sequence having at least 5, 10, or 20 alterations (e.g., substitutions, deletions or insertions (e.g., conservative substitutions)) relative to any one of SEQ ID NOs: 9, 14, 19, 23, 34, 38, 41, 46, 51, 59, 66, 75, 83, 91, 100.

Exemplary useful LC polypeptides which may be included in Activin A antibody agents disclosed herein are disclosed in Table 1 below.

TABLE 1
Exemplary light chain sequences for Activin A antibody agents

| | SEQ | | |
|---|---|---|---|
| | ID | | |
| CLONE | NO | Feature | SEQUENCE |
| Antibody A | 1 | LC CDR1 | RASQSVRSSYLA |
| VL | 2 | LC CDR2 | GASSRAT |
| germline | 3 | LC CDR3 | QQYVSYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 8 | VL amino | EIVLTQSPGTLSLSPGERATLSCRASQSVRSSYLAWYQQKPGQ |
| | | acid | APRLLIYGASSRATGIPDRFSGSGSGTDFTLTISRLEPEDFAVYY |
| | | | CQQYVSYPRTFGGGTKVEIK |
| | 9 | LC | EIVLTQSPGTLSLSPGERATLSCRASQSVRSSYLAWYQQKPGQ |
| | | | APRLLIYGASSRATGIPDRFSGSGSGTDFTLTISRLEPEDFAVYY |
| | | | CQQYVSYPRTFGGGTKVEIKRTVAAPSVFIFPPSDEQLKSGTAS |
| | | | VVCLLNNFYPREAKVQWKVDNALQSGNSQESVTEQDSKDSTY |
| | | | SLSSTLTLSKADYEKHKVYACEVTHQGLSSPVTKSFNRGEC |
| | 10 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGGAGCAGCTACTTAGCCTGGTACCAGCAGAAACCTG |
| | | | GCCAGGCTCCCAGGCTCCTCATCTATGGTGCATCCAGCAGG |
| | | | GCCACTGGCATCCCAGACAGGTTCAGTGGCAGTGGGTCTGG |
| | | | GACAGACTTCACTCTCACCATCAGCAGACTGGAGCCTGAAGA |
| | | | TTTTGCAGTGTATTACTGTCAGCAGTACGTCAGTTACCCTAG |
| | | | GACTTTTGGCGGAGGGACCAAGGTTGAGATCAAA |
| Antibody B | 11 | LC CDR1 | RASQSVSSSYLA |
| VL | 2 | LC CDR2 | GASSRAT |
| germline | 12 | LC CDR3 | QQYLSYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 13 | VL amino | EIVLTQSPGTLSLSPGERATLSCRASQSVSSSYLAWYQQKPGQ |
| | | acid | APRLLIYGASSRATGIPDRFSGSGSGTDFTLTISRLEPEDFAVYY |
| | | | CQQYLSYPRTFGGGTKVEIK |
| | 14 | LC | EIVLTQSPGTLSLSPGERATLSCRASQSVSSSYLAWYQQKPGQ |
| | | | APRLLIYGASSRATGIPDRFSGSGSGTDFTLTISRLEPEDFAVYY |
| | | | CQQYLSYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 15 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGCAGCAGCTACTTAGCCTGGTACCAGCAGAAACCTGG |
| | | | CCAGGCTCCCAGGCTCCTCATCTATGGTGCATCCAGCAGGG |
| | | | CCACTGGCATCCCAGACAGGTTCAGTGGCAGTGGGTCTGGG |
| | | | ACAGACTTCACTCTCACCATCAGCAGACTGGAGCCTGAAGAT |
| | | | TTTGCAGTGTATTACTGTCAGCAGTACCTGAGTTACCCTAGG |
| | | | ACTTTTGGCGGAGGGACCAAGGTTGAGATCAAA |
| Antibody C | 11 | LC CDR1 | RASQSVSSSYLA |
| VL | 16 | LC CDR2 | GASSRAG |
| germline | 17 | LC CDR3 | QQYVAYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 18 | VL amino | EIVLTQSPGTLSLSPGERATLSCRASQSVSSSYLAWYQQKPGQ |
| | | acid | APRLLIYGASSRAGGIPDRFSGSGSGTDFTLTISRLEPEDFAVYY |
| | | | CQQYVAYPRTFGGGTKVEIK |
| | 19 | LC | EIVLTQSPGTLSLSPGERATLSCRASQSVSSSYLAWYQQKPGQ |
| | | | APRLLIYGASSRAGGIPDRFSGSGSGTDFTLTISRLEPEDFAVYY |
| | | | CQQYVAYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 20 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGCAGCAGCTACTTAGCCTGGTACCAGCAGAAACCTGG |
| | | | CCAGGCTCCCAGGCTCCTCATCTATGGTGCATCCAGCAGGG |
| | | | CCGGAGGCATCCCAGACAGGTTCAGTGGCAGTGGGTCTGGG |
| | | | ACAGACTTCACTCTCACCATCAGCAGACTGGAGCCTGAAGAT |
| | | | TTTGCAGTGTATTACTGTCAGCAGTACGTCGCGTACCCTAGG |
| | | | ACTTTTGGCGGAGGGACCAAGGTTGAGATCAAA |
| Antibody D | 11 | LC CDR1 | RASQSVSSSYLA |
| VL | 2 | LC CDR2 | GASSRAT |
| germline | 21 | LC CDR3 | LQYGSYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 22 | VL amino | EIVLTQSPGTLSLSPGERATLSCRASQSVSSSYLAWYQQKPGQ |
| | | acid | APRLLIYGASSRATGIPDRFSGSGSGTDFTLTISRLEPEDFAVYY |
| | | | CLQYGSYPRTFGGGTKVEIK |
| | 23 | LC | EIVLTQSPGTLSLSPGERATLSCRASQSVSSSYLAWYQQKPGQ |
| | | | APRLLIYGASSRATGIPDRFSGSGSGTDFTLTISRLEPEDFAVYY |
| | | | CLQYGSYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 24 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGCAGCAGCTACTTAGCCTGGTACCAGCAGAAACCTGG |
| | | | CCAGGCTCCCAGGCTCCTCATCTATGGTGCATCCAGCAGGG |
| | | | CCACTGGCATCCCAGACAGGTTCAGTGGCAGTGGGTCTGGG |
| | | | ACAGACTTCACTCTCACCATCAGCAGACTGGAGCCTGAAGAT |
| | | | TTTGCAGTGTATTACTGTTTGCAGTACGGGAGTTACCCTAGG |
| | | | ACTTTTGGCGGAGGGACCAAGGTTGAGATCAAA |
| Antibody E | 1 | LC CDR1 | RASQSVRSSYLA |
| VL | 2 | LC CDR2 | GASSRAT |
| germline | 3 | LC CDR3 | QQYVSYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 8 | VL amino | EIVLTQSPGTLSLSPGERATLSCRASQSVRSSYLAWYQQKPGQ |
| | | acid | APRLLIYGASSRATGIPDRFSGSGSGTDFTLTISRLEPEDFAVYY |
| | | | CQQYVSYPRTFGGGTKVEIK |
| | 9 | LC | EIVLTQSPGTLSLSPGERATLSCRASQSVRSSYLAWYQQKPGQ |
| | | | APRLLIYGASSRATGIPDRFSGSGSGTDFTLTISRLEPEDFAVYY |
| | | | CQQYVSYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 10 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGGAGCAGCTACTTAGCCTGGTACCAGCAGAAACCTG |
| | | | GCCAGGCTCCCAGGCTCCTCATCTATGGTGCATCCAGCAGG |
| | | | GCCACTGGCATCCCAGACAGGTTCAGTGGCAGTGGGTCTGG |
| | | | GACAGACTTCACTCTCACCATCAGCAGACTGGAGCCTGAAGA |
| | | | TTTTGCAGTGTATTACTGTCAGCAGTACGTCAGTTACCCTAG |
| | | | GACTTTTGGCGGAGGGACCAAGGTTGAGATCAAA |
| Antibody F | 28 | LC CDR1 | RASQSVSSNLA |
| VL | 29 | LC CDR2 | GASDRAT |
| germline | 30 | LC CDR3 | IQYSNYPRT |
| VK3-15 | 31 | LC FR1 | EIVLTQSPATLSVSPGERATLSC |
| | 5 | LC FR2 | WYQQKPGQAPRLLIY |
| | 32 | LC FR3 | GIPARFSGSGSGTEFTLTISSLQSEDFAVYYC |
| | 7 | LC FR4 | FGGGTKVEIK |
| | 33 | VL amino | EIVLTQSPATLSVSPGERATLSCRASQSVSSNLAWYQQKPGQA |
| | | acid | PRLLIYGASDRATGIPARFSGSGSGTEFTLTISSLQSEDFAVYYCI |
| | | | QYSNYPRTFGGGTKVEIK |
| | 34 | LC | EIVLTQSPATLSVSPGERATLSCRASQSVSSNLAWYQQKPGQA |
| | | | PRLLIYGASDRATGIPARFSGSGSGTEFTLTISSLQSEDFAVYYCI |
| | | | QYSNYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 35 | VL DNA | GAAATAGTGTTGACGCAGTCTCCAGCCACCCTGTCTGTGTCT |
| | | | CCAGGGGAAAGAGCCACCCTCTCCTGCAGGGCCAGTCAGAG |
| | | | TGTTAGCAGCAACTTAGCCTGGTACCAGCAGAAACCTGGCCA |
| | | | GGCTCCCAGGCTCCTCATCTATGGTGCATCCGACAGGGCCA |
| | | | CTGGTATCCCAGCCAGGTTCAGTGGCAGTGGGTCTGGGACA |
| | | | GAGTTCACTCTCACCATCAGCAGCCTGCAGTCTGAAGATTTT |
| | | | GCAGTTTATTACTGTATTCAGTACTCCAACTACCCTAGGACTT |
| | | | TTGGCGGAGGGACCAAGGTTGAGATCAAA |
| Antibody G | 1 | LC CDR1 | RASQSVRSSYLA |
| VL | 2 | LC CDR2 | GASSRAT |
| germline | 36 | LC CDR3 | QQYSNYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 37 | VL amino | EIVLTQSPGTLSLSPGERATLSCRASQSVRSSYLAWYQQKPGQ |
| | | acid | APRLLIYGASSRATGIPDRFSGSGSGTDFTLTISRLEPEDFAVYY |
| | | | CQQYSNYPRTFGGGTKVEIK |
| | 38 | LC | EIVLTQSPGTLSLSPGERATLSCRASQSVRSSYLAWYQQKPGQ |
| | | | APRLLIYGASSRATGIPDRFSGSGSGTDFTLTISRLEPEDFAVYY |
| | | | CQQYSNYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 39 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGGAGCAGCTACTTAGCCTGGTACCAGCAGAAACCTG |
| | | | GCCAGGCTCCCAGGCTCCTCATCTATGGTGCATCCAGCAGG |
| | | | GCCACTGGCATCCCAGACAGGTTCAGTGGCAGTGGGTCTGG |
| | | | GACAGACTTCACTCTCACCATCAGCAGACTGGAGCCTGAAGA |
| | | | TTTTGCAGTGTATTACTGTCAGCAGTACTCCAACTACCCTAGG |
| | | | ACTTTTGGCGGAGGGACCAAGGTTGAGATCAAA |
| Antibody H | 1 | LC CDR1 | RASQSVRSSYLA |
| VL | 2 | LC CDR2 | GASSRAT |
| germline | 36 | LC CDR3 | QQYSNYPRT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 37 | VL amino | EIVLTQSPGTLSLSPGERATLSCRASQSVRSSYLAWYQQKPGQ |
| | | acid | APRLLIYGASSRATGIPDRFSGSGSGTDFTLTISRLEPEDFAVYY |
| | | | CQQYSNYPRTFGGGTKVEIK |
| | 38 | LC | EIVLTQSPGTLSLSPGERATLSCRASQSVRSSYLAWYQQKPGQ |
| | | | APRLLIYGASSRATGIPDRFSGSGSGTDFTLTISRLEPEDFAVYY |
| | | | CQQYSNYPRTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 39 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGGAGCAGCTACTTAGCCTGGTACCAGCAGAAACCTG |
| | | | GCCAGGCTCCCAGGCTCCTCATCTATGGTGCATCCAGCAGG |
| | | | GCCACTGGCATCCCAGACAGGTTCAGTGGCAGTGGGTCTGG |
| | | | GACAGACTTCACTCTCACCATCAGCAGACTGGAGCCTGAAGA |
| | | | TTTTGCAGTGTATTACTGTCAGCAGTACTCCAACTACCCTAGG |
| | | | ACTTTTGGCGGAGGGACCAAGGTTGAGATCAAA |
| Antibody I | 11 | LC CDR1 | RASQSVSSSYLA |
| VL | 43 | LC CDR2 | GASYRAT |
| germline | 44 | LC CDR3 | LQSGVFPYT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 45 | VL amino | EIVLTQSPGTLSLSPGERATLSCRASQSVSSSYLAWYQQKPGQ |
| | | acid | APRLLIYGASYRATGIPDRFSGSGSGTDFTLTISRLEPEDFAVYY |
| | | | CLQSGVFPYTFGGGTKVEIK |
| | 46 | LC | EIVLTQSPGTLSLSPGERATLSCRASQSVSSSYLAWYQQKPGQ |
| | | | APRLLIYGASYRATGIPDRFSGSGSGTDFTLTISRLEPEDFAVYY |
| | | | CLQSGVFPYTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 47 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGCAGCAGCTACTTAGCCTGGTACCAGCAGAAACCTGG |
| | | | CCAGGCTCCCAGGCTCCTCATCTATGGTGCATCCTACAGGG |
| | | | CCACTGGCATCCCAGACAGGTTCAGTGGCAGTGGGTCTGGG |
| | | | ACAGACTTCACTCTCACCATCAGCAGACTGGAGCCTGAAGAT |
| | | | TTTGCAGTGTATTACTGTTTGCAGTCCGGAGTCTTCCCTTACA |
| | | | CTTTTGGCGGAGGGACCAAGGTTGAGATCAAA |
| Antibody J | 11 | LC CDR1 | RASQSVSSSYLA |
| VL | 48 | LC CDR2 | GAFSRAT |
| germline | 49 | LC CDR3 | QQSGVFPYT |
| VK3-20 | 4 | LC FR1 | EIVLTQSPGTLSLSPGERATLSC |
| | 5 | LC FR2 | WYQQKPGQAPRLLIY |
| | 6 | LC FR3 | GIPDRFSGSGSGTDFTLTISRLEPEDFAVYYC |
| | 7 | LC FR4 | FGGGTKVEIK |
| | 50 | VL amino | EIVLTQSPGTLSLSPGERATLSCRASQSVSSSYLAWYQQKPGQ |
| | | acid | APRLLIYGAFSRATGIPDRFSGSGSGTDFTLTISRLEPEDFAVYY |
| | | | CQQSGVFPYTFGGGTKVEIK |
| | 51 | LC | EIVLTQSPGTLSLSPGERATLSCRASQSVSSSYLAWYQQKPGQ |
| | | | APRLLIYGAFSRATGIPDRFSGSGSGTDFTLTISRLEPEDFAVYY |
| | | | CQQSGVFPYTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 52 | VL DNA | GAAATTGTGTTGACGCAGTCTCCAGGCACCCTGTCTTTGTCT |
| | | | CCAGGGGAAAGAGCCACCCTCTCCTGCAGGGCCAGTCAGAG |
| | | | TGTTAGCAGCAGCTACTTAGCCTGGTACCAGCAGAAACCTGG |
| | | | CCAGGCTCCCAGGCTCCTCATCTATGGTGCATTCAGCAGGG |
| | | | CCACTGGCATCCCAGACAGGTTCAGTGGCAGTGGGTCTGGG |
| | | | ACAGACTTCACTCTCACCATCAGCAGACTGGAGCCTGAAGAT |
| | | | TTTGCAGTGTATTACTGTCAGCAGTCCGGAGTCTTCCCTTAC |
| | | | ACTTTTGGCGGAGGGACCAAGGTTGAGATCAAA |
| Antibody K | 53 | LC CDR1 | RASQSVSSYLA |
| VL | 54 | LC CDR2 | DASNRAT |
| germline | 55 | LC CDR3 | QQDSIFPPYT |
| VK3-11 | 56 | LC FR1 | EIVLTQSPATLSLSPGERATLSC |
| | 5 | LC FR2 | WYQQKPGQAPRLLIY |
| | 57 | LC FR3 | GIPARFSGSGSGTDFTLTISSLEPEDFAVYYC |
| | 7 | LC FR4 | FGGGTKVEIK |
| | 58 | VL amino | EIVLTQSPATLSLSPGERATLSCRASQSVSSYLAWYQQKPGQA |
| | | acid | PRLLIYDASNRATGIPARFSGSGSGTDFTLTISSLEPEDFAVYYC |
| | | | QQDSIFPPYTFGGGTKVEIK |
| | 59 | LC | EIVLTQSPATLSLSPGERATLSCRASQSVSSYLAWYQQKPGQA |
| | | | PRLLIYDASNRATGIPARFSGSGSGTDFTLTISSLEPEDFAVYYC |
| | | | QQDSIFPPYTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 60 | VL DNA | GAAATTGTGTTGACACAGTCTCCAGCCACCCTGTCTTTGTCT |
| | | | CCAGGGGAAAGAGCCACCCTCTCCTGCAGGGCCAGTCAGAG |
| | | | TGTTAGCAGCTACTTAGCCTGGTACCAACAGAAACCTGGCCA |
| | | | GGCTCCCAGGCTCCTCATCTATGATGCATCCAACAGGGCCA |
| | | | CTGGCATCCCAGCCAGGTTCAGTGGCAGTGGGTCTGGGACA |
| | | | GACTTCACTCTCACCATCAGCAGCCTAGAGCCTGAAGATTTT |
| | | | GCAGTTTATTACTGTCAGCAGGACAGTATCTTCCCGCCTTAC |
| | | | ACTTTTGGCGGAGGGACCAAGGTTGAGATCAAA |
| Antibody L | 28 | LC CDR1 | RASQSVSSNLA |
| VL | 61 | LC CDR2 | GASTRAT |
| germline | 62 | LC CDR3 | QQSDDWPWT |
| VK3-15 | 63 | LC FR1 | EIVLTQSPATLSVSPGERATLSC |
| | 5 | LC FR2 | WYQQKPGQAPRLLIY |
| | 32 | LC FR3 | GIPARFSGSGSGTEFTLTISSLQSEDFAVYYC |
| | 7 | LC FR4 | FGGGTKVEIK |
| | 65 | VL amino | EIVLTQSPATLSVSPGERATLSCRASQSVSSNLAWYQQKPGQA |
| | | acid | PRLLIYGASTRATGIPARFSGSGSGTEFTLTISSLQSEDFAVYYC |
| | | | QQSDDWPWTFGGGTKVEIK |
| | 66 | LC | EIVLTQSPATLSVSPGERATLSCRASQSVSSNLAWYQQKPGQA |
| | | | PRLLIYGASTRATGIPARFSGSGSGTEFTLTISSLQSEDFAVYYC |
| | | | QQSDDWPWTFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 67 | VL DNA | GAAATAGTGTTGACGCAGTCTCCAGCCACCCTGTCTGTGTCT |
| | | | CCAGGGGAAAGAGCCACCCTCTCCTGCAGGGCCAGTCAGAG |
| | | | TGTTAGCAGCAACTTAGCCTGGTACCAGCAGAAACCTGGCCA |
| | | | GGCTCCCAGGCTCCTCATCTATGGTGCATCCACCAGGGCCA |
| | | | CTGGTATTCCAGCCAGGTTCAGTGGCAGTGGGTCTGGGACA |
| | | | GAGTTCACTCTCACCATCAGCAGCCTGCAGTCTGAAGATTTT |
| | | | GCAGTTTATTACTGTCAGCAGTCCGACGACTGGCCTTGGACT |
| | | | TTTGGCGGAGGGACCAAGGTTGAGATCAAA |
| Antibody M | 68 | LC CDR1 | QASQDISNYLN |
| VL | 69 | LC CDR2 | DASNLET |
| germline | 70 | LC CDR3 | QQSDHLPFT |
| VK1-33 | 71 | LC FR1 | DIQMTQSPSSLSASVGDRVTITC |
| | 72 | LC FR2 | WYQQKPGKAPKLLIY |
| | 73 | LC FR3 | GVPSRFSGSGSGTDFTFTISSLQPEDIATYYC |
| | 7 | LC FR4 | FGGGTKVEIK |
| | 74 | VL amino | DIQMTQSPSSLSASVGDRVTITCQASQDISNYLNWYQQKPGKA |
| | | acid | PKLLIYDASNLETGVPSRFSGSGSGTDFTFTISSLQPEDIATYYC |
| | | | QQSDHLPFTFGGGTKVEIK |
| | 75 | LC | DIQMTQSPSSLSASVGDRVTITCQASQDISNYLNWYQQKPGKA |
| | | | PKLLIYDASNLETGVPSRFSGSGSGTDFTFTISSLQPEDIATYYC |
| | | | QQSDHLPFTFGGGTKVEIKRTVAAPSVFIFPPSDEQLKSGTASV |
| | | | VCLLNNFYPREAKVQWKVDNALQSGNSQESVTEQDSKDSTYS |
| | | | LSSTLTLSKADYEKHKVYACEVTHQGLSSPVTKSFNRGEC |
| | 76 | VL DNA | GACATCCAGATGACCCAGTCTCCATCCTCCCTGTCTGCATCT |
| | | | GTAGGAGACAGAGTCACCATCACTTGCCAGGCGAGTCAGGA |
| | | | CATTAGCAACTATTTAAATTGGTATCAGCAGAAACCAGGGAA |
| | | | AGCCCCTAAGCTCCTGATCTACGATGCATCCAATTTGGAAAC |
| | | | AGGGGTCCCATCAAGGTTCAGTGGAAGTGGATCTGGGACAG |
| | | | ATTTTACTTTCACCATCAGCAGCCTGCAGCCTGAAGATATTGC |
| | | | AACATATTACTGTCAGCAGTCCGACCACCTCCCTTTCACTTTT |
| | | | GGCGGAGGGACCAAGGTTGAGATCAAA |
| Antibody N | 68 | LC CDR1 | QASQDISNYLN |
| VL | 69 | LC CDR2 | DASNLET |
| germline | 70 | LC CDR3 | QQSDHLPFT |
| VK1-33 | 71 | LC FR1 | DIQMTQSPSSLSASVGDRVTITC |
| | 72 | LC FR2 | WYQQKPGKAPKLLIY |
| | 73 | LC FR3 | GVPSRFSGSGSGTDFTFTISSLQPEDIATYYC |
| | 7 | LC FR4 | FGGGTKVEIK |
| | 74 | VL amino | DIQMTQSPSSLSASVGDRVTITCQASQDISNYLNWYQQKPGKA |
| | | acid | PKLLIYDASNLETGVPSRFSGSGSGTDFTFTISSLQPEDIATYYC |
| | | | QQSDHLPFTFGGGTKVEIK |
| | 75 | LC | DIQMTQSPSSLSASVGDRVTITCQASQDISNYLNWYQQKPGKA |
| | | | PKLLIYDASNLETGVPSRFSGSGSGTDFTFTISSLQPEDIATYYC |
| | | | QQSDHLPFTFGGGTKVEIKRTVAAPSVFIFPPSDEQLKSGTASV |
| | | | VCLLNNFYPREAKVQWKVDNALQSGNSQESVTEQDSKDSTYS |
| | | | LSSTLTLSKADYEKHKVYACEVTHQGLSSPVTKSFNRGEC |
| | 76 | VL DNA | GACATCCAGATGACCCAGTCTCCATCCTCCCTGTCTGCATCT |
| | | | GTAGGAGACAGAGTCACCATCACTTGCCAGGCGAGTCAGGA |
| | | | CATTAGCAACTATTTAAATTGGTATCAGCAGAAACCAGGGAA |
| | | | AGCCCCTAAGCTCCTGATCTACGATGCATCCAATTTGGAAAC |
| | | | AGGGGTCCCATCAAGGTTCAGTGGAAGTGGATCTGGGACAG |
| | | | ATTTTACTTTCACCATCAGCAGCCTGCAGCCTGAAGATATTGC |
| | | | AACATATTACTGTCAGCAGTCCGACCACCTCCCTTTCACTTTT |
| | | | GGCGGAGGGACCAAGGTTGAGATCAAA |
| Antibody O | 28 | LC CDR1 | RASQSVSSNLA |
| VL | 61 | LC CDR2 | GASTRAT |
| germline | 80 | LC CDR3 | QQYNNWPIT |
| VK3-15 | 81 | LC FR1 | EIVMTQSPATLSVSPGERATLSC |
| | 5 | LC FR2 | WYQQKPGQAPRLLIY |
| | 32 | LC FR3 | GIPARFSGSGSGTEFTLTISSLQSEDFAVYYC |
| | 7 | LC FR4 | FGGGTKVEIK |
| | 82 | VL amino | EIVMTQSPATLSVSPGERATLSCRASQSVSSNLAWYQQKPGQA |
| | | acid | PRLLIYGASTRATGIPARFSGSGSGTEFTLTISSLQSEDFAVYYC |
| | | | QQYNNWPITFGGGTKVEIK |
| | 83 | LC | EIVMTQSPATLSVSPGERATLSCRASQSVSSNLAWYQQKPGQA |
| | | | PRLLIYGASTRATGIPARFSGSGSGTEFTLTISSLQSEDFAVYYC |
| | | | QQYNNWPITFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 84 | VL DNA | GAAATAGTGATGACGCAGTCTCCAGCCACCCTGTCTGTGTCT |
| | | | CCAGGGGAAAGAGCCACCCTCTCCTGCAGGGCCAGTCAGAG |
| | | | TGTTAGCAGCAACTTAGCCTGGTACCAGCAGAAACCTGGCCA |
| | | | GGCTCCCAGGCTCCTCATCTATGGTGCATCCACCAGGGCCA |
| | | | CTGGTATCCCAGCCAGGTTCAGTGGCAGTGGGTCTGGGACA |
| | | | GAGTTCACTCTCACCATCAGCAGCCTGCAGTCTGAAGATTTT |
| | | | GCAGTTTATTACTGTCAGCAGTATAATAACTGGCCTATCACCT |
| | | | TTGGCGGAGGGACCAAGGTTGAGATCAAA |
| Antibody P | 85 | LC CDR1 | RASQGIDSWLA |
| VL | 86 | LC CDR2 | AASSLQS |
| germline | 87 | LC CDR3 | QQVHSFPIT |
| VK1-12 | 88 | LC FR1 | DIQMTQSPSSVSASVGDRVTITC |
| | 72 | LC FR2 | WYQQKPGKAPKLLIY |
| | 89 | LC FR3 | GVPSRFSGSGSGTDFTLTISSLQPEDFATYYC |
| | 7 | LC FR4 | FGGGTKVEIK |
| | 90 | VL amino | DIQMTQSPSSVSASVGDRVTITCRASQGIDSWLAWYQQKPGKA |
| | | acid | PKLLIYAASSLQSGVPSRFSGSGSGTDFTLTISSLQPEDFATYYC |
| | | | QQVHSFPITFGGGTKVEIK |
| | 91 | LC | DIQMTQSPSSVSASVGDRVTITCRASQGIDSWLAWYQQKPGKA |
| | | | PKLLIYAASSLQSGVPSRFSGSGSGTDFTLTISSLQPEDFATYYC |
| | | | QQVHSFPITFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 92 | VL DNA | GACATCCAGATGACCCAGTCTCCATCTTCCGTGTCTGCATCT |
| | | | GTAGGAGACAGAGTCACCATCACTTGTCGGGCGAGTCAGGG |
| | | | TATTGACAGCTGGTTAGCCTGGTATCAGCAGAAACCAGGGAA |
| | | | AGCCCCTAAGCTCCTGATCTATGCTGCATCCAGTTTGCAAAG |
| | | | TGGGGTCCCATCAAGGTTCAGCGGCAGTGGATCTGGGACAG |
| | | | ATTTCACTCTCACCATCAGCAGCCTGCAGCCTGAAGATTTTG |
| | | | CAACTTATTACTGTCAGCAGGTACACAGTTTCCCTATCACTTT |
| | | | TGGCGGAGGGACCAAGGTTGAGATCAAA |
| Antibody Q | 93 | LC CDR1 | RSSQSLLHSNGYNYLD |
| VL | 94 | LC CDR2 | LGSNRAS |
| germline | 95 | LC CDR3 | MQALQTPIT |
| VK2-28 | 96 | LC FR1 | DIVMTQSPLSLPVTPGEPASISC |
| | 97 | LC FR2 | WYLQKPGQSPQLLIY |
| | 98 | LC FR3 | GVPDRFSGSGSGTDFTLKISRVEAEDVGVYYC |
| | 7 | LC FR4 | FGGGTKVEIK |
| | 99 | VL amino | DIVMTQSPLSLPVTPGEPASISCRSSQSLLHSNGYNYLDWYLQK |
| | | acid | PGQSPQLLIYLGSNRASGVPDRFSGSGSGTDFTLKISRVEAEDV |
| | | | GVYYCMQALQTPITFGGGTKVEIK |
| | 100 | LC | DIVMTQSPLSLPVTPGEPASISCRSSQSLLHSNGYNYLDWYLQK |
| | | | PGQSPQLLIYLGSNRASGVPDRFSGSGSGTDFTLKISRVEAEDV |
| | | | GVYYCMQALQTPITFGGGTKVEIKRTVAAPSVFIFPPSDEQLKS |
| | | | GTASVVCLLNNFYPREAKVQWKVDNALQSGNSQESVTEQDSK |
| | | | DSTYSLSSTLTLSKADYEKHKVYACEVTHQGLSSPVTKSFNRGE |
| | | | C |
| | 101 | VL DNA | GATATTGTGATGACTCAGTCTCCACTCTCCCTGCCCGTCACC |
| | | | CCTGGAGAGCCGGCCTCCATCTCCTGCAGGTCTAGTCAGAG |
| | | | CCTCCTGCATAGTAATGGATACAACTATTTGGATTGGTACCTG |
| | | | CAGAAGCCAGGGCAGTCTCCACAGCTCCTGATCTATTTGGGT |
| | | | TCTAATCGGGCCTCCGGGGTCCCTGACAGGTTCAGTGGCAG |
| | | | TGGATCAGGCACAGATTTTACACTGAAAATCAGCAGAGTGGA |
| | | | GGCTGAGGATGTTGGGGTTTATTACTGCATGCAAGCTCTACA |
| | | | AACTCCTATCACCTTTGGCGGAGGGACCAAGGTTGAGATCAA |
| | | | A |
| Antibody R | 93 | LC CDR1 | RSSQSLLHSNGYNYLD |
| VL | 94 | LC CDR2 | LGSNRAS |
| germline | 95 | LC CDR3 | MQALQTPIT |
| VK2-28 | 96 | LC FR1 | DIVMTQSPLSLPVTPGEPASISC |
| | 97 | LC FR2 | WYLQKPGQSPQLLIY |
| | 98 | LC FR3 | GVPDRFSGSGSGTDFTLKISRVEAEDVGVYYC |
| | 7 | LC FR4 | FGGGTKVEIK |
| | 99 | VL amino | DIVMTQSPLSLPVTPGEPASISCRSSQSLLHSNGYNYLDWYLQK |
| | | acid | PGQSPQLLIYLGSNRASGVPDRFSGSGSGTDFTLKISRVEAEDV |
| | | | GVYYCMQALQTPITFGGGTKVEIK |
| | 100 | LC | DIVMTQSPLSLPVTPGEPASISCRSSQSLLHSNGYNYLDWYLQK |
| | | | PGQSPQLLIYLGSNRASGVPDRFSGSGSGTDFTLKISRVEAEDV |
| | | | GVYYCMQALQTPITFGGGTKVEIK |
| | | | RTVAAPSVFIFPPSDEQLKSGTASVVCLLNNFYPREAKVQWKVD |
| | | | NALQSGNSQESVTEQDSKDSTYSLSSTLTLSKADYEKHKVYAC |
| | | | EVTHQGLSSPVTKSFNRGEC |
| | 101 | VL DNA | GATATTGTGATGACTCAGTCTCCACTCTCCCTGCCCGTCACC |
| | | | CCTGGAGAGCCGGCCTCCATCTCCTGCAGGTCTAGTCAGAG |
| | | | CCTCCTGCATAGTAATGGATACAACTATTTGGATTGGTACCTG |
| | | | CAGAAGCCAGGGCAGTCTCCACAGCTCCTGATCTATTTGGGT |
| | | | TCTAATCGGGCCTCCGGGGTCCCTGACAGGTTCAGTGGCAG |
| | | | TGGATCAGGCACAGATTTTACACTGAAAATCAGCAGAGTGGA |
| | | | GGCTGAGGATGTTGGGGTTTATTACTGCATGCAAGCTCTACA |
| | | | AACTCCTATCACCTTTGGCGGAGGGACCAAGGTTGAGATCAA |
| | | | A |

### Heavy Chain (e.g., Heavy Chain Variable Region) Polypeptides (HC Polypeptides)

The present disclosure provides polypeptides comprising heavy chain (HC) sequences (e.g., heavy chain variable region sequence(s)) that, for example, may be useful in antibody agents as described herein targeting Activin A; in some such embodiments, such provided polypeptides are useful and/or included in such antibody agents as described herein. In some embodiments, a HC polypeptide comprises at least one HC CDR of an Activin A antibody agent as provided in Table 2 or a sequence with at least 85% identity thereto. In some embodiments, a HC polypeptide comprises one, two or three HC CDRs (e.g., a HC CDR1, a HC CDR2 and/or a HC CDR3). In some embodiments, a HC polypeptide comprises a HC CDR1. In some embodiments, a HC polypeptide comprises a HC CDR2. In some embodiments, a HC polypeptide comprises a HC CDR3. In some embodiments, a HC polypeptide comprises a HC CDR1, a HC CDR2 and a HC CDR3.

In some embodiments, a HC polypeptide comprising a HC CDR1, a HC CDR2 and a HC CDR3 is capable of binding (e.g., specifically binding) to Activin A.

In some embodiments, a HC polypeptide further comprises one or more framework regions, and/or a heavy chain constant region, or a portion or a variant thereof (e.g., a CH1, CH2 and/or CH3 region). In some embodiments, a HC polypeptide comprises a CH1, a CH2 or a CH3 or a combination thereof. In some embodiments, a HC polypeptide comprises a CH2 and CH3, e.g., an Fc domain.

In some embodiments, a Fc domain comprises a mammalian Fc domain. In some embodiments, a Fc domain comprises a dog, a cat, a mouse, a rat, a rabbit, a primate, a cow, a horse, a sheep, or a human Fc domain. In some embodiments, a Fc domain comprises a human Fc domain. In some embodiments, a Fc domain comprises a dog Fc domain. In some embodiments, a Fc domain comprises a cat Fc domain. In some embodiments, an Fc domain is chosen from an Fc domain of an immunoglobulin isotype. In some embodiments, an immunoglobulin isotype comprises IgA, IgD, IgG, IgM, or IgE. In some embodiments, an Fc domain comprises an Fc domain of an IgG, e.g., a human IgG. In some embodiments, an IgG is or comprises IgG1, IgG2, IgG3, or IgG4.

In some embodiments, an Fc region is a wildtype Fc region, e.g., a wildtype human Fc region. In some embodiments, an Fc region comprises a variant, e.g., an Fc region comprising an addition, substitution, or deletion of at least one amino acid residue in an Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein Clq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, a HC polypeptide comprising a variant Fc region has one or more of the following properties: (1) reduced effector function (e.g., reduced ADCC, ADCP and/or CDC); (2) reduced binding to one or more Fc receptors; and/or (3) reduced binding to Clq complement. In some embodiments, the reduction in any one, or all of properties (1)-(3) is compared to an otherwise similar antibody with a wildtype Fc region. In some embodiments, an Activin A antibody agent comprising a variant Fc region has reduced affinity to a human Fc receptor, e.g., FcγR I, FcγR II and/or FcγR III. Exemplary Fc region variants are disclosed in Saunders K. O., (2019) Frontiers in Immunology; vol 10, Article 296, the entire contents of which is hereby incorporated by reference. For example, a Fc region variant is or comprises a modification provided in Table 3 of Saunders KO (2019). In some embodiments, a Fc region variant comprises Leu234Ala/Leu235Ala (LALA) mutation, a Leu235Glu (LE) mutation, a Ser228Pro/Leu235Glu (SPLE) mutation, Leu234Ala/Leu235Ala/Pro239Gly (LALA-PG) mutation, Pro 331 Ser/Leu234Glu/Leu235Phe™, Asp265Ala (DA) mutation, Leu235Ala/Gly237Ala (LAGA) mutation, or a combination thereof.

In some embodiments, a HC polypeptide disclosed herein comprises a Leu234Ala/Leu235Ala (LALA) mutation.

In some embodiments, a HC polypeptide disclosed herein comprises a Leu235Ala/Gly237Ala (LAGA) mutation.

In some embodiments, a Fc region variant comprises a mutation relative to a wildtype Fc region, e.g., a IgG1 FcR wildtype region. In some embodiments, the hinge and CH2 sequence of an IgG1 FcR wildtype region comprises the sequence of: CPPCPAPELLGGPSVFLFPPK (SEQ ID NO: 222).

In some embodiments, a Fc region variant comprises a LAGA mutation, e.g., as shown in bold in SEQ ID NO: 223: CPPCPAPELAGAPSVFLFPPK. In some embodiments, a HC polypeptide comprises an Fc region having a LAGA mutation, e.g., as provided in SEQ ID NO: 223.

In some embodiments, a Fc region variant comprises a FEGG mutation, e.g., as shown in bold in SEQ ID NO: 224: CPPCPAPEFEGGPSVFLFPPK. In some embodiments, a HC polypeptide comprises an Fc region having a FEGG mutation, e.g., as provided in SEQ ID NO: 224.

In some embodiments, a Fc region variant comprises a AAGG mutation, e.g., as shown in bold in SEQ ID NO: 225: CPPCPAPEAAGGPSVFLFPPK. In some embodiments, a HC polypeptide comprises an Fc region having a AAGG mutation, e.g., as provided in SEQ ID NO: 225.

In some embodiments, a Fc region variant comprises a AAGA mutation, e.g., as shown in bold in SEQ ID NO: 226: CPPCPAPEAAGAPSVFLFPPK. In some embodiments, an AAGA mutation is also referred to as Leu234Ala/Leu235Ala/Glu237Ala (LALAGA). In some embodiments, a HC polypeptide comprises an Fc region having an AAGA mutation, e.g., as provided in SEQ ID NO: 226.

In some embodiments, an Fc region variant comprising an Fc mutation (e.g., as described herein) has reduced binding (e.g., no binding) to a neonatal Fc receptor (FcRn), e.g., when compared to an otherwise similar Fc region without the relevant Fc mutation. In some embodiments, an Activin A antibody agent comprising an Fc region having an Fc mutation has reduced binding (e.g., no binding) to FcRn and reduced placental transfer, compared to an otherwise similar Activin A antibody agent with an Fc region without the relevant Fc mutation (e.g., as described herein).

In some embodiments, an Fc region variant comprising a LAGA mutation, a FEGG mutation, an AAGG mutation, an AAGA mutation, a LALA mutation or a combination thereof, has reduced binding (e.g., no binding) to a neonatal Fc receptor (FcRn), e.g., when compared to an otherwise similar Fc region without the relevant mutation (e.g., LAGA mutation, FEGG mutation, AAGG mutation, AAGA mutation, LALA mutation or combination thereof). In some embodiments, a GDF15 antibody agent comprising an Fc region having a LAGA mutation, a FEGG mutation, an AAGG mutation, an AAGA mutation, a LALA mutation or a combination thereof has reduced binding (e.g., no binding) to FcRn and reduced placental transfer, compared to an otherwise similar Activin A antibody agent with an Fc region without the relevant mutation (e.g., LAGA mutation, FEGG mutation, AAGG mutation, AAGA mutation, LALA mutation or combination thereof). In some embodiments, an Fc region variant comprising a I253A mutation, a H310A mutation, a H435R mutation, a H435A mutation or a combination thereof, has reduced binding (e.g., no binding) to a neonatal Fc receptor (FcRn), e.g., when compared to an otherwise similar Fc region without the relevant mutation (e.g., the relevant I253A mutation, H310A mutation, H435R mutation, H435A mutation or combination thereof). In some embodiments, a Activin A antibody agent comprising an Fc region having a I253A mutation, a H310A mutation, a H435R mutation, a H435A mutation, or a combination thereof has reduced binding (e.g., no binding) to FcRn and reduced placental transfer, compared to an otherwise similar Activin A antibody agent with an Fc region without the relevant mutation (e.g., the relevant I253A mutation, H310A mutation, H435R mutation, H435A mutation or combination thereof.

In some embodiments, a HC polypeptide disclosed herein further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, an HC polypeptide comprises a CH3 domain or a variant thereof. In some embodiments, a CH3 variant is characterized in that, when it is introduced into an HC polypeptide, a half-life of the HC polypeptide is extended without reducing one or more other desirable characteristics, such as neutralization potency, effector function, and/or developability. In some embodiments, an HC polypeptide having a CH3 variant has an extended half-life compared to an otherwise similar HC polypeptide without the relevant CH3 variant.

In some embodiments, a CH3 variant has an addition, substitution, or deletion of at least one amino acid residue compared to a reference CH3 domain, e.g., a wild-type CH3 domain.

In some embodiments, a CH3 variant has an amino acid residue at position 428 which differs from a reference CH3 domain, e.g., a wild-type CH3 domain. In some embodiments, a CH3 variant has an amino acid residue at position 434 which differs from a reference CH3 domain, e.g., a wild-type CH3 domain. In some embodiments, a CH3 variant has amino acid residues at positions 428 and 434 which differ from a reference CH3 domain, e.g., a wild-type CH3 domain.

In some embodiments, a CH3 variant has a leucine at position 428.

In some embodiments, a CH3 variant has an alanine at position 434.

In some embodiments, a CH3 variant has a leucine at position 428 and an alanine at position 434.

In some embodiments, an HC polypeptide comprising a CH3 variant is characterized in that, when it is administered to a subject, increased antibody dependent cellular cytotoxicity (ADCC) and/or antibody-dependent cellular phagocytosis (ADCP) is observed as compared to that observed when an HC polypeptide without the relevant CH3 variant is administered to a comparable subject. In some embodiments, increased ADCC is characterized by one or more of: increased surface expression of CD107a on natural killer (NK) cells, increased interferon γ (IFNγ) production by NK cells or increased tumor necrosis factor α (TNFα) production by NK cells. In some embodiments, increased ADCP is characterized by one or more of: co-localization of target cells and macrophages utilizing microscopy or flow cytometry; and the inclusion of a pH-sensitive dye to differentiate between cell-associated and internalized target cells.

In some embodiments, an HC polypeptide comprising a CH3 variant has improved developability as compared to an HC polypeptide without the relevant CH3 variant. In some embodiments, improving the developability of an HC polypeptide comprising a CH3 variant comprises increasing expression, increasing solubility, increasing covalent integrity, increasing conformational stability, increasing colloidal stability, decreasing poly-specificity, and/or decreasing immunogenicity of an HC polypeptide comprising a CH3 variant relative to an HC polypeptide without the relevant CH3 variant.

In some embodiments, disclosed herein are preparations of an antibody agent comprising a human constant region comprising a variant CH3 domain, wherein the antibody agent is characterized in that the neutralization potency and/or effector function of the antibody agent is comparable to that of an antibody agent comprising a parent CH3 domain, and/or wherein the antibody agent is characterized in that the developability of the antibody agent is increased relative to that of an antibody agent comprising a reference (e.g., parent) CH3 domain, wherein the variant CH3 domain differs from a parent CH3 domain at positions 428 and 434, and wherein the variant CH3 domain comprises a leucine at position 428 and an alanine at position 434. In some embodiments, the developability of the antibody agent comprises high level expression, high solubility, covalent integrity, conformational stability, colloidal stability, low poly-specificity, and/or low immunogenicity

According to IMGT, the CH3 domain is the amino acid positions (or simply referred to as “positions” herein) 341-446 (EU numbering). The term “CH3 domain” is used in a broad sense herein to refer to a heavy chain region comprising at least seven consecutive amino acid positions of the heavy chain positions 341-446 (EU numbering)). A CH3 domain reference sequence, corresponding to the amino acid positions 341-446 according to EU numbering, is provided herein as SEQ ID NO: 227, which is an exemplary amino acid sequence of a wild-type (WT) CH3 domain.

Exemplary CH3 domain reference sequence:

(SEQ ID NO: 227)
GQPREPQVYTLPPSRDELTKNQVSLTCLVKGFYPSDIAVEWESNGQ
PENNYKTTPPVLDSDGSFFLYSKLTVDKSRWQQGNVFSCSVMHEAL
HNHYTQKSLSLSPG.

In some embodiments, a HC polypeptide comprises: (i) a HC CDR1 sequence provided in Table 2, e.g., any one of SEQ ID NOs: 103, 113. 120, 125, 129, 134, 140, 149, 157, 163, 169, 179, 190, 196, 202 or 209; (ii) a sequence with at least 85%, 86%, 87%, 88%, 89%, 90%, 91%, 92%, 93%, 94%, 95%, 96%, 97%, 98%, 99% identity to a HC CDR1 sequence provided in Table 2, e.g., any one of SEQ ID NOs: 103, 113. 120, 125, 129, 134, 140, 149, 157, 163, 169, 179, 190, 196, 202 or 209; or (iii) a sequence having at least 5, 10, or 20 substitutions compared to a HC CDR1 sequence provided in Table 2, e.g., any one of SEQ ID NOs: 103, 113. 120, 125, 129, 134, 140, 149, 157, 163, 169, 179, 190, 196, 202 or 209.

In some embodiments, a HC polypeptide comprises: (i) a HC CDR2 sequence provided in Table 2, e.g., any one of SEQ ID NOs: 104, 114, 121, 130, 135, 141, 145, 150, 158, 164, 170, 180, 186, 191, 197, 203 or 210; (ii) a sequence with at least 85%, 86%, 87%, 88%, 89%, 90%, 91%, 92%, 93%, 94%, 95%, 96%, 97%, 98%, 99% identity to a HC CDR2 sequence provided in Table 2, e.g., any one of SEQ ID NOs: 104, 114, 121, 130, 135, 141, 145, 150, 158, 164, 170, 180, 186, 191, 197, 203 or 210; or (iii) a sequence having at least 5, 10, or 20 substitutions compared to a HC CDR2 sequence provided in Table 2, e.g., any one of SEQ ID NOs: 104, 114, 121, 130, 135, 141, 145, 150, 158, 164, 170, 180, 186, 191, 197, 203 or 210.

In some embodiments, a HC polypeptide comprises: (i) a HC CDR3 sequence provided in Table 2, e.g., any one of SEQ ID NOs: 105, 115, 151, 165, 171, 181, 192, 198, or 204; (ii) a sequence with at least 85%, 86%, 87%, 88%, 89%, 90%, 91%, 92%, 93%, 94%, 95%, 96%, 97%, 98%, 99% identity to a HC CDR3 sequence provided in Table 2, e.g., any one of SEQ ID NOs: 105, 115, 151, 165, 171, 181, 192, 198, or 204; or (iii) a sequence having at least 5, 10, or 20 substitutions compared to a HC CDR3 sequence provided in Table 2, e.g., any one of SEQ ID NOs: 105, 115, 151, 165, 171, 181, 192, 198, or 204.

In some embodiments, a HC polypeptide comprising a HC CDR1, a HC CDR2 and/or a HC CDR3 is able to specifically bind to Activin A.

In some embodiments, a HC polypeptide comprises: (i) an HC CDR1, HC CDR2, and HC CDR3 sequence provided in Table 2; (ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 2; (iii) a sequence having at least 5, 10, or 20 substitutions relative to an HC CDR1, HC CDR2, and HC CDR3 sequence provided in Table 2.

In some embodiments, a HC polypeptide comprises: (i) an HC CDR1 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; (ii) an HC CDR2 of SEQ ID NO: 104, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 104; and/or (iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, a HC polypeptide comprises: (i) an HC CDR1 of SEQ ID NO: 113, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 113; (ii) an HC CDR2 of SEQ ID NO: 114, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 114; and/or (iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, a HC polypeptide comprises: (i) an HC CDR1 of SEQ ID NO: 120, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 120; (ii) an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and/or (iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, a HC polypeptide comprises: (i) an HC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; (ii) an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and/or (iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, a HC polypeptide comprises: (i) an HC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; (ii) an HC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and/or (iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, a HC polypeptide comprises: (i) an HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134; (ii) an HC CDR2 of SEQ ID NO: 135, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 135; and/or (iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, a HC polypeptide comprises: (i) an HC CDR1 of SEQ ID NO: 140, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 140; (ii) an HC CDR2 of SEQ ID NO: 141, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 141; and/or (iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, a HC polypeptide comprises: (i) an HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134; (ii) an HC CDR2 of SEQ ID NO: 145, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 145; and/or (iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, a HC polypeptide comprises: (i) an HC CDR1 of SEQ ID NO: 149, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 149; (ii) an HC CDR2 of SEQ ID NO: 150, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 150; and/or (iii) an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

In some embodiments, a HC polypeptide comprises: (i) an HC CDR1 of SEQ ID NO: 157, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 157; (ii) an HC CDR2 of SEQ ID NO: 158, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 158; and/or (iii) an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

In some embodiments, a HC polypeptide comprises: (i) an HC CDR1 of SEQ ID NO: 163, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 163; (ii) an HC CDR2 of SEQ ID NO: 164, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 164; and/or (iii) an HC CDR3 of SEQ ID NO: 165, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 165.

In some embodiments, a HC polypeptide comprises: (i) an HC CDR1 of SEQ ID NO: 169, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 169; (ii) an HC CDR2 of SEQ ID NO: 170, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 170; and/or (iii) an HC CDR3 of SEQ ID NO: 171, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 171.

In some embodiments, a HC polypeptide comprises: (i) an HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179; (ii) an HC CDR2 of SEQ ID NO: 180, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 180; and/or (iii) an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

In some embodiments, a HC polypeptide comprises: (i) an HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179; (ii) an HC CDR2 of SEQ ID NO: 186, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 186; and/or (iii) an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

In some embodiments, a HC polypeptide comprises: (i) an HC CDR1 of SEQ ID NO: 190, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 190; (ii) an HC CDR2 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191; and/or (iii) an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

In some embodiments, a HC polypeptide comprises: (i) an HC CDR1 of SEQ ID NO: 196, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:196; (ii) an HC CDR2 of SEQ ID NO: 197, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 197; and/or (iii) an HC CDR3 of SEQ ID NO: 198, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 198.

In some embodiments, a HC polypeptide comprises: (i) an HC CDR1 of SEQ ID NO: 202, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:202; (ii) an HC CDR2 of SEQ ID NO: 203, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 203; and/or (iii) an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

In some embodiments, a HC polypeptide comprises: (i) an HC CDR1 of SEQ ID NO: 209, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:209; (ii) an HC CDR2 of SEQ ID NO: 210, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 210; and/or (iii) an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

In some embodiments, a HC polypeptide further comprises one or more framework regions (FR), e.g., as described herein. In some embodiments, such a HC polypeptide comprises one, two, three or four FRs, e.g., as described herein. In some embodiments, a FR comprises a HC FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a HC FR sequence as described herein, or a sequence having at least 5, 10 or 20 alterations relative to a HC FR sequence as described herein.

In some embodiments, a HC polypeptide comprises: (i) a FR sequence provided in Table 2; (ii) a sequence with at least 92%, 93%, 94%, 95%, 96%, 97%, 98%, or 99% identity to a FR sequence provided in Table 2; or (iii) a sequence having at least 5, 10, or 20 alterations (e.g., substitutions, deletions or insertions (e.g., conservative substitutions)) compared to a FR sequence provided in Table 2.

In some embodiments, a HC polypeptide comprises a HC FR1 provided in Table 2, a sequence with at least 92%, 93%, 94%, 95%, 96%, 97%, 98%, or 99% identity to a HC FR1 sequence provided in Table 2, or a sequence having at least 5, 10, or 20 alterations (e.g., substitutions, deletions or insertions (e.g., conservative substitutions)) compared to a HC FR1 sequence provided in Table 2.

In some embodiments, a HC polypeptide comprises a HC FR2 provided in Table 2, a sequence with at least 92%, 93%, 94%, 95%, 96%, 97%, 98%, or 99% identity to a HC FR2 sequence provided in Table 2, or a sequence having at least 5, 10, or 20 alterations (e.g., substitutions, deletions or insertions (e.g., conservative substitutions)) compared to a HC FR2 sequence provided in Table 2.

In some embodiments a HC polypeptide comprises a HC FR3 provided in Table 2, a sequence with at least 92%, 93%, 94%, 95%, 96%, 97%, 98%, or 99% identity to a HC FR3 sequence provided in Table 2, or a sequence having at least 5, 10, or 20 alterations (e.g., substitutions, deletions or insertions (e.g., conservative substitutions)) compared to a HC FR3 sequence provided in Table 2.

In some embodiments, a HC polypeptide comprises a HC FR4 provided in Table 2, a sequence with at least 92%, 93%, 94%, 95%, 96%, 97%, 98%, or 99% identity to a HC FR4 sequence provided in Table 2, or a sequence having at least 5, 10, or 20 alterations (e.g., substitutions, deletions or insertions (e.g., conservative substitutions)) compared to a HC FR4 sequence provided in Table 2.

In some embodiments, a HC polypeptide comprises a HC CDR1, a HC CDR2 and HC CDR3 provided in Table 2 or a sequence with at least 85% identity thereto, and a HC FR1, HC FR2, HCFR3 and a HC FR4 provided in Table 2 or a sequence with at least 92% identity thereto.

In some embodiments, a HC polypeptide comprises the sequence of SEQ ID NO: 110, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 110.

In some embodiments, a HC polypeptide comprises the sequence of SEQ ID NO: 117, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 117.

In some embodiments, a HC polypeptide comprises) the sequence of SEQ ID NO: 122, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 122.

In some embodiments, a HC polypeptide comprises the sequence of SEQ ID NO: 126, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 126.

In some embodiments, a HC polypeptide comprises the sequence of SEQ ID NO: 131, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 131.

In some embodiments, a HC polypeptide comprises the sequence of SEQ ID NO: 137, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 137.

In some embodiments, a HC polypeptide comprises the sequence of SEQ ID NO: 142, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 142.

In some embodiments, a HC polypeptide comprises the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146.

In some embodiments, a HC polypeptide comprises the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154.

In some embodiments, a HC polypeptide comprises the sequence of SEQ ID NO: 160, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 160.

In some embodiments, a HC polypeptide comprises the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166.

In some embodiments, a HC polypeptide comprises the sequence of SEQ ID NO: 176, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 176.

In some embodiments, a HC polypeptide comprises the sequence of SEQ ID NO: 183, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 183.

In some embodiments, a HC polypeptide comprises the sequence of SEQ ID NO: 187, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 187.

In some embodiments, a HC polypeptide comprises the sequence of SEQ ID NO: 193, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 193.

In some embodiments, a HC polypeptide comprises the sequence of SEQ ID NO: 199 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 199.

In some embodiments, a HC polypeptide comprises the sequence of SEQ ID NO: 206 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206.

In some embodiments, a HC polypeptide comprises the sequence of SEQ ID NO: 211 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 211.

In some embodiments, a HC polypeptide comprises a HC amino acid sequence provided in Table 2, e.g., any one of SEQ ID NOs: 111, 118, 123, 127, 132, 138, 143, 147, 155, 161, 167, 177, 184, 188, 194, 200, 207, 212; or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to a HC amino acid sequence provided in Table 2, e.g., any one of SEQ ID NOs: 111, 118, 123, 127, 132, 138, 143, 147, 155, 161, 167, 177, 184, 188, 194, 200, 207, 212.

In some embodiments, a HC polypeptide disclosed herein comprises a terminal lysine, e.g., as provided in Table 2. In some embodiments, a HC polypeptide disclosed herein does not comprise a terminal lysine.

Exemplary useful HC polypeptides which may be included in an Activin A antibody agents disclosed herein are disclosed in Table 2 below.

TABLE 2
Exemplary heavy chain polypeptide and nucleic acid sequences.

| | SEQ | | |
|---|---|---|---|
| | ID | | |
| CLONE | NO | FEATURE | SEQUENCE |
| Antibody A | 103 | HC CDR1 | YTFTGYYMH |
| VH germline | 104 | HC CDR2 | SINPSSGGTNYAQKFQG |
| VH1-02 | 105 | HC CDR3 | ARARDIAAAGLGAFDY |
| Lineage 1 | 106 | HC FR1 | QVQLVQSGAEVKKPGASVKVSCKASG |
| | 107 | HC FR2 | WVRQAPGQGLEWMG |
| | 108 | HC FR3 | RVTMTTDTSTSTAYMELRSLRSDDTAVYYC |
| | 109 | HC FR4 | WGQGTLVTVSS |
| | 110 | VH amino | QVQLVQSGAEVKKPGASVKVSCKASGYTFTGYYMH |
| | | acid | WVRQAPGQGLEWMGSINPSSGGTNYAQKFQGRVTM |
| | | | TTDTSTSTAYMELRSLRSDDTAVYYCARARDIAAAG |
| | | | LGAFDYWGQGTLVTVSS |
| | 111 | HC | QVQLVQSGAEVKKPGASVKVSCKASGYTFTGYYMH |
| | | | WVRQAPGQGLEWMGSINPSSGGTNYAQKFQGRVTM |
| | | | TTDTSTSTAYMELRSLRSDDTAVYYCARARDIAAAG |
| | | | LGAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 112 | VH DNA | CAGGTGCAGCTGGTGCAGTCTGGGGCTGAGGTGAA |
| | | | GAAGCCTGGGGCCTCAGTGAAGGTCTCCTGCAAGG |
| | | | CTTCTGGATACACCTTCACCGGCTACTATATGCACT |
| | | | GGGTGCGACAGGCCCCTGGACAAGGGCTTGAGTGG |
| | | | ATGGGAAGCATCAACCCTAGCAGTGGTGGCACAAA |
| | | | CTATGCACAGAAGTTTCAGGGCAGGGTCACCATGA |
| | | | CCACAGACACATCCACGAGCACAGCCTACATGGAG |
| | | | CTGAGGAGCCTGAGATCTGACGACACGGCGGTGTA |
| | | | CTACTGCGCAAGGGCTAGAGACATAGCAGCAGCAG |
| | | | GCTTGGGAGCTTTTGACTACTGGGGACAGGGTACA |
| | | | TTGGTCACCGTCTCCTCA |
| Antibody B | 113 | HC CDR1 | YTFIGYDMH |
| VH germline | 114 | HC CDR2 | VINPSSGGTNYAQKFQG |
| VH1-02 | 115 | HC CDR3 | ARARDIAALGLGAFDY |
| Lineage 1 | 106 | HC FR1 | QVQLVQSGAEVKKPGASVKVSCKASG |
| | 107 | HC FR2 | WVRQAPGQGLEWMG |
| | 116 | HC FR3 | RVTMTRDTSISTAYMELSRLRSDDTAVYYC |
| | 109 | HC FR4 | WGQGTLVTVSS |
| | 117 | VH amino | QVQLVQSGAEVKKPGASVKVSCKASGYTFIGYDMH |
| | | acid | WVRQAPGQGLEWMGVINPSSGGTNYAQKFQGRVTM |
| | | | TRDTSISTAYMELSRLRSDDTAVYYCARARDIAALGL |
| | | | GAFDYWGQGTLVTVSS |
| | 118 | HC | QVQLVQSGAEVKKPGASVKVSCKASGYTFIGYDMH |
| | | | WVRQAPGQGLEWMGVINPSSGGTNYAQKFQGRVTM |
| | | | TRDTSISTAYMELSRLRSDDTAVYYCARARDIAALGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 119 | VH DNA | CAGGTGCAGCTGGTGCAGTCTGGGGCTGAGGTGAA |
| | | | GAAGCCTGGGGCCTCAGTGAAGGTCTCCTGCAAGG |
| | | | CTTCTGGATACACCTTCATAGGCTACGACATGCACT |
| | | | GGGTGCGACAGGCCCCTGGACAAGGGCTTGAGTGG |
| | | | ATGGGAGTCATCAACCCTAGCAGTGGTGGCACAAA |
| | | | CTATGCACAGAAGTTTCAGGGCAGGGTCACCATGA |
| | | | CCAGGGACACGTCCATCAGCACAGCCTACATGGAG |
| | | | CTGAGCAGGCTGAGATCTGACGACACGGCGGTGTA |
| | | | CTACTGCGCAAGGGCTAGAGACATAGCAGCATTGG |
| | | | GCTTGGGAGCTTTTGACTACTGGGGACAGGGTACA |
| | | | TTGGTCACCGTCTCCTCA |
| Antibody C | 120 | HC CDR1 | YTFPGYHMH |
| VH germline | 121 | HC CDR2 | VINPSSGDTNYAQKFQG |
| VH1-02 | 115 | HC CDR3 | ARARDIAALGLGAFDY |
| Lineage 1 | 106 | HC FR1 | QVQLVQSGAEVKKPGASVKVSCKASG |
| | 107 | HC FR2 | WVRQAPGQGLEWMG |
| | 116 | HC FR3 | RVTMTRDTSISTAYMELSRLRSDDTAVYYC |
| | 109 | HC FR4 | WGQGTLVTVSS |
| | 122 | VH amino | QVQLVQSGAEVKKPGASVKVSCKASGYTFPGYHMH |
| | | acid | WVRQAPGQGLEWMGVINPSSGDTNYAQKFQGRVTM |
| | | | TRDTSISTAYMELSRLRSDDTAVYYCARARDIAALGL |
| | | | GAFDYWGQGTLVTVSS |
| | 123 | HC | QVQLVQSGAEVKKPGASVKVSCKASGYTFPGYHMH |
| | | | WVRQAPGQGLEWMGVINPSSGDTNYAQKFQGRVTM |
| | | | TRDTSISTAYMELSRLRSDDTAVYYCARARDIAALGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 124 | VH DNA | CAGGTGCAGCTGGTGCAGTCTGGGGCTGAGGTGAA |
| | | | GAAGCCTGGGGCCTCAGTGAAGGTCTCCTGCAAGG |
| | | | CTTCTGGATACACCTTCCCCGGCTACCACATGCACT |
| | | | GGGTGCGACAGGCCCCTGGACAAGGGCTTGAGTGG |
| | | | ATGGGAGTCATCAACCCTAGCAGTGGTGACACAAA |
| | | | CTATGCACAGAAGTTTCAGGGCAGGGTCACCATGA |
| | | | CCAGGGACACGTCCATCAGCACAGCCTACATGGAG |
| | | | CTGAGCAGGCTGAGATCTGACGACACGGCGGTGTA |
| | | | CTACTGCGCAAGGGCTAGAGACATAGCAGCATTGG |
| | | | GCTTGGGAGCTTTTGACTACTGGGGACAGGGTACA |
| | | | TTGGTCACCGTCTCCTCA |
| Antibody D | 125 | HC CDR1 | YTFFGYSMH |
| VH germline | 121 | HC CDR2 | VINPSSGDTNYAQKFQG |
| VH1-02 | 115 | HC CDR3 | ARARDIAALGLGAFDY |
| Lineage 1 | 106 | HC FR1 | QVQLVQSGAEVKKPGASVKVSCKASG |
| | 107 | HC FR2 | WVRQAPGQGLEWMG |
| | 116 | HC FR3 | RVTMTRDTSISTAYMELSRLRSDDTAVYYC |
| | 109 | HC FR4 | WGQGTLVTVSS |
| | 126 | VH amino | QVQLVQSGAEVKKPGASVKVSCKASGYTFFGYSMH |
| | | acid | WVRQAPGQGLEWMGVINPSSGDTNYAQKFQGRVTM |
| | | | TRDTSISTAYMELSRLRSDDTAVYYCARARDIAALGL |
| | | | GAFDYWGQGTLVTVSS |
| | 127 | HC | QVQLVQSGAEVKKPGASVKVSCKASGYTFFGYSMH |
| | | | WVRQAPGQGLEWMGVINPSSGDTNYAQKFQGRVTM |
| | | | TRDTSISTAYMELSRLRSDDTAVYYCARARDIAALGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 128 | VH DNA | CAGGTGCAGCTGGTGCAGTCTGGGGCTGAGGTGAA |
| | | | GAAGCCTGGGGCCTCAGTGAAGGTCTCCTGCAAGG |
| | | | CTTCTGGATACACCTTCTTCGGCTACTCCATGCACT |
| | | | GGGTGCGACAGGCCCCTGGACAAGGGCTTGAGTGG |
| | | | ATGGGAGTCATCAACCCTAGCAGTGGTGACACAAA |
| | | | CTATGCACAGAAGTTTCAGGGCAGGGTCACCATGA |
| | | | CCAGGGACACGTCCATCAGCACAGCCTACATGGAG |
| | | | CTGAGCAGGCTGAGATCTGACGACACGGCGGTGTA |
| | | | CTACTGCGCAAGGGCTAGAGACATAGCAGCATTGG |
| | | | GCTTGGGAGCTTTTGACTACTGGGGACAGGGTACA |
| | | | TTGGTCACCGTCTCCTCA |
| Antibody E | 129 | HC CDR1 | YTFHGYNMH |
| VH germline | 130 | HC CDR2 | SINPYSGYTIYAQKFQG |
| VH1-02 | 105 | HC CDR3 | ARARDIAAAGLGAFDY |
| Lineage 1 | 106 | HC FR1 | QVQLVQSGAEVKKPGASVKVSCKASG |
| | 107 | HC FR2 | WVRQAPGQGLEWMG |
| | 116 | HC FR3 | RVTMTRDTSISTAYMELSRLRSDDTAVYYC |
| | 109 | HC FR4 | WGQGTLVTVSS |
| | 131 | VH amino | QVQLVQSGAEVKKPGASVKVSCKASGYTFHGYNMH |
| | | acid | WVRQAPGQGLEWMGSINPYSGYTIYAQKFQGRVTM |
| | | | TRDTSISTAYMELSRLRSDDTAVYYCARARDIAAAGL |
| | | | GAFDYWGQGTLVTVSS |
| | 132 | HC | QVQLVQSGAEVKKPGASVKVSCKASGYTFHGYNMH |
| | | | WVRQAPGQGLEWMGSINPYSGYTIYAQKFQGRVTM |
| | | | TRDTSISTAYMELSRLRSDDTAVYYCARARDIAAAGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 133 | VH DNA | CAGGTGCAGCTGGTGCAGTCTGGGGCTGAGGTGAA |
| | | | GAAGCCTGGGGCCTCAGTGAAGGTCTCCTGCAAGG |
| | | | CTTCTGGATACACCTTCCACGGCTACAACATGCACT |
| | | | GGGTGCGACAGGCCCCTGGACAAGGGCTTGAGTGG |
| | | | ATGGGATCGATCAACCCTTACAGTGGTTACACAAT |
| | | | CTATGCACAGAAGTTTCAGGGCAGGGTCACCATGA |
| | | | CCAGGGACACGTCCATCAGCACAGCCTACATGGAG |
| | | | CTGAGCAGGCTGAGATCTGACGACACGGCGGTGTA |
| | | | CTACTGCGCAAGGGCTAGAGACATAGCAGCAGCAG |
| | | | GCTTGGGAGCTTTTGACTACTGGGGACAGGGTACA |
| | | | TTGGTCACCGTCTCCTCA |
| Antibody F | 134 | HC CDR1 | YTFTGYHIH |
| VH germline | 135 | HC CDR2 | VISAYSGDTNYAQKLQG |
| VH1-18 | 115 | HC CDR3 | ARARDIAALGLGAFDY |
| Lineage 4 | 106 | HC FR1 | QVQLVQSGAEVKKPGASVKVSCKASG |
| | 136 | HC FR2 | WVRQVPGQGLEWMG |
| | 108 | HC FR3 | RVTMTTDTSTSTAYMELRSLRSDDTAVYYC |
| | 109 | HC FR4 | WGQGTLVTVSS |
| | 137 | VH amino | QVQLVQSGAEVKKPGASVKVSCKASGYTFTGYHIHW |
| | | acid | VRQVPGQGLEWMGVISAYSGDTNYAQKLQGRVTMT |
| | | | TDTSTSTAYMELRSLRSDDTAVYYCARARDIAALGL |
| | | | GAFDYWGQGTLVTVSS |
| | 138 | HC | QVQLVQSGAEVKKPGASVKVSCKASGYTFTGYHIHW |
| | | | VRQVPGQGLEWMGVISAYSGDTNYAQKLQGRVTMT |
| | | | TDTSTSTAYMELRSLRSDDTAVYYCARARDIAALGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 139 | VH DNA | CAGGTTCAGCTGGTGCAGTCTGGAGCTGAGGTGAA |
| | | | GAAGCCTGGGGCCTCAGTGAAGGTCTCCTGCAAGG |
| | | | CTTCTGGTTACACCTTTACCGGATATCACATCCACT |
| | | | GGGTGCGACAGGTCCCTGGACAAGGGCTTGAGTGG |
| | | | ATGGGAGTCATCAGCGCTTACAGTGGTGACACAAA |
| | | | CTATGCACAGAAGCTCCAGGGCAGAGTCACCATGA |
| | | | CCACAGACACATCCACGAGCACAGCCTACATGGAG |
| | | | CTGAGGAGCCTGAGATCTGACGACACGGCGGTGTA |
| | | | CTACTGCGCAAGGGCTAGAGACATAGCAGCATTGG |
| | | | GCTTGGGAGCTTTTGACTACTGGGGACAGGGTACA |
| | | | TTGGTCACCGTCTCCTCA |
| Antibody G | 140 | HC CDR1 | YTFTSYYIH |
| VH germline | 141 | HC CDR2 | AIYAYSGDTNYAQKLQG |
| VH1-18 | 105 | HC CDR3 | ARARDIAAAGLGAFDY |
| Lineage 4 | 106 | HC FR1 | QVQLVQSGAEVKKPGASVKVSCKASG |
| | 107 | HC FR2 | WVRQAPGQGLEWMG |
| | 108 | HC FR3 | RVTMTTDTSTSTAYMELRSLRSDDTAVYYC |
| | 109 | HC FR4 | WGQGTLVTVSS |
| | 142 | VH amino | QVQLVQSGAEVKKPGASVKVSCKASGYTFTSYYIHW |
| | | acid | VRQAPGQGLEWMGAIYAYSGDTNYAQKLQGRVTMT |
| | | | TDTSTSTAYMELRSLRSDDTAVYYCARARDIAAAGL |
| | | | GAFDYWGQGTLVTVSS |
| | 143 | HC | QVQLVQSGAEVKKPGASVKVSCKASGYTFTSYYIHW |
| | | | VRQAPGQGLEWMGAIYAYSGDTNYAQKLQGRVTMT |
| | | | TDTSTSTAYMELRSLRSDDTAVYYCARARDIAAAGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | 144 | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | | VH DNA | CAGGTTCAGCTGGTGCAGTCTGGAGCTGAGGTGAA |
| | | | GAAGCCTGGGGCCTCAGTGAAGGTCTCCTGCAAGG |
| | | | CTTCTGGTTACACCTTTACCAGCTATTACATCCACT |
| | | | GGGTGCGACAGGCCCCTGGACAAGGGCTTGAGTGG |
| | | | ATGGGAGCCATCTACGCTTACAGTGGTGACACAAA |
| | | | CTATGCACAGAAGCTCCAGGGCAGAGTCACCATGA |
| | | | CCACAGACACATCCACGAGCACAGCCTACATGGAG |
| | | | CTGAGGAGCCTGAGATCTGACGACACGGCGGTGTA |
| | | | CTACTGCGCAAGGGCTAGAGACATAGCAGCAGCAG |
| | | | GCTTGGGAGCTTTTGACTACTGGGGACAGGGTACA |
| | | | TTGGTCACCGTCTCCTCA |
| Antibody H | 134 | HC CDR1 | YTFTGYHIH |
| VH germline | 145 | HC CDR2 | HISAYSGTTNYAQKLQG |
| VH1-18 | 105 | HC CDR3 | ARARDIAAAGLGAFDY |
| Lineage 4 | 106 | HC FR1 | QVQLVQSGAEVKKPGASVKVSCKASG |
| | 107 | HC FR2 | WVRQAPGQGLEWMG |
| | 108 | HC FR3 | RVTMTTDTSTSTAYMELRSLRSDDTAVYYC |
| | 109 | HC FR4 | WGQGTLVTVSS |
| | 146 | VH amino | QVQLVQSGAEVKKPGASVKVSCKASGYTFTGYHIHW |
| | | acid | VRQAPGQGLEWMGHISAYSGTTNYAQKLQGRVTMT |
| | | | TDTSTSTAYMELRSLRSDDTAVYYCARARDIAAAGL |
| | | | GAFDYWGQGTLVTVSS |
| | 147 | HC | QVQLVQSGAEVKKPGASVKVSCKASGYTFTGYHIHW |
| | | | VRQAPGQGLEWMGHISAYSGTTNYAQKLQGRVTMT |
| | | | TDTSTSTAYMELRSLRSDDTAVYYCARARDIAAAGL |
| | | | GAFDYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 148 | VH DNA | CAGGTTCAGCTGGTGCAGTCTGGAGCTGAGGTGAA |
| | | | GAAGCCTGGGGCCTCAGTGAAGGTCTCCTGCAAGG |
| | | | CTTCTGGTTACACCTTTACCGGATATCACATCCACT |
| | | | GGGTGCGACAGGCCCCTGGACAAGGGCTTGAGTGG |
| | | | ATGGGACACATCAGCGCTTACAGTGGTACCACAAA |
| | | | CTATGCACAGAAGCTCCAGGGCAGAGTCACCATGA |
| | | | CCACAGACACATCCACGAGCACAGCCTACATGGAG |
| | | | CTGAGGAGCCTGAGATCTGACGACACGGCGGTGTA |
| | | | CTACTGCGCAAGGGCTAGAGACATAGCAGCAGCAG |
| | | | GCTTGGGAGCTTTTGACTACTGGGGACAGGGTACA |
| | | | TTGGTCACCGTCTCCTCA |
| Antibody I | 149 | HC CDR1 | GTFSNYAIS |
| VH germline | 150 | HC CDR2 | YIIPFFGTANYAQKFQG |
| VH1-69 | 151 | HC CDR3 | ARGGSGYSTSWGSSLY |
| Lineage 10 | 152 | HC FR1 | QVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 154 | VH amino | QVQLVQSGAEVKKPGSSVKVSCKASGGTFSNYAISW |
| | | acid | VRQAPGQGLEWMGYIIPFFGTANYAQKFQGRVTITA |
| | | | DESTSTAYMELSSLRSEDTAVYYCARGGSGYSTSWG |
| | | | SSLYWGQGTLVTVSS |
| | 155 | HC | QVQLVQSGAEVKKPGSSVKVSCKASGGTFSNYAISW |
| | | | VRQAPGQGLEWMGYIIPFFGTANYAQKFQGRVTITA |
| | | | DESTSTAYMELSSLRSEDTAVYYCARGGSGYSTSWG |
| | | | SSLYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 156 | VH DNA | CAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCACCTTCAGCAACTATGCTATCTCCT |
| | | | GGGTGCGACAGGCCCCTGGACAAGGGCTTGAGTGG |
| | | | ATGGGATACATCATCCCTTTCTTCGGTACAGCAAAC |
| | | | TACGCACAGAAGTTCCAGGGCAGAGTCACGATTAC |
| | | | CGCGGACGAATCCACGAGCACAGCCTACATGGAGC |
| | | | TGAGCAGCCTGAGATCTGAGGACACGGCGGTGTAC |
| | | | TACTGCGCCAGAGGCGGTTCTGGATACAGCACGAG |
| | | | CTGGGGTTCAAGCCTATACTGGGGACAGGGTACAT |
| | | | TGGTCACCGTCTCCTCA |
| Antibody J | 157 | HC CDR1 | GTFSLYAIS |
| VH germline | 158 | HC CDR2 | GIIPYFGTANYAQKFQG |
| VH1-69 | 151 | HC CDR3 | ARGGSGYSTSWGSSLY |
| Lineage 10 | 159 | HC FR1 | EVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 160 | VH amino | EVQLVQSGAEVKKPGSSVKVSCKASGGTFSLYAISW |
| | | acid | VRQAPGQGLEWMGGIIPYFGTANYAQKFQGRVTITA |
| | | | DESTSTAYMELSSLRSEDTAVYYCARGGSGYSTSWG |
| | | | SSLYWGQGTLVTVSS |
| | 161 | HC | EVQLVQSGAEVKKPGSSVKVSCKASGGTFSLYAISW |
| | | | VRQAPGQGLEWMGGIIPYFGTANYAQKFQGRVTITA |
| | | | DESTSTAYMELSSLRSEDTAVYYCARGGSGYSTSWG |
| | | | SSLYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 162 | VH DNA | GAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCACCTTCAGCCTCTATGCAATCAGCT |
| | | | GGGTGCGACAGGCCCCTGGACAAGGGCTTGAGTGG |
| | | | ATGGGAGGGATCATCCCTTACTTCGGTACAGCAAA |
| | | | CTACGCACAGAAGTTCCAGGGCAGAGTCACGATTA |
| | | | CCGCGGACGAATCCACGAGCACAGCCTACATGGAG |
| | | | CTGAGCAGCCTGAGATCTGAGGACACGGCGGTGTA |
| | | | CTACTGCGCCAGAGGCGGTTCTGGATACAGCACTA |
| | | | GCTGGGGTTCAAGCCTATACTGGGGACAGGGTACA |
| | | | TTGGTCACCGTCTCCTCA |
| Antibody K | 163 | HC CDR1 | GTSSSYAIS |
| VH germline | 164 | HC CDR2 | YIIPAFGTANYAQKFQG |
| VH1-69 | 165 | HC CDR3 | ARPVRTYPLGKYFYH |
| Lineage 19 | 152 | HC FR1 | QVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 166 | VH amino | QVQLVQSGAEVKKPGSSVKVSCKASGGTSSSYAISW |
| | | acid | VRQAPGQGLEWMGYIIPAFGTANYAQKFQGRVTITA |
| | | | DESTSTAYMELSSLRSEDTAVYYCARPVRTYPLGKYF |
| | | | YHWGQGTLVTVSS |
| | 167 | HC | QVQLVQSGAEVKKPGSSVKVSCKASGGTSSSYAISW |
| | | | VRQAPGQGLEWMGYIIPAFGTANYAQKFQGRVTITA |
| | | | DESTSTAYMELSSLRSEDTAVYYCARPVRTYPLGKYF |
| | | | YHWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 168 | VH DNA | CAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCACCTCCAGCAGCTATGCTATCAGCT |
| | | | GGGTGCGACAGGCCCCTGGACAAGGGCTTGAGTGG |
| | | | ATGGGATACATCATCCCTGCCTTCGGTACAGCAAA |
| | | | CTACGCACAGAAGTTCCAGGGCAGAGTCACGATTA |
| | | | CCGCGGACGAATCCACGAGCACAGCCTACATGGAG |
| | | | CTGAGCAGCCTGAGATCTGAGGACACGGCGGTGTA |
| | | | CTACTGCGCCAGACCGGTGAGAACCTACCCTCTAG |
| | | | GCAAGTACTTCTATCACTGGGGACAGGGTACATTG |
| | | | GTCACCGTCTCCTCA |
| Antibody L | 169 | HC CDR1 | YSISSGYYWG |
| VH germline | 170 | HC CDR2 | SIYHSGSTYYNPSLKS |
| VH4-0B | 171 | HC CDR3 | AREAGSGYWYLSVYGMDV |
| Lineage 42 | 172 | HC FR1 | QVQLQESGPGLVKPSETLSLTCAVSG |
| | 173 | HC FR2 | WIRQPPGKGLEWIG |
| | 174 | HC FR3 | RVTISVDTSKNQFSLKLSSVTAADTAVYYC |
| | 175 | HC FR4 | WGQGTTVTVSS |
| | 176 | VH amino | QVQLQESGPGLVKPSETLSLTCAVSGYSISSGYYWGW |
| | | acid | IRQPPGKGLEWIGSIYHSGSTYYNPSLKSRVTISVDTS |
| | | | KNQFSLKLSSVTAADTAVYYCAREAGSGYWYLSVY |
| | | | GMDVWGQGTTVTVSS |
| | 177 | HC | QVQLQESGPGLVKPSETLSLTCAVSGYSISSGYYWGW |
| | | | IRQPPGKGLEWIGSIYHSGSTYYNPSLKSRVTISVDTS |
| | | | KNQFSLKLSSVTAADTAVYYCAREAGSGYWYLSVY |
| | | | GMDVWGQGTTVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 178 | VH DNA | CAGGTGCAGCTGCAGGAGTCGGGCCCAGGACTGGT |
| | | | GAAGCCTTCGGAGACCCTGTCCCTCACCTGCGCTGT |
| | | | CTCTGGTTACTCCATCAGCAGTGGTTACTACTGGGG |
| | | | CTGGATCCGGCAGCCCCCAGGGAAGGGGCTGGAGT |
| | | | GGATTGGGAGTATCTATCATAGTGGGAGCACCTAC |
| | | | TACAACCCGTCCCTCAAGAGTCGAGTCACCATATC |
| | | | AGTAGACACGTCCAAGAACCAGTTCTCCCTGAAGC |
| | | | TGAGTTCTGTGACCGCCGCAGACACGGCGGTGTAC |
| | | | TACTGCGCCAGAGAAGCCGGAAGCGGATACTGGTA |
| | | | CTTGAGTGTATACGGAATGGACGTATGGGGCCAGG |
| | | | GAACAACTGTCACCGTCTCCTCA |
| Antibody M | 179 | HC CDR1 | GSISSSDYYWG |
| VH germline | 180 | HC CDR2 | SIYYPANTYYNPSLKS |
| VH4-39 | 181 | HC CDR3 | ARGYWNYGMDV |
| Lineage 50 | 182 | HC FR1 | QLQLQESGPGLVKPSETLSLTCTVSG |
| | 173 | HC FR2 | WIRQPPGKGLEWIG |
| | 174 | HC FR3 | RVTISVDTSKNQFSLKLSSVTAADTAVYYC |
| | 175 | HC FR4 | WGQGTTVTVSS |
| | 183 | VH amino | QLQLQESGPGLVKPSETLSLTCTVSGGSISSSDYYWG |
| | | acid | WIRQPPGKGLEWIGSIYYPANTYYNPSLKSRVTISVDT |
| | | | SKNQFSLKLSSVTAADTAVYYCARGYWNYGMDVW |
| | | | GQGTTVTVSS |
| | 184 | HC | QLQLQESGPGLVKPSETLSLTCTVSGGSISSSDYYWG |
| | | | WIRQPPGKGLEWIGSIYYPANTYYNPSLKSRVTISVDT |
| | | | SKNQFSLKLSSVTAADTAVYYCARGYWNYGMDVW |
| | | | GQGTTVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 185 | VH DNA | CAGCTGCAGCTGCAGGAGTCGGGCCCAGGACTGGT |
| | | | GAAGCCTTCGGAGACCCTGTCCCTCACCTGCACTGT |
| | | | CTCTGGTGGCTCCATCAGCAGTAGTGACTACTACTG |
| | | | GGGCTGGATCCGCCAGCCCCCAGGGAAGGGGCTGG |
| | | | AGTGGATTGGGAGTATCTACTATCCCGCAAACACC |
| | | | TACTACAACCCGTCCCTCAAGAGTCGAGTCACCAT |
| | | | ATCCGTAGACACGTCCAAGAACCAGTTCTCCCTGA |
| | | | AGCTGAGTTCTGTGACCGCCGCAGACACGGCGGTG |
| | | | TACTACTGCGCCAGAGGATATTGGAACTATGGCAT |
| | | | GGATGTGTGGGGCCAGGGAACAACTGTCACCGTCT |
| | | | CCTCA |
| Antibody N | 179 | HC CDR1 | GSISSSDYYWG |
| VH germline | 186 | HC CDR2 | SIYYPASTYYNPSLKS |
| VH4-39 | 181 | HC CDR3 | ARGYWNYGMDV |
| Lineage 50 | 182 | HC FR1 | QLQLQESGPGLVKPSETLSLTCTVSG |
| | 173 | HC FR2 | WIRQPPGKGLEWIG |
| | 174 | HC FR3 | RVTISVDTSKNQFSLKLSSVTAADTAVYYC |
| | 175 | HC FR4 | WGQGTTVTVSS |
| | 187 | VH amino | QLQLQESGPGLVKPSETLSLTCTVSGGSISSSDYYWG |
| | | acid | WIRQPPGKGLEWIGSIYYPASTYYNPSLKSRVTISVDT |
| | | | SKNQFSLKLSSVTAADTAVYYCARGYWNYGMDVW |
| | | | GQGTTVTVSS |
| | 188 | HC | QLQLQESGPGLVKPSETLSLTCTVSGGSISSSDYYWG |
| | | | WIRQPPGKGLEWIGSIYYPASTYYNPSLKSRVTISVDT |
| | | | SKNQFSLKLSSVTAADTAVYYCARGYWNYGMDVW |
| | | | GQGTTVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 189 | VH DNA | CAGCTGCAGCTGCAGGAGTCGGGCCCAGGACTGGT |
| | | | GAAGCCTTCGGAGACCCTGTCCCTCACCTGCACTGT |
| | | | CTCTGGTGGCTCCATCAGCAGTAGTGACTACTACTG |
| | | | GGGCTGGATCCGCCAGCCCCCAGGGAAGGGGCTGG |
| | | | AGTGGATTGGGAGTATCTACTATCCCGCAAGCACC |
| | | | TACTACAACCCGTCCCTCAAGAGTCGAGTCACCAT |
| | | | ATCCGTAGACACGTCCAAGAACCAGTTCTCCCTGA |
| | | | AGCTGAGTTCTGTGACCGCCGCAGACACGGCGGTG |
| | | | TACTACTGCGCCAGAGGATATTGGAACTATGGCAT |
| | | | GGATGTGTGGGGCCAGGGAACAACTGTCACCGTCT |
| | | | CCTCA |
| Antibody O | 190 | HC CDR1 | GTFSNYDIN |
| VH germline | 191 | HC CDR2 | GIIPVFGTAAYAQKFQG |
| VH1-69 | 192 | HC CDR3 | AREARDGYRYAFQH |
| Lineage 7 | 152 | HC FR1 | QVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 193 | VH amino | QVQLVQSGAEVKKPGSSVKVSCKASGGTFSNYDINW |
| | | acid | VRQAPGQGLEWMGGIIPVFGTAAYAQKFQGRVTITA |
| | | | DESTSTAYMELSSLRSEDTAVYYCAREARDGYRYAF |
| | | | QHWGQGTLVTVSS |
| | 194 | HC | QVQLVQSGAEVKKPGSSVKVSCKASGGTFSNYDINW |
| | | | VRQAPGQGLEWMGGIIPVFGTAAYAQKFQGRVTITA |
| | | | DESTSTAYMELSSLRSEDTAVYYCAREARDGYRYAF |
| | | | QHWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 195 | VH DNA | CAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCACCTTCAGCAACTATGACATCAAC |
| | | | TGGGTGCGACAGGCCCCTGGACAAGGGCTTGAGTG |
| | | | GATGGGAGGGATCATCCCTGTCTTCGGTACAGCAG |
| | | | CCTACGCACAGAAGTTCCAGGGCAGAGTCACGATT |
| | | | ACCGCGGACGAATCCACGAGCACAGCCTACATGGA |
| | | | GCTGAGCAGCCTGAGATCTGAGGACACGGCGGTGT |
| | | | ACTACTGCGCCAGAGAAGCTAGAGACGGATACAG |
| | | | ATACGCCTTCCAACACTGGGGACAGGGTACATTGG |
| | | | TCACCGTCTCCTCA |
| Antibody P | 196 | HC CDR1 | GTFDDYAIS |
| VH germline | 197 | HC CDR2 | GIIPRFGIANYAQKFQG |
| VH1-69 | 198 | HC CDR3 | ARPPSSGSD |
| Lineage 16 | 152 | HC FR1 | QVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 199 | VH amino | QVQLVQSGAEVKKPGSSVKVSCKASGGTFDDYAISW |
| | | acid | VRQAPGQGLEWMGGIIPRFGIANYAQKFQGRVTITAD |
| | | | ESTSTAYMELSSLRSEDTAVYYCARPPSSGSDWGQGT |
| | | | LVTVSS |
| | 200 | HC | QVQLVQSGAEVKKPGSSVKVSCKASGGTFDDYAISW |
| | | | VRQAPGQGLEWMGGIIPRFGIANYAQKFQGRVTITAD |
| | | | ESTSTAYMELSSLRSEDTAVYYCARPPSSGSDWGQGT |
| | | | LVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 201 | VH DNA | CAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCACCTTCGACGACTATGCTATCAGCT |
| | | | GGGTGCGACAGGCCCCTGGACAAGGGCTTGAGTGG |
| | | | ATGGGAGGGATCATCCCTAGATTCGGTATCGCAAA |
| | | | CTACGCACAGAAGTTCCAGGGCAGAGTCACGATTA |
| | | | CCGCGGACGAATCCACGAGCACAGCCTACATGGAG |
| | | | CTGAGCAGCCTGAGATCTGAGGACACGGCGGTGTA |
| | | | CTACTGCGCCAGACCACCTAGTTCTGGTTCTGATTG |
| | | | GGGACAGGGTACATTGGTCACCGTCTCCTCA |
| Antibody Q | 202 | HC CDR1 | GSFSPYTWS |
| VH germline | 203 | HC CDR2 | GIIPISGTANTAQKFQG |
| VH1-69 | 204 | HC CDR3 | ARGGPLSGWRASYYY |
| Lineage 52 | 152 | HC FR1 | QVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 206 | VH amino | QVQLVQSGAEVKKPGSSVKVSCKASGGSFSPYTWSW |
| | | acid | VRQAPGQGLEWMGGIIPISGTANTAQKFQGRVTITAD |
| | | | ESTSTAYMELSSLRSEDTAVYYCARGGPLSGWRASY |
| | | | YYWGQGTLVTVSS |
| | 207 | HC | QVQLVQSGAEVKKPGSSVKVSCKASGGSFSPYTWSW |
| | | | VRQAPGQGLEWMGGIIPISGTANTAQKFQGRVTITAD |
| | | | ESTSTAYMELSSLRSEDTAVYYCARGGPLSGWRASY |
| | | | YYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 208 | VH DNA | CAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCTCGTTCAGCCCCTATACATGGAGCT |
| | | | GGGTGCGACAGGCCCCTGGACAAGGGCTTGAGTGG |
| | | | ATGGGAGGGATCATCCCTATCTCTGGTACAGCAAA |
| | | | CACGGCACAGAAGTTCCAGGGCAGAGTCACGATTA |
| | | | CCGCGGACGAATCCACGAGCACAGCCTACATGGAG |
| | | | CTGAGCAGCCTGAGATCTGAGGACACGGCGGTGTA |
| | | | CTACTGCGCCAGAGGTGGGCCTTTGAGCGGTTGGA |
| | | | GAGCCAGCTACTACTATTGGGGACAGGGTACATTG |
| | | | GTCACCGTCTCCTCA |
| Antibody R | 209 | HC CDR1 | GSFSPYTIS |
| VH germline | 210 | HC CDR2 | GIILTSGTANYAQKFQG |
| VH1-69 | 204 | HC CDR3 | ARGGPLSGWRASYYY |
| Lineage 52 | 152 | HC FR1 | QVQLVQSGAEVKKPGSSVKVSCKASG |
| | 107 | HC FR2 | WVRQAPGQGLEWMG |
| | 153 | HC FR3 | RVTITADESTSTAYMELSSLRSEDTAVYYC |
| | 109 | HC FR4 | WGQGTLVTVSS |
| | 211 | VH amino | QVQLVQSGAEVKKPGSSVKVSCKASGGSFSPYTISWV |
| | | acid | RQAPGQGLEWMGGIILTSGTANYAQKFQGRVTITAD |
| | | | ESTSTAYMELSSLRSEDTAVYYCARGGPLSGWRASY |
| | | | YYWGQGTLVTVSS |
| | 212 | HC | QVQLVQSGAEVKKPGSSVKVSCKASGGSFSPYTISWV |
| | | | RQAPGQGLEWMGGIILTSGTANYAQKFQGRVTITAD |
| | | | ESTSTAYMELSSLRSEDTAVYYCARGGPLSGWRASY |
| | | | YYWGQGTLVTVSS |
| | | | ASTKGPSVFPLAPSSKSTSGGTAALGCLVKDYFPEPVT |
| | | | VSWNSGALTSGVHTFPAVLQSSGLYSLSSVVTVPSSS |
| | | | LGTQTYICNVNHKPSNTKVDKKVEPKSCDKTHTCPPC |
| | | | PAPEAAGAPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPSREEMTKNQVSLTCLVKGFYPS |
| | | | DIAVEWESNGQPENNYKTTPPVLDSDGSFFLYSKLTV |
| | | | DKSRWQQGNVFSCSVMHEALHNHYTQKSLSLSPGK |
| | 213 | VH DNA | CAGGTGCAGCTGGTGCAGTCTGGGGCTGAGGTGAA |
| | | | GAAGCCTGGGTCCTCGGTGAAGGTCTCCTGCAAGG |
| | | | CTTCTGGAGGCAGTTTCAGCCCCTATACAATCAGCT |
| | | | GGGTGCGACAGGCCCCTGGACAAGGGCTTGAGTGG |
| | | | ATGGGAGGGATCATCCTGACTTCTGGTACAGCAAA |
| | | | CTACGCACAGAAGTTCCAGGGCAGAGTCACGATTA |
| | | | CCGCGGACGAATCCACGAGCACAGCCTACATGGAG |
| | | | CTGAGCAGCCTGAGATCTGAGGACACGGCGGTGTA |
| | | | CTACTGCGCCAGAGGTGGGCCTTTGAGCGGTTGGA |
| | | | GAGCCAGCTACTACTATTGGGGACAGGGTACATTG |
| | | | GTCACCGTCTCCTCA |

IgG
(SEQ ID NO: 214)
ASTKGPSVFPLAPSSKSTSGGTAALGCLVKDYFPEPVTVSWNSGAL
TSGVHTFPAVLQSSGLYSLSSVVTVPSSSLGTQTYICNVNHKPSN
TKVDKKVEPKSCDKTHTCPPCPAPEAAGAPSVFLFPPKPKDTLMI
SRTPEVTCVVVDVSHEDPEVKFNWYVDGVEVHNAKTKPREEQYNS
TYRVVSVLTVLHQDWLNGKEYKCKVSNKALPAPIEKTISKAKGQP
REPQVYTLPPSREEMTKNQVSLTCLVKGFYPSDIAVEWESNGQPE
NNYKTTPPVLDSDGSFFLYSKLTVDKSRWQQGNVFSCSVMHEALH
NHYTQKSLSLSPGK

In some embodiments, a HC polypeptide disclosed herein comprises an IgG sequence of SEQ ID NO: 214 or a sequence with at least 85% identity thereto.

### Activin A Antibody Agents Comprising a Light Chain Polypeptide and a Heavy Chain Polypeptide

In some embodiments, an Activin A antibody agent disclosed herein, e.g., a Activin A antibody agent polypeptide, comprises a light chain comprising a variable region comprising one, two or three LC CDRs and a heavy chain comprising a variable region comprising one, two or three HC CDRs. In some embodiments, an Activin A antibody agent comprises a light chain comprising a LC CDR1, a LC CDR2 and a LC CDR3, and a heavy chain comprising a HC CDR1, a HC CDR2 and HC CDR3.

In some embodiments, an Activin A antibody agent comprising a LC CDR1, a LC CDR2 and a LC CDR3, and a heavy chain comprising a HC CDR1, a HC CDR2 and HC CDR3 is able to specifically bind to Activin A, e.g., human, primate, or a domestic mammal Activin A (e.g., a dog, a cat, a horse, a sheep, a cow, a yak and/or a camel).

In some embodiments, an Activin A antibody agent comprises one, two, or three LC CDRs provided in Table 1 or a sequence with at least 85%, 86%, 87%, 88%, 89%, 90%, 91%, 92%, 93%, 94%, 95%, 96%, 97%, 98%, or 99% thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% thereto.

In some embodiments, an Activin A antibody agent comprises: (a) a light chain comprising: (i) an LC CDR1 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR3 provided in Table 1; and (b) a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR3 provided in Table 2.

In some embodiments, an Activin A antibody agent comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3; and (ii) a HC CDR1 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; an HC CDR2 of SEQ ID NO: 104, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 104; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, an Activin A antibody agent comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 12, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 12; and (ii) a HC CDR1 of SEQ ID NO: 113, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 113; an HC CDR2 of SEQ ID NO: 114, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 114; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, an Activin A antibody agent comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 16, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 16; and an LC CDR3 of SEQ ID NO: 17, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 17; and (ii) a HC CDR1 of SEQ ID NO: 120, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 120; an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, an Activin A antibody agent comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 21, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 21; and (ii) a HC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, an Activin A antibody agent comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3; and (ii) a HC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; an HC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, an Activin A antibody agent comprises: (i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; an LC CDR2 of SEQ ID NO: 29 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 29; and an LC CDR3 of SEQ ID NO: 30, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 30; and (ii) a HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134; an HC CDR2 of SEQ ID NO: 135, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 135; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

In some embodiments, an Activin A antibody agent comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 36, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 36; and (ii) a HC CDR1 of SEQ ID NO: 140, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 140; an HC CDR2 of SEQ ID NO: 141, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 141; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, an Activin A antibody agent comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 36, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 36; and (ii) a HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134; an HC CDR2 of SEQ ID NO: 145, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 145; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

In some embodiments, an Activin A antibody agent comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 43 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 43; and an LC CDR3 of SEQ ID NO: 44, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 44; and (ii) a HC CDR1 of SEQ ID NO: 149, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 149; an HC CDR2 of SEQ ID NO: 150, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 150; and an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

In some embodiments, an Activin A antibody agent comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 48 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 48; and an LC CDR3 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49; and (ii) a HC CDR1 of SEQ ID NO: 157, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 157; an HC CDR2 of SEQ ID NO: 158, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 158; and an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

In some embodiments, an Activin A antibody agent comprises: (i) an LC CDR1 of SEQ ID NO: 53, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 53; an LC CDR2 of SEQ ID NO: 54 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 54; and an LC CDR3 of SEQ ID NO: 55, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 55; and (ii) a HC CDR1 of SEQ ID NO: 163, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 163; an HC CDR2 of SEQ ID NO: 164, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 164; and an HC CDR3 of SEQ ID NO: 165, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 165.

In some embodiments, an Activin A antibody agent comprises: (i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and an LC CDR3 of SEQ ID NO: 62, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 62; and (ii) a HC CDR1 of SEQ ID NO: 169, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 169; an HC CDR2 of SEQ ID NO: 170, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 170; and an HC CDR3 of SEQ ID NO: 171, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 171.

In some embodiments, an Activin A antibody agent comprises: (i) an LC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; an LC CDR2 of SEQ ID NO: 69 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and an LC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70; and (ii) a HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179; an HC CDR2 of SEQ ID NO: 180, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 180; and an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

In some embodiments, an Activin A antibody agent comprises: (i) an LC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; an LC CDR2 of SEQ ID NO: 69 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and an LC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70; and (ii) a HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179; an HC CDR2 of SEQ ID NO: 186, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 186; and an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

In some embodiments, an Activin A antibody agent comprises: (i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and an LC CDR3 of SEQ ID NO: 80, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 80; and (ii) a HC CDR1 of SEQ ID NO: 190, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 190; an HC CDR2 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191; and an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

In some embodiments, an Activin A antibody agent comprises: (i) an LC CDR1 of SEQ ID NO:85, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 85; an LC CDR2 of SEQ ID NO: 86 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 86; and an LC CDR3 of SEQ ID NO: 87, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 87; and (ii) a HC CDR1 of SEQ ID NO: 196, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 196; an HC CDR2 of SEQ ID NO: 197, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 197; and an HC CDR3 of SEQ ID NO: 198, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 198.

In some embodiments, an Activin A antibody agent comprises: (i) an LC CDR1 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; an LC CDR2 of SEQ ID NO: 94 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and an LC CDR3 of SEQ ID NO: 95, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 95; and (ii) a HC CDR1 of SEQ ID NO: 202, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:202; an HC CDR2 of SEQ ID NO: 203, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 203; and an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

In some embodiments, an Activin A antibody agent comprises: (i) an LC CDR1 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; an LC CDR2 of SEQ ID NO: 94 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and an LC CDR3 of SEQ ID NO: 95, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 95; and (ii) a HC CDR1 of SEQ ID NO: 209, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:209; an HC CDR2 of SEQ ID NO: 210, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 210; and an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

In some embodiments, an Activin A antibody agent comprises a light chain polypeptide (LC polypeptide) as described herein.

In some embodiments, an Activin A antibody agent comprises a heavy chain polypeptide (HC polypeptide) as described herein. In some embodiments, a HC polypeptide in an Activin A antibody agent does not include a terminal lysine.

In some embodiments, an Activin A antibody agent comprises a light chain polypeptide (LC polypeptide) as described herein and a heavy chain polypeptide (HC polypeptide) as described herein. In some embodiments, a HC polypeptide in an Activin A antibody agent does not include a terminal lysine.

In some embodiments, an Activin A antibody agent comprises a light chain comprising a variable region (VL) comprising three LC CDRs and one or more framework regions (e.g., as described herein); and a heavy chain comprising a variable region (VH) comprising three HC CDRs and one or more framework regions (e.g., as described herein).

In some embodiments, a VL and/or a VH of an Activin A antibody agent further comprises one or more framework regions (FR), e.g., as described herein. In some embodiments, a VL and/or a VH of an Activin A antibody agent comprises one, two, three or four FRs, e.g., as described herein. In some embodiments, a FR comprises a FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a LC FR sequence as described herein, or a sequence having at least 5, 10 or 20 alterations relative to a LC FR sequence as described herein.

In some embodiments, a VL and/or a VH of an Activin A antibody agent comprises: (i) a FR sequence provided in Table 1 or Table 2; (ii) a sequence with at least 92%, 93%, 94%, 95%, 96%, 97%, 98%, or 99% identity to a FR sequence provided in Table 1 or 2; or (iii) a sequence having at least 5, 10, or 20 alterations (e.g., substitutions, deletions or insertions (e.g., conservative substitutions)) compared to a FR sequence provided in Table 1 or 2.

In some embodiments, a VL and/or a VH of an Activin A antibody agent comprises a FR1 provided in Table 1 or 2, a sequence with at least 92%, 93%, 94%, 95%, 96%, 97%, 98%, or 99% identity to a FR1 sequence provided in Table 1 or 2, or a sequence having at least 5, 10, or 20 alterations (e.g., substitutions, deletions or insertions (e.g., conservative substitutions)) compared to a FR1 sequence provided in Table 1 or 2.

In some embodiments, a VL and/or a VH of an Activin A antibody agent comprises a FR2 provided in Table 1 or 2, a sequence with at least 92%, 93%, 94%, 95%, 96%, 97%, 98%, or 99% identity to a FR2 sequence provided in Table 1 or 2, or a sequence having at least 5, 10, or 20 alterations (e.g., substitutions, deletions or insertions (e.g., conservative substitutions)) compared to a FR2 sequence provided in Table 1 or 2.

In some embodiments a VL and/or a VH of an Activin A antibody agent comprises a FR3 provided in Table 1 or 2, a sequence with at least 92%, 93%, 94%, 95%, 96%, 97%, 98%, or 99% identity to a FR3 sequence provided in Table 1 or 2, or a sequence having at least 5, 10, or 20 alterations (e.g., substitutions, deletions or insertions (e.g., conservative substitutions)) compared to a FR3 sequence provided in Table 1 or 2.

In some embodiments, a VL and/or a VH of an Activin A antibody agent comprises a FR4 provided in Table 1 or 2, a sequence with at least 92%, 93%, 94%, 95%, 96%, 97%, 98%, or 99% identity to a FR4 sequence provided in Table 1 or 2, or a sequence having at least 5, 10, or 20 alterations (e.g., substitutions, deletions or insertions (e.g., conservative substitutions)) compared to a FR4 sequence provided in Table 1 or 2.

In some embodiments an Activin A antibody agent comprises a VL comprising 3 LC CDRs and a LC FR1, LC FR2, LCFR3 and a LC FR4 of an Activin A antibody agent provided in Table 1 or a sequence with at least 92% identity thereto; and/or a VH comprising 3 HC CDRs and a HC FR1, HC FR2, HC FR3 and a HC FR4 of an Activin A antibody agent provided in Table 2 or a sequence with at least 92% identity thereto.

In some embodiments, an Activin A antibody agent comprises a VL sequence provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a VL sequence provided in Table 1; and a VH sequence provided in Table 2 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to a VH sequence provided in Table 2.

In some embodiments, an Activin A antibody agent comprises: the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; and the sequence of SEQ ID NO: 110, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 110.

In some embodiments, an Activin A antibody agent comprises: the sequence of SEQ ID NO: 13, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 13; and the sequence of SEQ ID NO: 117, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 117.

In some embodiments, an Activin A antibody agent comprises: the sequence of SEQ ID NO: 18, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 18; and the sequence of SEQ ID NO: 122, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 122.

In some embodiments, an Activin A antibody agent comprises: the sequence of SEQ ID NO: 22, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 22; and the sequence of SEQ ID NO: 126, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 126.

In some embodiments, an Activin A antibody agent comprises the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; and the sequence of SEQ ID NO: 131, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 131.

In some embodiments, an Activin A antibody agent comprises the sequence of SEQ ID NO: 33, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 33; and the sequence of SEQ ID NO:137, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO:137.

In some embodiments, an Activin A antibody agent comprises the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; and the sequence of SEQ ID NO: 142, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 142.

In some embodiments, an Activin A antibody agent comprises the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; and the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146.

In some embodiments, an Activin A antibody agent comprises the sequence of SEQ ID NO: 45, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 45; and the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154.

In some embodiments, an Activin A antibody agent comprises the sequence of SEQ ID NO: 50, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 50; and the sequence of SEQ ID NO: 160, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 160.

In some embodiments, an Activin A antibody agent comprises the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166.

In some embodiments, an Activin A antibody agent comprises the sequence of SEQ ID NO: 65, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 65; and the sequence of SEQ ID NO: 176, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 176.

In some embodiments, an Activin A antibody agent comprises the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; and the sequence of SEQ ID NO: 183, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 183.

In some embodiments, an Activin A antibody agent comprises the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; and the sequence of SEQ ID NO: 187, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 187.

In some embodiments, an Activin A antibody agent comprises the sequence of SEQ ID NO: 82, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 82; and the sequence of SEQ ID NO: 193, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 193.

In some embodiments, an Activin A antibody agent comprises the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90; and the sequence of SEQ ID NO: 199, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 199.

In some embodiments, an Activin A antibody agent comprises the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206.

In some embodiments, an Activin A antibody agent comprises the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 211, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 211.

In some embodiments, an Activin A antibody agent comprises: a light chain comprising three LC CDRs, one or more framework regions (e.g., as described herein) and a constant region; and a heavy chain comprising three HC CDRs, one or more framework regions (e.g., as described herein), and at least one constant region.

In some embodiments, a light chain constant region comprises a light chain kappa or a light chain lambda constant region.

In some embodiments, a light chain kappa constant region comprises the sequence of SEQ ID NO: 102, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 102.

In some embodiments, a heavy chain constant region comprises a CH1, CH2 and/or CH3. In some embodiments, at least one constant region comprises an Fc domain. In some embodiments, an Fc domain comprises a mammalian Fc domain. In some embodiments, an Fc domain comprises a dog, a cat, a mouse, a rat, a rabbit, a primate, a cow, a horse or a human Fc domain. In some embodiments, an Fc domain is chosen from an Fc domain of an immunoglobulin isotype. In some embodiments, an immunoglobulin isotype comprises IgA, IgD, IgG, IgM, or IgE. In some embodiments, an Fc domain comprises an Fc domain of an IgG, e.g., a human IgG. In some embodiments, an IgG is or comprises IgG1, IgG2, IgG3, or IgG4.

In some embodiments, an Activin A antibody agent disclosed herein comprises an Fc region, e.g., as described herein. In some embodiments, an Fc region is a wildtype Fc region, e.g., a wildtype human Fc region. In some embodiments, an Fc region comprises a variant, e.g., an Fc region comprising an addition, substitution, or deletion of at least one amino acid residue in an Fc region which results in, e.g., reduced or ablated affinity for at least one Fc receptor.

In some embodiments, an Activin A antibody agent comprises one or more mutations in a constant region of an antibody. In some embodiments, an Activin A antibody agent comprises one or more mutations in a constant region as disclosed in U.S. Pat. No. 5,624,821, the entire contents of which is hereby incorporated by reference.

In some embodiments, an Activin A antibody agent comprising a variant Fc region. In some embodiments, a Fc region variant comprises Leu234Ala/Leu235Ala (LALA) mutation, a Leu235Glu (LE) mutation, a Ser228Pro/Leu235Glu (SPLE) mutation, Leu234Ala/Leu235Ala/Pro239Gly (LALA-PG) mutation, Pro 331 Ser/Leu234Glu/Leu235Phe (TM), Asp265Ala (DA) mutation, Leu235Ala/Gly237Ala (LAGA) mutation, or a combination thereof.

In some embodiments, an Activin A antibody agent disclosed herein comprises a Leu234Ala/Leu235Ala (LALA) mutation.

In some embodiments, an Activin A antibody agent disclosed herein comprises a Leu235Ala/Gly237Ala (LAGA) mutation.

In some embodiments, a Fc region variant comprises a mutation relative to a wildtype Fc region, e.g., a IgG1 FcR wildtype region. In some embodiments, the hinge and CH2 sequence of an IgG1 FcR wildtype region comprises the sequence of: CPPCPAPELLGGPSVFLFPPK (SEQ ID NO: 222).

In some embodiments, a Fc region variant comprises a LAGA mutation, e.g., as shown in bold in SEQ ID NO: 223: CPPCPAPELAGAPSVFLFPPK. In some embodiments, an Activin A antibody agent comprises an Fc region having a LAGA mutation, e.g., as provided in SEQ ID NO: 223.

In some embodiments, a Fc region variant comprises a FEGG mutation, e.g., as shown in bold in SEQ ID NO: 224: CPPCPAPEFEGGPSVFLFPPK. In some embodiments, an Activin A antibody agent comprises an Fc region having a FEGG mutation, e.g., as provided in SEQ ID NO: 224.

In some embodiments, a Fc region variant comprises a AAGG mutation, e.g., as shown in bold in SEQ ID NO: 225: CPPCPAPEAAGGPSVFLFPPK. In some embodiments, an Activin A antibody agent comprises an Fc region having a AAGG mutation, e.g., as provided in SEQ ID NO: 225.

In some embodiments, a Fc region variant comprises a AAGA mutation, e.g., as shown in bold in SEQ ID NO: 226: CPPCPAPEAAGAPSVFLFPPK. In some embodiments, an AAGA mutation is also referred to as Leu234Ala/Leu235Ala/Glu237Ala (LALAGA). In some embodiments, an Activin A antibody agent comprises an Fc region having an AAGA mutation, e.g., as provided in SEQ ID NO: 226.

In some embodiments, a Fc region variant comprising an Fc mutation has reduced binding (e.g., no binding) to a neonatal Fc receptor (FcRn), e.g., when compared to an otherwise similar Fc region without an Fc mutation. In some embodiments, a Activin A antibody agent comprising an Fc region having an Fc mutation has reduced binding (e.g., no binding) to FcRn and reduced placental transfer, compared to an otherwise similar Activin A antibody agent with an Fc region without an Fc mutation.

In some embodiments, a Fc region variant comprising a LAGA mutation, a FEGG mutation, an AAGG mutation, an AAGA mutation, a LALA mutation or a combination thereof, has reduced binding (e.g., no binding) to a neonatal Fc receptor (FcRn), e.g., when compared to an otherwise similar Fc region without a LAGA mutation, a FEGG mutation, an AAGG mutation, an AAGA mutation, a LALA mutation or a combination thereof.

In some embodiments, a Activin A antibody agent comprising an Fc region having a LAGA mutation, a FEGG mutation, an AAGG mutation, an AAGA mutation, a LALA mutation or a combination thereof, has reduced binding (e.g., no binding) to FcRn and reduced placental transfer, compared to an otherwise similar Activin A antibody agent with an Fc region without a LAGA mutation, a FEGG mutation, an AAGG mutation, an AAGA mutation, a LALA mutation or a combination thereof.

In some embodiments, a Fc region variant comprising a 1253A mutation, a H310A mutation, a H435R mutation, a H435A mutation or a combination thereof, has reduced binding (e.g., no binding) to a neonatal Fc receptor (FcRn), e.g., when compared to an otherwise similar Fc region without a I253A mutation, a H310A mutation, a H435R mutation, a H435A mutation or a combination thereof.

In some embodiments, a Activin A antibody agent comprising an Fc region having a I253A mutation, a H310A mutation, a H435R mutation, a H435A mutation or a combination thereof, has reduced binding (e.g., no binding) to FcRn and reduced placental transfer, compared to an otherwise similar Activin A antibody agent with an Fc region without a 1253A mutation, a H310A mutation, a H435R mutation, a H435A mutation or a combination thereof.

In some embodiments, an Activin A antibody agent disclosed herein comprises an IgG sequence of SEQ ID NO: 214 or a sequence with at least 85% identity thereto.

In some embodiments, an Activin A antibody agent disclosed herein further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, an Activin A antibody agent comprises a heavy chain (HC) provided in Table 2 (or a sequence having at least 85% identity thereto) and a light chain (HC) provided in Table 1 (or a sequence having at least 85% identity thereto).

In some embodiments, an Activin A antibody agent comprises: (i) a HC polypeptide comprising a HC amino acid sequence provided in Table 2, e.g., any one of SEQ ID NOs: 111, 118, 123, 127, 132, 138, 143, 147, 155, 161, 167, 177, 184, 188, 194, 200, 207, 212; or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to a HC amino acid sequence provided in Table 2, e.g., any one of SEQ ID NOs: 111, 118, 123, 127, 132, 138, 143, 147, 155, 161, 167, 177, 184, 188, 194, 200, 207, 212; and (ii) a LC polypeptide comprising an LC sequence provided in Table 1, e.g., any one of SEQ ID NOs: 9, 14, 19, 23, 34, 38, 46, 51, 59, 66, 75, 83, 91, 100, or a sequence with at least 85%, 86%, 87%, 88%, 89%, 90%, 91%, 92%, 93%, 94%, 95%, 96%, 97%, 98%, 99% identity thereto; or a sequence having at least 5, 10, or 20 alterations (e.g., substitutions, deletions or insertions (e.g., conservative substitutions)) relative to any one of SEQ ID NOs: 9, 14, 19, 23, 34, 38, 46, 51, 59, 66, 75, 83, 91, 100.

### Bispecific or Multispecific Activin A Antibody Agents

Disclosed herein, among other things, are bispecific or multispecific Activin A antibody agents and compositions comprising the same. In some embodiments, a bispecific Activin A antibody agent comprises a first binding specificity for Activin A and a second binding specificity for a second antigen. In some embodiments, a second antigen is other than Activin A. In some embodiments, a second antigen is a member of the TGFbeta superfamily.

In some embodiments of a Activin A bispecific antibody agent, a single chain Activin A polypeptide (e.g., a Activin A light chain polypeptide or a Activin A heavy chain polypeptide) provides binding specificity to Activin A.

In some embodiments, a Activin A bispecific antibody agent comprises a light chain (LC) polypeptide comprising a LC CDR1, a LC CDR2, and a LC CDR3, e.g., as provided in Table 1). In some embodiments, a Activin A bispecific antibody agent comprising a LC polypeptide with a LC CDR1, a LC CDR2, and a LC CDR3 binds to Activin A.

In some embodiments, a Activin A bispecific antibody agent comprises a heavy chain (HC) polypeptide comprising a HC CDR1, a HC CDR2, and a HC CDR3, e.g., as provided in Table 2). In some embodiments, a Activin A bispecific antibody agent comprising a HC polypeptide with a HC CDR1, a HC CDR2, and a HC CDR3 binds to Activin A.

In some embodiments, a Activin A bispecific antibody agent comprises an Activin A antibody agent comprising a heavy chain (HC) comprising a HC CDR1, a HC CDR2, and a HC CDR3, e.g., as provided in Table 2; and a light chain (LC) comprising a LC CDR1, a LC CDR2, and a LC CDR3, e.g., as provided in Table 1.

In some embodiments, a Activin A bispecific antibody agent is or comprises: a heterodimer, a Crossmab, a DVD-Ig, a 2 in 1 IgG, an IgG-sc-FV, an scFv-scFv, a BiTE, a DART, a diabody, a Fab-scFv fusion, a Fab-Fab fusion, a tandem antibody, or any other art recognized formats for an antibody having dual-specificity.

### Characterization of Activin A Antibody Agents

Activin A antibody agents disclosed herein specifically bind to Activin A and have one or more characteristics disclosed herein, e.g., high binding affinity, favorable binding kinetics, binding specificity, favorable pharmacokinetics, reduced self-aggregation, favorable expression profile (e.g., in mammalian cells), and/or stability.

In some embodiments, a polypeptide provided herein, e.g., a LC polypeptide and/or a HC polypeptide, is characterized by including in an Activin A antibody agent.

In some embodiments, an Activin A antibody agent described herein binds to human Activin A. In some embodiments, an Activin A antibody agent described herein binds to Activin A from a species having high homology with human Activin A, e.g., as provided in Table 14. In some embodiments, an Activin A antibody agent described herein binds to primate Activin A. In some embodiments, an Activin A antibody agent described herein binds to a domestic animal Activin A, e.g., a dog, a cat, a ferret, a horse, a cow, a pig, a sheep, a yak or a camel Activin A.

In some embodiments, an Activin A antibody agent or an Activin A polypeptide binds to human Activin A with a binding affinity (K_{D}) of about 5 pM to about 1000 pM. In some embodiments, an Activin A antibody agent or an Activin A polypeptide binds to human Activin A with a binding affinity (K_{D}) of about 5 pM, about 6 pM, about 7 pM, about 8 pM, about 9 pM, about 10 pM, about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 150 pM, about 200 pM, about 250 pM, about 300 pM, about 350 pM, about 400 pM, about 450 pM, about 500 pM, about 550 pM, about 600 pM, about 650 pM, about 700 pM, about 750 pM, about 800 pM, about 850 pM, about 900 pM, about 950 pM, about 1000 pM.

In some embodiments, an Activin A antibody agent or an Activin A polypeptide binds to human Activin A with a binding affinity (K_{D}) of about 5 pM to about 1000 pM, about 6 pM to about 1000 pM, about 7 pM to about 1000 pM, about 8 pM to about 1000 pM, about 9 pM to about 1000 pM, about 10 pM to about 1000 pM, about 15 pM to about 1000 pM, about 20 pM to about 1000 pM, about 25 pM to about 1000 pM, about 30 pM to about 1000 pM, about 35 pM to about 1000 pM, about 40 pM to about 1000 pM, about 45 pM to about 1000 pM, about 50 pM to about 1000 pM, about 55 pM to about 1000 pM, about 60 pM to about 1000 pM, about 65 pM to about 1000 pM, about 70 pM to about 1000 pM, about 75 pM to about 1000 pM, about 80 pM to about 1000 pM, about 85 pM to about 1000 pM, about 90 pM to about 1000 pM, about 95 pM to about 1000 pM, about 100 pM to about 1000 pM, about 150 pM to about 1000 pM, about 200 pM to about 1000 pM, about 250 pM to about 1000 pM, about 300 pM to about 1000 pM, about 350 pM to about 1000 pM, about 400 pM to about 1000 pM, about 450 pM to about 1000 pM, about 500 pM to about 1000 pM, about 550 pM to about 1000 pM, about 600 pM to about 1000 pM, about 650 pM to about 1000 pM, about 700 pM to about 1000 pM, about 750 pM to about 1000 pM, about 800 pM to about 1000 pM, about 850 pM to about 1000 pM, about 900 pM to about 1000 pM, about 950 pM to about 1000 pM.

In some embodiments, an Activin A antibody agent or an Activin A polypeptide binds to human Activin A with a binding affinity (K_{D}) of about 5 pM to about 1000 pM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 45 pM, about 5 pM to about 40 pM, about 5 pM to about 35 pM, about 5 pM to about 30 pM, about 5 pM to about 25 pM, about 5 pM to about 20 pM, about 5 pM to about 19 pM, about 5 pM to about 18 pM, about 5 pM to about 17 pM, about 5 pM to about 16 pM, about 5 pM to about 15 pM, about 5 pM to about 14 pM, about 5 pM to about 13 pM, about 5 pM to about 12 pM, about 5 pM to about 11 pM, about 5 pM to about 10 pM, about 5 pM to about 9 pM, about 5 pM to about 8 pM, about 5 pM to about 7 pM, about 5 pM to about 6 pM.

In some embodiments, binding of an Activin A polypeptide agent or an Activin A polypeptide to Activin A is measured using a Surface Plasmon Resonance assay (e.g., Biacore assay) or an Octet assay as described herein. In some embodiments, binding is measured in a Fab format. In some embodiments, binding is measured in an IgG format.

In some embodiments, a binding affinity is determined with a binding affinity determining assay such as an Octet assay or a comparable assay

In some embodiments, an Activin A antibody agent does not bind to or has minimal binding affinity for one or more TGFbeta super family members other than Activin A. In some embodiments, an Activin A antibody agent does not bind to or has minimal binding affinity for any one or all or a combination of GDNF, GDF8, GDF10, GDF11, BMP9, BMP10, GDF15, or Activin B.

In some embodiments, an Activin A antibody agent disclosed herein binds to one or more members of the TGFbeta super family in addition to Activin A. In some embodiments, a an Activin A antibody agent disclosed herein binds to Activin A and also binds to: Activin B, or GDF11, or both.

In some embodiments, an Activin A binding agent which binds to Activin A and Activin B does not modulate an activity and/or level of Activin B, e.g., when characterized in an assay that evaluates an Activin B activity and/or level.

In some embodiments, an Activin A binding agent which binds to Activin A and GDF11 does not modulate an activity and/or level of GDF11, e.g., when characterized in an assay that evaluates an GDF11 activity and/or level.

In some embodiments, an Activin A antibody agent has high specificity for Activin A and low polyreactivity, e.g., as measured with a poly-specificity reagent (PSR) or a comparable reagent that measures antibody binding specificity. In some embodiments an Activin A antibody agent has a clean PSR score of less than 0.1. In some embodiments, an Activin A antibody agent has a low PSR score of between 0.1 to 0.33. In some embodiments, an Activin A antibody agent has a low PSR score of about 0.1. In some embodiments, an Activin A antibody agent has a low PSR score of about 0.2. In some embodiments, an Activin A antibody agent has a low PSR score of about 0.22. In some embodiments, an Activin A antibody agent has a low PSR score of about 0.24. In some embodiments, an Activin A antibody agent has a low PSR score of about 0.26. In some embodiments, an Activin A antibody agent has a low PSR score of about 0.28. In some embodiments, an Activin A antibody agent has a low PSR score of about 0.3. In some embodiments, an Activin A antibody agent has a low PSR score of about 0.31. In some embodiments, an Activin A antibody agent has a low PSR score of about 0.32. In some embodiments, an Activin A antibody agent has a low PSR score of about 0.33.

In some embodiments, an Activin A antibody agent has low hydrophobicity as measured in a HIC assay or a comparable assay that measures hydrophobicity. In some embodiments, an Activin A antibody agent has a HIC retention time of less than 10.5 minutes indicating a clean to low HIC. In some embodiments, an Activin A antibody agent has a retention time of less than 10.5 minutes, less than 10 minutes, or less than 9.5 minutes. In some embodiments, an Activin A antibody agent has a retention time of about 9.4 minutes, about 9.5 minutes, about 9.6 minutes, about 9.7 minutes, about 9.8 minutes, about 9.9 minutes, about 10 minutes, about 10.1 minutes, about 10.2 minutes, about 10.3 minutes, about 10.4 minutes or about 10.5 minutes.

In some embodiments, an Activin A antibody agent has a retention time of between 10.5 to 11.5 minutes indicating a medium HIC. In some embodiments, an Activin A antibody agent has a retention time of about 10.5 minutes. In some embodiments, an Activin A antibody agent has a retention time of about 10.6 minutes. In some embodiments, an Activin A antibody agent has a retention time of about 10.7 minutes. In some embodiments, an Activin A antibody agent has a retention time of about 10.8 minutes. In some embodiments, an Activin A antibody agent has a retention time of about 10.9 minutes. In some embodiments, an Activin A antibody agent has a retention time of about 11 minutes. In some embodiments, an Activin A antibody agent has a retention time of about 11.1 minutes. In some embodiments, an Activin A antibody agent has a retention time of about 11.2 minutes. In some embodiments, an Activin A antibody agent has a retention time of about 11.3 minutes. In some embodiments, an Activin A antibody agent has a retention time of about 11.4 minutes. In some embodiments, an Activin A antibody agent has a retention time of about 11.5 minutes.

In some embodiments, an Activin A antibody agent has a retention time of about 12 minutes. In some embodiments, an Activin A antibody agent has a retention time of about 12.5 minutes.

In some embodiments, an Activin A antibody agent is produced in a bacterial cell, e.g., E. coli.

In some embodiments, an Activin A antibody agent is produced in a yeast cell, e.g., S. cerevisiae or S. pombe.

In some embodiments, an Activin A antibody agent is produced in an insect cell, e.g., Sf9.

In some embodiments, an Activin A antibody agent is produced in a mammalian cell. In some embodiments, a mammalian cell is chosen from a CHO cell, a COS cell, a HEK-293 cell, an NS0 cell, a PER.C6 cell, or an Sp2.0 cell.

In some embodiments, an Activin A antibody agent can be produced at a concentration of about 10 mg/L to about 20,000 mg/L. In some embodiments, an Activin A antibody agent can be produced at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L, about 150 mg/L, about 200 mg/L, about 250 mg/L, about 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L, about 900 mg/L, about 950 mg/L, about 1000 mg/L, about 2000 mg/L, about 2000 mg/L, about 3000 mg/L, about 4000 mg/L, about 5000 mg/L, about 6000 mg/L, about 7000 mg/L, about 8000 mg/L, about 9000 mg/L, about 10,000 mg/L, about 15,000 mg/L or about 20,000 mg/L. In some embodiments, an Activin A antibody agent can be produced at a concentration of more than 100 mg/L, more than 200 mg/L, more than 500 mg/L, more than 1000 mg/L, more than 2000 mg/L, more than 3000 mg/L, more than 4000 mg/L, more than 5000 mg/L, more than 6000 mg/L, more than 7000 mg/L, more than 8000 mg/L, more than 9000 mg/L, more than 10,000 mg/L. In some embodiments, an Activin A antibody agent can be produced at a concentration of about 1000 to 20,000 mg/L, about 2000 to 20,000 mg/L, about 5000 to 20,000 mg/L, about 6000 to 20,000 mg/L, about 7000 to 20,000 mg/L, about 8000 to 20,000 mg/L, about 9000 to 20,000 mg/L, 10,000 to 20,000 mg/L or about 15,000 to 20,000 mg/L.

In some embodiments, an Activin A antibody agent has a melting temperature (Tm) of about 65° C. to about 85° C., about 65° C. to about 80° C., about 65° C. to about 75° C., about 65° C. to about 70° C., about 70° C. to about 85° C., about 75° C. to about 80° C. In some embodiments, an Activin A antibody agent has a melting temperature (Tm) of about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., or about 83° C.

In some embodiments, an Activin A antibody agent has low self-association as measured with an AC-SINS assay or a comparable assay that measures self-association. In some embodiments, an Activin A antibody agent has an AC-SINS score less than 5, or between 5 and 20 indicating low self-association. In some embodiments, an Activin A antibody agent has an AC-SINS score of about 0.5. In some embodiments, an Activin A antibody agent has an AC-SINS score of about 0.6. In some embodiments, an Activin A antibody agent has an AC-SINS score of about 0.7. In some embodiments, an Activin A antibody agent has an AC-SINS score of about 0.8. In some embodiments, an Activin A antibody agent has an AC-SINS score of about 0.9. In some embodiments, an Activin A antibody agent has an AC-SINS score of about 1. In some embodiments, an Activin A antibody agent has an AC-SINS score of about 2. In some embodiments, an Activin A antibody agent has an AC-SINS score of about 3. In some embodiments, an Activin A antibody agent has an AC-SINS score of about 4. In some embodiments, an Activin A antibody agent has an AC-SINS score of about 5. In some embodiments, an Activin A antibody agent has an AC-SINS score of about 6. In some embodiments, an Activin A antibody agent has an AC-SINS score of about 7. In some embodiments, an Activin A antibody agent has an AC-SINS score of about 8. In some embodiments, an Activin A antibody agent has an AC-SINS score of about 9. In some embodiments, an Activin A antibody agent has an AC-SINS score of about 10. In some embodiments, an Activin A antibody agent has an AC-SINS score of about 11. In some embodiments, an Activin A antibody agent has an AC-SINS score of about 12. In some embodiments, an Activin A antibody agent has an AC-SINS score of about 13. In some embodiments, an Activin A antibody agent has an AC-SINS score of about 14. In some embodiments, an Activin A antibody agent has an AC-SINS score of about 15. In some embodiments, an Activin A antibody agent has an AC-SINS score of about 16. In some embodiments, an Activin A antibody agent has an AC-SINS score of about 17. In some embodiments, an Activin A antibody agent has an AC-SINS score of about 18. In some embodiments, an Activin A antibody agent has an AC-SINS score of about 19. In some embodiments, an Activin A antibody agent has an AC-SINS score of about 20.

In some embodiments, an Activin A antibody agent has an AC-SINS score of more than 20. In some embodiments, an Activin A antibody agent has an AC-SINS score of about 21, about 22, about 23, about 24, about 25, about 26, about 27, about 28, about 29 or about 30.

Nucleic Acids Encoding Activin A Antibody Agents and/or Provided Polypeptides

The present disclosure, among other things, provides nucleic acids encoding Activin A antibody agents described herein, or polypeptides provided herein (e.g., LC polypeptides and/or HC polypeptides). The present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g. a mammalian cell for production of an Activin A antibody agent described herein, or polypeptides provided herein (e.g., LC polypeptides and/or HC polypeptides).

Nucleic acids encoding an Activin A antibody agent, or polypeptides provided herein (e.g., LC polypeptides and/or HC polypeptides) may be modified to include codons that are optimized for expression in a particular cell type or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding an Activin A antibody agent described herein, or polypeptides provided herein (e.g., LC polypeptides and/or HC polypeptides), in whole or in part, may include an altered sequence to optimize codon usage for a particular cell type (e.g., a eukaryotic or prokaryotic cell). For example, a coding sequence for a humanized heavy (or light) chain variable region as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), the pcDNA3 series (Invitrogen) or the pEX series (Clontech, Palo Alto, Calif.). Bacteriophage vectors, such as λGT10, λGT11, λZapII (Stratagene), λEMBL4, and λNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding an Activin A antibody agent described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding an Activin A antibody agent described herein, or polypeptides provided herein (e.g., LC polypeptides and/or HC polypeptides), under control of transcriptional and/or translational control signals. These methods may include in vitro recombinant DNA and synthetic techniques and in vivo recombination (see, e.g., Ausubel, supra; or Sambrook, supra).

In some embodiments, a nucleic acid encoding an Activin A antibody agent described herein or polypeptides provided herein (e.g., LC polypeptides and/or HC polypeptides), is or comprises DNA.

In some embodiments, a nucleic acid encoding an Activin A antibody agent described herein or polypeptides provided herein (e.g., LC polypeptides and/or HC polypeptides), is or comprises RNA, e.g., messenger RNA.

### Pharmaceutical Compositions

The present disclosure, among other things, provides pharmaceutical compositions that comprise or otherwise deliver an Activin A antibody agent; typically, such pharmaceutical compositions comprise an active agent (e.g., an antibody agent or portion thereof, or a nucleic acid that encodes such antibody agent or portion thereof, etc.) one or more pharmaceutically or physiologically acceptable carriers, diluents, or excipients.

When “a therapeutically effective amount, “an immunologically effective amount,” “an anti-immune response effective amount,” or “an immune response-inhibiting effective amount” is indicated, a precise amount of a pharmaceutical composition that comprises or delivers an Activin A antibody agent described herein can be determined by a physician with consideration, for example, of individual differences in age, weight, immune response, and condition of the patient (subject).

In some embodiments, pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

In some embodiments, pharmaceutical compositions described herein may be administered in a manner appropriate to the disease, disorder, or condition to be treated or prevented. In some embodiments, quantity and/or frequency of administration may be determined by such factors as condition of a patient, and/or type and/or severity of a patient's disease, disorder, or condition, although appropriate dosages may be determined by clinical trials.

In some embodiments, a pharmaceutical composition provided by the present disclosure may be in a form such as, for example, liquid, semi-solid and solid dosage forms, such as liquid solutions (e.g., injectable and infusible solutions), dispersions or suspensions, liposomes, and suppositories. Typically, pharmaceutical compositions that comprise or deliver antibody agents are injectable or infusible solutions; in some such embodiments, such compositions can be formulated for administration intravenously, subcutaneously, intradermally, intratumorally, intranodally, intramedullary, intramuscularly, transarterially, sublingually, intranasally, topically or intraperitoneally. In some embodiments, provided pharmaceutical compositions are formulated for intravenous administration. In some embodiments, provided pharmaceutical compositions are formulated for subcutaneous administration.

Pharmaceutical compositions described herein can be formulated for administration by using infusion techniques that are commonly known in the field (See, e.g., Rosenberg et al., New Eng. J. of Med. 319:1676, 1988, which is hereby incorporated by reference in its entirety).

In some embodiments, pharmaceutical compositions described herein are administered in combination with (e.g., before, simultaneously, or following) an additional therapy for a symptom, disease or disorder, e.g., a SOC therapy for a symptom, disease or disorder. In some embodiments, pharmaceutical compositions described herein may be administered before or following surgery.

In some embodiments, a dosage of any aforementioned therapy to be administered to a subject will vary with a disease, disorder, or condition being treated and based on a specific subject. Scaling of dosages for human administration can be performed according to art-accepted practices.

Exemplary Identification, Characterization and/or Production of Activin A Antibody Agents or Components (e.g., Polypeptide Elements or Portions) Thereof

The present disclosure, among other things, provides production, identification, and/or characterization of an Activin A antibody agent described herein, or polypeptides provided herein (e.g., LC polypeptides and/or HC polypeptides). In some embodiments an Activin A antibody agent described herein is identified, characterized, and/or produced using a display technology, such as yeast display, phage display, or ribosome display. In some embodiments, an Activin A antibody agent described herein is identified, characterized and/or producing using hybridoma technology. In some embodiments, identification and/or characterization of a provided antibody agent utilizes library screening (e.g., of a hybridoma library, a phage library, a ribosome library, a yeast library, etc.

Phage library based methods for identifying, characterizing, and/or producing antibodies are known in the art (as described in, e.g., Ladner et al. U.S. Pat. No. 5,223,409; Kang et al. International Publication No. WO 92/18619; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; each of which his hereby incorporated by reference in its entirety).

Yeast library based methods for identifying, characterizing, and/or producing antibodies are known in the art, e.g., as described in U.S. Pat. No. 8,691,730 and Chao G. et al (2006) Nature Protocols 1(2): 755-68, each of which his hereby incorporated by reference in its entirety.

In some embodiments, an Activin A antibody agent described herein may be derived from another species (e.g., a species other than human). A humanized antibody is an antibody (typically produced by recombinant DNA technology), in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary or desirable, one or more residues in human FRs can be changed to residues at corresponding positions in a murine antibody so as to preserve binding affinity of the humanized antibody to a target. Such a change is sometimes called “back mutation.” Similarly, forward mutations may be made to revert back to murine sequence for a desired reason, e.g. stability or affinity to a target. Those skilled in the art are aware that humanized antibodies generally are less likely to elicit an immune response in humans as compared to chimeric human antibodies because the former contain considerably fewer non-human components.

Technologies for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., γl for CH and κ for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

In some embodiments, an Activin A antibody agent described herein comprises or is a human antibody. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147(1): 86-95, (1991), each of which is incorporated herein by reference in its entirety.

Also disclosed herein is a method of making an Activin A antibody agent, comprising culturing a host cell comprising a heterologous nucleic acid encoding an Activin A antibody polypeptide or combination thereof, under a condition wherein an Activin A antibody polypeptide or combination thereof (e.g., an Activin A polypeptide agent) is expressed by said host cell. In some such embodiments, the heterologous nucleic acid is or comprises a vector comprising an Activin A antibody agent nucleic acid sequence.

In some embodiments, a host cell is a yeast cell, a bacterial cell, a mammalian cell or an insect cell.

In some embodiments, a host cell is a mammalian cell. In some embodiments, a mammalian cell is chosen from a CHO cell, a COS cell, a HEK-293 cell, an NSO cell, a PER.C6 cell, or an Sp2.0 cell.

### Exemplary Uses of Activin A Antibody Agents or Components (e.g., Polypeptide Elements or Portions) Thereof

Polypeptides disclosed herein, e.g., a LC polypeptide and/or a HC polypeptide, can be included in an Activin A antibody agent.

Activin A antibody agents are useful in a variety of contexts, including in research, diagnosis, and therapy. In some embodiments, an Activin A antibody agent disclosed herein can be used as a reference agent and/or a reagent in research, e.g., to understand Activin A biology and/or biological processes directly or indirectly related to Activin A. In some embodiments, an Activin A antibody agent disclosed herein can be used as a reference agent and/or a reagent in diagnosis and/or treatment (e.g., patient selection).

This disclosure provides methods of using an Activin A antibody agent for, e.g., inhibiting Activin A (e.g., reducing an activity and/or level of Activin A) in a cell, tissue or subject (e.g., in a subject or in a sample from a subject). In some embodiments, Activin A comprises free and/or active Activin A. In some embodiments, Activin A is or comprises free Activin A. In some embodiments, Activin A is or comprises active Activin A. In some embodiments, Activin A is or comprises free and active Activin A.

In some embodiments, an increased level of Activin A is about 500 pg/ml or more. In some embodiments, a level and/or activity of Activin A is evaluated in a subject, e.g., via imaging, or in a sample from a subject, e.g., a tissue sample (e.g., a biopsy), or a bodily fluid sample (e.g., a blood, plasma, serum, urine, CSF, saliva or other bodily fluid).

In some embodiments, an Activin A antibody agent disclosed herein is characterized in that when administered to a subject it reduces a level and/or activity of Activin A, e.g., as compared to before administration of an Activin A antibody agent. In some embodiments, reduced Activin A level and/or activity is assessed in a subject, e.g., via imaging, or in a sample from a subject, e.g., a tissue sample (e.g., a biopsy), or a bodily fluid sample (e.g., a blood, plasma, serum, urine, CSF, saliva or other bodily fluid).

In some embodiments, an Activin A antibody agent reduces an Activin A level to less than 500 pg/ml. In some embodiments, an Activin A antibody agent reduces an Activin A level to at least 1%-90% less than before administration of an Activin A antibody agent.

Also disclosed herein are uses of an Activin A antibody agent (or a composition comprising the same) for ameliorating (e.g., reducing) one or more symptoms associated with a disease or disorder, or one or more symptoms associated with (e.g., induced by) a therapy for a disease or disorder. In some embodiments, a symptom associated with a disease or disorder, or a symptom associated with (e.g., induced by) a therapy for a disease or disorder is weight loss, loss of appetite, fatigue, muscle mass loss, fat mass loss, lean mass loss, lean mass atrophy, bone loss, anemia, or fibrosis or combinations thereof.

### Indications

Among other things, the present disclosure provides insights regarding usefulness of provided Activin A antibody agents in various contexts and/or indications, including in certain contexts and/or indications where therapeutic targeting of Activin A has not previously been proposed.

In some embodiments, an Activin A antibody agent as described herein (e.g., an anti-Activin A antibody) may be useful in treating and/or preventing a disease or disorder associated with increased levels of Activin A. In some embodiments, an increased level of Activin A is a level of about 500 pg/ml or more, e.g., as evaluated in a sample from a subject, e.g., a blood, plasma, serum or urine sample. In some embodiments, Activin A comprises free and/or active Activin A. In some embodiments, Activin A comprises free Activin A. In some embodiments, Activin A comprises active Activin A. In some embodiments, Activin A comprises free and active Activin A.

In some embodiments, an Activin A antibody agent as described herein (e.g., an anti-Activin A antibody) may be useful in preventing and/or reversing mass loss in a subject. In some embodiments, mass comprises fat mass, lean mass, muscle mass, bone mass, organ mass loss, or a combination thereof.

In some embodiments, an Activin A antibody agent as described herein (e.g., an anti-Activin A antibody) may be useful in preventing and/or reversing loss of functional muscle mass and/or loss of muscle strength in a subject.

In some embodiments, an Activin A antibody agent as described herein (e.g., an anti-Activin A antibody) prevents and/or reverses mass loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, mass loss is or comprises fat mass loss.

In some embodiments, mass loss is or comprises lean mass loss.

In some embodiments, mass loss is or comprises bone mass loss.

In some embodiments, mass loss is or comprises loss of functional muscle mass.

In some embodiments, mass loss is or comprises loss of muscle strength.

In some embodiments, mass loss is or comprises muscle mass loss. In some embodiments, muscle is chosen from: cardiac muscle, smooth muscle, skeletal muscle, or a combination thereof. In some embodiments, a subject having muscle mass loss has a disease or disorder characterized by decreased muscle mass and/or strength. In some embodiments, a disease or disorder characterized by decreased muscle mass and/or strength is chosen from: sarcopenia, cachexia, muscle injury, muscle wasting/atrophy, cancer, obesity, diabetes, arthritis, multiple sclerosis, muscular dystrophy, immobility, ICU patients, elderly, amyotrophic lateral sclerosis, Parkinson's disease, osteoporosis, osteoarthritis, osteopenia, a metabolic syndrome, chronic renal failure, renal fibrosis, or chronic obstructive pulmonary disease. In some embodiments, a metabolic syndrome comprises a disease or disorder chosen from: diabetes, obesity, nutritional disorders, organ atrophy, chronic obstructive pulmonary disease, or anorexia.

In some embodiments, mass loss is or comprises organ mass loss, e.g., heart, kidney, liver, and/or pancreas.

In some embodiments, an Activin A antibody agent as described herein (e.g., an anti-Activin A antibody) may be useful in preventing and/or reversing weight loss in a subject.

In some embodiments, weight loss is involuntary weight loss.

In some embodiments, an Activin A antibody agent prevents and/or reverses weight loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A antibody agent as described herein (e.g., an anti-Activin A antibody) may be useful in preventing and/or reversing muscle atrophy in a subject. In some embodiments, a muscle is chosen from: cardiac muscle, smooth muscle, skeletal muscle, or a combination thereof.

In some embodiments, an Activin A antibody agent prevents and/or reverses muscle atrophy by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A antibody agent as described herein (e.g., an anti-Activin A antibody) may be useful in preventing and/or reversing senescence in a subject.

In some embodiments, an Activin A antibody agent prevents and/or reverses senescence by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments of any of the methods disclosed herein, mass loss, weight loss, muscle atrophy, and/or senescence is a symptom of a disease or disorder.

In some embodiments of any of the methods disclosed herein, mass loss, weight loss, muscle atrophy, and/or senescence is not a symptom of a disease or disorder.

In some embodiments, an Activin A antibody agent as described herein (e.g., an anti-Activin A antibody) may be useful in preventing and/or reversing liver damage in a subject.

In some embodiments, liver damage comprises: liver necrosis, liver fibrosis, liver inflammation, liver degeneration, cirrhosis (e.g., alcoholic cirrhosis or non-alcoholic cirrhosis) alteration in liver function, elevation one or more liver enzymes, or a combination thereof.

In some embodiments, one or more liver enzymes comprise ALT, AST or both.

In some embodiments, liver damage is a symptom of liver disease.

In some embodiments, liver disease is or comprises cirrhosis.

In some embodiments, an Activin A antibody agent prevents and/or reverses liver damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A antibody agent reverses liver damage. In some embodiments, liver damage is reversed in a subject having liver fibrosis, e.g., acute liver fibrosis or chronic liver fibrosis. In some embodiments, liver damage is reversed in a subject having cirrhosis, e.g., alcoholic cirrhosis or non-alcoholic cirrhosis. In some embodiments, liver damage is reversed in an aging subject. In some embodiments, liver damage is reversed in a subject having liver necrosis, e.g., acute necrosis or chronic necrosis.

In some embodiments, an Activin A antibody agent induces liver regeneration. In some embodiments, liver regeneration is induced in a subject having liver fibrosis, e.g., acute liver fibrosis or chronic liver fibrosis. In some embodiments, liver regeneration is induced in a subject having cirrhosis, e.g., alcoholic cirrhosis or non-alcoholic cirrhosis. In some embodiments, liver regeneration is induced in an aging subject. In some embodiments, liver regeneration is induced in a subject having liver necrosis, e.g., acute necrosis or chronic necrosis.

In some embodiments, administration of an Activin A antibody agent promotes liver regeneration in a subject receiving a partial hepatectomy, e.g., to remove one or more liver metastases. In some embodiments, an Activin A antibody agent prevents reoccurrence of liver metastases in a subject.

In some embodiments, an Activin A antibody agent as described herein (e.g., an anti-Activin A antibody) may be useful in preventing and/or reversing kidney damage in a subject.

In some embodiments, kidney damage comprises: kidney fibrosis, kidney inflammation, alteration in kidney function, or a combination thereof.

In some embodiments, alteration in kidney function is or comprises proteinuria.

In some embodiments, kidney damage is or comprises tubular degeneration.

In some embodiments, kidney damage is a symptom of kidney disease.

In some embodiments, kidney disease is or comprises acute kidney disease or chronic kidney disease.

some embodiments, an Activin A antibody agent prevents and/or reverses kidney damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A antibody agent as described herein (e.g., an anti-Activin A antibody) may be useful in preventing and/or reversing cachexia in a subject.

In some embodiments, cachexia is induced by a tumor. In some embodiments, cachexia comprises loss of appetite, muscle loss, fat mass loss, weight loss, or a combination thereof.

In some embodiments, an Activin A antibody agent prevents and/or reverses cachexia by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A antibody agent as described herein (e.g., an anti-Activin A antibody) may be useful in preventing and/or reversing hypertension in a subject.

In some embodiments, an Activin A antibody agent prevents and/or reverses hypertension by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A antibody agent as described herein (e.g., an anti-Activin A antibody) may be useful in preventing and/or reversing pre-eclampsia in a subject.

some embodiments, an Activin A antibody agent prevents and/or reverses pre-eclampsia by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments of any of the methods disclosed herein, cachexia, mass loss, weight loss, muscle atrophy, senescence, liver damage, kidney damage, chemoresistance and/or hypertension is induced by a therapy for a disease or disorder. In some embodiments, a therapy for a disease or disorder comprises a standard of care, e.g., as described herein.

A few reports have noted a role for Activin A in chemoresistance. For example, Paajanen J et al., (2020) Clin lung Cancer 21(3) e142-e150 reported higher Activin A levels in patients who did not have a partial response or stable disease in response to chemotherapy suggesting a role for Activin A with systemic chemoresistance. Marini K D et al., (2018) Sci Transl Med 10(451) eaat3504. doi: 10.1126 reported a role for Activin A in resistance to chemotherapy.

Furthermore, Li K. et al., (2021) Theranostics 11(7): 3196-3212 showed that Activin A promoted pancreatic cancer stemness and blocking Activin A signaling could overcome gemcitabine resistance. Another report by Lonardo E et al., (2011) Cell Stem Cell 4; 9(5): 433-46 reported that inhibiting the ALK4/7 receptor which is a Nodal/Activin receptor in cancer stem cells abrogated the self-renewal capacity of the cells, reduced in vivo tumorigenicity and reversed resistance to gemcitabine. These reports demonstrate a role for Activin A in Gemcitabine-resistance in vivo.

The present disclosure teaches that provided Activin A antibody agents may be useful to prevent and/or treat chemoresistance in a subject having a cancer. Those skilled in the art, reading the present disclosure in light of the art, will appreciate such use as described herein.

In some embodiments, an Activin A antibody agent as described herein (e.g., an anti-Activin A antibody) may be useful in preventing, treating and/or reversing chemoresistance in a subject. In some embodiments, chemoresistance is associated with Activin A expression. In some embodiments, chemoresistance is reduced, treated, and/or prevented in a subject having cancer who has been or is being administered a therapy to treat a cancer (e.g., a chemotherapy as described herein).

In some embodiments, a cancer comprises a colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer. In some embodiments, a cancer is a colorectal cancer.

In some embodiments, an Activin A antibody agent as described herein (e.g., an anti-Activin A antibody) may be useful in preventing and/or treating a SARS-COV-2 infection. In some embodiments, a SARS-COV-2 infection is or comprises a COVID-19 disease. In some embodiments, a SARS-COV-2 infection comprises acute respiratory distress syndrome (ARDS).

In some embodiments of any of the methods disclosed herein, cachexia, mass loss, weight loss, muscle atrophy, senescence, liver damage, kidney damage, chemoresistance and/or hypertension is reduced relative to a comparator.

In some embodiments, a comparator comprises an otherwise similar cell, tissue or subject not administered an Activin A pharmaceutical composition or administered an Activin A inhibitor a different Activin A antibody agent.

In some embodiments, an Activin A antibody agent as described herein (e.g., an anti-Activin A antibody) may be useful in preventing and/or treating cancer in a subject.

In some embodiments, an Activin A antibody agent as described herein (e.g., an anti-Activin A antibody) may be useful in preventing and/or treating a cancer having a TP53 and/or SMAD4 mutation, or reducing metastases in a subject having such a cancer. Without wishing to be bound by any particular theory, it is proposed that a cancer having a TP53 and/or a SMAD4 mutation can be treated and/or prevented with an Activin A antibody agent by blocking the activity of Activin A. Without wishing to be bound by any particular theory, it is proposed that metastases (e.g., liver metastases) from a cancer having a TP53 and/or a SMAD4 mutation can be treated and/or prevented with an Activin A antibody agent by blocking the activity of Activin A. As demonstrated in Example 6 herein, an Activin A antibody agent blocks tumor growth and prevents metastases in an exemplary TRP53/SMAD4 mutant MSI model (MC38 model).

The present disclosure therefore demonstrates prevention and/or treatment of tumor growth in a tumor having a TP53 and/or SMAD4 mutation, and prevention and/or treatment of metastases from such a tumor. Accordingly, the present disclosure teaches that anti-Activin A antibodies can be used to prevent and/or reverse tumor growth and/or metastases in patients having a tumor with a TP53 mutation and/or a SMAD4 mutation.

In some embodiments, a cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

In some embodiments, a cancer is colorectal cancer.

In some embodiments of any of the methods disclosed herein, a subject has an increased level and/or activity of Activin A.

In some embodiments of any of the methods disclosed herein, a method further comprises determining a level and/or activity of Activin A in a sample from the subject.

In some embodiments, an increased level and/or activity of Activin A is determined relative to a comparator. In some embodiments, a comparator comprises a predetermined reference sample such as a sample obtained from an otherwise similar subject who does not have a disease or disorder, or a symptom of a disease or disorder.

In some embodiments, an Activin A antibody agent as described herein (e.g., an anti-Activin A antibody) may be useful in preventing and/or reducing migration of cells in vivo.

In some embodiments of any of the methods disclosed herein, a subject has a condition or disorder associated with increased Activin A. In some embodiments of any of the methods disclosed herein, a condition or disorder is a hyperproliferative disorder, e.g., a cancer.

In some embodiments, a method prevents and/or reduces migration of cells from a primary cancer to one or more secondary sites. In some embodiments, migration of cells to one or more secondary sites is or comprises metastasis.

In some embodiments, a method prevents and/or reduces migration of cells by at least 5%, 10%, 20%, 30%, 40%, 50%, 60%, 70%, 80%, 90%, 95% or 99%.

In some embodiments, prevention and/or reduction cell migration is assessed relative to a comparator. In some embodiments, a comparator comprises an otherwise similar subject not administered an Activin A pharmaceutical composition or administered an Activin A inhibitor or administered a different Activin A antibody agent.

In some embodiments, an Activin A antibody agent as described herein (e.g., an anti-Activin A antibody) may be useful in reducing metastasis, comprising administering to a subject an Activin A antibody agent.

In some embodiments, a cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

In some embodiments, a cancer is colorectal cancer.

In some embodiments, method reduces metastasis by at least 5%, 10%, 20%, 30%, 40%, 50%, 60%, 70%, 80%, 90%, 95% or 99%. In some embodiments, reduction of metastasis is assessed relative to a comparator. In some embodiments, a comparator comprises an otherwise similar subject not administered an Activin A pharmaceutical composition or administered an Activin A inhibitor or administered a different Activin A antibody agent.

In some embodiments, an Activin A antibody agent as described herein (e.g., an anti-Activin A antibody) may be useful in treating and/or preventing disorders of the hypothalamic pituitary gonadal axis (e.g., associated with increased FSH levels). In some embodiments, a subject having a disorder of the hypothalamic pituitary gonadal axis has an increased level of Activin A, e.g., as compared to a subject who does not have a disorder of the hypothalamic pituitary gonadal axis In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

At least one report has noted a role for Activin A in disorders of the hypothalamic pituitary gonadal axis (Namwanje and Brown (2016), Cold Spring Harbor Perspectives in Biology 8(7): a021881). In the hypothalamic-pituitary-gonadal (HPG) axis, Activins, including Activin A is an activator of pituitary FSH production and release (Namwanje and Brown 2016).

The present disclosure teaches that provided Activin A antibody agents may be useful to treat disorders of the hypothalamic pituitary gonadal axis. Those skilled in the art, reading the present disclosure in light of the art, will appreciate such use as described herein.

In some embodiments, an Activin A antibody agent as described herein (e.g., an anti-Activin A antibody) may be useful in treating and/or preventing fibrodysplasia ossificans progressive (FOP). In some embodiments, a subject having fibrodysplasia ossificans progressive has an increased level of Activin A, e.g., as compared to a subject who does not have fibrodysplasia ossificans progressive. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in fibrodysplasia ossificans progressive (FOP). For example, Lin H. et al. noted that Activin A has a prominent role in FOP and taught that “Activin A induces heterotopic bone formation via mutant ALK2, while inhibition of Activin A blocks spontaneous and trauma-induced HO,” whereby HO refers to heterotropic ossification (see Lin H. et al., (2019) Biosci Rep 39(8): BSR20190377). FOP is known to cause progressive HO. Another report noted a role for Activin A in enhancing mTOR signaling in the pathogenesis of FOP (see Hino K et al. (2017) Journal of Clinical Investigation 127(9); 3339-3352). A further report teaches “the role of Activin A and inflammation in HO” (see Alessi Wolken D M et al., (2017) Bone vol. 109, pp. 210-217).

The present disclosure teaches that provided Activin A antibody agents may be useful to treat and/or prevent fibrodysplasia ossificans progressive (FOP). Those skilled in the art, reading the present disclosure in light of the art, will appreciate such use as described herein.

In some embodiments, an Activin A antibody agent as described herein (e.g., an anti-Activin A antibody) may be useful in treating and/or preventing Pulmonary Arterial Hypertension (PAH) or hypertension. In some embodiments, a subject having Pulmonary Arterial Hypertension (PAH) or hypertension has an increased level of Activin A, e.g., as compared to a subject who does not have Pulmonary Arterial Hypertension (PAH) or hypertension. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in Pulmonary Arterial Hypertension (PAH) or hypertension. For example, one report notes that “anti-Activin A antibody reduced PAH PAVSMC growth without affecting canonical (Smads) or non-canonical (Akt, ERK1/2, p38 MAPK) effectors” (see Kurdyashova T V et al., (2018) Int J Mol Sci. 19(10): 2957). Another report teaches a role for Activin A in pulmonary vascular disease by using ACTRIIA-Fc, an Activin ligand trap (See Yung L M et al., (2020) Sci Transl. Med 12(543) eaaz5660). Additional reports noting a role for Activin A in Pulmonary Arterial Hypertension (PAH) or hypertension include Ryanto G. R. T. et al., (2021) Nature Communications 12:1720; and Tsai Y. et al., Am J Hypertens. 2018 Feb. 9; 31(3): 369-374).

The present disclosure teaches that provided Activin A antibody agents may be useful to treat Pulmonary Arterial Hypertension (PAH) or hypertension. Those skilled in the art, reading the present disclosure in light of the art, will appreciate such use as described herein.

In some embodiments, an Activin A antibody agent as described herein (e.g., an anti-Activin A antibody) may be useful in treating and/or preventing Anorexia-Cachexia Disorders associated with chronic diseases. In some embodiments, chronic diseases include COPD, chronic kidney disease, chronic heart failure, congestive heart failure, cancer, sarcopenia, muscular dystrophy (e.g., Duchenne's muscular dystrophy) elderly and muscle immobility, ICU patients (e.g., loss of body weight, loss of food intake, fat mass loss, muscle wasting, loss of functional muscle mass, loss of muscle strength, bone loss, anemia and/or fatigue). In some embodiments, Anorexia-Cachexia Disorders associated with chronic diseases include diseases with loss of food intake, fat mass loss, muscle wasting, loss functional muscle, loss of muscle strength, bone loss, anemia, fatigue, or a combination thereof.

In some embodiments, a subject having Anorexia-Cachexia Disorders associated with chronic diseases. has an increased level of Activin A, e.g., as compared to a subject who does not have Anorexia-Cachexia Disorders associated with chronic diseases. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in Anorexia-Cachexia Disorders associated with chronic diseases. For example, Chen et al., notes that “modified activin prodomains promote significant increases in muscle mass” (see Chen J L et al. (2015) Mol Ther. 23(3): 434-444). In the cancer setting, one report teaches “high ActA level is an independent prognosis factor of survival in cancer patients” and that “ActA seems to influence survival by contributing to the development of cachexia and loss of skeletal muscle mass” (see Loumaye A et al., J Cachexia Sarcopenia Muscle. 2017 October; 8(5): 768-777). Additional reports in the cancer setting describing the role of Activin A in muscle wasting include the following: Walton K L et al., Endocrinology. 2019 Oct. 1; 160(10): 2417-2426; Li Q et al., Mol Hum Reprod. 2007 September; 13(9): 675-83; Zhou X et al., Cell. 2010 Aug. 20; 142(4): 531-43; Huot J R et al. J Cachexia Sarcopenia Muscle. 2020 December; 11(6): 1779-1798; and Zhong X et al. J Cachexia Sarcopenia Muscle. 2019 October; 10(5): 1083-1101.

For muscle wasting or atrophy in chronic liver diseases, Kim J. Life (Basel) 2021 Mar. 17; 11(3): 250, notes treatment options for sarcopenia in chronic liver disease. Addition reports noting a role for Activin A in muscle wasting and/or loss of muscle mass include: Lee S J et al., Mol Endocrinol. 2010 October; 24(10): 1998-2008; Coerver K A et al., Mol Endocrinol. 1996 May; 10(5): 534-43; and Cipriano S C et al., Endocrinology, Volume 141, Issue 7, July 2000, Pages 2319-2327.

For a role of Activin A in muscle wasting in COPD, a report teaches that “COPD patients had upregulated Activin A expression” and that Actigin A expression in COPD patients negatively correlated with fat-free mass index and body mass index (See Zhou G et al., Clinics (Sao Paulo). 2019; 74: e981.

In chronic kidney disease a report by Solgana F et al., J Clin Invest, 2021 Jun. 1; 131(11): e135821, notes that “activin A is a key factor in CKD-induced cachexia.” An additional report teaches that systemic activation of Activin A signaling causes chronic kidney disease-mineral bone disorder (CKD-MBD) (see, Sugatani T. Int J Mol Sci. 2018 September; 19(9): 2490).

In Duchenne's muscular dystrophy, at least one report teaches that treating mice with AcRIIB-Fc results in increased bone mass in femurs (See Puolakkainen T. et al., BMC Musculoskelet Disord. 2017; 18:20).

The present disclosure teaches that provided Activin A antibody agents may be useful to treat and/or prevent Anorexia-Cachexia Disorders associated with chronic diseases, e.g., as described herein. Those skilled in the art, reading the present disclosure in light of the art, will appreciate such use as described herein.

In some embodiments, an Activin A antibody agent as described herein (e.g., an anti-Activin A antibody) may be useful in treating and/or preventing anemia. In some embodiments, a subject having anemia has an increased level of Activin A, e.g., as compared to a subject who does not have anemia. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in anemia. For example, one report notes that an activin receptor IIA ligand trap “may act to rescue growth differentiation factor 11/Activin A-induced inhibition of late-stage erythropoiesis” (See Carrancio S. et al. Br J Haematol. 2014 June; 165(6): 870-882). An additional report notes the use of activin receptor type IIB fusion protein ligand trap to treat ineffective erythropoiesis (See Kubasch A S et al., Blood Adv. 2021 Mar. 9; 5(5): 1565-1575). Yet another report teaches the “basis for biological activity of activin receptor ligand trap” in erythroid maturation and to alleviate anemia (Verma S. et al. J Clin Invest. 2020 Feb. 3; 130(2): 582-589).

The present disclosure teaches that provided Activin A antibody agents may be useful to treat and/or prevent anemia. Those skilled in the art, reading the present disclosure in light of the art, will appreciate such use as described herein.

In some embodiments, an Activin A antibody agent as described herein (e.g., an anti-Activin A antibody) may be useful in treating and/or preventing metabolic disorders (e.g., obesity, type 2 diabetes, and/or metabolic syndrome). In some embodiments, a subject having a metabolic disorder has an increased level of Activin A, e.g., as compared to a subject who does not have a metabolic disorder. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in metabolic disorders. For example, one report notes “functional relationship between activin signaling and mitochondrial energy metabolism and further support the rationale to target this signaling pathway for the medical treatment of cachexia, obesity, and diabetes” (See Li L et al., Endocrinology. 2009 August; 150(8): 3521-3529). A report by Peng L et al., teaches a “dose-dependent association between serum activin A levels, age, and metabolic syndrome, suggests activin A may be a biomarker of overall cardiometabolic risk” (Peng L. et al., Exp Gerontol. 2018 Oct. 1; 111:197-20). In type 2 diabetes and obesity an article by Heymsfield S B et al., JAMA Netw Open. 2021; 4(1): e2033457, notes that “blockade of the activin receptor with bimagrumab [an ActRII blocker] could provide a novel pharmacologic approach for managing patients with type 2 diabetes with excess adiposity.”

The present disclosure teaches that provided Activin A antibody agents may be useful to treat and/or prevent metabolic disorders (e.g., obesity, type 2 diabetes, and/or metabolic syndrome). Those skilled in the art, reading the present disclosure in light of the art, will appreciate such use as described herein.

In some embodiments, an Activin A antibody agent as described herein (e.g., an anti-Activin A antibody) may be useful in treating and/or preventing inflammatory disorders.

In some embodiments, inflammatory disorders include sepsis, endotoxemia, ARDS (Acute Respiratory Distress Syndrome)/cytokine Storm, COVID, allergic asthma, atopic dermatitis, acute and/or chronic pancreatitis, preeclampsia, inflammatory bowel diseases (e.g., Crohn's disease or ulcerative colitis), acute kidney injury, or acute liver injury.

In some embodiments, a subject having an inflammatory disorder has an increased level of Activin A, e.g., as compared to a subject who does not have an inflammatory disorder. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in inflammation and inflammatory disorders. For example, a report by Philips D J et al., Cytokine Growth Factor Rev. 2009 April; 20(2): 153-64, is a review article teaching the role of Activin A in inflammation and notes that “Activin modulates several aspects of the inflammatory response, including release of pro-inflammatory cytokines, nitric oxide production and immune cell activity.” For a report on the role of Activin A in sepsis, see Lee J K et al., Respirology. 2016 July; 21(5): 891-7.

For a report on the role of Activin A in endotoxemia, see Jones K L et al., Proc Natl Acad Sci 2007 Oct. 9; 104(41): 16239-44

For reports on the role of Activin A in ARDS or cytokine storm see: Kim J et al BMC Pulm Med. 2019 Jun. 25; 19(1): 115; Apostolou E et al., Am J Respir Crit Care Med. 2012 Feb. 15; 185(4): 382-91; or Linko R et al., BMC Infect Dis. 2014 May 10; 14:253.

For a report on the role of Activin A in COVID see McAleavy M et al., https://doi.org/10.1101/2021.02.04.429815, or Synolaki E et al. J Infect Dis. 2021 Feb. 24: jiab 108.

For a report on the role of Activin A in allergic asthma see Hardy C L et al., Clin Exp Allergy. 2006 July; 36(7): 941-50 or Hardy C L et al. Clin Exp Allergy. 2015 October; 45(10): 1510-1522.

For a report on the role of Activin A in atopic dermatitis Vittorakis S et al., Biomed Res Int. 2014; 2014:231036

For a report on the role of Activin A in acute or chronic pancreatitis, see Thomas A L et al., Clin Transl Gastroenterol. 2020 May; 11(5): e00152; or Staudacher J J et al., Sci Rep. 2017 Oct. 6; 7(1): 12786.

For a report on the role of Activin A in preeclampsia see Lim R et al., Am J Obstet Gynecol. 2015 January; 212(1): 86.e1-12.

For a report on the role of Activin A in inflammatory bowel diseases see, Hubner G et al., Lab Invest. 1997 October; 77(4): 311-8; or Dohi T et al., Gastroenterology. 2005 February; 128(2): 411-23

For a report on the role of Activin A in acute kidney injury, see Takei Y et al., PLOS One. 2019; 14(10): e0223703; Mehta and Krepinsky Current Opinion in Nephrology and Hypertension: January 2020-Volume 29-Issue 1-p 136-144; or Takahashi S et al., Sci Rep. 2018; 8: 5176.

For a report on the role of Activin A in acute liver injury, see Hughes and Evans Eur J Gastroenterol Hepatol. 2003 February; 15(2): 127-31; or Wang D et al., World J Gastroenterol. 2013 Jun. 28; 19(24): 3802-3809.

The present disclosure teaches that provided Activin A antibody agents may be useful to treat and/or prevent inflammatory disorders, e.g., sepsis, endotoxemia, ARDS (Acute Respiratory Distress Syndrome)/cytokine Storm, COVID, allergic asthma, atopic dermatitis, acute and/or chronic pancreatitis, preeclampsia, inflammatory bowel diseases (e.g., Crohn's disease or ulcerative colitis), acute kidney injury, or acute liver injury. Those skilled in the art, reading the present disclosure in light of the art, will appreciate such use as described herein.

In some embodiments, an Activin A antibody agent as described herein (e.g., an anti-Activin A antibody) may be useful in treating and/or preventing auto-immune disorders (e.g., SLE or rheumatoid arthritis). In some embodiments, a subject having an auto-immune disorder has an increased level of Activin A, e.g., as compared to a subject who does not have an auto-immune disorder. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of

Activin A to less than about 500 pg/ml. In some embodiments, administration of an Activin A antibody agent prevents an increase of Activin A level to more than about 500 pg/ml.

At least one report has noted a role for Activin A in auto-immune disorders. The report by El-Gendi et al., teaches that increased serum levels of Activin A in patients with rheumatic diseases (RA, SLE and osteoarthritis) and that “serum levels correlated positively with disease activity parameters of RA and SLE” (See El-Gendi S S et al., Int J Rheum Dis. 2010 August; 13(3): 273-9).

The present disclosure teaches that provided Activin A antibody agents may be useful to treat and/or prevent auto-immune disorders (e.g., SLE or rheumatoid arthritis). Those skilled in the art, reading the present disclosure in light of the art, will appreciate such use as described herein.

In some embodiments, an Activin A antibody agent as described herein (e.g., an anti-Activin A antibody) may be useful in treating and/or preventing chronic diseases, e.g., associated with fibrosis. In some embodiments, chronic diseases associated with fibrosis include: lung fibrosis, liver fibrosis, kidney fibrosis, systemic sclerosis, uterine fibroids, endometriosis, and/or heart failure. In some embodiments, a subject having a chronic disease associated with fibrosis has an increased level of Activin A, e.g., as compared to a subject who does not have a chronic disease associated with fibrosis. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in chronic diseases associated with fibrosis. In lung fibrosis, the following reports noted a role for Activin A in lung fibrosis: Matsuse T et al., Am J Pathol. 1996 March; 148(3): 707-13; Aoki F et al., Am J Respir Crit Care Med. 2005 Sep. 15; 172(6): 713-20; Ohga E et al., Biochem Biophys Res Commun. 1996 Nov. 12; 228(2): 391-6; or Matsuse T et al., Am J Respir Cell Mol Biol. 1995 July; 13(1): 17-24.

For a role of Activin A in liver apoptosis, fibrosis and/or cirrhosis, see Patella S et al., Am J Physiol Gastrointest Liver Physiol. 2006 January; 290(1): G137-44; De Bleser P J et al., Hepatology. 1997 October; 26(4): 905-12; Kiagiadaki F et al., Biochimica et Biophysica Acta (BBA)-Molecular Basis of Disease; Volume 1864, Issue 3, March 2018, Pages 891-899; Wada W et al., Endocrinology. 2004 June; 145(6): 2753-9; Ding Z et al., Int J Mol Sci. 2016 Mar. 22; 17(3): 408; Zhnag H et al., Int J Mol Med. 2018 July; 42(1): 279-289; Sugiyama M et al., Gastroenterology. 1998 March; 114(3): 550-8; Huang X et al., World J Gastroenterol. 2001 February; 7(1): 37-41; or Gold E J et al., Mol Cell Endocrinol. 2003 Mar. 28; 201(1-2): 143-53.

For a role of Activin A in Hepatitis C see Refaat B et al., Gastroenterology Research and Practice, vol. 2014, Article ID 628683.

For a role of Activin A in kidney fibrosis (Alport, or PKD) see Ren X et al., Nephrology (Carlton). 2009 April; 14(3): 311-20; Sugatani T., Int J Mol Sci. 2018 Aug. 23; 19(9): 2490; or Leonhard W N et al., J Am Soc Nephrol. 2016 December; 27(12): 3589-3599.

For a role of Activin A in systemic sclerosis see Takagi K et al., J Autoimmun. 2011 May; 36(3-4): 181-8; or Ly T et al., Biomolecules. 2020 Apr. 14; 10(4): 609.

For a role of Activin A in uterine fibroids see Bao H et al., Biochem Biophys Res Commun. 2018 Oct. 2; 504(2): 447-45; or Protic O et al., Semin Reprod Med. 2017 November; 35(6): 499-509.

For a role of Activin A in endometriosis see Kasai K et al., J Med Invest. 2019; 66(1.2): 123-127; or Zhang Z et al., Cell Commun Signal. 2019 May 17; 17(1): 45.

For a role of Activin A in heart failure see Roh J D et al., Sci Transl Med. 2019 Mar. 6; 11(482).

The present disclosure teaches that provided Activin A antibody agents may be useful to treat and/or prevent chronic diseases, e.g., associated with fibrosis. In some embodiments, chronic diseases associated with fibrosis include: lung fibrosis, liver fibrosis, kidney fibrosis, systemic sclerosis, uterine fibroids, Endometriosis, and/or heart failure. Those skilled in the art, reading the present disclosure in light of the art, will appreciate such use as described herein.

In some embodiments, an Activin A antibody agent as described herein (e.g., an anti-Activin A antibody) may be useful in treating and/or preventing cancer, e.g., as described herein. In some embodiments, administration of an Activin A antibody agent to a subject having a cancer can prevent and/or reverse any one, all or a combination of Activin A induced characteristics: tumor cell growth, migration, invasion and metastasis, angiogenesis, stemness and drug resistance, or regulation of antitumor immunity. In some embodiments, a cancer is chosen from colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

In some embodiments, a subject having a cancer has an increased level of Activin A, e.g., as compared to a subject who does not have a cancer. In some embodiments, an increased level of Activin A is a level of about 500 pg/ml or more, e.g., as evaluated in a sample from a subject, e.g., a blood, plasma, serum or urine sample. In some embodiments, administration of an Activin A antibody agent reduces a level of Activin A to less than about 500 pg/ml. In some embodiments, administration of an Activin A antibody agent prevents an increase of Activin A level to more than about 500 pg/ml.

Several reports have noted a role for Activin A in cancer and cancer treatment-related toxicities. For reports that teach a role for Activin A in, e.g., inducing tumor cell growth, migration, angiogenesis, stemness and drug resistance, regulation of antitumor immunity, see: Deli A et al., World J Gastroenterol. 2008 Mar. 21; 14(11): 1699-1709; Ries A et al., Expert Opin Ther Targets. 2020 October; 24(10): 985-996; Guo and Liu, Journal of International Medical Research Volume 49 Issue 6; Hoda M A et al., Br J Cancer. 2012 Dec. 4; 107(12): 1978-86; Seder C W et al., J Thorac Oncol. 2009 April; 4(4): 455-62; Dignass A U et al., Scand J Gastroenterol. 2002 August; 37(8): 936-43; Jung B H et al., Gastroenterology. 2007 February; 132(2): 633-44; Chang K et al., Ann Surg Oncol. 2010 July; 17(7): 1945-56; Tamminen J A et al., Exp Cell Res. 2015 Mar. 1; 332(1): 102-15; Dean M et al., Cancer Lett. 2017 Apr. 10; 391:114-124; Yi Y et al., Exp Cell Res 2019 Sep. 15; 382(2): 111471; Portale F et al., Haematologica. 2019 March; 104(3): 533-545; Bao H et al., Biochem Biophys Res Commun. 2020 Aug. 20; 529(2): 379-385; Wagner K et al., Gastroenterology. 2004 June; 126(7): 1828-43.

For a report teaching a role of Activin A in chemotherapy-induced nephropathy, see Koken E et al., Bratisl Lek Listy. 2020; 121(2): 143-150, which teaches that administration of Follistatin (an Activin blocker) “showed a considerable nephroprotective effect against cisplatin-induced nephrotoxicity by preventing renal functional and structural abnormalities, apoptosis and inflammation.”

For reports teaching a role of Activin A in cancer metastasis, see Staudacher J J et al., Sci Rep. 2017; 7:5569; Leto G et al., Clin Exp Metastasis. 2006; 23(2): 117-22; Nomura M et al., Biochem Biophys Res Commun. 2013 Jan. 4; 430(1): 340-6; Kalli M et al., Front Oncol. 2019 Feb. 5; 9:32; or Hyuga S et al., Cancer Lett. 2000 May 29; 153(1-2): 137-43.

For a report on Activin A polymorphism and treatment with regrofenib, see Miyamoto Y et al., PLOS One. 2020 Sep. 24; 15(9): e0239439.

For a report summarizing findings on “the multifaceted anti-cachectic effects of ACVR2 blockade in preclinical models of cancer, as well as in combination with anticancer treatments,” see Hulmi J J et al., Cells. 2021 Feb. 28; 10(3): 516.

For a report on immune escape mechanisms driven by Activin A including promotion of T reg cell mediated immunosuppression following radiation therapy, see De Martino M et al., Cancer Immunol Res. 2021 January; 9(1): 89-102.

The present disclosure teaches that provided Activin A antibody agents may be useful to treat and/or prevent cancer, e.g., as described herein. One of skill in the art would also understand that an antibody agent disclosed herein can be used to prevent and/or reverse any one, all or a combination of Activin A induced characteristics: tumor cell growth, migration, invasion and metastasis, angiogenesis, stemness and drug resistance, or regulation of antitumor immunity. In some embodiments, a cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer. Those skilled in the art, reading the present disclosure in light of the art, will appreciate such use as described herein.

In some embodiments, an Activin A antibody agent as described herein (e.g., an anti-Activin A antibody) may be useful in treating and/or preventing aging and/or senescence. In some embodiments, an aging subject or a subject who has senescence has an increased level of Activin A, e.g., as compared to a subject who is not aging or does not have senescence. In some embodiments, an increased level of Activin A is a level of about 500 pg/ml or more, e.g., as evaluated in a sample from a subject, e.g., a blood, plasma, serum or urine sample. In some embodiments, administration of an anti-Activin A antibody agent reduces a level of Activin A to less than about 500 pg/ml. In some embodiments, administration of an anti-Activin A antibody agent prevents an increase of Activin A level to more than about 500 pg/ml.

Several reports have noted a role for Activin A in aging and/or senescence. For example a report by Barrios-Silva L V et al., note a role for Activin A in normal human aging (See Barrios-Silva L V et al., Physiol Rep. 2018 September; 6(17): e13823). Another report teaches that Activin A is a Senescence-Associated Secretory Phenotype (SASP) protein (See Schafer M J et al., JCI Insight. 2020 Jun. 18; 5(12): e133668. Yet another report noted increased plasman Activin A levels in diabetic patients with diabetic kidney disease and suggest a role for “Activin A as a diagnostic marker and therapeutic target in DKD” (See Bian X et al., BMJ Open Diabetes Res Care. 2019 Dec. 15; 7(1): e000720. A report by Roh D J et al., teaches that Activin A induces up-regulation of the proteasome pathway in mammalian cardiomyocytes and that “increased activin/ActRII signaling links aging and HF pathobiology” (See Roh D J et al., Sci Transl Med. 2019 Mar. 6; 11(482)).

The present disclosure teaches that provided Activin A antibody agents may be useful to treat and/or prevent aging and/or senescence. Those skilled in the art, reading the present disclosure in light of the art, will appreciate such use as described herein.

In some embodiments of any of the methods disclosed herein, an Activin A antibody agent is characterized in that when administered to a subject it reduces a level and/or activity of Activin A relative to a comparator. In some embodiments, a level Activin A is reduced in a blood, plasma, serum or urine sample. In some embodiments, an Activin A antibody agent reduces a level of Activin A to less than 500 pg/mL.

In some embodiments, Activin A is or comprises free and/or active Activin A. In some embodiments, Activin A is or comprises free Activin A. In some embodiments, Activin A is or comprises active Activin A. In some embodiments, Activin A is or comprises free and active Activin A.

### Dosing Regimens

Those skilled in the art will be able to determine, according to known methods, the appropriate amount, dose or dosage of an Activin A antibody agent, to administer to a patient, taking into account factors such as age, weight, general health, the route of administration, the nature of the symptom, disease or disorder requiring treatment, and the presence of other medications. For example, various dosing regimens for antibodies are disclosed in Hendrikx J et al. (2017) Oncologist 22(10): 1212-1221, PMID: 28754722, the entire contents of which is hereby incorporated by reference.

In some embodiments, an Activin A antibody agent is administered at a fixed dose, i.e. independent of body weight. In some embodiments, a fixed dose reduces interpatient variability, e.g., efficacy and/or PK/PD parameters.

In some embodiments, an Activin A antibody agent is administered at a fixed dose of about of 0.1 mg to about 2000 mg. In some embodiments, an Activin A antibody agent is administered at a fixed dose of about 0.1 mg, about 0.2 mg, about 0.25 mg, about 0.5 mg, about 1 mg, about 5 mg, about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, or about 2000 mg. In some embodiments, an Activin A antibody agent is administered intravenously (IV) or subcutaneously (SC) at a fixed dose of about 0.25 mg to about 2000 mg.

In some embodiments, an Activin A antibody agent administered at a fixed dose is administered daily, weekly or monthly. In some embodiments, an Activin A antibody agent administered at a fixed dose is administered once a week, once every 2 weeks, once every 3 weeks or once every 4 weeks.

In some embodiments, an Activin A antibody agent is administered based on body weight, e.g., in a mg/kg dosing. In some embodiments, an Activin A antibody agent is administered at a dose of about 0.025 mg/kg to about 20 mg/kg. In some embodiments, an Activin A antibody agent is administered at a dose of about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg. In some embodiments, an Activin A antibody agent is administered intravenously (IV) or subcutaneously (SC) at a dose of about 0.025 mg/kg to about 20 mg/kg.

In some embodiments, an Activin A antibody agent is administered at an initial dose. In some embodiments, an initial dose may be followed by one or more subsequent doses. In some embodiments, one or more subsequent dose may be administered daily, weekly, or monthly, or at other intervals in between. In some embodiments, a dosing regimen disclosed herein may be repeated for one or more times.

### Combination Therapies

In some embodiments, an Activin A antibody agent disclosed herein, or a composition comprising the same is administered in combination with an additional agent, e.g., additional therapy. In some embodiments, an additional therapy comprises a therapy for a disease or disorder, e.g., a standard of care (SOC) therapy, for a symptom, disease or disorder. In some embodiments, an Activin A antibody agent is administered before, concurrently with or after administration of an additional therapy, e.g., a SOC therapy. In some embodiments of concurrent administration of an Activin A antibody agent disclosed herein, or a composition comprising the same and an additional therapy, an Activin A antibody agent disclosed herein, or a composition comprising the same is administered first followed by an additional therapy. In some embodiments of concurrent administration of an Activin A antibody agent disclosed herein, or a composition comprising the same and an additional therapy, an additional therapy is administered first followed by an Activin A antibody agent disclosed herein, or a composition comprising the same.

In some embodiments, when a subject has cancer, an additional therapy, e.g., a SOC, comprises one or more of surgery, chemotherapy, radiation therapy, small molecule therapy, targeted therapy such as antibody therapy, immunotherapy, hormonal therapy, stem cell based therapy or other therapies, e.g., as are known in the field and appreciated by one with skill in the art. In some particular embodiments, an additional therapy may be or comprise checkpoint inhibitor therapy, angiogenesis inhibitor therapy, etc.

In some embodiments, when a subject has weight loss, an additional therapy, e.g., a SOC, comprises nutrition management, nutritional supplements, and/or an appetite stimulant.

In some embodiments, when a subject has loss of appetite, an additional therapy, e.g., a SOC comprises an appetite stimulant, e.g., a supplement such as Zinc, a cannabinoid, a synthetic progestin, a testosterone derivative, and/or a steroid.

In some embodiments, when a subject has chronic kidney disease, an additional therapy, e.g., a SOC comprises a treatment for one or more complications associated with chronic kidney disease. In some embodiments, a SOC for chronic kidney disease comprises one or more of: high blood pressure treatment; therapy to reduce cholesterol levels; therapy to treat anemia; therapy to strengthen bones; a low protein diet; dialysis; or kidney transplant.

In some embodiments, when a subject has chronic heart disease, an additional therapy, e.g., a SOC comprises an angiotensin-converting enzyme (ACE) inhibitor; an Angiotensin II receptor blocker; a beta blocker; a diuretic; an aldosterone antagonist; digoxin; an inotrope; a hydralazine and isosorbide dinitrate; or surgery.

In some embodiments, when a subject has COPD, an additional therapy, e.g., a SOC comprises a bronchodilator; an inhaled steroid; a combination of a bronchodilator and an inhaled steroid; an oral steroid; theophylline; antibiotics; oxygen therapy; ventilation therapy or surgery.

In some embodiments, when a subject has peripheral arterial hypertension, an additional therapy, e.g., a SOC comprises vasodilators, guanylate cyclase stimulators, endothelin receptor antagonists, PDE5 inhibitors, calcium channel blockers, warfarin, digoxin, diuretics, oxygen therapy, or a combination thereof.

In some embodiments, when a subject has acute kidney injury, an additional therapy, e.g., a SOC comprises one or more of: fluid resuscitation, avoidance of nephrotoxic medications and contrast media exposure, correction of electrolyte imbalances, or renal replacement therapy (e.g., dialysis).

In some embodiments, when a subject has a liver disease (e.g., as described herein), an additional therapy, e.g., a SOC comprises a treatment for one or more symptoms and/or complications associated with liver disease. In some embodiments, when a subject has cirrhosis, an additional therapy, e.g., a SOC comprises one or more of: weight management, abstinence from alcohol, medications to control hepatitis, medications to control symptoms of cirrhosis (e.g., primary biliary cirrhosis), or liver transplant. In some embodiments, when a subject has acute liver failure, an additional therapy, e.g., a SOC comprises one or more of: medications to reverse poisoning (e.g., acetylcysteine if injury is due to acetaminophen poisoning), medications to relieve cerebral edema, liver transplant, blood transfusions, medications to prevent severe bleeding, or nutritional support.

### Diagnostic Technologies

In some embodiments, an Activin A antibody agent, or a polypeptide disclosed herein (e.g., a LC polypeptide and/or a HC polypeptide), or a composition that comprises and/or delivers the same may be used to detect the presence of Activin A, in vivo or in vitro. By correlating the presence or level of these proteins with a medical condition, one of skill in the art can diagnose the associated medical condition. The medical conditions that may be diagnosed by the presently disclosed Activin A antibody agents or LC and/or HC polypeptides are set forth above.

Such detection methods are well known in the art and include ELISA, radioimmunoassay, immunoblot, Western blot, immunofluorescence, immunoprecipitation, and other comparable techniques. A Activin A antibody agent or a polypeptide disclosed herein (e.g., a LC polypeptide and/or a HC polypeptide), may further be provided in a diagnostic kit that incorporates one or more of these techniques to detect a protein (e.g., Activin A). Such a kit may contain other components, packaging, instructions, or other material to aid the detection of the protein and use of the kit.

Where the antibodies are intended for diagnostic purposes, it may be desirable to modify them, for example, with a ligand group (such as biotin) or a detectable marker group (such as a fluorescent group, a radioisotope or an enzyme). If desired, the antibodies (whether polyclonal or monoclonal) may be labeled using conventional techniques. Suitable labels include fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable labels may include biotin and avidin or streptavidin, IgG and protein A, and the numerous receptor-ligand couples known in the art. Other permutations and possibilities will be readily apparent to those of ordinary skill in the art, and are considered as equivalents within the scope of the instant invention.

Accordingly, disclosed herein is a method comprising, assessing a level and/or activity of Activin A in a sample from a subject, and administering an Activin A pharmaceutical composition to the subject if the level of Activin A is higher than a comparator.

In some embodiments, a level of Activin A is evaluated with an Activin A antibody agent or a polypeptide disclosed herein (e.g., a LC polypeptide and/or a HC polypeptide). In some embodiments, an increased level and/or activity of Activin A is determined relative to a comparator. In some embodiments, a comparator comprises a predetermined reference sample such as a sample obtained from an otherwise similar subject who does not have a condition, disease or disorder, or a symptom of a disease or disorder.

### EXAMPLES

The following examples are provided so as to further illustrate to the skilled artisan particular ways in which provided methods and/or compositions described herein may be made and/or used, and are not intended to limit the scope of the present disclosure.

### Example 1: Identification of Anti-Activin Antibodies

This Example demonstrates the identification of anti-Activin antibodies (e.g., Activin antibody agents) from human synthetic yeast libraries.

### Materials and Methods

### Antigen Preparation

Antigens were biotinylated using the EZ-Link Sulfo-NHS-Biotinylation Kit (Thermo Scientific, Cat #21425). The antigens were concentrated to ~1 mg/mL and buffer exchanged into PBS before addition of 1:7.5 molar ratio biotinylation reagent. The mixture was held at 4 C overnight prior to another buffer exchange to remove free biotin in the solution. Biotinylation was confirmed through streptavidin sensor binding of the labeled proteins on a ForteBio.

### Naïve Library Selections

Eight naïve human synthetic yeast libraries each of ~10⁹ diversity were propagated as previously described (see, e.g., Y. Xu et al, PEDS 26(10), 663-70(2013); WO2009036379; WO2010105256; and WO2012009568.)

For the first two rounds of selection, a magnetic bead sorting technique utilizing the Miltenyi MACS system was performed, as previously described (see, e.g., Siegel et al, J Immunol Methods 286(1-2), 141-153(2004).) Briefly, yeast cells (~10¹⁰ cells/library) were incubated with 10 nM biotinylated human Activin A-Fc fusion for 30 min at 30° C. in wash buffer (phosphate-buffered saline (PBS)/0.1% bovine serum albumin (BSA)). After washing once with 40 mL ice-cold wash buffer, the cell pellet was resuspended in 20 mL wash buffer, and Streptavidin MicroBeads (500 μl) were added to the yeast and incubated for 15 min at 4° C. Next the yeast were pelleted, resuspended in 5 mL wash buffer, and loaded onto a Miltenyi LS column. After the 5 mL were loaded, the column was washed 3 times with 3 mL wash buffer. The column was then removed from the magnetic field, and the yeast were eluted with 5 mL of growth media and then grown overnight.

The following rounds of selection were performed using flow cytometry (FACS). Yeast were pelleted, washed three times with wash buffer, and incubated at 30° C. with either 10 nM biotinylated human Activin A-Fc fusion, one of 10 nM biotinylated cyno ACTIVIN A-Fc fusion or 10 nM biotinylated mouse Activin A-Fc fusion in order to obtain species cross-reactivity, or with a polyspecificity reagent (PSR) to remove non-specific antibodies from the selection. For the PSR depletion, the libraries were incubated with a 1:10 dilution of biotinylated PSR reagent as previously described (see, e.g., Y. Xu et al, PEDS 26(10), 663-70(2013)). Yeast were then washed twice with wash buffer and stained with goat F(ab′)2 anti-human kappa-FITC (LC-FITC) diluted 1:100 (Southern Biotech, Cat #2062-02) and either Streptavidin-AF633 (SA-633) diluted 1:500 (Life Technologies, Cat #S21375) or Extravidin-phycoerthyrin (EA-PE) diluted 1:50 (Sigma-Aldrich, Cat #E4011), secondary reagents for 15 min at 4° C. After washing twice with ice-cold wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast were plated and individual colonies were picked for characterization.

### Antibody Optimization

Optimization of antibodies was performed via a light chain batch shuffle, and then by introducing diversities into the heavy chain variable region using approaches described below.

Light chain batch shuffle: Heavy chains from the naïve output were used to prepare light chain diversification libraries. Selections were performed on these libraries as described above, i.e., with one round of MACS and four rounds of FACS. In the different FACS selection rounds, the libraries were evaluated for, e.g., PSR binding, species cross-reactivity, and affinity pressure by antigen titration. Sorting was performed in order to obtain a population with the desired characteristics. Individual colonies from each terminal FACS selection round were picked for sequencing and characterization.

CDRH1 and CDRH2 selection: The CDRH3 of a single antibody was recombined into a premade library with CDRH1 and CDRH2 variants of a diversity of ~108 and selections were performed with one round of MACS and four rounds of FACS as described in the naïve discovery. For each FACS round the libraries were looked at for PSR binding and affinity pressure, and sorting was performed in order to obtain a population with the desired characteristics. For these selections, affinity pressures were applied by preincubating the biotinylated antigen with parental IgG or Fab for 30 minutes and then applying that precomplexed mixture to the yeast library for a length of time which would allow the selection to reach an equilibrium. The higher affinity antibodies were then able to be sorted.

CDRH3 selection: Oligos were ordered from IDT which comprised the CDRH3 as well as a flanking region on either side of the CDRH3. Each oligo variegated two amino acids in the CDRH3 via NNK diversity. The CDRH3 oligos were recombined with heavy chain FR1-FR3 variable regions containing selected variants from the CDRH1 and CDRH2 selections. Selections were performed similar to previous cycles using FACS sorting for four rounds. For each FACS round the libraries were looked at for PSR binding and affinity pressure, and sorting was performed in order to obtain a population with the desired characteristics. Affinity pressures for these selections were performed as described above in the CDRH1 and CDRH2 selection.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 3.5.

### Results

This process resulted in the identification of human anti-Activin antibodies including clones A-R. The selected antibodies had a low PSR score as provided in Table 3. The PSR score was determined by normalizing values against a set of control IgGs whereby a clean PSR is a score of less than 0.1, a low PSR is a score between 0.1 and 0.33, a medium PSR is a score between 0.33 and 0.66 and a high PSR is a score between 0.66 and 1. The data provided in Table 3 shows that the tested anti-Activin antibodies had low PSR scores indicating high specificity to the Activin A antigen and minimal polyreactivity.

TABLE 3
PSR scores

| | Poly-Specificity Reagent |
|---|---|
| Clone | (PSR) Score (0-1) |
| A | 0.00 |
| B | 0.00 |
| C | 0.00 |
| D | 0.01 |
| E | 0.37 |
| F | 0.02 |
| G | 0.00 |
| H | 0.10 |
| I | 0.06 |
| J | 0.00 |
| K | 0.00 |
| L | 0.01 |
| M | 0.11 |
| N | 0.11 |
| O | 0.00 |
| P | 0.00 |
| Q | 0.1 |
| R | 0.01 |

### Example 2: Characterization of Anti-Activin Antibodies

This Example describes the evaluation of binding kinetics, binding specificity, stability and aggregation behavior for anti-Activin antibodies identified in Example 1.

### Materials and Methods

### ForteBio Octet KD Measurements

ForteBio affinity measurements were performed on an Octet HTX generally as previously described (see, e.g., Estep et al, Mabs 5(2), 270-278 (2013)). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model.

### ForteBio Octet Epitope Binning

Epitope binning was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM human Activin A-Fc antigen followed by a second anti-Activin antibody. Additional binding by the second antibody after antigen association indicates an unoccupied epitope (non-competitor), while no binding indicates epitope blocking (competitor). In studies assessing selectivity, human Activin A-Fc was replaced with Activin B, BMP-9, BMP-10, GDF-15-Fc, GDNF, GDF-8, GDF-10 or GDF-11.

### Size Exclusion Chromatography

A TSKgel SuperSW mAb HTP column (22855) was used for fast SEC analysis of yeast and mammalian produced mAbs at 0.4 mL/min with a cycle time of 6 min/run. 200 mM Sodium Phosphate and 250 mM Sodium Chloride was used as the mobile phase.

### Dynamic Scanning Fluorimetry (DSF)

10 μL of 20× Sypro Orange is added to 20 μL of 0.2-1 mg/mL mAb or Fab solution. A RT-PCR instrument (BioRad CFX96 RT PCR) is used to ramp the sample plate temperature from 40 to 95° C. at 0.5° C. increment, with 2 min equilibrate at each temperature. The negative of first derivative for the raw data is used to extract Tm.

### HIC (Hydrophobic Interaction Chromatography)

The methodology for this assay was described previously (see Estep P, et al. (2015) An alternative assay to hydrophobic interaction chromatography for high-throughput characterization of monoclonal antibodies. MAbs 7(3): 553-561). In brief, 5 pg IgG samples (1 mg/mL) were spiked in with a mobile phase A solution (1.8 M ammonium sulfate and 0.1 M sodium phosphate at pH 6.5) to achieve a final ammonium sulfate concentration of about 1 M before analysis. A Sepax Proteomix HIC butyl-NP5 column was used with a linear gradient of mobile phase A and mobile phase B solution (0.1 M sodium phosphate, pH 6.5) over 20 min at a flow rate of 1 mL/min with UV absorbance monitoring at 280 nm.

### Surface Plasmon Resonance Kinetics Experiments

Kinetic analysis was conducted at 25° C. in HBS-EP+ running buffer (10 mM HEPES pH 7.4, 150 mM NaCl, 3 mM EDTA, 0.05% Surfactant P20) using a Biacore 8K optical biosensor (Cytiva, Marlborough, MA). The sample compartment was maintained at 10° C. for the duration of the experiment. The Biacore 8K can analyze up to 8 separate interactions in a single cycle.

Each experiment cycle began with an injection (300 s at 2 μL/min) overflow cells 1 and 2 of a 1:100 solution of biotin CAPture reagent (Cytiva) in running buffer. This was followed by an injection (45 s at 10.0 μL/min) of biotinylated human ACTIVIN A (5 nM) overflow cell 2. Upon capture of ACTIVIN A to the sensor surface, a single concentration of Fab ranging from 0.111-81.0 nM was injected (300 s at 30 μL/min) overflow cells 1 and 2. The dissociation of the antibodies were monitored for 3420 s. Finally, an injection (120 s at 10 μL/min) of regeneration solution (6 M Guanidine-HCl in 0.25 M NaOH) overflow cells 1 and 2 prepared the sensor surface for another cycle. Several blank buffer samples were injected (300 s at 30 μL/min) overflow cells 1 and 2 throughout the duration of the experiment and used for reference surface subtraction—each were monitored for the same amount of dissociation time (3420 s) and regenerated under the same conditions as those for the Fab sample injections.

The resulting sensorgrams were cropped to include only the association and dissociation steps of the experiment. This cropped data was subsequently aligned, double reference subtracted, and then non-linear least squares fit to a 1:1 binding model using Biacore Insight Evaluation software version 3.0.11.15423. (Myszka D G. Improving biosensor analysis. J Mol Recognit. 1999; 12(5): 279-84. pmid: 10556875).

### AC-SINS

The AC-SINS assay was performed as described previously (Sule et al, Biophysical Journal 101, 1749-1757 (2011), Liu et al, mAbs 6(2), 483-492 (2014)). In short, gold nanoparticles (15705; Ted Pella Inc.) were coated with 80% capturing anti-human goat IgG Fc (109-005-098; Jackson ImmunoResearch) and 20% with polyclonal goat nonspecific antibody (005-000-003; Jackson ImmunoResearch). The antibodies of interest were then incubated with the particles for 2 h and the wavelength shift was measured using Molecular Devices SpectraMax M2 with SoftMax Pro6 software. The self-interacting clones show a higher wavelength shift away from the PBS sample.

### Transient CHO Expression

Transient CHO expression was performed using standard methods known in the art. In general, CHO-K1 cells grown to about 4×10*6 cells/mL were pelleted and resuspended in transfection medium. DNA plasmids (1.5 ug total DNA/mL) were incubated with PEIpro (1:2 final, PolyPlus, Cat #115-100) in transfection medium at room temperature before addition to the CHO-K1 cell suspension. Transfected cultures were fed and maintained at 32° C. or 37° C., shaking until supernatant was harvested (at day 9) for purification.

### Functional Assay Using the Activin 2B Receptor/SMAD Reporter Assay

Compound information: Antibodies provided in HEPES buffer (25 mM HEPES, 150 mM NaCl pH 7.3). It was assumed that the molecular mass of the compounds is 150,000.

Antibody treatment condition: 100 nM, 10 nM, 1 nM, 100 pM, 10 pM, 1 pM, 0.1 pM, 0.01 pM, vehicle control 1 μM single concentration.

### Storage: Stocks are Stored in −80° C. Freezer.

Protocol: HEK-Activin 2B Receptor/SMAD reporter cell line clone 2 was plated in white 96 well plates at 10K cells per well. After overnight incubation the medium was removed and replaced with 90 μL DMEM containing 0.2% FBS, 2 mM L-glutamine and 50 mM HEPES pH7.4 and cells were incubated for 4 h. Activin A was diluted in medium at 10× final concentration. A final concentration of an EC₈₀ of Activin A was used to assess the inhibitory activity of mAbs. This medium was used to prepare dilutions of the mAb at 10× the final required concentration. Activin A/mAb mixtures were incubated for 15 minutes at room temperature before adding 10 μL to the cells. Cells were incubated at 37° C. for 16 hours. Luciferase activity was detected by adding Bright Glo reagent and reading in an En Vision plate reader.

### Results:

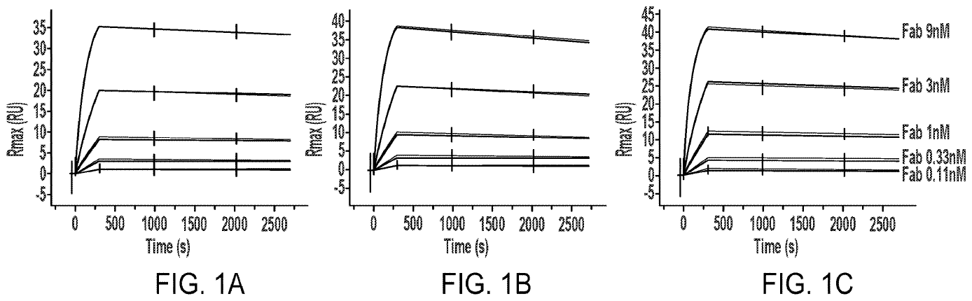

The anti-Activin antibodies identified in Example 1 were characterized. First, binding kinetics of the anti-Activin antibodies was assessed with a Surface Plasmon Resonance assay (Biacore). The binding kinetics of the anti-Activin antibodies are shown in Table 4 and representative graphs for Clones B-D are provided in FIGS. 1A-1C.

The data shows that the anti-Activin antibodies are potent and bind to Activin A with binding affinities within a range of about 6 to about 1000 pM as assessed by Biacore, with most showing affinities within a range of about 6 to about 150 pM and many within a range of about 6 or about 10 to about 100 pM. As can be seen from Table 4 below, 2 of the 11(18%) tested antibodies had affinities around 6-10 pM (e.g., about 6.17 pM, about 8.13 pM, about 9.8 pM, about); 3 of the 11(27%) have affinities below about 20 pM (e.g., between about 6-10 pM and about 15-20 pM); 5 of the 11(45%) have affinities below about 50 pM (e.g., between about 6-10 pm and about 40-50 pM), and 9 of the 11(81%) have affinities below about 120 pM (e.g., between about 9-10 pM and 80-120 pM).

TABLE 4
Binding kinetics of anti-Activin antibodies

| | Biacore Fab K_{D} | | | |
|---|---|---|---|---|
| | biotinylated | | | |
| | Human Activin A | | | |
| | Fab K_{D} (M) | kₒₙ | k_{off} | Rmax |
| Clone | Monovalent | (1/Ms) | (1/s) | (RU) |
| B | 9.81E−12 | 2.31E+06 | 2.27E−05 | 40.14 |
| | 6.17E−12 | 2.56E+06 | 1.58E−05 | 38.1 |
| C | 1.79E−11 | 2.53E+06 | 4.52E−05 | 42.77 |
| | 3.34E−11 | 8.21E+05 | 2.74E−05 | 39.8 |
| D | 9.86E−12 | 3.06E+06 | 3.02E−05 | 43.87 |
| | 8.13E−12 | 2.66E+06 | 2.16E−05 | 44.9 |
| E | 7.79E−11 | 2.23E+06 | 1.74E−04 | 40.56 |
| F | 4.31E−11 | 1.68E+06 | 7.26E−05 | 47.59 |
| G | 8.49E−11 | 1.46E+06 | 1.24E−04 | 32.5 |
| H | 9.60E−10 | 3.86E+05 | 3.71E−04 | 33.3 |
| I | 6.16E−11 | 1.20E+06 | 7.38E−05 | 34.86 |
| J | 1.18E−10 | 8.19E+05 | 9.69E−05 | 31.25 |
| K | 3.67E−11 | 3.25E+06 | 1.19E−04 | 32.11 |
| Q | 2.40E−10 | 7.28E+05 | 1.75E−04 | 21.7 |

Binding affinity and epitope binning assays were performed with a ForteBio instrument as described herein. The results of the binding affinity (Octet) for all antibodies tested are provided in Table 5 and the results of epitope binning experiments for all antibodies tested are provided in Table 6.

TABLE 5
Binding affinity for anti-Activin antibodies

| | Octet IgG K_{D} | Octet Fab K_{D} |
|---|---|---|
| | Human Activin | Human Activin |
| | A Fc (M) | A Fc (M) |
| Clone | Avid | Monovalent |
| A | 8.10E−10 | 2.02E−09 |
| B | 5.27E−10 | 3.09E−10 |
| C | 4.77E−10 | 2.29E−10 |
| D | 5.15E−10 | 5.38E−10 |
| E | 5.51E−10 | 7.90E−10 |
| F | 4.92E−10 | 4.19E−10 |
| G | 6.80E−10 | 1.09E−09 |
| H | 6.66E−10 | 1.86E−09 |
| I | 5.36E−10 | 8.57E−10 |
| J | 6.60E−10 | 1.60E−09 |
| K | 4.30E−10 | 9.25E−10 |
| L | 1.57E−09 | 4.14E−09 |
| M | 1.00E−09 | 4.53E−09 |
| N | 1.26E−09 | 4.85E−09 |
| O | 4.42E−10 | 1.92E−09 |
| P | 1.28E−09 | 1.53E−09 |
| Q | 5.85E−10 | 1.75E−09 |
| R | 6.00E−10 | 2.90E−09 |

TABLE 6
Epitope binning data for anti-Activin antibodies

| | | | | | Octet IgG | Octet IgG | Octet IgG | Octet IgG |
|---|---|---|---|---|---|---|---|---|
| | Octet IgG | Octet IgG | Octet IgG | Octet IgG | K_{D} | K_{D} | K_{D} | K_{D} |
| | K_{D} | K_{D} | K_{D} | K_{D} | Human | Human | Human | Human |
| | Activin B | BMP9 | BMP10 | GDF15 Fc | GDNF | GDF8 | GDF10 | GDF11 |
| Clone | (M) Avid | (M) Avid | (M) Avid | (M) Avid | (M) Avid | (M) Avid | (M) Avid | (M) Avid |
| A | N.B. | N.B. | N.B. | N.B. | N.B. | N.B. | N.B. | N.B. |
| B | 4.68E−08 | N.B. | N.B. | N.B. | N.B. | N.B. | N.B. | 2.10E−08 |
| C | 1.48E−07 | N.B. | N.B. | N.B. | N.B. | N.B. | N.B. | 1.27E−08 |
| D | 1.53E−07 | N.B. | N.B. | N.B. | N.B. | N.B. | N.B. | 2.34E−08 |
| E | P.F. | N.B. | N.B. | N.B. | N.B. | N.B. | N.B. | 2.73E−08 |
| F | 8.01E−08 | N.B. | N.B. | N.B. | N.B. | N.B. | N.B. | 1.87E−08 |
| G | N.B. | N.B. | N.B. | N.B. | N.B. | N.B. | N.B. | N.B. |
| H | P.F. | N.B. | N.B. | N.B. | N.B. | N.B. | N.B. | N.B. |
| I | N.B. | N.B. | N.B. | N.B. | N.B. | N.B. | N.B. | N.B. |
| J | N.B. | N.B. | N.B. | N.B. | N.B. | N.B. | N.B. | N.B. |
| K | N.B. | N.B. | N.B. | N.B. | N.B. | N.B. | N.B. | N.B. |
| L | 2.41E−07 | N.B. | N.B. | N.B. | N.B. | N.B. | N.B. | N.B. |
| M | 9.58E−09 | N.B. | N.B. | N.B. | N.B. | N.B. | N.B. | N.B. |
| N | 6.98E−09 | N.B. | N.B. | N.B. | N.B. | N.B. | N.B. | N.B. |
| O | N.B. | N.B. | N.B. | N.B. | N.B. | N.B. | N.B. | N.B. |
| P | P.F. | N.B. | N.B. | N.B. | N.B. | N.B. | N.B. | N.B. |
| Q | N.B. | N.B. | N.B. | N.B. | N.B. | N.B. | N.B. | N.B. |
| R | N.B. | N.B. | N.B. | N.B. | N.B. | N.B. | N.B. | N.B. |

P.F. indicates a binder with a Poor Fit according to a 1:1 Binding model.
N.B. indicates a non-binder under the conditions tested.

As demonstrated in Tables 4 and 5, the anti-Activin A antibodies bound to human Activin A with a high affinity. Some anti-Activin A antibodies showed detectable binding to one or both of Activin B and GDF-11 under the conditions tested (Table 6).

Further, the data in Table 6 shows that the anti-Activin A antibodies are highly specific to Activin A. For example, the binding affinity of anti-Activin A antibodies in an IgG format to human Activin A was in the range of 4.3×10(−10)M to 1.57×10(−9)M (see Table 5). Seven anti-Activin A antibodies (in an IgG format) showed binding to Activin B with a binding affinity (compared to Activin A binding) in the range of 6.98×10(−9)M to 2.41×10(−7)M. Eleven anti-Activin A antibodies did not show detectable binding to Activin B, under the conditions tested. Five anti-Activin A antibodies (in an IgG format) showed binding to GDF-11 with a binding affinity (compared to Activin A binding) in the range of 1.27×10(−8)M to 2.73×10(−8)M. Thirteen anti-Activin A antibodies did not show detectable binding to GDF-11, under the conditions tested.

The seven anti-Activin A antibodies which showed binding to Activin B had an at least 5.5-fold to 3000-fold lower affinity for Activin B as compared to Activin A (compare binding affinities in IgG format in Tables 5 and 6). The five anti-Activin A antibodies which showed binding to GDF-11 had an at least 200-fold to 350-fold lower affinity for GDF-11 as compared to Activin A (compare binding affinities in IgG format in Tables 5 and 6).

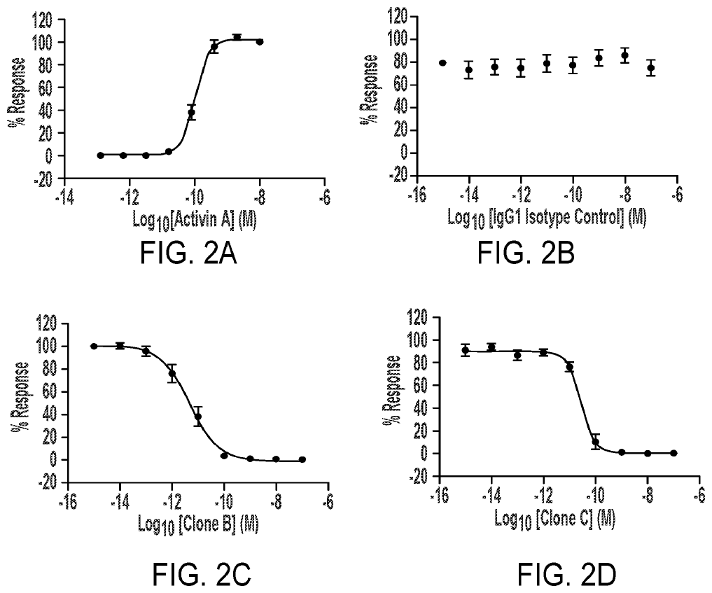

All of the Activin A antibodies had a strong inhibitory effect against Activin A as measured in an Activin IIB Receptor/SMAD reporter assay (see Table 7 and FIGS. 2A-2D).

TABLE 7
Functional selectivity data for Activin A antibody agents

| Clone | Activin A-ActRIIB SMAD IC₅₀ pM | |
|---|---|---|
| A | 323.8 pM; 282 pM; 361 pM | |
| B | 0.3 pM, 1.2 pM, 3 pM; 14.8 pM; | |
| | 17.5 pM; 42 pM, 0.01 pM | |
| C | 0.1 pM, 10 pM, 16.2 pM, 2.4 pM; | |
| | 14.8 pM; 99.5 pM; 0.59 pM | |
| D | 0.1 pM, 10 pM, 3.7 pM, 2.0 pM | |
| E | 16.5 | pM |
| F | 0.1 pM, 10 pM, 1.2 pM, 0.4 pM | |
| G | 15.81 | pM |
| H | 36.9 | pM |
| I | 1.09 | pM |
| J | 41.72 | pM |
| K | 0.03 pM; 1.2 pM, 60.5 pM | |
| L | 200.3 | pM |
| M | 718.7 | pM |
| N | 240.8 | pM |
| O | 5400 | pM |

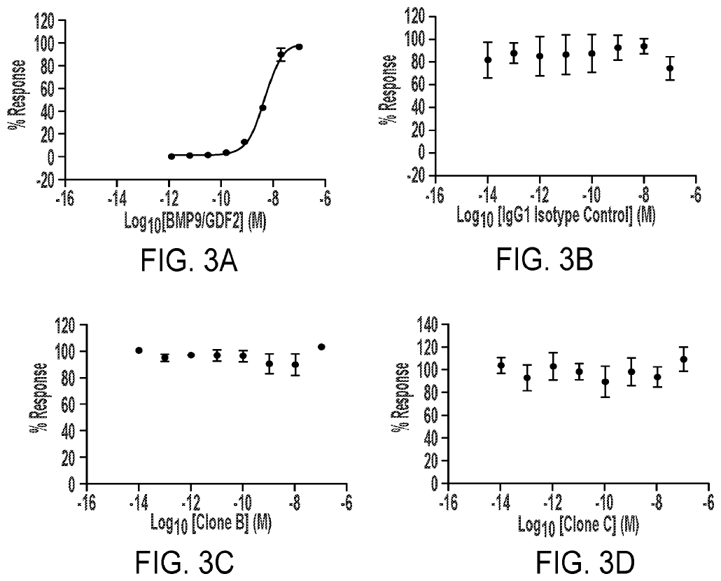

None of the anti-Activin A antibodies functionally inhibited GDF11, and none showed more than minimal inhibitory activity against Activin B, when assessed as described herein (e.g., as measured in an Activin IIB Receptor/SMAD reporter assay) (Table 8). BMP9 or BMP10 were also not inhibited by any of the anti-Activin A antibodies (Table 8 and FIGS. 3A-3D for BMP9).

TABLE 8
Functional selectivity data for Activin A antibody agents

| | Activin B- | | GDF-11- | BMP9 - | BMP10 - |
|---|---|---|---|---|---|
| | ActRIIB | | ActRIIB | ActRIIB | ActRIIB |
| | SMAD | | SMAD | SMAD; IgG | SMAD; IgG |
| Clone | IC50 pM | | IC50 pM | IC50 (pM) | IC50 (pM) |
| A | >100000 | pM | | | |
| B | >100000 pM, 9828 | | ND | ND, ND | ND |
| | pM; 8710 pM, ND, | | | | |
| | ND, ND | | | | |
| C | >100,000 pM, ND, | | ND | ND, ND | ND |
| | ND, ND, ND, ND | | | | |
| D | 19000 pM, 3700 pM | | | | |
| E | 83600 | pM | | | |
| F | 24900 | pM | | | |
| G | >100,000 | pM | | | |
| H | >100,000 | pM | | | |
| I | 52370 pM; ND, ND | | ND | ND | ND |
| J | 60840 | pM | | | |
| K | 30700 pM; ND, ND | | ND | ND | ND |
| L | >100000 | pM | | | |

| | |
|---|---|
| P | N/D |
| Q | N/D |
| R | N/D |

ND: Not detected

Binding of a subset of anti-Activin A antibodies to Activin A, Activin B, and GDF11 in the presence or absence of human serum albumin (HSA) was evaluated to further analyze binding specificity of the antibodies. As shown in Table 9, anti-Activin antibodies clones B and Q bound to Activin A with similar affinity in the presence or absence or 5% HSA. For both clones, binding to Activin B or GDF-11 was significantly reduced in the presence of 5% HSA. Of note, human blood commonly contains 5-10% human serum albumin and albumin concentration can alter binding characteristics of antibodies.

TABLE 9

| | | | |
|---|---|---|---|
| Name | with 0.1% BSA | with 0.1% HSA | with 5% HSA |

Human Activin A Fc KD (M)

| | | | |
|---|---|---|---|
| Clone B | 1.38E−10 | 1.38E−10 | 2.17E−10 |
| Clone Q | 1.28E−10 | 1.56E−10 | 1.89E−10 |

Human GDF-11 Fc KD (M)

| | | | |
|---|---|---|---|
| Clone B | 1.89E−08 | 1.83E−08 | P.F. |
| Clone Q | N.B. | N.B. | N.B. |

Human Activin B Fc KD (M)

| | | | |
|---|---|---|---|
| Clone B | 3.37E−09 | 2.96E−09 | 1.00E−08 |
| Clone Q | N.B. | N.B. | N.B. |

Next, hydrophobicity of the anti-Activin A antibodies was tested using a HIC assay as described herein. Typically, hydrophobic antibodies show self-association and thus low hydrophobicity is preferred. The results of this assay for the anti-Activin A antibodies are shown in Table 10. Retention time of less than 10.5 minutes indicates a clean to low HIC, retention time of 10.5 to 11.5 indicates medium HIC and a retention time of more than 11.5 minutes indicates high HIC. The data in Table 10 demonstrates that all the anti-Activin A antibodies tested had low to medium hydrophobicity and are thus expected to have low self-association, with most of the clones showing retention times between 8 minutes to about 12 minutes. 10 of the 18 antibodies had retention times of less than 10.5 minutes (e.g., about 8.7 minutes to about 10.2 minutes). 17 of the 18 antibodies had retention times of less than 11.5 minutes.

TABLE 10
HIC Retention Time

| | HIC Retention |
|---|---|
| Clone | Time (min) |
| A | 8.7 |
| B | 9.7 |
| C | 9.3 |
| D | 9.4 |
| E | 9.6 |
| F | 9.8 |
| G | 9.1 |
| H | 9.1 |
| I | 11.5 |
| J | 12.4 |
| K | 10.7 |
| L | 11.2 |
| M | 11.1 |
| N | 11.2 |
| 0 | 10.6 |
| P | 10.8 |
| Q | 10.2 |
| R | 10.1 |

Stability of the anti-Activin antibodies was assessed with a DSF assay as described herein. The data shown in Table 11 shows that the melting temperature for the Fab fragments tested was between 69.5° C. to 82° C.

TABLE 11
Melting temperature for Fab fragments

| | Fab Tₘ by DSF |
|---|---|
| Clone | (° C.) |
| A | 77.5 |
| B | 81.0 |
| C | 81.0 |
| D | 78.5 |
| E | 74.0 |
| F | 77.0 |
| G | 78.0 |
| H | 78.5 |
| I | 73.0 |
| J | 72.5 |
| K | 69.5 |
| L | 78.5 |
| M | 80.5 |
| N | 80.0 |
| O | 76.5 |
| P | 70.5 |
| Q | 82 |
| R | 81 |

As a further characterization, self-association of a subset of anti-Activin antibodies was evaluated using the AC-SINS assay described herein using a CHO mammalian expression system. Transient CHO expression was first performed using standard methods, followed by purification of the resulting antibodies. The purified antibodies were then subjected to an AC-SINS assay to evaluate self-interaction of the antibodies. The data from this experiment is provided in Table 12 and shows that the Clone A and Clone B anti-Activin A antibodies had low AC-SINS values and thus have a low likelihood of self-associating.

TABLE 12
AC-SINS

| | AC-SINS |
|---|---|
| Clone | Δλmax (nM) |
| A | 0.43 |
| B | 1.31 |
| E | 27.62 |

Taken together, the data provided in this Example indicates that the human anti-Activin A antibodies identified in Example 1 have suitable “developability” characteristics and supports the development of these antibodies as therapeutics.

### Example 3: Expression of Activin a Antibody Agents in a Human Cell Line

This Example describes expression of anti-Activin antibody Clones A-E identified in Example 1 in a HEK293 expression system.

### Methods:

HEK293 cells were used to supply a transient expression of antibodies. Heavy and light chain of the antibody were individually cloned into an expression vector under CMV promotor. Equal amount of the heavy and light chain DNA constructs were mixed with polyethyleneimine (PEI). Transfection of HEK293 cells was performed by adding the PEI/DNA mixture to the suspension cells in exponential growth phase. The cells were then incubated at 37° C., 8% CO2 and 120 rpm for up to 7 days. The culture was harvested by centrifuging the cell culture fluid and filtering the supernatant using a 0.2 pm depth filter.

SEC-HPLC was used to determine the molecular size variants in purified antibodies. Samples were diluted to 1-2 mg/mL with mobile phase and filtered through a 0.22 μm cellulose acetate membrane before analysis by YMC-Pack Diol column or equivalent. After peak integration, the main peak, aggregates (HMW), and fragments (LMW) were calculated based on the area normalization method.

### Results

As shown in Table 13, all antibodies tested were successfully expressed and purified in the HEK293 transient expression system. High expression of all clones was observed, with antibody concentrations in the range of about 61-310 mg/L. Two of the five (40%) antibodies tested had antibody concentrations of above about 300 mg/L. Three of the five (60%) antibodies had antibody concentrations of above about 190 mg/L. Four of the five (80%) antibodies tested had antibody concentrations of above about 120 mg/L.

TABLE 13
Antibody concentration and SEC peak

| | HEX-293 | HEK-293 produced |
|---|---|---|
| Clone | expression mg/L | SEC Main Peak (%) |
| A | 61, 86.7, 95.8 | |
| | Average 81.1 | |
| B | 300.8 | 96.1 |
| C | 233.2, 147. | 95.9, 96.9 |
| | Average 190.1 | |
| D | 310.3 | 99.4 |
| E | 142.8, 121.8. | 99.9 |
| | Average 132.3 | |

### Example 4: Pharmacokinetics of Anti-Activin A Antibodies

This Example describes pharmacokinetic analysis of exemplary anti-Activin A antibodies identified in Example 1, in mice and primates.

### Methods

TABLE 14
IV Protocol in Primates

| Category | Item | Description |
|---|---|---|
| Species | Cynomolgus Monkey | Male, Naïve cynomolgus (3-3.5 1 g), N = 3 |
| Formulation | Antibodies formulated in PBS | |
| In-life | mAb, IV, 5 mg/kg | Dosing named Day 0 |
| | 1 mL/kg, N = 3 | PK sampling at predose, 2 hr, 8 hr post |
| | | dosing on Day 0, then daily on Day 1 (24 |
| | | hr), 2, 3, 7, 10, 14, 21, 28 and 35. 12 |
| | | time points. Serial bleeding from the |
| | | same animal for serum only |
| Bioanalytical | Method development | Bioanalytical method development using |
| | | generic ELISA for antibody, AP run. |
| | Sample analysis | 36 serum samples for mAb, using generic |
| | | ELISA |

### PK Protocol in Mice:

| | | | | | Dose | | |
|---|---|---|---|---|---|---|---|
| | | Test | Dose | Conc | volume | | |
| Group | N/sex | Article | (mg/kg) | (mg/mL) | (mL/kg) | Vehicle | Route |
| 1 | 18/M | Clone B | 1 | 2.5 | 0.4 | Sterile PBS | IV |
| 2 | 18/M | Clone B | 10 | 2.5 | 4 | Sterile PBS | SC |
| 1 | 18/M | Clone C | 1 | 2.5 | 0.4 | Sterile PBS | IV |
| 2 | 18/M | Clone C | 10 | 2.5 | 4 | Sterile PBS | SC |

Formulation: Anti-Activin A antibodies were diluted as appropriate to achieve the final concentrations. The doses were stored at −20° C.±5° C. upon arrival and following dose administration. The doses were brought to ambient temperature prior to dose administration.

Dosing: IV doses were delivered directly through the tail vein with an appropriately sized needle. SC doses were administered to the intrascapular region. Dose volumes were adjusted for the weight of the animal on the morning of dose administration.

Sample collection: Each mouse had up to 2 blood samples collected. Blood samples were collected from 3 mice at each specified time point with preference to the retro orbital sinus (first sample) or cardiac puncture (second or only sample) while anesthetized with inhaled Isoflurane. Blood samples were kept on ice until processing.

| | | | Volume/time | Collection Times |
|---|---|---|---|---|
| Group | Matrix | Anticoagulant | point | (h postdose) |
| A11 | Blood/ | K2EDTA | ~0.2 mL (retro | 0, 1, 2, 6, 8, 24, 48, |
| | Plasma | | orbital) or ~1 mL | 144, 312, 480, and 696 |
| | | | (cardiac puncture) | |

Sample Processing: All blood samples were centrifuged within 1 h of collection at settings of 3200 RPM, 10 min, and 5° C. Plasma samples were directly transferred into cluster tubes and stored at −20±5° C. until shipment.

### Results:

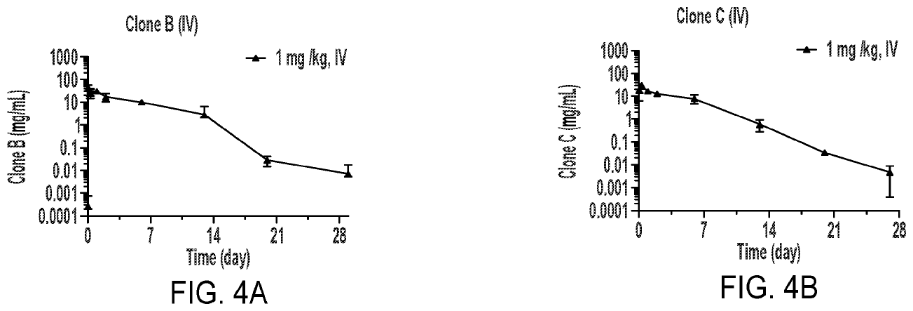

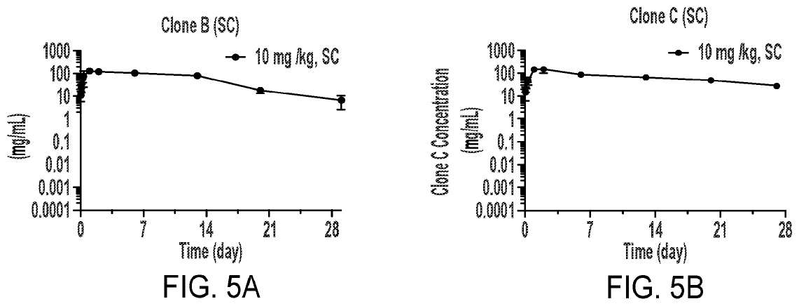

FIGS. 4A-B shows the mean serum concentration-time profile of antibody Clone B or Clone C dosed intravenously at a dose of 1 mg/kg to male C57BL/6 Mice (N=3/timepoint). The antibody concentration in serum was stable for one week. High concentrations were obtained for the duration of the 4 week experiment. FIGS. 5A-5B show the mean serum concentration-time profile of antibody Clones B or C dosed subcutaneously at a dose of 10 mg/kg to male C57BL/6 Mice (N=3/timepoint). The antibody concentration in serum peaked at about 1 hour post dosing and remained relatively stable for the rest of the experiment (up to 28 days post administration).

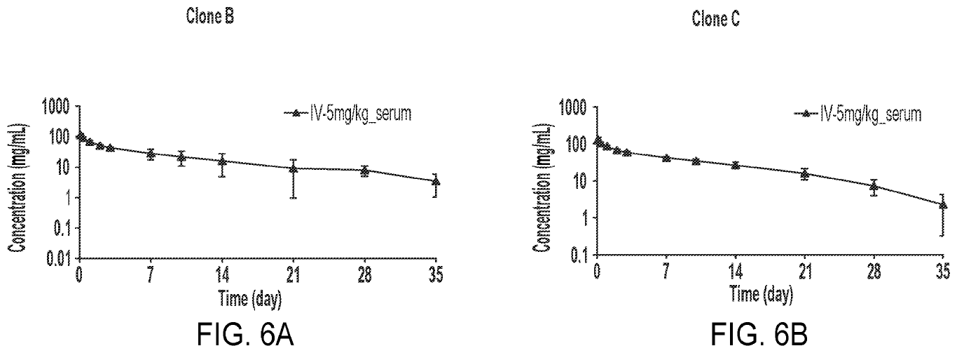

In monkeys dosed intravenously with antibody Clone B or Clone C at a dose of 5 mg/kg, similar favorable pharmacokinetics were observed. As shown in FIGS. 6A-6B, the antibody concentration in the serum was about 100 μg/mL immediately post-dosing and remained at a high concentration throughout the course of the experiment (up to 35 days).

This data shows that the exemplary anti-Activin antibodies tested (Clones B and C) had favorable pharmacokinetic properties in vivo in mice and primates suggesting their therapeutic utility.

### Example 5: Preventative and Therapeutic Activity of Anti-Activin A Antibody In Vivo

Methods: Male C57BL/6J mice (28 g upon arrival) were housed in polypropylene cages with free access to standard rodent chow and filtered tap water. Upon arrival, mice were weighed. Animals were maintained on a normal light/dark cycle (lights on: 07:00-19:00). Animals were acclimatized to the facility for approximately one week prior to use and were handled prior to the onset of procedures. The experiment was performed in the light phase of the light/dark cycle.

The week after arrival, all animals began a 3-day daily handling protocol where each animal was picked up but not dosed or weighed. On Day 0 animals were allocated to dosing groups based on body weight. The allocation was undertaken by a statistician and ensured that groups were balanced as closely as possible for these variables. To assess the effect of anti-Activin A antibody on baseline body weight and on preventing Activin A-induced weight loss, two studies were used to i) prevent and reverse a high dose of AAV-INHBA (Activin A) and ii) reverse a lower dose of AAV-Activin A which increased circulating Activin A levels comparable to patient reported levels and enabled chronic studies. Mice were administered a single dose of either IgG₁ isotype control (10 mg/Kg, PBS, subcutaneously) or anti-Activin A antibody (1 or 10 mg/Kg, PBS, subcutaneously).

AAV dosing was undertaken on Day 1. Dosing is not time critical but started approximately 10:00 am. In the high dose AAV-Activin A study, mice were dosed by the intravenous route and received either control vector (1.4×10¹² GC/mL, Vector Biolabs) or AAV8-Cag-h-INHBA (2.9×10¹² GC/mL, Vector Biolabs). Subsequent to dosing, mice were observed and weighed daily (beginning circa 10:00 am). Once non-mAb animals achieved 10% weight loss (Day 4) the anti-Activin A antibody Clone C was administered and efficacy of the antibody was assessed in therapeutic model. In the prevention studies, anti-Activin A antibody Clone C was administered on Day 0 and the animals were dosed with AAV on Day 1. Mice either received vehicle (IgG₁ isotype control, PBS subcutaneously) or anti-Activin A antibody Clone C (10 mg/Kg, PBS, subcutaneously). During the vehicle or antibody dosing phases, mice were weighed daily and food and water intake monitored. The High dose Activin A study was terminated at Day 6-7 due to meeting IACUC weight loss limits.

In the lower dose AAV-INHBA (Activin A) study, mice were dosed by the intravenous route and received either control null vector (9.6×10¹¹ GC/mL (0.1 mL), Vector Biolabs) or AAV8-Cag-h-INHBA (9.6×10¹¹ GC/mL (0.1 mL), Vector Biolabs). Mice were weighed daily and once 20% weight loss was achieved, the IgG1 isotype control, anti-Activin A antibody Clone C or anti-Activin A antibody Clone B was administered and efficacy of the antibody was assessed in a therapeutic mode. Mice were continued to be weighed daily.

All animals were killed by a schedule 1 method (increasing exposure to CO2 and confirmation of death by cervical dislocation). The blood was taken by cardiac puncture into an EDTA-coated tube and stored at −80° C. prior to analysis. Body organs were removed and weighed.

Plasma AST was measured using the Cobas C111 automated Chemistry analyzer and according to the manufacturer's instructions for estimation of Alkaline phosphatase or analyzed by IDEXX.

Plasma ALT was measured using the Cobas C111 automated Chemistry analyzer and according to the manufacturer's instructions for Alanine transaminase (ALT) estimation or analyzed by IDEXX.

For histology, liver and kidneys were collected at termination and placed into 10% NBF. All tissues were fixed for approximately 3 days prior to processing to wax block (FFPE). The tissues samples were sectioned at 5 μm thick and the slides stained using a method for Sirius Red. Stained slides were blinded and independently scored for fibrosis by a histopathologist.

Gene expression studies: The total RNA was isolated from liver (5-10 mg of tissue) using MagMAX™-96 Total RNA Isolation Kit (AM1830) performed on the KingFisher Flex instrument (Thermofisher). 2 μL sample of the RNA was pipetted out into a fresh 96-well plate and the quality as well as quantity of RNA was established by Nanodrop (260/280 ratio between 1.8-2.0 was considered as good quality of RNA). Complementary DNA (cDNA) was prepared using Meridian Bioscience SensiFAST cDNA Synthesis Kit (BIO-65054) using StepOne™ real-time PCR system (Thermofisher). Mouse gene expression was performed using TaqMan assays purchased from Thermofisher on the target genes and two housekeeping genes (GAPDH and HPRT; Assay ID: Mm03302249_g1 and Assay ID: Mm03024075_m1, respectively) for normalization. All genes were analysed using CFX384 real-time PCR detection system (Biorad) in a 384-well format. Efficiency test was performed on all primers before running the qPCR (acceptable range considered for efficiency test was within of 90-110%).

### Example 5.1: Anti-Activin a Antibody Prevents and Stops Activin A-Induced Weight Loss In Vivo

This Example describes a preventative effect and a therapeutic effect on weight loss with an exemplary anti-Activin A antibody in a mouse model of weight loss induced by Activin A overexpression. The anti-Activin A antibody prevented weight loss and stopped weight loss induced by an adeno-associated vector (AAV) mediated expression of Activin A (AAV-Activin A).

Results: The results demonstrate that a single dose of an anti-Activin A antibody (Clone C) prevented, reversed, or stopped the following biological effects in mice: weight loss, muscle loss, organ mass loss.

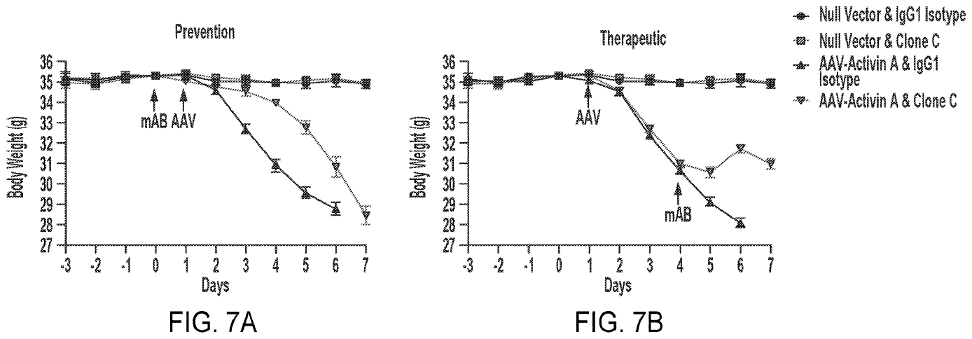

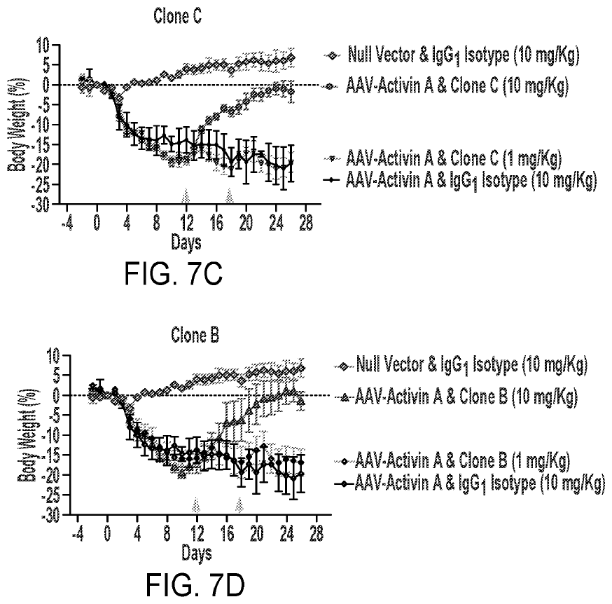

With respect to the effect of the anti-Activin antibody on weight loss, FIG. 7A shows that in a prevention setting, a single administration of anti-Activin A antibody (Clone C), prevents weight loss induced by AAV-Activin A (compare purple inverted triangles with solid black triangles). FIG. 7B shows that in a therapeutic setting, a single administration of anti-Activin A antibody (Clone C) on Day 4, after the animals have achieved about 10% weight loss, stopped the weight loss as compared to control animals.

A similar effect in the therapeutic setting was observed with administration of an additional exemplary anti-Activin A antibody (Clone B). As shown in, FIGS. 7C-7D, when mice achieved 20% weight loss, administration of a single dose of Clone C (FIG. 7C) or Clone B (FIG. 7D) resulted in stopping and reversing of weight loss induced by AAV-Activin A and administration of the second dose of Clone C (FIG. 7C) or Clone B (FIG. 7D) resulted in full reversal of weight loss induced by AAV-Activin A. The observed stopping of weight loss and reversal of weight loss induced by AAV-Activin A with administration of exemplary anti-Activin A antibodies was compared to weight loss induced by AAV-Activin A in animals administered control antibodies.

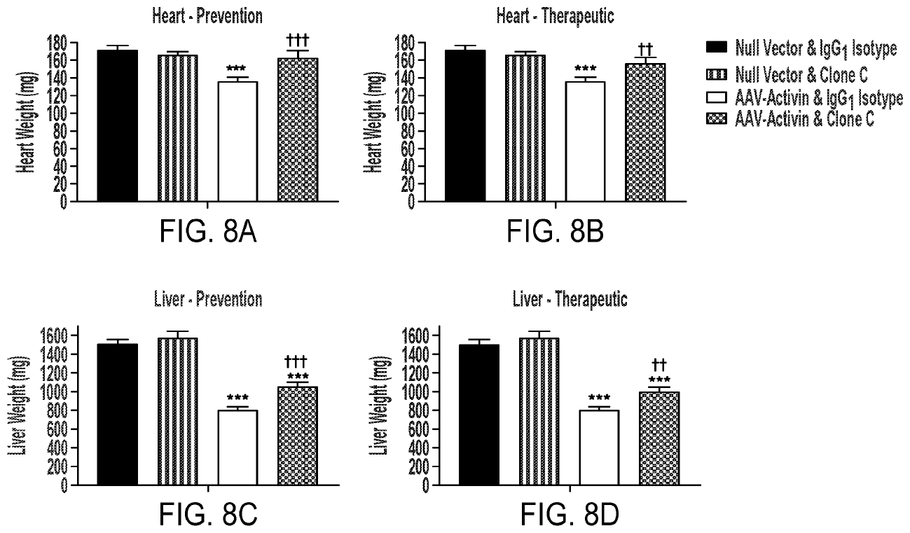

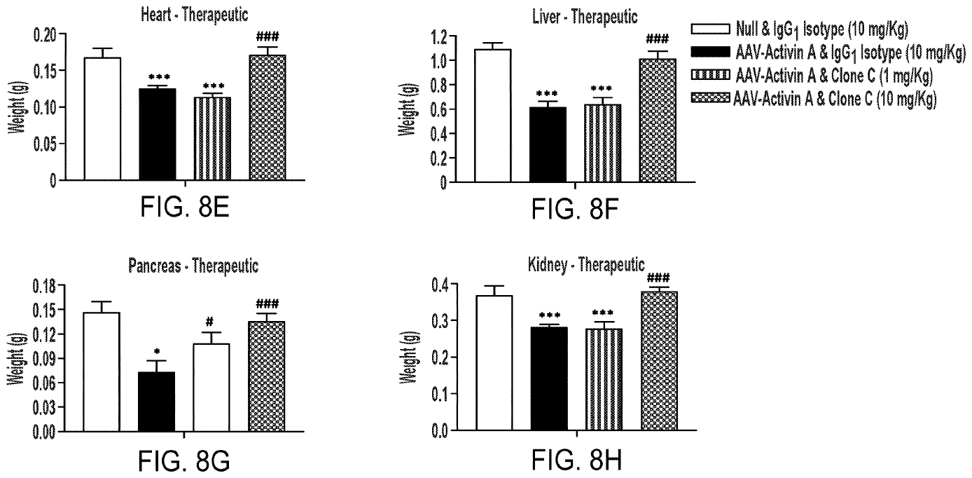

This data demonstrates that administration of an anti-Activin A antibody can prevent weight loss in a prevention setting. An anti-Activin A antibody can also stop and reverse weight loss in a therapeutic setting. FIGS. 8A and 8B show that a single dose of Clone C can prevent and reverse heart muscle loss. FIG. 8A from the prevention setting shows prevention of heart muscle loss by about 20% in animals dosed with a single dose of Clone C. FIG. 8B from the therapeutic setting shows reversal of heart muscle loss by about 20% with a single dose of Clone C.

Similar results were observed in the liver of animals dosed with a single dose of Clone C in a prevention setting or a therapeutic setting. FIG. 8C from the prevention setting, shows reduction of liver mass loss 6 days after administration of a single dose of Clone C. FIG. 8D from the therapeutic setting shows reversal of Activin A induced liver mass loss within 2 days of administration of a single dose of Clone C.

Additionally, Activin A induced atrophy of the heart, liver, pancreas, and kidneys was reversed with administration of two doses of Clone C (FIGS. 8E-8H). The reversal in atrophy was dose-dependent. Administration of two doses of Clone C at 1 mg/kg was sufficient to reverse atrophy of the pancreas, while reversal of heart, liver and kidney atrophy was observed with administration of 10 mg/kg of Clone C.

This data demonstrates that administration of an anti-Activin A antibody can prevent and/or reverse muscle loss and organ mass loss such as those induced by Activin A.

### Example 5.2: Anti-Activin A Antibody Prevents Activin A-Induced Liver Damage In Vivo

This Example describes a prevention and reversal of liver damage with an exemplary anti-Activin A antibody in a mouse model of liver damage induced by Activin A overexpression.

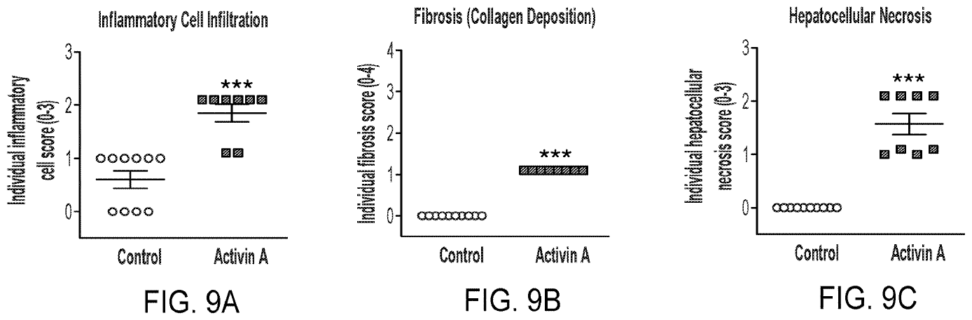

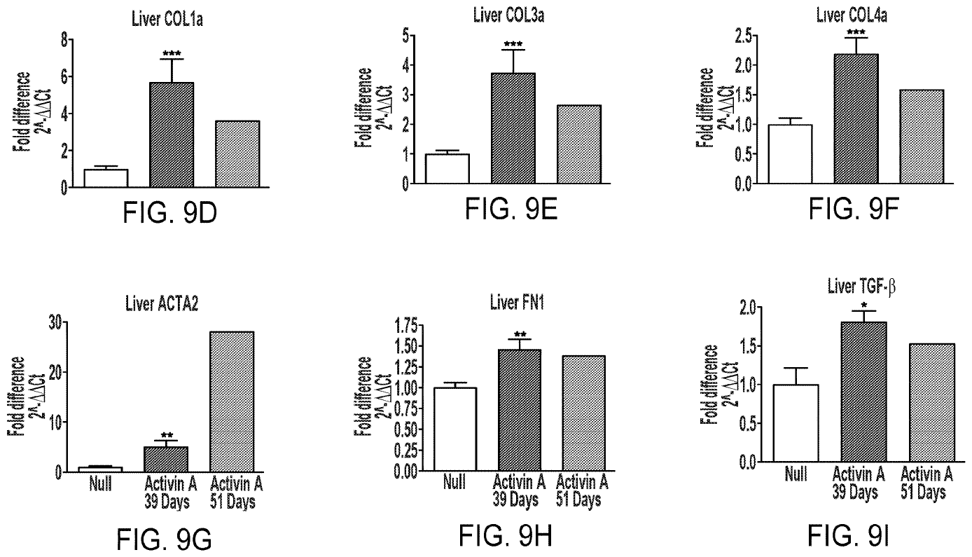

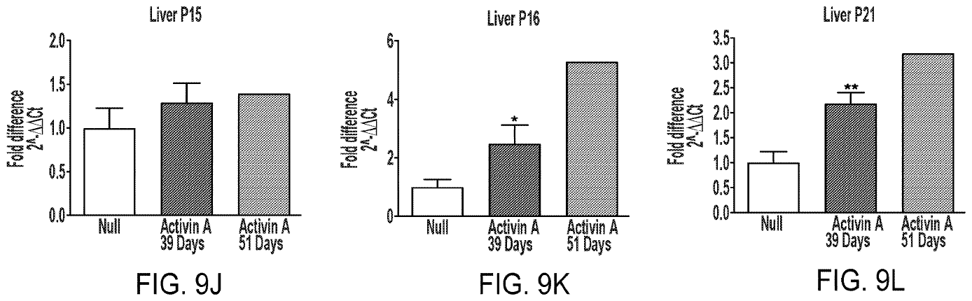

FIGS. 9A-9C show that acute Activin A overexpression induced liver weight loss attributed to acute liver injury. FIG. 9A shows increased inflammatory cell inflammation, FIG. 9B shows fibrosis, and FIG. 9C shows hepatocellular necrosis in mice overexpressing Activin A.

When Activin A was expressed chronically, an increase in fibrotic genes was observed. FIGS. 9D-9I show increased expression of Collagen (FIGS. 9D-9F), smooth muscle actin (FIG. 9G), fibronectin (FIG. 9H) and TGF-β (FIG. 9I) at 39 days and 51 days post-Activin A overexpression. Additionally, increased expression of p16 (FIG. 9K) and p21 (FIG. 9L) was also observed in liver cells after chronic overexpression of Activin A, indicating Activin-A induced senescence in the liver.

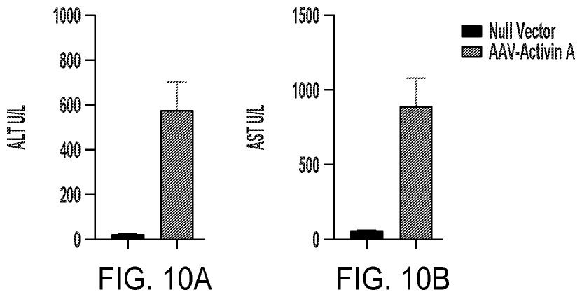

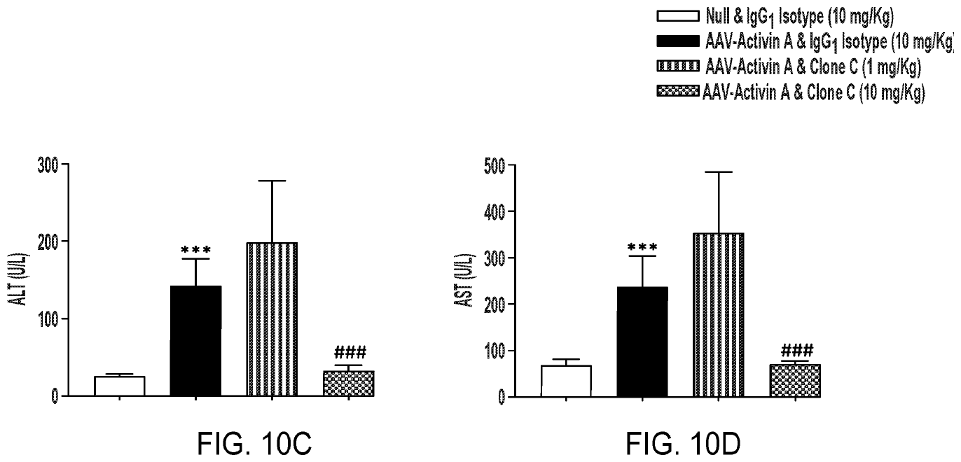

The results in FIG. 10A and FIG. 10B also show that acute overexpression of high dose Activin A (within 6 days) increases liver enzymes by about 10-20 fold. FIG. 10A shows an increased in ALT in animals administered AAV-Activin A and FIG. 10B shows an increased in AST in animals administered AAV-Activin A. Blocking Activin A with exemplary anti-Activin A antibodies in animals administered AAV-Activin A resulted in a full reversal of liver damage. FIGS. 10C-10D show full reversal ALT and AST levels in animals administered AAV-Activin A and treated with exemplary anti-Activin A antibodies as compared to animals administered AAV-Activin A and treated with control antibodies (FIGS. 10C-10D).

This data demonstrates that anti-Activin A antibodies can be used to treat and/or prevent liver disorders and induce liver regeneration, e.g., disorders associated with liver damage, fibrosis and/or cirrhosis which can be associated with elevated levels of Activin A.

### Example 5.3: Activin A Induces Kidney Damage In Vivo

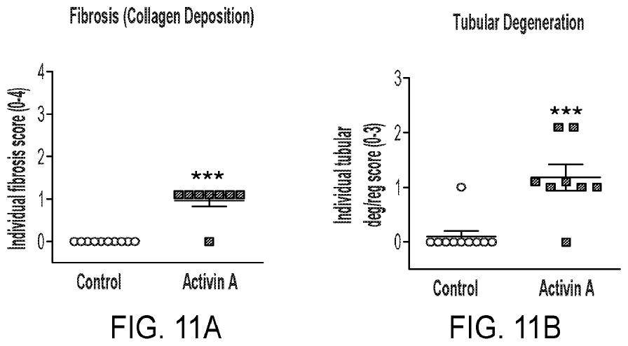

In addition, acute overexpression of Activin A also caused fibrosis and tubular degeneration in the kidney of mice (See FIG. 11A for kidney fibrosis, and FIG. 11B for tubular degeneration). This data demonstrates that anti-Activin A antibodies can be used to treat and/or prevent kidney diseases, e.g., acute kidney injury or chronic kidney disease which can be associated with elevated levels of Activin A.

Summary: In summary, the data provided in Example 5 (including Examples 5.1, 5.2, and 5.3) demonstrates that a single or repeat dose of an anti-Activin A antibody (specifically, in this example, Clone C) prevented, reversed, or stopped Activin-A induced: weight loss, muscle loss, organ mass loss, liver damage, and/or kidney damage in mice.

Without wishing to be bound by any particular theory, it is proposed that the observed prevention and/or reversal of weight loss arises from the activity of anti-Activin A antibodies in preventing and reversing organ atrophy. Anti-Activin A antibodies are anti-catabolic, pro-anabolic, anti-inflammatory and anti-fibrotic mechanisms across organs via inhibition of SMAD 2/3 signaling and non-canonical SMAD-4 independent signaling. The present disclosure therefore demonstrates prevention and/or reversal of weight loss induced by overexpression of Activin A and teaches that anti-Activin A antibodies can be used to prevent and/or reverse weight loss in patients having increased levels of Activin A.

Without wishing to be bound by any particular theory, it is proposed that the observed prevention and/or reversal of muscle loss arises from the activity of anti-Activin A antibodies in preventing and reversing muscle atrophy. Anti-Activin A antibodies are anti-catabolic, pro-anabolic, anti-inflammatory and anti-fibrotic mechanisms due to inhibition of SMAD 2/3 signaling and non-canonical SMAD-4 independent signaling. The present disclosure therefore demonstrates prevention and/or reversal of muscle loss induced by overexpression of Activin A and teaches that anti-Activin A antibodies can be used to prevent and/or reverse muscle loss in patients having increased levels of Activin A.

### Example 6: In Vivo Anti-Cancer Activity of Anti-Activin A Antibody

This Example describes in vivo activity of an exemplary anti-Activin A antibody in an intrasplenically implanted MC38 model of colorectal cancer. Among other things, this Example documents inhibition of metastasis.

Methods: Metastatic MC38 Mouse In Vivo Model. 16-week-old male C57BL/6 mice (The Jackson Laboratory, Bar Harbor, ME, USA) were group-housed (4 per cage). Animals were injected with intrasplenic injection of 1.25×10⁵ MC38 tumor cells in sterile saline (MC38, n=16) or an isovolumetric intrasplenic injection of vehicle (Sham, n=8) as per Huot et al., (2021) Int. J. Mol. Sci. 22(3): 1486. Sham and one MC38 group (n=8) received a subcutaneous injection of IgG1 isotype control (20 mg/Kg in PBS) on Day-2 prior to implantation and dosed every 4 days. Another MC38 group was dosed with Clone C (20 mg/Kg in PBS) on Day-2 prior to implantations, and dosed every 4 days. Food intake and body weight were measured daily and on day 18 post MC38 cell implantation, animals were euthanized under light isoflurane anesthesia. Following euthanasia tissues and tumors were harvested and weighed. Mice carcasses were weighed.

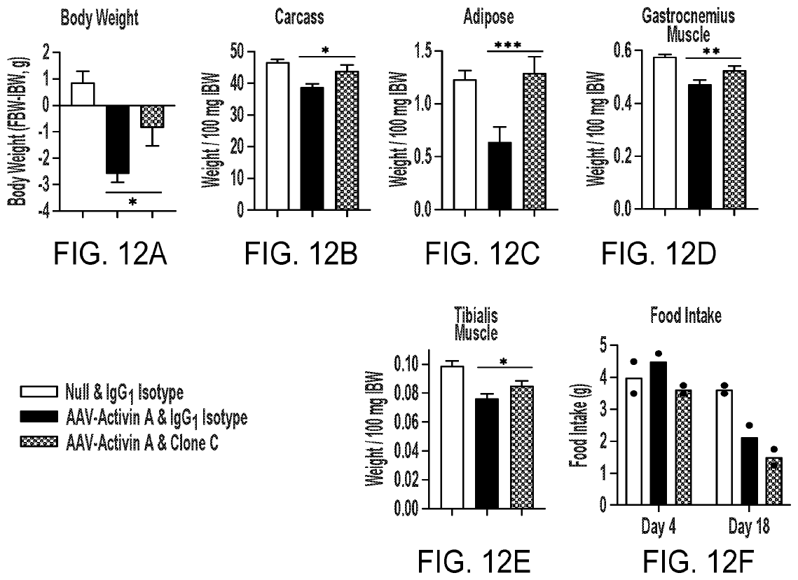

Results: In this MC38 model of colorectal cancer, the exemplary anti-Activin A antibody prevented cancer-induced weight loss, adipose loss and muscle loss in mice as compared to sham treated mice or isotype control treated mice (FIGS. 12A-12E). Notably, there was no effect on food intake with or without treatment with the exemplary anti-Activin A antibody (FIG. 12F). This data demonstrates that anti-Activin A antibodies can be used to prevent cancer-induced weight loss, adipose loss, and/or muscle loss in cancer patients.

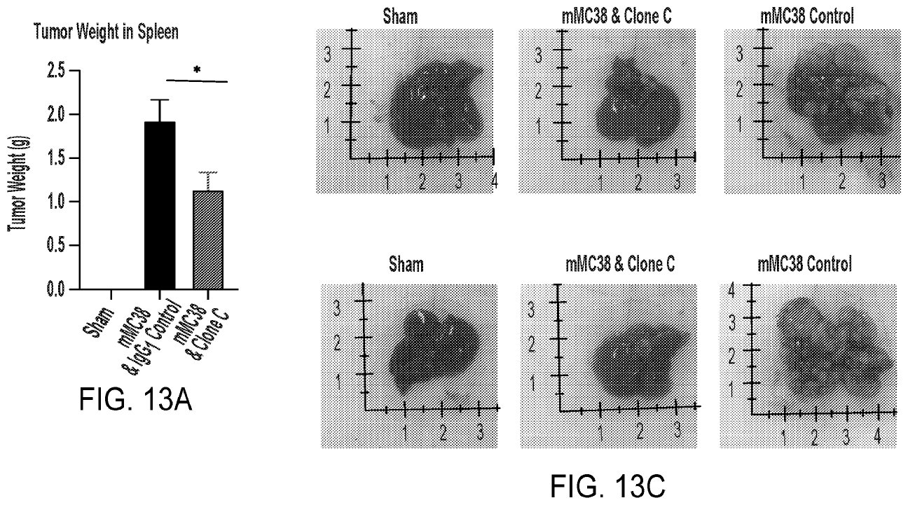

Further as shown in FIG. 13A, the exemplary anti-Activin A antibody prevented growth of the primary colorectal tumor in the spleen in mice implanted intrasplenically with MC38 colorectal cancer cells. In addition, the exemplary anti-Activin A antibody also prevented liver metastases in mice implanted with intrasplenic MC38 colorectal cancer cells (FIGS. 13B-13C). This data demonstrates that the anti-Activin A antibody can prevent: (1) growth of cancer cells; and/or (2) migration, invasion and/or colonization of cancer cells, e.g., to the liver thus reducing metastases in the liver. This data also suggests a therapeutic efficacy for anti-Activin A antibodies in reducing and/or preventing cancer growth and/or cancer metastases.

Without wishing to be bound by any particular theory, it is proposed that the observed inhibition of tumor growth arises from the activity of Anti-Activin A antibodies in preventing tumor growth. The present disclosure therefore demonstrates inhibition of growth of colorectal cancer tumor with an anti-Activin A antibody and teaches that anti-Activin A antibodies can be used to treat and/or eliminate cancer.

Without wishing to be bound by any particular theory, it is proposed that the observed metastasis inhibition arises from the activity of Anti-Activin A antibodies in preventing cell migration, e.g., extravasation out of a tissue or organ into the bloodstream or lymphatic system, and/or intravasation into a secondary tissue or organ to establish a metastasis. The present disclosure therefore demonstrates inhibition of metastasis from a colorectal cancer tumor with an anti-Activin A antibody and teaches that anti-Activin A antibodies can be used to inhibit and/or reduce metastasis in other cancers.

### Example 7: Prevention of Chemotherapy-Induced Nephrotoxicity with Anti-Activin A Antibody

This Example describes in vivo activity of an exemplary Anti-Activin A antibody in preventing Cisplatin-induced nephrotoxicity.

Methods: Male Wistar rats 6-8 weeks old of age, purchased from Charles River Labs, were acclimatized for five days. Rats were housed on a 12 hour light/dark cycle (lights on at 7:00 AM), with one rat per cage in a ventilated cage rack system. Rats received standard rodent chow and water ad libitum. Following acclimation, all rats were subjected to baseline bodyweight and randomized into treatment groups. Vehicle (PBS/Sham), IgG1 isotype control or Clone C and was administered to the rats subcutaneously (SC) at 20 mg/kg one day prior to the start of Cisplatin treatment and on Day 3 of Cisplatin treatment. Cisplatin (Sigma Cat No. 479306) was formulated in saline at 2 mg/mL and used to induce nephritis by intra-peritoneal (IP) injection on Day 1(5 mg/kg). On day five of the study, blood was collected. Serum was isolated for analysis of blood urea nitrogen (BUN)_and creatinine levels. Serum creatinine, serum BUN, were determined using commercially available ELISA kits as per manufacturer's instructions.

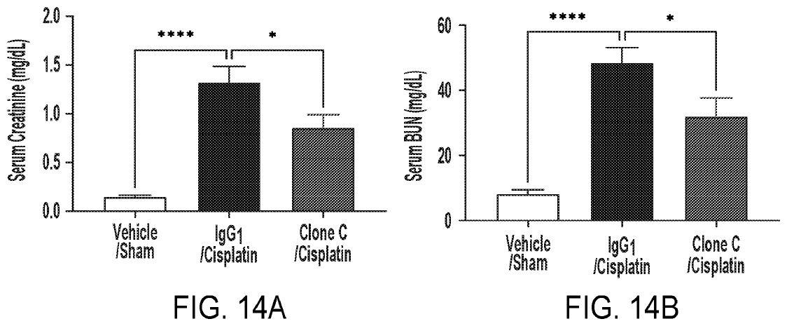

Results: As shown in FIGS. 14A-14B, administration of an anti-Activin A antibody to animals prior to treatment with Cisplatin reduced serum creatinine and serum blood urea nitrogen levels 5 days after cisplatin treatment as compared to rats administered a control antibody prior to cisplatin treatment.

Without wishing to be bound by any particular theory, it is proposed that the observed prevention of chemotherapy-induced nephrotoxicity arises from the activity of anti-Activin A antibodies in preventing and reversing nephrotoxicity. The present disclosure therefore demonstrates prevention of Cisplatin-induced nephrotoxicity with an anti-Activin A antibody and teaches that anti-Activin A antibodies can be used to inhibit and/or reduce chemotherapy-induced toxicity, including nephrotoxicity, such as in the treatment of cancers.

### Example 8: Organ Atrophy, Bone Loss and Increased Water Intake with Activin A

This Example describes liver cell atrophy and apoptosis in vivo, as well as bone loss and increased water intake induced by chronic Activin A expression.

Methods: Male C57BL/6 mice (28 g at delivery) were dosed by the intravenous route and received either control null vector (9.6×10¹¹ GC/mL (0.1 mL), Vector Biolabs) or AAV8-Cag-h-INHBA (9.6×10¹¹ GC/mL (0.1 mL), Vector Biolabs). Mice were weighed daily and ad libitum water intake was recorded 6 days pre-AAV administration and 39-51 days post-AAV administration. Body composition including bone density was measured at baseline and post AAV-administration using Dual-energy X-ray absorptiometry (DEXA). Mice achieved 20% weight loss that was maintained throughout the duration of the study. Upon termination liver and femurs were harvested. Livers were snap frozen for gene expression studies. Femurs were fixed for 3 days in 10% neutral buffered formalin and then transferred to PBS containing 0.1% sodium azide and stored at 4° C. X-ray Computed Tomography was used to assess the microstructure of mouse femurs. The scans were conducted at 12 microns resolution. Bones were scanned using an X-ray energy of 75 kV & 110 μA current using a GE v|tome|x M 240 kV CT system. Data was reconstructed then processed using VGStudioMAX (Volume Graphics GmbH) software. Measurements from the trabecular bone (on a 0.7×0.7×3 mm volume at distal end of femur) included trabecular bone volume, trabecular bone volume/total volume, trabecular number, trabecular thickness & trabecular spacing.

Gene expression studies: The total RNA was isolated from liver (5-10 mg of tissue) using MagMAX™-96 Total RNA Isolation Kit (AM1830) performed on the KingFisher Flex instrument (Thermofisher). 2 μL sample of the RNA was pipetted out into a fresh 96-well plate and the quality as well as quantity of RNA was established by Nanodrop (260/280 ratio between 1.8-2.0 was considered as good quality of RNA). Complementary DNA (cDNA) was prepared using Meridian Bioscience SensiFAST cDNA Synthesis Kit (BIO-65054) using StepOne™ real-time PCR system (Thermofisher). Mouse gene expression was performed using TaqMan assays purchased from Thermofisher on the target genes and two housekeeping genes (GAPDH and HPRT; Assay ID: Mm03302249_g1 and Assay ID: Mm03024075_m1, respectively) for normalization. All genes were analysed using CFX384 real-time PCR detection system (Biorad) in a 384-well format. Efficiency test was performed on all primers before running the qPCR (acceptable range considered for efficiency test was within of 90-110%).

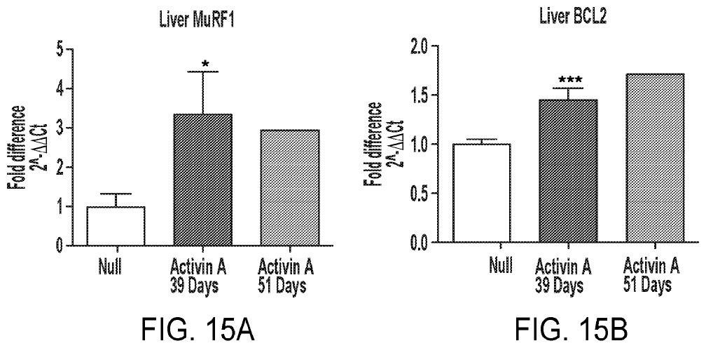

Results: FIGS. 15A-15B show Activin A induced cell atrophy and apoptosis as measured by expression of MuRF1 (FIG. 15A) and BCL2 (FIG. 15B) in the liver. MuRF1 was used as a measure for cell atrophy, and BCL2 was used as a marker of apoptosis. Chronic Activin A expression for 39 days and 51 days resulted in increased MuRF1 expression in the liver as compared to control animals (FIG. 15A). Increased BCL2 was also observed with chronic expression of Activin A compared to control animals (FIG. 15B).

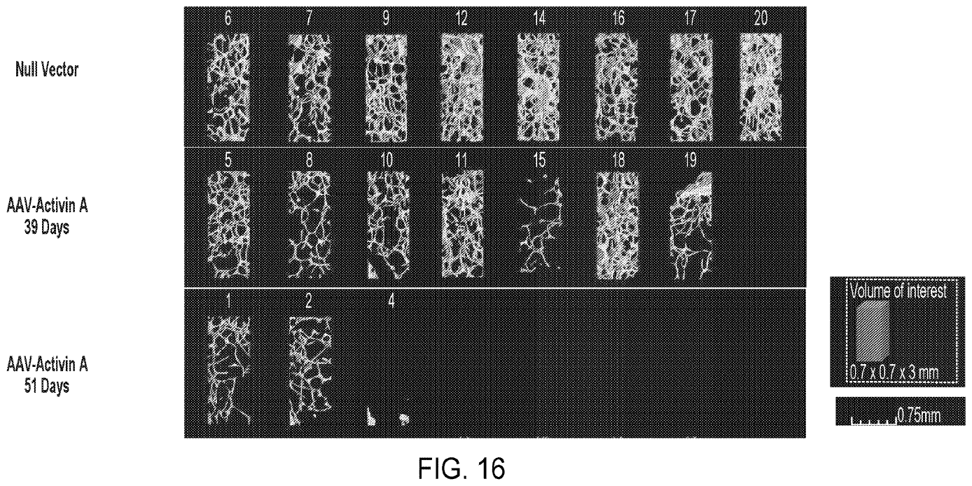

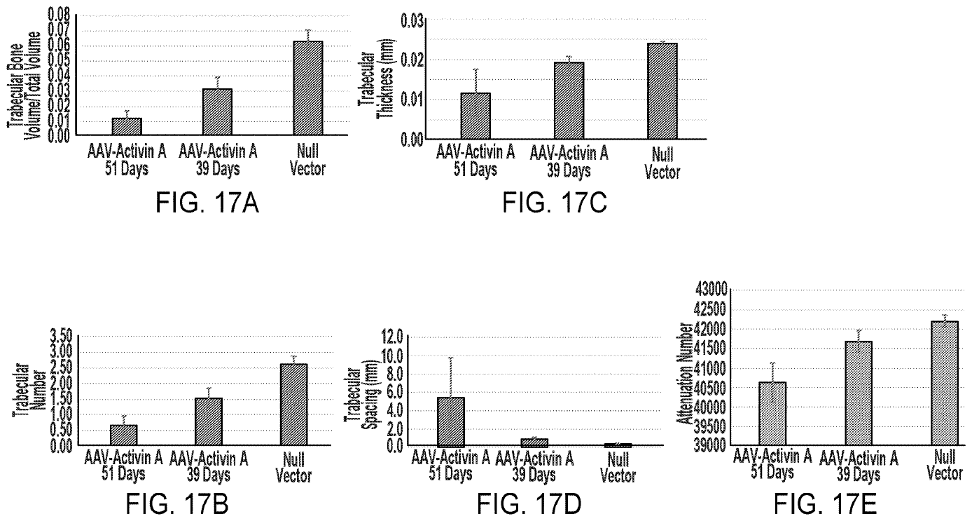

Chronic Activin A expression also resulted in bone loss in mice. As shown in FIG. 16, femurs from mice overexpressing Activin A had reduced bone density as compared to control animals. The amount of bone loss observed was higher at 51 days compared to 39 days of chronic Activin A expression. FIGS. 17A-17E provide a quantification of the bone loss shown in FIG. 16.

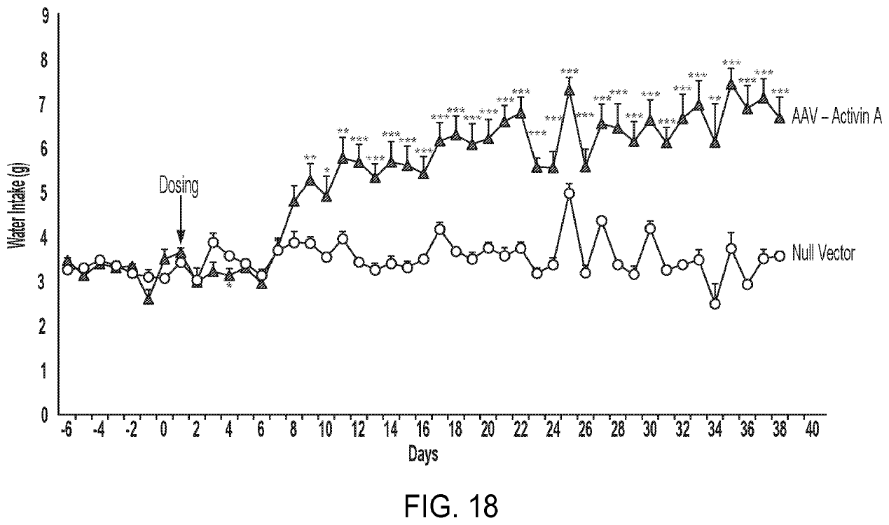

In addition, chronic expression of Activin A caused a sustained increase in water intake in mice. FIG. 18 shows increased water intake in AAV-Activin A administered mice as compared to control mice administered a null vector. Increased water intake results in increase blood volume and is a surrogate for hypertension.

This data demonstrates that anti-Activin A antibodies can be used to treat and/or prevent: cell atrophy, cell apoptosis, bone loss, thirst, dehydration and/or hypertension which can be associated with elevated levels of Activin A.

### EXEMPLARY EMBODIMENTS

Embodiment 1. An antibody agent comprising a polypeptide that binds to Activin A comprising at least one light chain complementarity determining region (LC CDR) and/or at least one heavy chain complementary determining region (HC CDR).

Embodiment 2. The antibody agent of embodiment 1, wherein the antibody agent is or comprises: (i) an intact IgA, IgG, IgD, IgE or IgM antibody; (ii) an antibody fragment; (iii) a single domain antibody; (iv) a single chain Fv; or (v) a polypeptide comprising antigen binding domain fused to a Fc domain.

Embodiment 3. The antibody agent of embodiment 1 or 2, comprising: (i) one, two, or three LC CDRs; or (ii) one, two or three HC CDRs.

Embodiment 4. The antibody agent of any one of embodiments 1-3, comprising one, two, or three LC CDRs and one, two, or three HC CDRs.

Embodiment 5. The antibody agent of any one of the preceding embodiments, comprising:
(i) an LC CDR1 sequence provided in Table 1, e.g., any one of SEQ ID NOs: 1, 11, 28, 53, 68, 85, or 93;
(ii) a sequence with at least 85%, 86%, 87%, 88%, 89%, 90%, 91%, 92%, 93%, 94%, 95%, 96%, 97%, 98%, 99% identity to an LC CDR1 sequence provided in Table 1, e.g., any one of SEQ ID NOs: 1, 11, 28, 53, 68, 85, or 93; or
(iii) a sequence having at least 5, 10, or 20 substitutions relative to an LC CDR1 sequence provided in Table 1, e.g., any one of SEQ ID NOs: 1, 11, 28, 53, 68, 85, or 93.

Embodiment 6. The antibody agent of any one of the preceding embodiments comprising:
(i) an LC CDR2 sequence provided in Table 1, e.g., any one of SEQ ID NOs: 2, 16, 29, 43, 48, 54, 61, 69, 86 or 94;
(ii) a sequence with at least 85%, 86%, 87%, 88%, 89%, 90%, 91%, 92%, 93%, 94%, 95%, 96%, 97%, 98%, 99% identity to an LC CDR2 sequence provided in Table 1 e.g., any one of SEQ ID NOs: 2, 16, 29, 43, 48, 54, 61, 69, 86 or 94; or
(iii) a sequence having at least 5, 10, or 20 substitutions relative to an LC CDR2 sequence provided in Table 1, e.g., any one of SEQ ID NOs: 2, 16, 29, 43, 48, 54, 61, 69, 86 or 94.

Embodiment 7. The antibody agent of any one of the preceding embodiments, comprising:
(i) an LC CDR3 sequence provided in Table 1, e.g., any one of SEQ ID NOs: 3, 12, 17, 21, 30, 36, 44, 49, 55, 62, 70, 80, 87 or 95;
(ii) a sequence with at least 85%, 86%, 87%, 88%, 89%, 90%, 91%, 92%, 93%, 94%, 95%, 96%, 97%, 98%, 99% identity to an LC CDR3 sequence provided in Table 1, e.g., any one of SEQ ID NOs: 3, 12, 17, 21, 30, 36, 44, 49, 55, 62, 70, 80, 87 or 95; or
(iii) a sequence having at least 5, 10, or 20 substitutions relative to an LC CDR3 sequence provided in Table 1, e.g., any one of SEQ ID NOs: 3, 12, 17, 21, 30, 36, 44, 49, 55, 62, 70, 80, 87 or 95.

Embodiment 8. The antibody agent of any one of the preceding embodiments, wherein the antibody agent comprising a LC CDR1, LC CDR2 and/or LC CDR3 is capable of binding specifically to Activin A.

Embodiment 9. The antibody agent of any one of the preceding embodiments, comprising:
(i) an LC CDR1, LC CDR2, and LC CDR3 sequence provided in Table 1;
(ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or
(iii) a sequence having at least 5, 10, or 20 substitutions relative to an LC CDR1, LC CDR2, and LC CDR3 sequence provided in Table 1.

Embodiment 10. The antibody agent of any one of embodiments 1-9, comprising:
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1;
(ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or
(iii) an LC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3.

Embodiment 11. The antibody agent of any one of embodiments 1-9, comprising:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11;
(ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or
(iii) an LC CDR3 of SEQ ID NO: 12, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 12.

Embodiment 12. The antibody agent of any one of embodiments 1-9, comprising:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11;
(ii) an LC CDR2 of SEQ ID NO: 16, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 16; and/or
(iii) an LC CDR3 of SEQ ID NO: 17, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 17.

Embodiment 13. The antibody agent of any one of embodiments 1-9, comprising:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11;
(ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or
(iii) an LC CDR3 of SEQ ID NO: 21, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 21.

Embodiment 14. The antibody agent of any one of embodiments 1-9, comprising:
(i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28;
(ii) an LC CDR2 of SEQ ID NO: 29 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 29; and/or
(iii) an LC CDR3 of SEQ ID NO: 30, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 30.

Embodiment 15. The antibody agent of any one of embodiments 1-9, comprising:
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1;
(ii) an LC CDR2 of SEQ ID NO: 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or
(iii) an LC CDR3 of SEQ ID NO: 36, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 36.

Embodiment 16. The antibody agent of any one of embodiments 1-9, comprising:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11;
(ii) an LC CDR2 of SEQ ID NO: 43 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 43; and/or
(iii) an LC CDR3 of SEQ ID NO: 44, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 44.

Embodiment 17. The antibody agent of any one of embodiments 1-9, comprising:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11;
(ii) an LC CDR2 of SEQ ID NO: 48 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 48; and/or
(iii) an LC CDR3 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49.

Embodiment 18. The antibody agent of any one of embodiments 1-9, comprising:
(i) an LC CDR1 of SEQ ID NO: 53, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 53;
(ii) an LC CDR2 of SEQ ID NO: 54 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 54; and/or
(iii) an LC CDR3 of SEQ ID NO: 55, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 55.

Embodiment 19. The antibody agent of any one of embodiments 1-9, comprising:
(i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28;
(ii) an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and/or
(iii) an LC CDR3 of SEQ ID NO: 62, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 62.

Embodiment 20. The antibody agent of any one of embodiments 1-9, comprising:
(i) an LC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68;
(ii) an LC CDR2 of SEQ ID NO: 69 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and/or
(iii) an LC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70.

Embodiment 21. The antibody agent of any one of embodiments 1-9, comprising:
(i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28;
(ii) an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and/or
(iii) an LC CDR3 of SEQ ID NO: 80, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 80.

Embodiment 22. The antibody agent of any one of embodiments 1-9, comprising:
(i) an LC CDR1 of SEQ ID NO:85, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 85;
(ii) an LC CDR2 of SEQ ID NO: 86 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 86; and/or
(iii) an LC CDR3 of SEQ ID NO: 87, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 87.

Embodiment 23. The antibody agent of any one of embodiments 1-9, comprising:
(i) an LC CDR1 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93;
(ii) an LC CDR2 of SEQ ID NO: 94 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and/or
(iii) an LC CDR3 of SEQ ID NO: 95, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 95.

Embodiment 24. The antibody agent of any one of the preceding embodiments, comprising:
(i) a HC CDR1 sequence provided in Table 2, e.g., any one of SEQ ID NOs: 103, 113. 120, 125, 129, 134, 140, 149, 157, 163, 169, 179, 190, 196, 202 or 209;
(ii) a sequence with at least 85%, 86%, 87%, 88%, 89%, 90%, 91%, 92%, 93%, 94%, 95%, 96%, 97%, 98%, 99% identity to a HC CDR1 sequence provided in Table 2, e.g., any one of SEQ ID NOs: 103, 113. 120, 125, 129, 134, 140, 149, 157, 163, 169, 179, 190, 196, 202 or 209; or
(iii) a sequence having at least 5, 10, or 20 substitutions compared to a HC CDR1 sequence provided in Table 2, e.g., any one of SEQ ID NOs: 103, 113. 120, 125, 129, 134, 140, 149, 157, 163, 169, 179, 190, 196, 202 or 209.

Embodiment 25. The antibody agent of any one of the preceding embodiments, comprising:
(i) a HC CDR2 sequence provided in Table 2, e.g., any one of SEQ ID NOs: 104, 114, 121, 130, 135, 141, 145, 150, 158, 164, 170, 180, 186, 191, 197, 203 or 210;
(ii) a sequence with at least 85%, 86%, 87%, 88%, 89%, 90%, 91%, 92%, 93%, 94%, 95%, 96%, 97%, 98%, 99% identity to a HC CDR2 sequence provided in Table 2, e.g., any one of SEQ ID NOs: 104, 114, 121, 130, 135, 141, 145, 150, 158, 164, 170, 180, 186, 191, 197, 203 or 210; or
(iii) a sequence having at least 5, 10, or 20 substitutions compared to a HC CDR2 sequence provided in Table 2, e.g., any one of SEQ ID NOs: 104, 114, 121, 130, 135, 141, 145, 150, 158, 164, 170, 180, 186, 191, 197, 203 or 210.

Embodiment 26. The antibody agent of any one of the preceding embodiments comprising:
(i) a HC CDR3 sequence provided in Table 2, e.g., any one of SEQ ID NOs: 105, 115, 151, 165, 171, 181, 192, 198, or 204;
(ii) a sequence with at least 85%, 86%, 87%, 88%, 89%, 90%, 91%, 92%, 93%, 94%, 95%, 96%, 97%, 98%, 99% identity to a HC CDR3 sequence provided in Table 2, e.g., any one of SEQ ID NOs: 105, 115, 151, 165, 171, 181, 192, 198, or 204; or
(iii) a sequence having at least 5, 10, or 20 substitutions compared to a HC CDR3 sequence provided in Table 2, e.g., any one of SEQ ID NOs: 105, 115, 151, 165, 171, 181, 192, 198, or 204.

Embodiment 27. The antibody agent of any one of the preceding embodiments, wherein the antibody agent comprising a HC CDR1, a HC CDR2 and/or a HC CDR3 is able to specifically bind to Activin A.

Embodiment 28. The antibody agent of any one of embodiments 1-27, comprising:
(i) an HC CDR1, HC CDR2, and HC CDR3 sequence provided in Table 1;
(ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 1;
(iii) a sequence having at least 5, 10, or 20 substitutions relative to an HC CDR1, HC CDR2, and HC CDR3 sequence provided in Table 1.

Embodiment 29. The antibody agent of any one of embodiments 1-28, comprising:
(i) an HC CDR1 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103;
(ii) an HC CDR2 of SEQ ID NO: 104, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 104; and/or
(iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 30. The antibody agent of any one of embodiments 1-28, comprising:
(i) an HC CDR1 of SEQ ID NO: 113, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 113;
(ii) an HC CDR2 of SEQ ID NO: 114, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 114; and/or
(iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 31. The antibody agent of any one of embodiments 1-28, comprising:
(i) an HC CDR1 of SEQ ID NO: 120, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 120;
(ii) an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and/or
(iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 32. The antibody agent of any one of embodiments 1-28, comprising:
(i) an HC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125;
(ii) an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and/or
(iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 33. The antibody agent of any one of embodiments 1-28, comprising:
(i) an HC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129;
(ii) an HC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and/or
(iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 34. The antibody agent of any one of embodiments 1-28, comprising:
(i) an HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134;
(ii) an HC CDR2 of SEQ ID NO: 135, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 135; and/or
(iii) an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 35. The antibody agent of any one of embodiments 1-28, comprising:
(i) an HC CDR1 of SEQ ID NO: 140, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 140;
(ii) an HC CDR2 of SEQ ID NO: 141, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 141; and/or
(iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 36. The antibody agent of any one of embodiments 1-28, comprising:
(i) an HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134;
(ii) an HC CDR2 of SEQ ID NO: 145, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 145; and/or
(iii) an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 37. The antibody agent of any one of embodiments 1-28, comprising:
(i) an HC CDR1 of SEQ ID NO: 149, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 149;
(ii) an HC CDR2 of SEQ ID NO: 150, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 150; and/or
(iii) an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

Embodiment 38. The antibody agent of any one of embodiments 1-28, comprising:
(i) an HC CDR1 of SEQ ID NO: 157, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 157;
(ii) an HC CDR2 of SEQ ID NO: 158, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 158; and/or
(iii) an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

Embodiment 39. The antibody agent of any one of embodiments 1-28, comprising:
(i) an HC CDR1 of SEQ ID NO: 163, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 163;
(ii) an HC CDR2 of SEQ ID NO: 164, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 164; and/or
(iii) an HC CDR3 of SEQ ID NO: 165, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 165.

Embodiment 40. The antibody agent of any one of embodiments 1-28, comprising:
(i) an HC CDR1 of SEQ ID NO: 169, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 169;
(ii) an HC CDR2 of SEQ ID NO: 170, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 170; and/or
(iii) an HC CDR3 of SEQ ID NO: 171, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 171.

Embodiment 41. The antibody agent of any one of embodiments 1-28 comprising:
(i) an HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179;
(ii) an HC CDR2 of SEQ ID NO: 180, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 180; and/or
(iii) an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

Embodiment 42. The antibody agent of any one of embodiments 1-28, comprising:
(i) an HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179;
(ii) an HC CDR2 of SEQ ID NO: 186, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 186; and/or
(iii) an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

Embodiment 43. The antibody agent of any one of embodiments 1-28, comprising:
(i) an HC CDR1 of SEQ ID NO: 190, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 190;
(ii) an HC CDR2 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191; and/or
(iii) an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

Embodiment 44. The antibody agent of any one of embodiments 1-28, comprising:
(i) an HC CDR1 of SEQ ID NO: 196, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:196;
(ii) an HC CDR2 of SEQ ID NO: 197, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 197; and/or
(iii) an HC CDR3 of SEQ ID NO: 198, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 198.

Embodiment 45. The antibody agent of any one of embodiments 1-28, comprising:
(i) an HC CDR1 of SEQ ID NO: 202, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:202;
(ii) an HC CDR2 of SEQ ID NO: 203, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 203; and/or
(iii) an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

Embodiment 46. The antibody agent of any one of embodiments 1-28, comprising:
(i) an HC CDR1 of SEQ ID NO: 209, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:209;
(ii) an HC CDR2 of SEQ ID NO: 210, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 210; and/or
(iii) an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

Embodiment 47. The antibody agent of any one of the preceding embodiments, comprising one, two, or three LC CDRs provided in Table 1 or a sequence with at least 85%, 86%, 87%, 88%, 89%, 90%, 91%, 92%, 93%, 94%, 95%, 96%, 97%, 98%, or 99% thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% thereto.

Embodiment 48. The antibody agent of embodiment 47, wherein the antibody agent comprises:
(a) a light chain comprising:
(i) an LC CDR1 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR1 provided in Table 1;
(ii) an LC CDR2 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR2 provided in Table 1; and/or
(iii) an LC CDR3 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR3 provided in Table 1; and
(b) a heavy chain comprising:
(i) a HC CDR1 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR1 provided in Table 2;
(ii) a HC CDR2 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR2 provided in Table 2; and/or
(iii) a HC CDR3 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR3 provided in Table 2.

Embodiment 49. The antibody agent of embodiment 47 or 48, wherein the antibody agent comprises:
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3; and
(ii) a HC CDR1 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; an HC CDR2 of SEQ ID NO: 104, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 104; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 50. The antibody agent of embodiment 47 or 48, wherein the antibody agent comprises:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 12, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 12; and
(ii) a HC CDR1 of SEQ ID NO: 113, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 113; an HC CDR2 of SEQ ID NO: 114, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 114; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 51. The antibody agent of embodiment 47 or 48, wherein the antibody agent comprises:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 16, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 16; and an LC CDR3 of SEQ ID NO: 17, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 17; and
(ii) a HC CDR1 of SEQ ID NO: 120, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 120; an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 52. The antibody agent of embodiment 47 or 48, wherein the antibody agent comprises:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 21, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 21; and
(ii) a HC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; an HC CDR2 of SEQ ID NO: 121, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 121; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 53. The antibody agent of embodiment 47 or 48, wherein the antibody agent comprises
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3; and
(ii) a HC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; an HC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 54. The antibody agent of embodiment 47 or 48, wherein the antibody agent comprises
(i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; an LC CDR2 of SEQ ID NO: 29 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 29; and an LC CDR3 of SEQ ID NO: 30, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 30; and
(ii) a HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134; an HC CDR2 of SEQ ID NO: 135, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 135; and an HC CDR3 of SEQ ID NO: 115, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 115.

Embodiment 55. The antibody agent of embodiment 47 or 48, wherein the antibody agent comprises
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 36, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 36; and
(ii) a HC CDR1 of SEQ ID NO: 140, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 140; an HC CDR2 of SEQ ID NO: 141, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 141; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 56. The antibody agent of embodiment 47 or 48, wherein the antibody agent comprises
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an LC CDR2 of SEQ ID NO: 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an LC CDR3 of SEQ ID NO: 36, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 36; and
(ii) a HC CDR1 of SEQ ID NO: 134, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 134; an HC CDR2 of SEQ ID NO: 145, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 145; and an HC CDR3 of SEQ ID NO: 105, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 105.

Embodiment 57. The antibody agent of embodiment 47 or 48, wherein the antibody agent comprises
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 43 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 43; and an LC CDR3 of SEQ ID NO: 44, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 44; and
(ii) a HC CDR1 of SEQ ID NO: 149, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 149; an HC CDR2 of SEQ ID NO: 150, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 150; and an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

Embodiment 58. The antibody agent of embodiment 47 or 48, wherein the antibody agent comprises
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; an LC CDR2 of SEQ ID NO: 48 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 48; and an LC CDR3 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49; and
(ii) a HC CDR1 of SEQ ID NO: 157, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 157; an HC CDR2 of SEQ ID NO: 158, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 158; and an HC CDR3 of SEQ ID NO: 151, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 151.

Embodiment 59. The antibody agent of embodiment 47 or 48, wherein the antibody agent comprises
(i) an LC CDR1 of SEQ ID NO: 53, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 53; an LC CDR2 of SEQ ID NO: 54 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 54; and an LC CDR3 of SEQ ID NO: 55, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 55; and
(ii) a HC CDR1 of SEQ ID NO: 163, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 163; an HC CDR2 of SEQ ID NO: 164, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 164; and an HC CDR3 of SEQ ID NO: 165, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 165.

Embodiment 60. The antibody agent of embodiment 47 or 48, wherein the antibody agent comprises
(i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and an LC CDR3 of SEQ ID NO: 62, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 62; and
(ii) a HC CDR1 of SEQ ID NO: 169, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 169; an HC CDR2 of SEQ ID NO: 170, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 170; and an HC CDR3 of SEQ ID NO: 171, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 171.

Embodiment 61. The antibody agent of embodiment 47 or 48, wherein the antibody agent comprises
(i) an LC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; an LC CDR2 of SEQ ID NO: 69 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and an LC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70; and
(ii) a HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179; an HC CDR2 of SEQ ID NO: 180, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 180; and an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

Embodiment 62. The antibody agent of embodiment 47 or 48, wherein the antibody agent comprises
(i) an LC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; an LC CDR2 of SEQ ID NO: 69 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and an LC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70; and
(ii) a HC CDR1 of SEQ ID NO: 179, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 179; an HC CDR2 of SEQ ID NO: 186, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 186; and an HC CDR3 of SEQ ID NO: 181, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 181.

Embodiment 63. The antibody agent of embodiment 47 or 48, wherein the antibody agent comprises
(i) an LC CDR1 of SEQ ID NO: 28, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 28; an LC CDR2 of SEQ ID NO: 61 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 61; and an LC CDR3 of SEQ ID NO: 80, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 80; and
(ii) a HC CDR1 of SEQ ID NO: 190, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 190; an HC CDR2 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191; and an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

Embodiment 64. The antibody agent of embodiment 47 or 48, wherein the antibody agent comprises
(i) an LC CDR1 of SEQ ID NO:85, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 85; an LC CDR2 of SEQ ID NO: 86 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 86; and an LC CDR3 of SEQ ID NO: 87, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 87; and
(ii) a HC CDR1 of SEQ ID NO: 196, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 196; an HC CDR2 of SEQ ID NO: 197, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 197; and an HC CDR3 of SEQ ID NO: 198, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 198.

Embodiment 65. The antibody agent of embodiment 47 or 48, wherein the antibody agent comprises
(i) an LC CDR1 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; an LC CDR2 of SEQ ID NO: 94 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and an LC CDR3 of SEQ ID NO: 95, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 95; and
(ii) a HC CDR1 of SEQ ID NO: 202, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:202; an HC CDR2 of SEQ ID NO: 203, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 203; and an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

Embodiment 66. The antibody agent of embodiment 47 or 48, wherein the antibody agent comprises
(i) an LC CDR1 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; an LC CDR2 of SEQ ID NO: 94 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and an LC CDR3 of SEQ ID NO: 95, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 95; and
(ii) a HC CDR1 of SEQ ID NO: 209, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO:209; an HC CDR2 of SEQ ID NO: 210, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 210; and an HC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

Embodiment 67. The antibody agent of any one of the preceding embodiments, wherein the antibody agent comprises a light chain comprising a variable region (VL) comprising at least one LC CDR provided in Table 1 or a sequence with at least 85%, 86%, 87%, 88%, 89%, 90%, 91%, 92%, 93%, 94%, 95%, 96%, 97%, 98%, or 99% thereto; and at least one framework region (FR) provided in Table 1 or a sequence with at least 92% identity thereto.

Embodiment 68. The antibody agent of embodiment 67, wherein the VL region comprises one, two or three LC CDRs provided in Table 1 or a sequence with at least 85%, 86%, 87%, 88%, 89%, 90%, 91%, 92%, 93%, 94%, 95%, 96%, 97%, 98%, or 99% thereto, and one, two, three or four FR regions provided in Table 1 or a sequence with at least 92% identity thereto.

Embodiment 69. The antibody agent of embodiment 67 or 68, wherein the FR region comprises:
(i) a FR sequence provided in Table 1;
(ii) a sequence with at least 92%, 93%, 94%, 95%, 96%, 97%, 98%, or 99% identity to a FR sequence provided in Table 1; or
(iii) a sequence having at least 5, 10, or 20 substitutions compared to a FR sequence provided in Table 1.

Embodiment 70. The antibody agent of any one of embodiments 67-69, comprising:
(i) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8;
(ii) the sequence of SEQ ID NO: 13, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 13;
(iii) the sequence of SEQ ID NO: 18, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 18;
(iv) the sequence of SEQ ID NO: 22, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 22;
(v) the sequence of SEQ ID NO: 33, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 33;
(vi) the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37;
(vii) the sequence of SEQ ID NO: 45, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 45;
(viii) the sequence of SEQ ID NO: 50, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 50;
(ix) the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58;
(x) the sequence of SEQ ID NO: 65, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 65;
(xi) the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74;
(xii) the sequence of SEQ ID NO: 82, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 82;
(xiii) the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90; or
(xiv) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99.

Embodiment 71. The antibody agent of any one of embodiments 67-70, wherein the light chain further comprises a sequence for a constant region (CL).

Embodiment 72 The antibody agent of embodiment 71, wherein the light chain comprises a kappa CL or a lambda CL.

Embodiment 73. The antibody agent of any one of the preceding embodiments, wherein the antibody agent comprises a heavy chain comprising a variable region (VH) comprising at least one HC CDR provided in Table 2 or a sequence with at least 85%, 86%, 87%, 88%, 89%, 90%, 91%, 92%, 93%, 94%, 95%, 96%, 97%, 98%, or 99% thereto, and at least one framework region (FR) provided in Table 2 or a sequence with at least 92% identity thereto.

Embodiment 74 The antibody agent of embodiment 73, wherein the VH region one, two or three HC CDRs provided in Table 2 or a sequence with at least 85%, 86%, 87%, 88%, 89%, 90%, 91%, 92%, 93%, 94%, 95%, 96%, 97%, 98%, or 99% thereto, and one, two, three or four FR regions provided in Table 2 or a sequence with at least 92% identity thereto.

Embodiment 75. The antibody agent of embodiment 73 or 74, wherein the FR region comprises:
(i) a FR sequence provided in Table 2;
(ii) a sequence with at least 92%, 93%, 94%, 95%, 96%, 97%, 98%, or 99% identity to a FR sequence provided in Table 2; or
(iii) a sequence having at least 5, 10, or 20 substitutions compared to a FR sequence provided in Table 2.

Embodiment 76. The antibody agent of any one of embodiments 73-75, comprising:
(i) the sequence of SEQ ID NO: 110, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 110;
(ii) the sequence of SEQ ID NO: 117, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 117;
(iii) the sequence of SEQ ID NO: 122, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 122;
(iv) the sequence of SEQ ID NO: 126, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 126;
(v) the sequence of SEQ ID NO: 131, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 131;
(vi) the sequence of SEQ ID NO: 137, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 137;
(vii) the sequence of SEQ ID NO: 142, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 142;
(viii) the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146;
(ix) the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154;
(x) the sequence of SEQ ID NO: 160, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 160;
(xi) the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166;
(xii) the sequence of SEQ ID NO: 176, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 176;
(xiii) the sequence of SEQ ID NO: 183, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 183;
(xiv) the sequence of SEQ ID NO: 187, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 187;
(xv) the sequence of SEQ ID NO: 193, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 193;
(xvi) the sequence of SEQ ID NO: 199 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 199;
(xvii) the sequence of SEQ ID NO: 206 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206; or
(xviii) the sequence of SEQ ID NO: 211 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 211.

Embodiment 77. The antibody agent of any one of embodiments 73-76, wherein the heavy chain region further comprises a sequence for at least one constant region (CH).

Embodiment 78. The antibody agent of embodiment 77, wherein the at least one constant region comprises an Fc domain.

Embodiment 79. The antibody agent of embodiment 78, wherein the Fc domain comprises a mammalian Fc domain, e.g., a mouse, a rat, a rabbit, a primate, a human, or a domestic animal Fc domain (e.g., a dog, a cat, a cow, or a horse Fc domain).

Embodiment 80. The antibody agent of embodiment 78 or 79, wherein the Fc domain is chosen from an Fc domain of an immunoglobulin isotype.

Embodiment 81. The antibody agent of embodiment 80, wherein the immunoglobulin isotype comprises IgA, IgG, IgM, or IgE.

Embodiment 82. The antibody agent of embodiment 80 or 81, wherein the Fc domain comprises an Fc domain of an IgG, e.g., a human IgG.

Embodiment 83. The antibody agent of embodiment 82, wherein the IgG is or comprises IgG1, IgG2, IgG3, or IgG4.

Embodiment 84. The antibody agent of embodiment 82 or 83, wherein the Fc domain comprises one or more modifications.

Embodiment 85. The antibody agent of embodiment 84, wherein the one or more modifications modulates one or more properties of the antibody agent.

Embodiment 86. The antibody agent of embodiment 84 or 85, wherein the one or more modifications comprises:
(i) a LAGA mutation, a FEGG mutation, an AAGG mutation, an AAGA mutation, a LALA mutation or a combination thereof;
(ii) a I253A mutation, a H310A mutation, a H435R mutation, a H435A mutation or a combination thereof. (iii) a CH3 variant, optionally wherein the CH3 variant comprises a leucine at position 428 and/or an alanine at position 434; and/or
(iv) a combination of or all of (i)-(iii).

Embodiment 87. The antibody agent of any the preceding embodiments, wherein the antibody agent comprises a VL comprising any one, two or three CDRs provided in Table 1 or a sequence with at least 85%, 86%, 87%, 88%, 89%, 90%, 91%, 92%, 93%, 94%, 95%, 96%, 97%, 98%, or 99% thereto and a VH comprising any one two or three CDRs provided in Table 2 or a sequence with at least 85%, 86%, 87%, 88%, 89%, 90%, 91%, 92%, 93%, 94%, 95%, 96%, 97%, 98%, or 99% thereto.

Embodiment 88. The antibody agent of any one of the preceding embodiments, wherein the antibody agent comprises a VL provided in Table 1 or a sequence with at least 85% thereto and a VH provided in Table 2 or a sequence with at least 85% thereto.

Embodiment 89. The antibody agent of embodiment 87 or 88, wherein the antibody agent comprises:
(i) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; and the sequence of SEQ ID NO: 110, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 110;
(ii) the sequence of SEQ ID NO: 13, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 13; and the sequence of SEQ ID NO: 117, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 117;
(iii) the sequence of SEQ ID NO: 18, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 18; and the sequence of SEQ ID NO: 122, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 122;
(iv) the sequence of SEQ ID NO: 22, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 22; and the sequence of SEQ ID NO: 126, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 126;
(v) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8; and the sequence of SEQ ID NO: 131, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 131;
(vi) the sequence of SEQ ID NO: 33, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 33; and the sequence of SEQ ID NO: 137, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO:137;
(vii) the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; and the sequence of SEQ ID NO: 142, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 142;
(viii) the sequence of SEQ ID NO: 37, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 37; and the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146;
(ix) the sequence of SEQ ID NO: 45, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 45; and the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154;
(x) the sequence of SEQ ID NO: 50, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 50; and the sequence of SEQ ID NO: 160, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 160;
(xi) the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166;
(xii) the sequence of SEQ ID NO: 65, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 65; and the sequence of SEQ ID NO: 176, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 176;
(xiii) the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; and the sequence of SEQ ID NO: 183, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 183;
(xiv) the sequence of SEQ ID NO: 74, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 74; and the sequence of SEQ ID NO: 187, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 187;
(xv) the sequence of SEQ ID NO: 82, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 82; and the sequence of SEQ ID NO: 193, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 193;
(xvi) the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90; and the sequence of SEQ ID NO: 199, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 199;
(xvii) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206; or
(xviii) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 211, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 211.

Embodiment 90. The antibody agent of any one of the preceding embodiments, wherein the antibody agent comprises:
(i) a light chain (LC) comprising:
(a) one, two or three LC CDRs provided in Table 1 or a sequence with at least 85%, 86%, 87%, 88%, 89%, 90%, 91%, 92%, 93%, 94%, 95%, 96%, 97%, 98%, or 99% thereto;
(b) at least one FR provided in Table 1 or sequence with at least 92% identity thereto;
(c) a constant region (CL); and
(ii) a heavy chain (HC) comprising:
   (a) one, two or three HC CDRs provided in Table 2 or a sequence with at least 85%, 86%, 87%, 88%, 89%, 90%, 91%, 92%, 93%, 94%, 95%, 96%, 97%, 98%, or 99% thereof;
   (b) at least one FR provided in Table 1 or a sequence with at least 92% identity thereto;
   (c) at least one constant region.

Embodiment 91. The antibody agent of embodiment 90, wherein the antibody agent comprises:
(i) the sequence of SEQ ID NO: 9, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 9; and the sequence of SEQ ID NO: 111, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 111;
(ii) the sequence of SEQ ID NO: 14, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 14; and the sequence of SEQ ID NO: 118, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 118;
(iii) the sequence of SEQ ID NO: 19, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 19; and the sequence of SEQ ID NO: 123, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 123;
(iv) the sequence of SEQ ID NO: 23, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 23; and the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127;
(v) the sequence of SEQ ID NO: 9, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 9; and the sequence of SEQ ID NO: 132, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 132;
(vi) the sequence of SEQ ID NO: 34, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 34; and the sequence of SEQ ID NO: 138, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 138;
(vii) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38; and the sequence of SEQ ID NO: 143, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143;
(viii) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38; and the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147;
(ix) the sequence of SEQ ID NO: 46, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 46; and the sequence of SEQ ID NO: 155, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155;
(x) the sequence of SEQ ID NO: 51, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 51; and the sequence of SEQ ID NO: 161, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 161;
(xi) the sequence of SEQ ID NO: 59, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 59; and the sequence of SEQ ID NO: 167, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 167;
(xii) the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66; and the sequence of SEQ ID NO: 177, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 177;
(xiii) the sequence of SEQ ID NO: 75, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 75; and the sequence of SEQ ID NO: 184, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 184;
(xiv) the sequence of SEQ ID NO: 75, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 75; and the sequence of SEQ ID NO: 188, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 188;
(xv) the sequence of SEQ ID NO: 83, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 83; and the sequence of SEQ ID NO: 194, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 194;
(xvi) the sequence of SEQ ID NO: 91, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 91; and the sequence of SEQ ID NO: 200, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 200;
(xvii) the sequence of SEQ ID NO: 100, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 100; and the sequence of SEQ ID NO: 207, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 207; or
(xviii) the sequence of SEQ ID NO: 100, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 100; and the sequence of SEQ ID NO: 212, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 212.

Embodiment 92. The antibody agent of embodiment 90, wherein the antibody agent comprises:
(i) the sequence of SEQ ID NO: 9, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 9; and the sequence of SEQ ID NO: 111 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 111 without a terminal lysine;
(ii) the sequence of SEQ ID NO: 14, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 14; and the sequence of SEQ ID NO: 118 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 118 without a terminal lysine;
(iii) the sequence of SEQ ID NO: 19, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 19; and the sequence of SEQ ID NO: 123 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 123 without a terminal lysine;
(iv) the sequence of SEQ ID NO: 23, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 23; and the sequence of SEQ ID NO: 127 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127 without a terminal lysine;
(v) the sequence of SEQ ID NO: 9, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 9; and the sequence of SEQ ID NO: 132 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 132 without a terminal lysine;
(vi) the sequence of SEQ ID NO: 34, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 34; and the sequence of SEQ ID NO: 138 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 138 without a terminal lysine;
(vii) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38; and the sequence of SEQ ID NO: 143 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143 without a terminal lysine;
(viii) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38; and the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine;
(ix) the sequence of SEQ ID NO: 46, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 46; and the sequence of SEQ ID NO: 155 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155 without a terminal lysine;
(x) the sequence of SEQ ID NO: 51, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 51; and the sequence of SEQ ID NO: 161 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 161 without a terminal lysine;
(xi) the sequence of SEQ ID NO: 59, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 59; and the sequence of SEQ ID NO: 167 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 167 without a terminal lysine;
(xii) the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66; and the sequence of SEQ ID NO: 177 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 177 without a terminal lysine;
(xiii) the sequence of SEQ ID NO: 75, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 75; and the sequence of SEQ ID NO: 184 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 184 without a terminal lysine;
(xiv) the sequence of SEQ ID NO: 75, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 75; and the sequence of SEQ ID NO: 188 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 188 without a terminal lysine;
(xv) the sequence of SEQ ID NO: 83, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 83; and the sequence of SEQ ID NO: 194 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 194 without a terminal lysine;
(xvi) the sequence of SEQ ID NO: 91, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 91; and the sequence of SEQ ID NO: 200 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 200 without a terminal lysine;
(xvii) the sequence of SEQ ID NO: 100, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 100; and the sequence of SEQ ID NO: 207 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 207 without a terminal lysine; or
(xviii) the sequence of SEQ ID NO: 100, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 100; and the sequence of SEQ ID NO: 212 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 212 without a terminal lysine.

Embodiment 93. The agent of any one of the preceding embodiments, further comprising a second binding specificity optionally wherein, the second binding specificity confers binding to an antigen other than human Activin A.

Embodiment 94. The antibody agent of embodiment 93, wherein the antibody agent comprising an Activin A binding specificity and a second binding specificity is chosen from: a heterodimer, a Crossmab, a DVD-Ig, a 2 in 1 IgG, an IgG-sc-FV, an scFv-scFv, a BiTE, a DART, a diabody, a Fab-scFv fusion, a Fab-Fab fusion, or a tandem antibody.

Embodiment 95. The antibody agent of any one of the preceding embodiments, wherein the antibody agent binds to human Activin A with a binding affinity (K_{D}) of about 6.17×10(−12)M to about 960×10(−12)M, e.g., with a Fab format.

Embodiment 96. The antibody agent of embodiment 95, wherein the binding affinity is determined with a binding affinity determining assay such as a surface plasmon resonance assay, an Octet assay or a comparable assay.

Embodiment 97. The antibody agent of any one of the preceding embodiments, characterized in that when tested in an assay that evaluates Activin A activity and/or level, the antibody agent reduces Activin A activity and/or level, or prevents an increase in Activin A activity and/or level relative to a comparator.

Embodiment 98. The antibody agent of embodiment 97, wherein Activin A is or comprises free and/or active Activin A.

Embodiment 99. The antibody agent of embodiment 98, wherein Activin A is or comprises free Activin A.

Embodiment 100. The antibody agent of embodiment 98, wherein Activin A is or comprises active Activin A.

Embodiment 101. The antibody agent of embodiment 98, wherein Activin A is or comprises free and active Activin A.

Embodiment 102. The antibody agent of any one of embodiments 97-101, wherein the comparator is or comprises a sample that is not contacted with an Activin A antibody agent disclosed herein.

Embodiment 103. The antibody agent of any one of embodiments 97-102, wherein the antibody agent reduces a plasma, blood, serum, and/or urine level of Activin A.

Embodiment 104. The antibody agent of embodiment 103, wherein the antibody agent reduces the level of Activin A to less than about 500 pg/mL.

Embodiment 105. The antibody agent of any one of embodiments 97-102, wherein the antibody agent prevents an increase in Activin A levels in plasma, blood, serum, and/or urine.

Embodiment 106. The antibody agent of embodiment 101, wherein the antibody agent prevents an increase in Activin A level above about 500 pg/mL.

Embodiment 107. The antibody agent of any one of embodiments 97-107, wherein the antibody agent reduces, e.g., inhibits, an Activin A activity.

Embodiment 108. The antibody agent of any one of embodiments 97-107, wherein the antibody agent reduces, e.g., inhibits, the activity and/or level of Activin A (e.g., plasma, blood, serum, and/or urine Activin A, e.g., free and/or active Activin A) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 109. The antibody agent of embodiment 108, wherein the antibody agent inhibits the activity and/or level of Activin A (e.g., plasma, blood, serum, and/or urine Activin A, e.g., free and/or active Activin A) by 100%.

Embodiment 110. The antibody agent of embodiment 108 or 109, wherein inhibition of Activin A activity comprises inhibiting binding of Activin A to an Activin receptor (e.g., a Type II receptor, a Type I receptor or both).

Embodiment 111. The antibody agent of embodiment 110, wherein inhibiting binding of Activin A to an Activin receptor reduces, e.g., inhibits, an Activin receptor activity and/or an Activin receptor mediated signaling pathway.

Embodiment 112. The antibody agent of embodiment 110 or 111, wherein an Activin receptor is or comprises a Type II receptor.

Embodiment 113. The antibody agent of embodiment 112, wherein the Type II receptor is or comprises ACTRIIB or ACTRIIA, or both.

Embodiment 114. The antibody agent of embodiment 110 or 111, wherein an Activin receptor is or comprises a Type I receptor.

Embodiment 115. The antibody agent of embodiment 114, wherein the Type I receptor is or comprises Type I ACTRIA, ACTRIB, ACTRIC, or a combination thereof.

Embodiment 116. The antibody agent of any one of embodiments 111-115, wherein an Activin receptor mediated signaling pathway comprises one or more of: (i) a SMAD pathway, (ii) an ERK/MAPK pathway, (iii) a p38 MAPK pathway, (iv) NOX-2 dependent signaling; or (v) Snail and/or Slug mediated pathways.

Embodiment 117. The antibody agent of any one of the preceding embodiments, characterized in that when tested in an assay that evaluates activation of an Activin receptor, the antibody agent does not activate one or more signaling pathways activated by the Activin receptor.

Embodiment 118. The antibody agent of any one of the preceding embodiments, characterized in that when tested in an assay that evaluates activation of an Activin receptor, the antibody agent reduces, e.g., inhibits, Activin A signaling via the Activin receptor, e.g., as assessed in an ActivinRIIB receptor/SMAD reporter assay in Example 2.

Embodiment 119. The antibody agent of embodiment 118, wherein the antibody agent reduces Activin A signaling via the Activin receptor by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 120. The antibody agent of embodiment 118 or 119, the antibody agent inhibits Activin A signaling via the Activin receptor by about 100%.

Embodiment 121. The antibody agent of any one of the preceding embodiments, wherein the antibody agent does not bind to or has minimal binding affinity for one or more TGFbeta super family members other than Activin A.

Embodiment 122. The antibody agent of any one of the preceding embodiments, wherein the antibody agent does not bind to or has minimal binding affinity for any one, or all, or a combination of Activin B, BMP-9, BMP-10, GDF-15, GDNF, GDF-8, GDF-10 or GDF-11.

Embodiment 123. The antibody agent of any one of the preceding embodiments, wherein the antibody agent binds to Activin A and one or both of Activin B and GDF-11.

Embodiment 124. The antibody agent of embodiment 122 or 123, which:
(i) binds to Activin B and is characterized in that when tested in an assay that evaluates Activin B activity and/or level, the antibody agent does not modulate Activin B activity and/or level; and/or
(ii) binds to GDF-11 and is characterized in that when tested in an assay that evaluates GDF-11 activity and/or level, the antibody agent does not modulate GDF-11 activity and/or level.

Embodiment 125. The antibody agent of any one of the preceding embodiments, wherein the antibody agent has a K_{ON}, a K_{OFF} or a K_{D} provided in Table 4 as measured in a surface plasmon resonance assay.

Embodiment 126. The antibody agent of any one of the preceding embodiments, wherein the antibody agent competes for binding with a reference antibody agent that binds to Activin A.

Embodiment 127. The antibody agent of any one of the preceding embodiments, wherein the antibody agent has high specificity for Activin A and low polyreactivity, as measured with a poly-specificity reagent (PSR) or a comparable reagent that measures antibody binding specificity.

Embodiment 128. The antibody agent of embodiment 127, wherein the antibody agent has a clean PSR score of less than 0.1, or has a low PSR score of between 0.1 to 0.33.

Embodiment 129. The antibody agent of any one of the preceding embodiments, wherein the antibody agent has low hydrophobicity as measured in a HIC assay or a comparable assay that measures hydrophobicity.

Embodiment 130. The antibody agent of embodiment 129, wherein antibody agent has a HIC retention time of less than 10.5 minutes indicating a clean to low HIC, or a retention time of between 10.5 to 11.5 indicating a medium HIC.

Embodiment 131. The antibody agent of any one of the preceding embodiments, wherein the antibody agent has low self-association as measured with an AC-SINS assay or a comparable assay that measures self-association.

Embodiment 132. The antibody agent of embodiment 131, wherein the antibody agent has an AC-SINS score between 5 and 20 indicating low self-association.

Embodiment 133. The antibody agent of any one of the preceding embodiments, wherein the antibody agent has a melting temperature (Tm) of about 69.5° C. to about 82° C.

Embodiment 134. The antibody agent of any one of the preceding embodiments, wherein the antibody agent is produced in a bacterial cell, a yeast cell or a mammalian cell.

Embodiment 135. The antibody agent of any one of the preceding embodiments, wherein the antibody agent is produced in a mammalian cell.

Embodiment 136. The antibody agent of embodiment 135, wherein the mammalian cell is a CHO cell.

Embodiment 137. The antibody agent of any one of the preceding embodiments, wherein the antibody agent can be produced at a concentration of about 1000 to 20,000 mg/L, about 2000 to 20,000 mg/L, about 5000 to 20,000 mg/L, about 6000 to 20,000 mg/L, about 7000 to 20,000 mg/L, about 8000 to 20,000 mg/L, about 9000 to 20,000 mg/L, 10,000 to 20,000 mg/L or about 15,000 to 20,000 mg/L.

Embodiment 138. An isolated nucleic acid encoding an antibody agent of any one of embodiments 1-137.

Embodiment 139. An isolated nucleic acid comprising an Activin A variable heavy (VH) sequence chosen from a VH DNA sequence provided in Table 2 or a sequence with at least 85%, 86%, 87%, 88%, 89%, 90%, 91%, 92%, 93%, 94%, 95%, 96%, 97%, 98%, or 99% thereto.

Embodiment 140. The isolated nucleic acid of embodiment 139, wherein the VH sequence comprises the nucleic acid sequence of SEQ ID NO: 112, SEQ ID NO: 119, SEQ ID NO: 124, SEQ ID NO: 128, SEQ ID NO: 133, SEQ ID NO: 139, SEQ ID NO: 144, SEQ ID NO: 148, SEQ ID NO: 156, SEQ ID NO: 162, SEQ ID NO: 168, SEQ ID NO: 178, SEQ ID NO: 185, SEQ ID NO: 189, SEQ ID NO: 195, SEQ ID NO: 201; SEQ ID NO: 208, SEQ ID NO: 213 or a nucleic acid sequence with at least 85%, 86%, 87%, 88%, 89%, 90%, 91%, 92%, 93%, 94%, 95%, 96%, 97%, 98%, or 99% thereto.

Embodiment 141. An isolated nucleic acid comprising an Activin A variable light (VL) sequence chosen from a VL DNA sequence provided in Table 1, or a sequence with at least 85%, 86%, 87%, 88%, 89%, 90%, 91%, 92%, 93%, 94%, 95%, 96%, 97%, 98%, or 99% thereto.

Embodiment 142. The isolated nucleic acid of embodiment 141, wherein the VL sequence comprises the nucleic acid sequence of SEQ ID NO: 10, SEQ ID NO: 15, SEQ ID NO: 20, or SEQ ID NO: 24, SEQ ID NO: 35, SEQ ID NO: 39, SEQ ID NO: 47, SEQ ID NO: 52, SEQ ID NO: 60, SEQ ID NO: 67, SEQ ID NO: 76, SEQ ID NO: 84, SEQ ID NO: 92, SEQ ID NO: 101, or a sequence with at least 85%, 86%, 87%, 88%, 89%, 90%, 91%, 92%, 93%, 94%, 95%, 96%, 97%, 98%, or 99% thereto.

Embodiment 143. An isolated nucleic acid comprising an Activin A antibody agent sequence, wherein the nucleic acid comprises:
(a) a variable heavy (VH) sequence chosen from: SEQ ID NO: 112, SEQ ID NO: 119, SEQ ID NO: 124, SEQ ID NO: 128, SEQ ID NO: 133, SEQ ID NO: 139, SEQ ID NO: 144, SEQ ID NO: 148, SEQ ID NO: 156, SEQ ID NO: 162, SEQ ID NO: 168, SEQ ID NO: 178, SEQ ID NO: 185, SEQ ID NO: 189, SEQ ID NO: 195, SEQ ID NO: 201; SEQ ID NO: 208, SEQ ID NO: 213, or a sequence with at least 85%, 86%, 87%, 88%, 89%, 90%, 91%, 92%, 93%, 94%, 95%, 96%, 97%, 98%, or 99% thereto; and
(b) a variable light (VL) sequence chosen from: SEQ ID NO: 10, SEQ ID NO: 15, SEQ ID NO: 20, or SEQ ID NO: 24, SEQ ID NO: 35, SEQ ID NO: 39, SEQ ID NO: 47, SEQ ID NO: 52, SEQ ID NO: 60, SEQ ID NO: 67, SEQ ID NO: 76, SEQ ID NO: 84, SEQ ID NO: 92, SEQ ID NO: 101, or a sequence with at least 85%, 86%, 87%, 88%, 89%, 90%, 91%, 92%, 93%, 94%, 95%, 96%, 97%, 98%, or 99% thereto.

Embodiment 144. The isolated nucleic acid of embodiment 143, wherein the Activin A antibody agent further comprises a light chain constant region (CL) and/or at least one heavy chain constant region.

Embodiment 145. A vector comprising the nucleic acid of any one of embodiments 138-144.

Embodiment 146. A host cell comprising the vector of embodiment 145.

Embodiment 147. The host cell of embodiment 146, wherein said host cell is a yeast cell, a bacterial cell, a mammalian cell or an insect cell.

Embodiment 148. The host cell of embodiment 147, wherein the mammalian cell is chosen from a CHO cell, a COS cell, a HEK-293 cell, an NSO cell, a PER.C6 cell, or an Sp2.0 cell.

Embodiment 149. A method of making an antibody agent which binds to Activin A, comprising culturing the host cell of any one of embodiments 146-148 under a condition wherein the Activin A antibody agent is expressed by said host cell.

Embodiment 150. A composition comprising an Activin A antibody agent polypeptide of any one of embodiments 1-137.

Embodiment 151. A pharmaceutical composition comprising an Activin A antibody agent polypeptide of embodiment 150.

Embodiment 152. The pharmaceutical composition of embodiment 151, comprising an excipient and/or a pharmaceutically acceptable carrier.

Embodiment 153. The pharmaceutical composition of embodiment 151 or 152, formulated in one or more unit dosage forms.

Embodiment 154. A method comprising contacting a pharmaceutical composition of any one of embodiments 151-153 to a cell, tissue or subject.

Embodiment 155. The method of embodiment 154, wherein contacting comprises administering the Activin A pharmaceutical composition to the cell, tissue or subject.

Embodiment 156. The method of embodiment 154 or 155, wherein the method is a treatment method or a prevention method.

Embodiment 157. The method of any one of embodiments 154-156, wherein the subject has a condition or disorder associated with increased Activin A.

Embodiment 158. The method of embodiment 157, wherein Activin A comprises free and/or active Activin A.

Embodiment 159. The method of embodiment 158, wherein Activin A is or comprises free Activin A.

Embodiment 160. The method of embodiment 158, wherein Activin A is or comprises active Activin A.

Embodiment 161. The method of embodiment 158, wherein Activin A is or comprises free and active Activin A.

Embodiment 162. The method of any one of embodiments 157-161, wherein increased Activin A comprises a level of about 500 pg/ml or more, e.g., as evaluated in a sample from a subject.

Embodiment 163. The method of embodiment 162, wherein the sample is or comprises blood, plasma, serum, urine, or a combination thereof.

Embodiment 164. The method of any one of embodiments 157-163, wherein the condition or disorder is chosen from (i) disorders of the hypothalamic pituitary gonadal axis (e.g., with increased FSH levels); (ii) fibrodysplasia ossificans progressive (FOP); (iii) pulmonary arterial hypertension and/or hypertension; (iv) Anorexia-Cachexia Disorders associated with chronic diseases such as cancer, chronic kidney disease, chronic heart failure, COPD, elderly and muscle immobility, ICU patient (e.g., loss of body weight, loss of food intake, fat mass loss, muscle wasting, loss of functional muscle, loss of muscle strength, and bone loss, anemia and/or fatigue); (v) anemia; (vi) metabolic disorders (e.g., obesity, type 2 diabetes, and/or metabolic syndrome); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis); (viii) auto-immune disorders (e.g., SLE or rheumatoid arthritis); (ix) fibrotic diseases (e.g. idiopathic pulmonary fibrosis, NASH, liver cirrhosis, chronic kidney disease, Alport Syndrome, Uterine Fibroids, Endometriosis) (x) cancer (xi) cancer-treatment related toxicities (e.g., chemotherapy-induced nephrotoxicity); (xii) aging (e.g., senescence) or (xiii) chemoresistance.

Embodiment 165. The method of any one of embodiments 154-164, wherein the method ameliorates a symptom of a disorder in a subject.

Embodiment 166. The method of embodiment 165, wherein the symptom is weight loss, loss of appetite, fatigue, muscle mass loss, fat mass loss, lean mass loss, lean mass atrophy, bone loss, anemia, senescence, hypertension, chemoresiostance and/or fibrosis.

Embodiment 167. A method of inhibiting Activin A, comprising:
contacting a cell, tissue or subject with an Activin A pharmaceutical composition of any one of embodiments 151-153 to a cell, tissue or subject,
thereby inhibiting Activin A in the cell, tissue or subject.

Embodiment 168. The method of embodiment 167, wherein inhibition of Activin A comprises:
(a) a reduction in activity, level, and/or stability of Activin A; and/or
(b) preventing an increase in level of Activin A.

Embodiment 169. The method of embodiment 168, wherein Activin A comprises free and/or active Activin A.

Embodiment 170. The method of embodiment 169, wherein Activin A is or comprises free Activin A.

Embodiment 171. The method of embodiment 169, wherein Activin A is or comprises active Activin A.

Embodiment 172. The method of embodiment 169, wherein Activin A is or comprises free and active Activin A.

Embodiment 173. The method of any one of embodiments 167-172, wherein contacting comprises administering the Activin A pharmaceutical composition to the cell, tissue or subject.

Embodiment 174. The method of any one of embodiments 167-173, wherein reducing the level of Activin A comprises reducing it to less than 500 pg/mL.

Embodiment 175. The method of any one of embodiments 168-174, wherein the level of Activin A is reduced in a blood, plasma, serum, and/or urine sample.

Embodiment 176. The method of embodiment 168 or 169, wherein preventing an increase in the level of Activin A comprises preventing an increase in Activin A level above about 500 pg/mL.

Embodiment 177. The method of any one of embodiments 168-176, wherein inhibition of Activin A is assessed relative to a comparator.

Embodiment 178. The method of embodiment 177, wherein the comparator comprises an otherwise similar cell, tissue or subject not administered an Activin A pharmaceutical composition or administered an Activin A inhibitor or administered a different Activin A antibody agent.

Embodiment 179. The method of any one of embodiments 167-178, wherein Activin A is inhibited, e.g., the level of Activin A is reduced, by at least 5%, 10%, 20%, 30%, 40%, 50%, 60%, 70%, 80%, 90%, 95% or 99%.

Embodiment 180. The method of any one of embodiments 167-179, wherein the activity of Activin A comprises one or more, or all, or any combination of the following: (a) homodimerization; (b) binding to an Activin receptor; (c) modulation of an Activin receptor mediated signaling pathway.

Embodiment 181. The method of embodiment 180, wherein the Activin receptor signaling pathway comprises one or more of: (i) a SMAD pathway, (ii) an ERK/MAPK pathway, (iii) a p38 MAPK pathway, (iv) NOX-2 dependent signaling; or (v) Snail and/or Slug mediated pathways.

Embodiment 182. The method of embodiment 180, wherein administration of an Activin A antibody agent inhibits one or more Activin A activities provided in (a)-(c).

Embodiment 183. A method of preventing and/or reversing mass loss in a subject, comprising administering to the subject an Activin A pharmaceutical composition of any one of embodiments 151-153.

Embodiment 184. The method of embodiment 183, wherein mass comprises fat mass, lean mass, muscle mass, bone mass, organ mass loss, or a combination thereof.

Embodiment 185. The method of embodiment 184, wherein the Activin A pharmaceutical composition prevents and/or reverses mass loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 186. The method of embodiment 184 or 185, wherein the mass loss is or comprises fat mass loss.

Embodiment 187. The method of embodiment 184 or 185, wherein the mass loss is or comprises lean mass loss.

Embodiment 188. The method of embodiment 184 or 185, wherein the mass loss is or comprises muscle mass loss.

Embodiment 189. The method of embodiment 184 or 185, wherein the mass loss is or comprises loss of functional muscle mass.

Embodiment 190. The method of embodiment 184 or 185, wherein the mass loss is or comprises loss of muscle strength.

Embodiment 191. The method of any one of embodiments 188-190, wherein the muscle is chosen from: cardiac muscle, smooth muscle, skeletal muscle, or a combination thereof.

Embodiment 192. The method of embodiment 191, wherein the subject having muscle mass loss has a disease or disorder characterized by decreased muscle mass and/or strength.

Embodiment 193. The method of embodiment 192, wherein the disease or disorder characterized by decreased muscle mass and/or strength is chosen from: sarcopenia, cachexia, muscle injury, muscle wasting/atrophy, muscle immobility, bed-ridden patients, ICU patients, cancer, obesity, diabetes, arthritis, multiple sclerosis, muscular dystrophy, amyotrophic lateral sclerosis, Parkinson's disease, osteoporosis, osteoarthritis, osteopenia, a metabolic syndrome, chronic renal failure, renal fibrosis, chronic heart failure, or chronic obstructive pulmonary disease.

Embodiment 194. The method of embodiment 193, wherein the metabolic syndrome comprises a disease or disorder chosen from: diabetes, obesity, nutritional disorders, organ atrophy, chronic obstructive pulmonary disease, or anorexia.

Embodiment 195. The method of embodiment 194, wherein the mass loss is or comprises bone mass loss.

Embodiment 196. A method of preventing and/or reversing weight loss in a subject, comprising administering to the subject an Activin A pharmaceutical composition of any one of embodiments 151-153.

Embodiment 197. The method of embodiment 196, wherein the weight loss is involuntary, e.g., induced by cachexia.

Embodiment 198. The method of embodiment 196 or 197, wherein the Activin A pharmaceutical composition prevents and/or reverses weight loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 199. A method of preventing and/or reversing atrophy, e.g., muscle atrophy, in a subject, comprising administering to the subject an Activin A pharmaceutical composition of any one of embodiments 151-153.

Embodiment 200. The method of embodiment 199, wherein the muscle is chosen from: cardiac muscle, smooth muscle, skeletal muscle, or a combination thereof.

Embodiment 201. The method of embodiment 199 or 200, wherein the Activin A pharmaceutical composition prevents and/or reverses muscle atrophy by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 202. A method of preventing and/or reversing senescence in a subject, comprising administering to the subject an Activin A pharmaceutical composition of any one of embodiments 151-153.

Embodiment 203. The method of embodiment 202, wherein the Activin A pharmaceutical composition prevents and/or reverses senescence by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 204. The method of any one of embodiments 183-203, wherein the mass loss, weight loss, muscle atrophy, and/or senescence is a symptom of a disease or disorder.

Embodiment 205. The method of embodiment 204, wherein mass loss comprises loss of functional muscle loss, and/or loss of muscle strength.

Embodiment 206. The method of any one of embodiments 183-204, wherein the mass loss, weight loss, muscle atrophy, and/or senescence is not a symptom of a disease or disorder.

Embodiment 207. A method of preventing and/or reversing liver damage in a subject, comprising administering to the subject an Activin A pharmaceutical composition of any one of embodiments 151-153.

Embodiment 208. The method of embodiment 207, wherein liver damage comprises: acute liver injury, liver necrosis, liver fibrosis, liver degeneration, liver cirrhosis (e.g., alcoholic cirrhosis or non-alcoholic cirrhosis), liver inflammation, alteration in liver function, elevation one or more liver enzymes, or a combination thereof.

Embodiment 209. The method of embodiment 207 or 208, wherein the one or more liver enzymes comprise ALT, AST or both.

Embodiment 210. The method of any one of embodiments 207-209, wherein liver damage is a symptom of liver disease.

Embodiment 211. The method of embodiment 210, wherein liver disease is or comprises NAFLD, NASH, cirrhosis or cancer.

Embodiment 212. The method of any one of embodiments 207-210, wherein the Activin A pharmaceutical composition prevents and/or reverses liver damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 213. A method of preventing and/or reversing kidney damage in a subject, comprising administering to the subject an Activin A pharmaceutical composition of any one of embodiments 151-153.

Embodiment 214. The method of embodiment 213, wherein kidney damage comprises: acute kidney injury, kidney fibrosis, kidney inflammation, alteration in kidney function, or a combination thereof.

Embodiment 215. The method of embodiment 213 or 214, wherein alteration in kidney function is or comprises proteinuria.

Embodiment 216. The method of embodiment 213 or 214, wherein the kidney damage is or comprises tubular degeneration.

Embodiment 217. The method of any one of embodiments 213-216, wherein the kidney damage is a symptom of kidney disease.

Embodiment 218. The method of any one of embodiments 213-217, wherein kidney disease is or comprises: acute kidney injury, chemotherapy-induced nephropathy, cancer-treatment-induced nephropathy, chronic kidney disease, diabetic nephropathy, Alport Syndrome, chronic kidney disease mineral bone disorder, tubulointerstitial nephritis, Focal Segmental Glomerulosclerosis, IgA nephropathy, Autosomal dominant polycystic kidney disease.

Embodiment 219. The method of any one of embodiments 213-218, wherein the Activin A pharmaceutical composition prevents and/or reverses kidney damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 220. The method of any one of embodiments 184-219, wherein the mass loss, weight loss, muscle atrophy, senescence, liver damage, and/or kidney damage is induced by a therapy for a disease or disorder (e.g., a SOC for a disease or disorder).

Embodiment 221. The method of any one of embodiments 184-220, wherein mass loss, weight loss, muscle atrophy, senescence, liver damage, and/or kidney damage is reduced relative to a comparator.

Embodiment 222. The method of embodiment 221, wherein the comparator comprises an otherwise similar cell, tissue or subject not administered an Activin A pharmaceutical composition or administered an Activin A inhibitor a different Activin A antibody agent.

Embodiment 223. A method of preventing and/or treating a SARS-COV-2 infection comprising administering to the subject an Activin A pharmaceutical composition of any one of embodiments 151-153.

Embodiment 224. The method of embodiment 223, wherein the SARS-COV-2 infection is or comprises a COVID-19 disease.

Embodiment 225. The method of embodiment 223 or 224, wherein the SARS-COV-2 infection comprises acute respiratory distress syndrome (ARDS).

Embodiment 226. A method of preventing and/or treating cancer in a subject, comprising administering to the subject an Activin A pharmaceutical composition of any one of embodiments 151-153.

Embodiment 227. The method of any one of embodiments 154-226, wherein the subject has an increased level and/or activity of Activin A.

Embodiment 228. The method of any one of embodiments 154-227, further comprising determining a level and/or activity of Activin A in a sample from the subject.

Embodiment 229. The method of embodiment 228, wherein an increased level and/or activity of Activin A is determined relative to a comparator.

Embodiment 230. The method of embodiment 229, wherein a comparator comprises a predetermined reference sample such as a sample obtained from an otherwise similar subject who does not have a disease or disorder, or a symptom of a disease or disorder.

Embodiment 231. The method of any one of embodiments 154-230, wherein the Activin A pharmaceutical composition is characterized in that when administered to the subject it reduces the level and/or activity of Activin A relative to a comparator.

Embodiment 232. The method of embodiment 231, wherein the level Activin A is reduced in a blood, plasma, serum or urine sample.

Embodiment 233. The method of embodiment 232, wherein Activin A is or comprises free and/or active Activin A.

Embodiment 234. The method of embodiment 233, wherein Activin A is or comprises free Activin A.

Embodiment 235. The method of embodiment 233, wherein Activin A is or comprises active Activin A.

Embodiment 236. The method of embodiment 233, wherein Activin A is or comprises free and active Activin A.

Embodiment 237. The method of any one of embodiments 231-236, wherein the Activin A pharmaceutical reduces the level of Activin A to less than 500 pg/mL.

Embodiment 238. A method of preventing and/or reducing migration of cells in vivo, comprising: administering a pharmaceutical composition of any one of embodiments 151-153 to a subject.

Embodiment 239. The method of embodiment 238, wherein the subject has a condition or disorder associated with increased Activin A.

Embodiment 240. The method of embodiment 239, wherein the condition or disorder is a hyperproliferative disorder, e.g., a cancer.

Embodiment 241. The method of any one of embodiments 238-240, wherein the method prevents and/or reduces migration of cells from a primary cancer to one or more secondary sites.

Embodiment 242. The method of embodiment 241, wherein migration of cells to one or more secondary sites is or comprises metastasis.

Embodiment 243. The method of any one of embodiments 238-242, wherein the method prevents and/or reduces migration of cells by at least 5%, 10%, 20%, 30%, 40%, 50%, 60%, 70%, 80%, 90%, 95% or 99%.

Embodiment 244. The method of any one of embodiments 238-243, wherein prevention and/or reduction cell migration is assessed relative to a comparator.

Embodiment 245. The method of embodiment 244, wherein the comparator comprises an otherwise similar subject not administered an Activin A pharmaceutical composition or administered an Activin A inhibitor or administered a different Activin A antibody agent.

Embodiment 246. The method of any one of embodiments 239-245, wherein increased Activin A comprises a level of about 500 pg/ml or more, e.g., as evaluated in a sample from a subject.

Embodiment 247. The method of embodiment 246, wherein the sample is or comprises blood, plasma, serum, urine, or a combination thereof.

Embodiment 248. The method of any one of embodiments 238-247, wherein Activin A is or comprises free and/or active Activin A.

Embodiment 249. A method of reducing and/or preventing metastasis, the method comprising the step of administering to a subject with a tumor a pharmaceutical composition of any one of embodiments 151-153.

Embodiment 250. The method of embodiment 240 or 249, wherein the cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

Embodiment 251. The method of embodiment 250, wherein the cancer is colorectal cancer.

Embodiment 252. The method of embodiment 250 or 251, wherein the method reduces metastasis by at least 5%, 10%, 20%, 30%, 40%, 50%, 60%, 70%, 80%, 90%, 95% or 99%.

Embodiment 253. The method of any one of embodiments 249-252, wherein reduction of metastasis is assessed relative to a comparator.

Embodiment 254. The method of embodiment 253, wherein the comparator comprises an otherwise similar subject not administered an Activin A pharmaceutical composition or administered an Activin A inhibitor or administered a different Activin A antibody agent.

Embodiment 255. The method of any one of embodiments 249-254, wherein the subject having cancer has increased Activin A levels.

Embodiment 256. The method of any one of embodiments 249-254, wherein increased Activin A comprises a level of about 500 pg/ml or more, e.g., as evaluated in a sample from the subject.

Embodiment 257. The method of embodiment 256, wherein the sample is or comprises blood, plasma, serum, urine, or a combination thereof.

Embodiment 258. The method of any one of embodiments 238-257, wherein Activin A is or comprises free and/or active Activin A.

Embodiment 259. A method of treating and/or preventing cachexia, the method comprising the step of administering to a subject with a tumor a pharmaceutical composition of any one of embodiments 151-153.

Embodiment 260. The method of embodiment 259, wherein cachexia comprises loss of appetite, muscle loss, fat mass loss, weight loss, or a combination thereof.

Embodiment 261. The method of embodiment 259 or 260, wherein the cachexia is induced by cancer.

Embodiment 262. A method of treating and/or preventing chemotherapy induced toxicity, the method comprising the step of administering to a subject with a tumor a pharmaceutical composition of any one of embodiments 151-153.

Embodiment 263. The method of embodiment 262, wherein the toxicity is or comprises nephrotoxicity and/or hepatotoxicity.

Embodiment 264. The method of embodiment 262 or 263, wherein the chemotherapy is a Cisplatin based chemotherapy.

Embodiment 265. The method of embodiment 262 or 263, wherein the chemotherapy is not a Cisplatin based chemotherapy.

Embodiment 266. The method of any one of embodiments 259-265, wherein the cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

Embodiment 267. The method of any one of embodiments 259-265, wherein the cancer is colorectal cancer.

Embodiment 268. The method of any one of embodiments 259-267, wherein the subject having cancer has increased Activin A levels.

Embodiment 269. The method of any one of embodiments 259-268, wherein increased Activin A comprises a level of about 500 pg/ml or more, e.g., as evaluated in a sample from the subject.

Embodiment 270. The method of embodiment 269, wherein the sample is or comprises blood, plasma, serum, urine, or a combination thereof.

Embodiment 271. The method of any one of embodiments 238-270, wherein Activin A is or comprises free and/or active Activin A.

Embodiment 272. A method, comprising
assessing a level and/or activity of Activin A in a sample from a subject,
administering an Activin A pharmaceutical composition to the subject if the level of Activin A is higher than a comparator.

Embodiment 273. The method of embodiment 272, wherein an increased level of Activin A comprises at least 500 pg/mL or more as assessed in a sample from a subject.

Embodiment 274. The method of embodiment 272, wherein an increased level and/or activity of Activin A is determined relative to a comparator.

Embodiment 275. The method of embodiment 274, wherein a comparator comprises a predetermined reference sample such as a sample obtained from an otherwise similar subject who does not have a disease or disorder, or a symptom of a disease or disorder.

Embodiment 276. The method of any one of embodiments 272-276, wherein the Activin A pharmaceutical composition is characterized in that when administered to the subject it reduces the level and/or activity of Activin A relative to a comparator.

Embodiment 277. The method of embodiment 276, wherein the comparator comprises an otherwise similar cell, tissue or subject not administered an Activin A pharmaceutical composition or administered an Activin A inhibitor or a different Activin A antibody agent.

Embodiment 278. The method of any one of embodiments 154-277, wherein the subject is a mammal.

Embodiment 279. The method of embodiment 278, wherein the mammal is a human.

Embodiment 280. The method of embodiment 279, wherein the human is an adult.

Embodiment 281. The method of embodiment 279, wherein the human is a child.

Embodiment 282. The method of embodiment 278, wherein the mammal is a domestic animal.

Embodiment 283. The method of embodiment 282, wherein the domestic animal is chosen from: a cat, a dog, a ferret, a horse, a cow, a sheep, a pig, a Bactrian camel, or a yak.

Embodiment 284. The method of any one of embodiments 226-258, wherein the cancer has a TP53 mutation and/or a SMAD4 mutation.

Embodiment 285. A method of promoting liver regeneration in a subject, comprising administering to the subject an Activin A pharmaceutical composition as described herein.

Embodiment 286. A method of preventing reoccurrence of metastases in a liver in a subject having cancer, comprising administering to the subject an Activin A pharmaceutical composition as described herein.

Embodiment 287. The method of embodiment 285 or 286, wherein the subject is receiving, has received or will be receiving a partial hepatectomy.

Embodiment 288. The method of any one of embodiments 285-287, wherein the subject: has liver damage (e.g., as described herein), has cancer (e.g., as described herein), is an aging subject, or any combination thereof.

Embodiment 289. A method of reducing and/or preventing chemoresistance in a subject having cancer, comprising administering to the subject an Activin A pharmaceutical composition as described herein.

Embodiment 290. The method of embodiment 289, wherein the chemoresistance is induced by a therapy administered to the subject to treat the cancer.

1. An antibody agent comprising a polypeptide that binds to Activin A comprising a light chain complementarity determining region 1 (LC CDR1), an LC CDR2, and an LC CDR3 provided in Table 1, and a heavy chain complementary determining region 1 (HC CDR1), an HC CDR2, and an HC CDR3 provided in Table 2.

2. The antibody agent of claim 1, wherein the antibody agent is or comprises:
(i) an intact IgA, IgG, IgD, IgE or IgM antibody;
(ii) an antibody fragment;
(iii) a single chain Fv; or
(iv) a polypeptide comprising an antigen binding domain fused to a Fc domain.

3-10. (canceled)

11. The antibody agent of claim 1, wherein the antibody agent comprises:
(i) an LC CDR1 of SEQ ID NO: 1, or a sequence with at least 85% identity thereto, or a sequence having at least 5, 10, or 20 substitutions relative to SEQ ID NO: 1; an LC CDR2 of SEQ ID NO: 2, or a sequence with at least 85% identity thereto or a sequence having at least 5, 10, or 20 substitutions relative to SEQ ID NO: 2; and an LC CDR3 of SEQ ID NO: 3, or a sequence with at least 85% identity thereto or a sequence having at least 5, 10, or 20 substitutions relative to SEQ ID NO: 3; and a HC CDR1 of SEQ ID NO: 103, or a sequence with at least 85% identity thereto or a sequence having at least 5, 10, or 20 substitutions relative to SEQ ID NO: 103; an HC CDR2 of SEQ ID NO: 104, or a sequence with at least 85% identity thereto or a sequence having at least 5, 10, or 20 substitutions relative to SEQ ID NO: 104; and an HC CDR3 of SEQ ID NO: 105, or a sequence with at least 85% identity thereto or a sequence having at least 5, 10, or 20 substitutions relative to SEQ ID NO: 105;
(ii) an LC CDR1 of SEQ ID NO: 11, or a sequence with at least 85% identity thereto or a sequence having at least 5, 10, or 20 substitutions relative to SEQ ID NO: 11; an LC CDR2 of SEQ ID NO: 2, or a sequence with at least 85% identity thereto or a sequence having at least 5, 10, or 20 substitutions relative to SEQ ID NO: 2; and an LC CDR3 of SEQ ID NO: 12, or a sequence with at least 85% identity thereto or a sequence having at least 5, 10, or 20 substitutions relative to SEQ ID NO: 12; and an HC CDR1 of SEQ ID NO: 113, or a sequence with at least 85% identity thereto or a sequence having at least 5, 10, or 20 substitutions relative to SEQ ID NO: 113; an HC CDR2 of SEQ ID NO: 114, or a sequence with at least 85% identity thereto or a sequence having at least 5, 10, or 20 substitutions relative to SEQ ID NO: 114; and an HC CDR3 of SEQ ID NO: 115, or a sequence with at least 85% identity thereto or a sequence having at least 5, 10, or 20 substitutions relative to SEQ ID NO: 115;
(iii) an LC CDR1 of SEQ ID NO: 11, or a sequence with at least 85% identity thereto or a sequence having at least 5, 10, or 20 substitutions relative to SEQ ID NO: 11; an LC CDR2 of SEQ ID NO: 16, or a sequence with at least 85% identity thereto or a sequence having at least 5, 10, or 20 substitutions relative to SEQ ID NO: 16; and an LC CDR3 of SEQ ID NO: 17, or a sequence with at least 85% identity thereto or a sequence having at least 5, 10, or 20 substitutions relative to SEQ ID NO: 17; and an HC CDR1 of SEQ ID NO: 120, or a sequence with at least 85% identity thereto or a sequence having at least 5, 10, or 20 substitutions relative to SEQ ID NO: 120; an HC CDR2 of SEQ ID NO: 121, or a sequence with at least 85% identity thereto or a sequence having at least 5, 10, or 20 substitutions relative to SEQ ID NO: 121; and an HC CDR3 of SEQ ID NO: 115, or a sequence with at least 85% identity thereto or a sequence having at least 5, 10, or 20 substitutions relative to SEQ ID NO: 115;
(iv) an LC CDR1 of SEQ ID NO: 11, or a sequence with at least 85% identity thereto or a sequence having at least 5, 10, or 20 substitutions relative to SEQ ID NO: 11; an LC CDR2 of SEQ ID NO: 2, or a sequence with at least 85% identity thereto or a sequence having at least 5, 10, or 20 substitutions relative to SEQ ID NO: 2; and an LC CDR3 of SEQ ID NO: 21, or a sequence with at least 85% identity thereto or a sequence having at least 5, 10, or 20 substitutions relative to SEQ ID NO: 21; and an HC CDR1 of SEQ ID NO: 125, or a sequence with at least 85% identity thereto or a sequence having at least 5, 10, or 20 substitutions relative to SEQ ID NO: 125; an HC CDR2 of SEQ ID NO: 121, or a sequence with at least 85% identity thereto or a sequence having at least 5, 10, or 20 substitutions relative to SEQ ID NO: 121; and an HC CDR3 of SEQ ID NO: 115, or a sequence with at least 85% identity thereto or a sequence having at least 5, 10, or 20 substitutions relative to SEQ ID NO: 115;
(v) an LC CDR1 of SEQ ID NO: 1, or a sequence with at least 85% identity thereto or a sequence having at least 5, 10, or 20 substitutions relative to SEQ ID NO: 1; an LC CDR2 of SEQ ID NO: 2, or a sequence with at least 85% identity thereto or a sequence having at least 5, 10, or 20 substitutions relative to SEQ ID NO: 2; and an LC CDR3 of SEQ ID NO: 3, or a sequence with at least 85% identity thereto or a sequence having at least 5, 10, or 20 substitutions relative to SEQ ID NO: 3; and an HC CDR1 of SEQ ID NO: 129, or a sequence with at least 85% identity thereto or a sequence having at least 5, 10, or 20 substitutions relative to SEQ ID NO: 129; an HC CDR2 of SEQ ID NO: 130, or a sequence with at least 85% identity thereto or a sequence having at least 5, 10, or 20 substitutions relative to SEQ ID NO: 130; and an HC CDR3 of SEQ ID NO: 105, or a sequence with at least 85% identity thereto or a sequence having at least 5, 10, or 20 substitutions relative to SEQ ID NO: 105;
(vi) an LC CDR1 of SEQ ID NO: 28, or a sequence with at least 85% identity thereto or a sequence having at least 5, 10, or 20 substitutions relative to SEQ ID NO: 28; an LC CDR2 of SEQ ID NO: 29 or a sequence with at least 85% identity thereto or a sequence having at least 5, 10, or 20 substitutions relative to SEQ ID NO: 29; and an LC CDR3 of SEQ ID NO: 30, or a sequence with at least 85% identity thereto or a sequence having at least 5, 10, or 20 substitutions relative to SEQ ID NO: 30; and an HC CDR1 of SEQ ID NO: 134, or a sequence with at least 85% identity thereto or a sequence having at least 5, 10, or 20 substitutions relative to SEQ ID NO: 134; an HC CDR2 of SEQ ID NO: 135, or a sequence with at least 85% identity thereto or a sequence having at least 5, 10, or 20 substitutions relative to SEQ ID NO: 135; and an HC CDR3 of SEQ ID NO: 115, or a sequence with at least 85% identity thereto or a sequence having at least 5, 10, or 20 substitutions relative to SEQ ID NO: 115;
(vii) an LC CDR1 of SEQ ID NO: 1, or a sequence with at least 85% identity thereto or a sequence having at least 5, 10, or 20 substitutions relative to SEQ ID NO: 1; an LC CDR2 of SEQ ID NO: 2 or a sequence with at least 85% identity thereto or a sequence having at least 5, 10, or 20 substitutions relative to SEQ ID NO: 2; and an LC CDR3 of SEQ ID NO: 36, or a sequence with at least 85% identity thereto or a sequence having at least 5, 10, or 20 substitutions relative to SEQ ID NO: 36; and an HC CDR1 of SEQ ID NO: 140, or a sequence with at least 85% identity thereto or a sequence having at least 5, 10, or 20 substitutions relative to SEQ ID NO: 140; an HC CDR2 of SEQ ID NO: 141, or a sequence with at least 85% identity thereto or a sequence having at least 5, 10, or 20 substitutions relative to SEQ ID NO: 141; and an HC CDR3 of SEQ ID NO: 105, or a sequence with at least 85% identity thereto or a sequence having at least 5, 10, or 20 substitutions relative to SEQ ID NO: 105;
(viii) an LC CDR1 of SEQ ID NO: 1, or a sequence with at least 85% identity thereto or a sequence having at least 5, 10, or 20 substitutions relative to SEQ ID NO: 1; an LC CDR2 of SEQ ID NO: 2 or a sequence with at least 85% identity thereto or a sequence having at least 5, 10, or 20 substitutions relative to SEQ ID NO: 2; and an LC CDR3 of SEQ ID NO: 36, or a sequence with at least 85% identity thereto or a sequence having at least 5, 10, or 20 substitutions relative to SEQ ID NO: 36; and an HC CDR1 of SEQ ID NO: 134, or a sequence with at least 85% identity thereto or a sequence having at least 5, 10, or 20 substitutions relative to SEQ ID NO: 134; an HC CDR2 of SEQ ID NO: 145, or a sequence with at least 85% identity thereto or a sequence having at least 5, 10, or 20 substitutions relative to SEQ ID NO: 145; and an HC CDR3 of SEQ ID NO: 105, or a sequence with at least 85% identity thereto or a sequence having at least 5, 10, or 20 substitutions relative to SEQ ID NO: 105;
(ix) an LC CDR1 of SEQ ID NO: 11, or a sequence with at least 85% identity thereto or a sequence having at least 5, 10, or 20 substitutions relative to SEQ ID NO: 11; an LC CDR2 of SEQ ID NO: 43 or a sequence with at least 85% identity thereto or a sequence having at least 5, 10, or 20 substitutions relative to SEQ ID NO: 43; and an LC CDR3 of SEQ ID NO: 44, or a sequence with at least 85% identity thereto or a sequence having at least 5, 10, or 20 substitutions relative to SEQ ID NO: 44; and an HC CDR1 of SEQ ID NO: 149, or a sequence with at least 85% identity thereto or a sequence having at least 5, 10, or 20 substitutions relative to SEQ ID NO: 149; an HC CDR2 of SEQ ID NO: 150, or a sequence with at least 85% identity thereto or a sequence having at least 5, 10, or 20 substitutions relative to SEQ ID NO: 150; and an HC CDR3 of SEQ ID NO: 151, or a sequence with at least 85% identity thereto or a sequence having at least 5, 10, or 20 substitutions relative to SEQ ID NO: 151;
(x) an LC CDR1 of SEQ ID NO: 11, or a sequence with at least 85% identity thereto or a sequence having at least 5, 10, or 20 substitutions relative to SEQ ID NO: 11; an LC CDR2 of SEQ ID NO: 48 or a sequence with at least 85% identity thereto or a sequence having at least 5, 10, or 20 substitutions relative to SEQ ID NO: 48; and an LC CDR3 of SEQ ID NO: 49, or a sequence with at least 85% identity thereto or a sequence having at least 5, 10, or 20 substitutions relative to SEQ ID NO: 49; and an HC CDR1 of SEQ ID NO: 157, or a sequence with at least 85% identity thereto or a sequence having at least 5, 10, or 20 substitutions relative to SEQ ID NO: 157; an HC CDR2 of SEQ ID NO: 158, or a sequence with at least 85% identity thereto or a sequence having at least 5, 10, or 20 substitutions relative to SEQ ID NO: 158; and an HC CDR3 of SEQ ID NO: 151, or a sequence with at least 85% identity thereto or a sequence having at least 5, 10, or 20 substitutions relative to SEQ ID NO: 151;
(xi) an LC CDR1 of SEQ ID NO: 53, or a sequence with at least 85% identity thereto or a sequence having at least 5, 10, or 20 substitutions relative to SEQ ID NO: 53; an LC CDR2 of SEQ ID NO: 54 or a sequence with at least 85% identity thereto or a sequence having at least 5, 10, or 20 substitutions relative to SEQ ID NO: 54; and an LC CDR3 of SEQ ID NO: 55, or a sequence with at least 85% identity thereto or a sequence having at least 5, 10, or 20 substitutions relative to SEQ ID NO: 55; and an HC CDR1 of SEQ ID NO: 163, or a sequence with at least 85% identity thereto or a sequence having at least 5, 10, or 20 substitutions relative to SEQ ID NO: 163; an HC CDR2 of SEQ ID NO: 164, or a sequence with at least 85% identity thereto or a sequence having at least 5, 10, or 20 substitutions relative to SEQ ID NO: 164; and an HC CDR3 of SEQ ID NO: 165, or a sequence with at least 85% identity thereto or a sequence having at least 5, 10, or 20 substitutions relative to SEQ ID NO: 165;
(xii) an LC CDR1 of SEQ ID NO: 28, or a sequence with at least 85% identity thereto or a sequence having at least 5, 10, or 20 substitutions relative to SEQ ID NO: 28; an LC CDR2 of SEQ ID NO: 61 or a sequence with at least 85% identity thereto or a sequence having at least 5, 10, or 20 substitutions relative to SEQ ID NO: 61; and an LC CDR3 of SEQ ID NO: 62, or a sequence with at least 85% identity thereto or a sequence having at least 5, 10, or 20 substitutions relative to SEQ ID NO: 62; and an HC CDR1 of SEQ ID NO: 169, or a sequence with at least 85% identity thereto or a sequence having at least 5, 10, or 20 substitutions relative to SEQ ID NO: 169; an HC CDR2 of SEQ ID NO: 170, or a sequence with at least 85% identity thereto or a sequence having at least 5, 10, or 20 substitutions relative to SEQ ID NO: 170; and an HC CDR3 of SEQ ID NO: 171, or a sequence with at least 85% identity thereto or a sequence having at least 5, 10, or 20 substitutions relative to SEQ ID NO: 171;
(xiii) an LC CDR1 of SEQ ID NO: 68, or a sequence with at least 85% identity thereto or a sequence having at least 5, 10, or 20 substitutions relative to SEQ ID NO: 68; an LC CDR2 of SEQ ID NO: 69 or a sequence with at least 85% identity thereto or a sequence having at least 5, 10, or 20 substitutions relative to SEQ ID NO: 69; and an LC CDR3 of SEQ ID NO: 70, or a sequence with at least 85% identity thereto or a sequence having at least 5, 10, or 20 substitutions relative to SEQ ID NO: 70; and an HC CDR1 of SEQ ID NO: 179, or a sequence with at least 85% identity thereto or a sequence having at least 5, 10, or 20 substitutions relative to SEQ ID NO: 179; an HC CDR2 of SEQ ID NO: 180, or a sequence with at least 85% identity thereto or a sequence having at least 5, 10, or 20 substitutions relative to SEQ ID NO: 180; and an HC CDR3 of SEQ ID NO: 181, or a sequence with at least 85% identity thereto or a sequence having at least 5, 10, or 20 substitutions relative to SEQ ID NO: 181;
(xiv) an LC CDR1 of SEQ ID NO: 68, or a sequence with at least 85% identity thereto or a sequence having at least 5, 10, or 20 substitutions relative to SEQ ID NO: 68; an LC CDR2 of SEQ ID NO: 69 or a sequence with at least 85% identity thereto or a sequence having at least 5, 10, or 20 substitutions relative to SEQ ID NO: 69; and an LC CDR3 of SEQ ID NO: 70, or a sequence with at least 85% identity thereto or a sequence having at least 5, 10, or 20 substitutions relative to SEQ ID NO: 70; and an HC CDR1 of SEQ ID NO: 179, or a sequence with at least 85% identity thereto or a sequence having at least 5, 10, or 20 substitutions relative to SEQ ID NO: 179; an HC CDR2 of SEQ ID NO: 186, or a sequence with at least 85% identity thereto or a sequence having at least 5, 10, or 20 substitutions relative to SEQ ID NO: 186; and an HC CDR3 of SEQ ID NO: 181, or a sequence with at least 85% identity thereto or a sequence having at least 5, 10, or 20 substitutions relative to SEQ ID NO: 181;
(xv) an LC CDR1 of SEQ ID NO: 28, or a sequence with at least 85% identity thereto or a sequence having at least 5, 10, or 20 substitutions relative to SEQ ID NO: 28; an LC CDR2 of SEQ ID NO: 61 or a sequence with at least 85% identity thereto or a sequence having at least 5, 10, or 20 substitutions relative to SEQ ID NO: 61; and an LC CDR3 of SEQ ID NO: 80, or a sequence with at least 85% identity thereto or a sequence having at least 5, 10, or 20 substitutions relative to SEQ ID NO: 80; and an HC CDR1 of SEQ ID NO: 190, or a sequence with at least 85% identity thereto or a sequence having at least 5, 10, or 20 substitutions relative to SEQ ID NO: 190; an HC CDR2 of SEQ ID NO: 191, or a sequence with at least 85% identity thereto or a sequence having at least 5, 10, or 20 substitutions relative to SEQ ID NO: 191; and an HC CDR3 of SEQ ID NO: 192, or a sequence with at least 85% identity thereto or a sequence having at least 5, 10, or 20 substitutions relative to SEQ ID NO: 192;
(xvi) an LC CDR1 of SEQ ID NO:85, or a sequence with at least 85% identity thereto or a sequence having at least 5, 10, or 20 substitutions relative to SEQ ID NO: 85; an LC CDR2 of SEQ ID NO: 86 or a sequence with at least 85% identity thereto or a sequence having at least 5, 10, or 20 substitutions relative to SEQ ID NO: 86; and an LC CDR3 of SEQ ID NO: 87, or a sequence with at least 85% identity thereto or a sequence having at least 5, 10, or 20 substitutions relative to SEQ ID NO: 87; and an HC CDR1 of SEQ ID NO: 196, or a sequence with at least 85% identity thereto or a sequence having at least 5, 10, or 20 substitutions relative to SEQ ID NO: 196; an HC CDR2 of SEQ ID NO: 197, or a sequence with at least 85% identity thereto or a sequence having at least 5, 10, or 20 substitutions relative to SEQ ID NO: 197; and an HC CDR3 of SEQ ID NO: 198, or a sequence with at least 85% identity thereto or a sequence having at least 5, 10, or 20 substitutions relative to SEQ ID NO: 198;
(xvii) an LC CDR1 of SEQ ID NO: 93, or a sequence with at least 85% identity thereto or a sequence having at least 5, 10, or 20 substitutions relative to SEQ ID NO: 93; an LC CDR2 of SEQ ID NO: 94 or a sequence with at least 85% identity thereto or a sequence having at least 5, 10, or 20 substitutions relative to SEQ ID NO: 94; and an LC CDR3 of SEQ ID NO: 95, or a sequence with at least 85% identity thereto or a sequence having at least 5, 10, or 20 substitutions relative to SEQ ID NO: 95; and an HC CDR1 of SEQ ID NO: 202, or a sequence with at least 85% identity thereto or a sequence having at least 5, 10, or 20 substitutions relative to SEQ ID NO:202; an HC CDR2 of SEQ ID NO: 203, or a sequence with at least 85% identity thereto or a sequence having at least 5, 10, or 20 substitutions relative to SEQ ID NO: 203; and an HC CDR3 of SEQ ID NO: 204, or a sequence with at least 85% identity thereto or a sequence having at least 5, 10, or 20 substitutions relative to SEQ ID NO: 204; or
(xviii) an LC CDR1 of SEQ ID NO: 93, or a sequence with at least 85% identity thereto or a sequence having at least 5, 10, or 20 substitutions relative to SEQ ID NO: 93; an LC CDR2 of SEQ ID NO: 94 or a sequence with at least 85% identity thereto or a sequence having at least 5, 10, or 20 substitutions relative to SEQ ID NO: 94; and an LC CDR3 of SEQ ID NO: 95, or a sequence with at least 85% identity thereto or a sequence having at least 5, 10, or 20 substitutions relative to SEQ ID NO: 95; and an HC CDR1 of SEQ ID NO: 209, or a sequence with at least 85% identity thereto or a sequence having at least 5, 10, or 20 substitutions relative to SEQ ID NO:209; an HC CDR2 of SEQ ID NO: 210, or a sequence with at least 85% identity thereto or a sequence having at least 5, 10, or 20 substitutions relative to SEQ ID NO: 210; and an HC CDR3 of SEQ ID NO: 204, or a sequence with at least 85% identity thereto or a sequence having at least 5, 10, or 20 substitutions relative to SEQ ID NO: 204.

12-29. (canceled)

30. The antibody agent of claim 1, wherein the antibody agent comprises:
(a) a light chain and the light chain comprises a variable region comprising an LC CDR1, an LC CDR2 and an LC CDR3 provided in Table 1 and a sequence for a constant region (CL),
(b) a heavy chain and the heavy chain comprises a variable region comprising an HC CDR1, an HC CDR2 and an HC CDR3 provided in Table 2 and a sequence for at least one constant region (CH), wherein the at least one constant region comprises an Fc domain, or
(c) both (a) and (b).

31-35. (canceled)

36. The antibody agent of claim 1, wherein the antibody agent comprises:
(i) the sequence of SEQ ID NO: 8, or a sequence with at least 85% identity thereto, or a sequence having at least 5, 10, or 20 substitutions relative to SEQ ID NO: 8; and the sequence of SEQ ID NO: 110, or a sequence with at least 85% identity thereto, or a sequence having at least 5, 10, or 20 substitutions relative to SEQ ID NO: 110;
(ii) the sequence of SEQ ID NO: 13, or a sequence with at least 85% identity thereto, or a sequence having at least 5, 10, or 20 substitutions relative to SEQ ID NO: 13; and the sequence of SEQ ID NO: 117, or a sequence with at least 85% identity thereto, or a sequence having at least 5, 10, or 20 substitutions relative to SEQ ID NO: 117;
(iii) the sequence of SEQ ID NO: 18, or a sequence with at least 85% identity thereto, or a sequence having at least 5, 10, or 20 substitutions relative to SEQ ID NO: 18; and the sequence of SEQ ID NO: 122, or a sequence with at least 85% identity thereto, or a sequence having at least 5, 10, or 20 substitutions relative to SEQ ID NO: 122;
(iv) the sequence of SEQ ID NO: 22, or a sequence with at least 85% identity thereto, or a sequence having at least 5, 10, or 20 substitutions relative to SEQ ID NO: 22; and the sequence of SEQ ID NO: 126, or a sequence with at least 85% identity thereto, or a sequence having at least 5, 10, or 20 substitutions relative to SEQ ID NO: 126;
(v) the sequence of SEQ ID NO: 8, or a sequence with at least 85% identity thereto, or a sequence having at least 5, 10, or 20 substitutions relative to SEQ ID NO: 8; and the sequence of SEQ ID NO: 131, or a sequence with at least 85% identity thereto, or a sequence having at least 5, 10, or 20 substitutions relative to SEQ ID NO: 131;
(vi) the sequence of SEQ ID NO: 33, or a sequence with at least 85% identity thereto, or a sequence having at least 5, 10, or 20 substitutions relative to SEQ ID NO: 33; and the sequence of SEQ ID NO: 137, or a sequence with at least 85% identity thereto, or a sequence having at least 5, 10, or 20 substitutions relative to SEQ ID NO:137;
(vii) the sequence of SEQ ID NO: 37, or a sequence with at least 85% identity thereto, or a sequence having at least 5, 10, or 20 substitutions relative to SEQ ID NO: 37; and the sequence of SEQ ID NO: 142, or a sequence with at least 85% identity thereto, or a sequence having at least 5, 10, or 20 substitutions relative to SEQ ID NO: 142;
(viii) the sequence of SEQ ID NO: 37, or a sequence with at least 85% identity thereto, or a sequence having at least 5, 10, or 20 substitutions relative to SEQ ID NO: 37; and the sequence of SEQ ID NO: 146, or a sequence with at least 85% identity thereto, or a sequence having at least 5, 10, or 20 substitutions relative to SEQ ID NO: 146;
(ix) the sequence of SEQ ID NO: 45, or a sequence with at least 85% identity thereto, or a sequence having at least 5, 10, or 20 substitutions relative to SEQ ID NO: 45; and the sequence of SEQ ID NO: 154, or a sequence with at least 85% identity thereto, or a sequence having at least 5, 10, or 20 substitutions relative to SEQ ID NO: 154;
(x) the sequence of SEQ ID NO: 50, or a sequence with at least 85% identity thereto, or a sequence having at least 5, 10, or 20 substitutions relative to SEQ ID NO: 50; and the sequence of SEQ ID NO: 160, or a sequence with at least 85% identity thereto, or a sequence having at least 5, 10, or 20 substitutions relative to SEQ ID NO: 160;
(xi) the sequence of SEQ ID NO: 58, or a sequence with at least 85% identity thereto, or a sequence having at least 5, 10, or 20 substitutions relative to SEQ ID NO: 58; and the sequence of SEQ ID NO: 166, or a sequence with at least 85% identity thereto, or a sequence having at least 5, 10, or 20 substitutions relative to SEQ ID NO: 166;
(xii) the sequence of SEQ ID NO: 65, or a sequence with at least 85% identity thereto, or a sequence having at least 5, 10, or 20 substitutions relative to SEQ ID NO: 65; and the sequence of SEQ ID NO: 176, or a sequence with at least 85% identity thereto, or a sequence having at least 5, 10, or 20 substitutions relative to SEQ ID NO: 176;
(xiii) the sequence of SEQ ID NO: 74, or a sequence with at least 85% identity thereto, or a sequence having at least 5, 10, or 20 substitutions relative to SEQ ID NO: 74; and the sequence of SEQ ID NO: 183, or a sequence with at least 85% identity thereto, or a sequence having at least 5, 10, or 20 substitutions relative to SEQ ID NO: 183;
(xiv) the sequence of SEQ ID NO: 74, or a sequence with at least 85% identity thereto, or a sequence having at least 5, 10, or 20 substitutions relative to SEQ ID NO: 74; and the sequence of SEQ ID NO: 187, or a sequence with at least 85% identity thereto, or a sequence having at least 5, 10, or 20 substitutions relative to SEQ ID NO: 187;
(xv) the sequence of SEQ ID NO: 82, or a sequence with at least 85% identity thereto, or a sequence having at least 5, 10, or 20 substitutions relative to SEQ ID NO: 82; and the sequence of SEQ ID NO: 193, or a sequence with at least 85% identity thereto, or a sequence having at least 5, 10, or 20 substitutions relative to SEQ ID NO: 193;
(xvi) the sequence of SEQ ID NO: 90, or a sequence with at least 85% identity thereto, or a sequence having at least 5, 10, or 20 substitutions relative to SEQ ID NO: 90; and the sequence of SEQ ID NO: 199, or a sequence with at least 85% identity thereto, or a sequence having at least 5, 10, or 20 substitutions relative to SEQ ID NO: 199;
(xvii) the sequence of SEQ ID NO: 99, or a sequence with at least 85% identity thereto, or a sequence having at least 5, 10, or 20 substitutions relative to SEQ ID NO: 99; and the sequence of SEQ ID NO: 206, or a sequence with at least 85% identity thereto, or a sequence having at least 5, 10, or 20 substitutions relative to SEQ ID NO: 206; or
(xviii) the sequence of SEQ ID NO: 99, or a sequence with at least 85% identity thereto, or a sequence having at least 5, 10, or 20 substitutions relative to SEQ ID NO: 99; and the sequence of SEQ ID NO: 211, or a sequence with at least 85% identity thereto, or a sequence having at least 5, 10, or 20 substitutions relative to SEQ ID NO: 211.

37. (canceled)

38. The antibody agent of claim 1, wherein the antibody agent comprises:
(i) the sequence of SEQ ID NO: 9, or a sequence with at least 85% identity thereto, or a sequence having at least 5, 10, or 20 substitutions relative to SEQ ID NO: 9; and the sequence of SEQ ID NO: 111, or a sequence with at least 85% identity thereto, or a sequence having at least 5, 10, or 20 substitutions relative to SEQ ID NO: 111;
(ii) the sequence of SEQ ID NO: 14, or a sequence with at least 85% identity thereto, or a sequence having at least 5, 10, or 20 substitutions relative to SEQ ID NO: 14; and the sequence of SEQ ID NO: 118, or a sequence with at least 85% identity thereto, or a sequence having at least 5, 10, or 20 substitutions relative to SEQ ID NO: 118;
(iii) the sequence of SEQ ID NO: 19, or a sequence with at least 85% identity thereto, or a sequence having at least 5, 10, or 20 substitutions relative to SEQ ID NO: 19; and the sequence of SEQ ID NO: 123, or a sequence with at least 85% identity thereto, or a sequence having at least 5, 10, or 20 substitutions relative to SEQ ID NO: 123;
(iv) the sequence of SEQ ID NO: 23, or a sequence with at least 85% identity thereto, or a sequence having at least 5, 10, or 20 substitutions relative to SEQ ID NO: 23; and the sequence of SEQ ID NO: 127, or a sequence with at least 85% identity thereto, or a sequence having at least 5, 10, or 20 substitutions relative to SEQ ID NO: 127;
(v) the sequence of SEQ ID NO: 9, or a sequence with at least 85% identity thereto, or a sequence having at least 5, 10, or 20 substitutions relative to SEQ ID NO: 9; and the sequence of SEQ ID NO: 132, or a sequence with at least 85% identity thereto, or a sequence having at least 5, 10, or 20 substitutions relative to SEQ ID NO: 132;
(vi) the sequence of SEQ ID NO: 34, or a sequence with at least 85% identity thereto, or a sequence having at least 5, 10, or 20 substitutions relative to SEQ ID NO: 34; and the sequence of SEQ ID NO: 138, or a sequence with at least 85% identity thereto, or a sequence having at least 5, 10, or 20 substitutions relative to SEQ ID NO: 138;
(vii) the sequence of SEQ ID NO: 38, or a sequence with at least 85% identity thereto, or a sequence having at least 5, 10, or 20 substitutions relative to SEQ ID NO: 38; and the sequence of SEQ ID NO: 143, or a sequence with at least 85% identity thereto, or a sequence having at least 5, 10, or 20 substitutions relative to SEQ ID NO: 143;
(viii) the sequence of SEQ ID NO: 38, or a sequence with at least 85% identity thereto, or a sequence having at least 5, 10, or 20 substitutions relative to SEQ ID NO: 38; and the sequence of SEQ ID NO: 147, or a sequence with at least 85% identity thereto, or a sequence having at least 5, 10, or 20 substitutions relative to SEQ ID NO: 147;
(ix) the sequence of SEQ ID NO: 46, or a sequence with at least 85% identity thereto, or a sequence having at least 5, 10, or 20 substitutions relative to SEQ ID NO: 46; and the sequence of SEQ ID NO: 155, or a sequence with at least 85% identity thereto, or a sequence having at least 5, 10, or 20 substitutions relative to SEQ ID NO: 155;
(x) the sequence of SEQ ID NO: 51, or a sequence with at least 85% identity thereto, or a sequence having at least 5, 10, or 20 substitutions relative to SEQ ID NO: 51; and the sequence of SEQ ID NO: 161, or a sequence with at least 85% identity thereto, or a sequence having at least 5, 10, or 20 substitutions relative to SEQ ID NO: 161;
(xi) the sequence of SEQ ID NO: 59, or a sequence with at least 85% identity thereto, or a sequence having at least 5, 10, or 20 substitutions relative to SEQ ID NO: 59; and the sequence of SEQ ID NO: 167, or a sequence with at least 85% identity thereto, or a sequence having at least 5, 10, or 20 substitutions relative to SEQ ID NO: 167;
(xii) the sequence of SEQ ID NO: 66, or a sequence with at least 85% identity thereto, or a sequence having at least 5, 10, or 20 substitutions relative to SEQ ID NO: 66; and the sequence of SEQ ID NO: 177, or a sequence with at least 85% identity thereto, or a sequence having at least 5, 10, or 20 substitutions relative to SEQ ID NO: 177;
(xiii) the sequence of SEQ ID NO: 75, or a sequence with at least 85% identity thereto, or a sequence having at least 5, 10, or 20 substitutions relative to SEQ ID NO: 75; and the sequence of SEQ ID NO: 184, or a sequence with at least 85% identity thereto, or a sequence having at least 5, 10, or 20 substitutions relative to SEQ ID NO: 184;
(xiv) the sequence of SEQ ID NO: 75, or a sequence with at least 85% identity thereto, or a sequence having at least 5, 10, or 20 substitutions relative to SEQ ID NO: 75; and the sequence of SEQ ID NO: 188, or a sequence with at least 85% identity thereto, or a sequence having at least 5, 10, or 20 substitutions relative to SEQ ID NO: 188;
(xv) the sequence of SEQ ID NO: 83, or a sequence with at least 85% identity thereto, or a sequence having at least 5, 10, or 20 substitutions relative to SEQ ID NO: 83; and the sequence of SEQ ID NO: 194, or a sequence with at least 85% identity thereto, or a sequence having at least 5, 10, or 20 substitutions relative to SEQ ID NO: 194;
(xvi) the sequence of SEQ ID NO: 91, or a sequence with at least 85% identity thereto, or a sequence having at least 5, 10, or 20 substitutions relative to SEQ ID NO: 91; and the sequence of SEQ ID NO: 200, or a sequence with at least 85% identity thereto, or a sequence having at least 5, 10, or 20 substitutions relative to SEQ ID NO: 200;
(xvii) the sequence of SEQ ID NO: 100, or a sequence with at least 85% identity thereto, or a sequence having at least 5, 10, or 20 substitutions relative to SEQ ID NO: 100; and the sequence of SEQ ID NO: 207, or a sequence with at least 85% identity thereto, or a sequence having at least 5, 10, or 20 substitutions relative to SEQ ID NO: 207; or
(xviii) the sequence of SEQ ID NO: 100, or a sequence with at least 85% identity thereto, or a sequence having at least 5, 10, or 20 substitutions relative to SEQ ID NO: 100; and the sequence of SEQ ID NO: 212, or a sequence with at least 85% identity thereto, or a sequence having at least 5, 10, or 20 substitutions relative to SEQ ID NO: 212.

39. The antibody agent of claim 1, wherein the antibody agent binds to human Activin A with a binding affinity (K_{D}) of about 6.17×10(−12)M to about 960×10(−12)M.

40-41. (canceled)

42. The antibody agent of claim 1, wherein:
(i) the antibody agent reduces a plasma, blood, serum, or urine level of Activin A, wherein the antibody agent reduces the level of Activin A to less than about 500 pg/mL;
(ii) the antibody agent prevents an increase in Activin A levels in plasma, blood, serum, or urine, wherein the antibody agent prevents an increase in Activin A level above about 500 pg/mL; or
(iii) antibody agent reduces, an Activin A activity, by about 5% or more.

43. The antibody agent of claim 1, characterized in that when tested in an assay that evaluates activation of an Activin receptor, the antibody agent:
(i) does not activate one or more signaling pathways activated by the Activin receptor; or
(ii) reduces Activin A signaling via the Activin receptor; and
wherein the antibody agent does not bind to or has minimal binding affinity for one or more TGFbeta super family members other than Activin A.

44. (canceled)

45. The antibody agent of claim 1, wherein the antibody agent is produced in a bacterial cell, a yeast cell or a mammalian cell.

46-47. (canceled)

48. An isolated nucleic acid comprising an Activin A antibody agent sequence, wherein the nucleic acid comprises:
(a) a variable heavy (VH) sequence chosen from: SEQ ID NO: 112, SEQ ID NO: 119, SEQ ID NO: 124, SEQ ID NO: 128, SEQ ID NO: 133, SEQ ID NO: 139, SEQ ID NO: 144, SEQ ID NO: 148, SEQ ID NO: 156, SEQ ID NO: 162, SEQ ID NO: 168, SEQ ID NO: 178, SEQ ID NO: 185, SEQ ID NO: 189, SEQ ID NO: 195, SEQ ID NO: 201; SEQ ID NO: 208, SEQ ID NO: 213, or a sequence with at least 85% identity thereto; and
(b) a variable light (VL) sequence chosen from: SEQ ID NO: 10, SEQ ID NO: 15, SEQ ID NO: 20, or SEQ ID NO: 24, SEQ ID NO: 35, SEQ ID NO: 39, SEQ ID NO: 47, SEQ ID NO: 52, SEQ ID NO: 60, SEQ ID NO: 67, SEQ ID NO: 76, SEQ ID NO: 84, SEQ ID NO: 92, SEQ ID NO: 101, or a sequence with at least 85% identity thereto.

49. A vector comprising the nucleic acid of claim 48.

50. A host cell comprising the vector of claim 49.

51. A method of making an antibody agent which binds to Activin A, comprising culturing the host cell of claim 50 under a condition wherein the Activin A antibody agent is expressed by said host cell.

52. A composition comprising an Activin A antibody agent polypeptide of claim 1.

53. A pharmaceutical composition comprising an Activin A antibody agent polypeptide of claim 1, and an excipient.

54. (canceled)

55. A method comprising
delivering a pharmaceutical composition of claim 53 to a cell, tissue or subject.

56. The method of claim 55, wherein contacting comprises administering the Activin A pharmaceutical composition to the cell, tissue or subject.

57. The method of claim 55, wherein the method is a treatment method, and wherein the subject has a condition or disorder associated with increased Activin A.

58-65. (canceled)

66. A method of preventing and/or reversing:
(i) mass loss in a subject;
(ii) weight loss in a subject;
(iii) senescence in a subject;
(iv) liver damage in a subject;
(v) a combination of or all of (i)-(iv),
wherein the method comprises administering to the subject an pharmaceutical composition of claim 53.

67-69. (canceled)

70. A method of:
(i) promoting liver regeneration in a subject, or
(ii) preventing reoccurrence of metastases in a liver in a subject having cancer, the method comprising administering to the subject the pharmaceutical composition of claim 53, wherein
(a) the subject is receiving, has received or will be receiving a partial hepatectomy, or
(b) the subject: has liver damage, has cancer, is an aging subject, or any combination thereof.

71-73. (canceled)

74. A method of:
(i) reducing and/or preventing chemoresistance in a subject having cancer;
(ii) preventing and/or reversing kidney damage in a subject;
(iii) preventing and/or treating a SARS-COV-2 infection;
(iv) preventing and/or treating cachexia in a subject;
(v) preventing and/or treating cancer in a subject; or
(vi) preventing and/or treating metastasis in a subject,
wherein the method comprises administering to the subject the pharmaceutical composition of claim 53.

75-83. (canceled)

84. A method, comprising
assessing a level or activity of Activin A in a sample from a subject,
administering an Activin A pharmaceutical composition to the subject if the level of Activin A is higher than a comparator.

85-86. (canceled)
